(19) United States
(12) Patent Application Publication
(10) Pub. No.: US 2026/0108528 A1
(43) Pub. Date: Apr. 23, 2026

(54) METHODS OF TREATING A RAS PROTEIN-RELATED DISEASE OR DISORDER

(71) Applicant: Revolution Medicines, Inc., Redwood City, CA (US)

(72) Inventors: Jingjing JIANG, Redwood City, CA (US); Lingyan JIANG, San Mateo, CA (US); Benjamin MALDONATO, Los Gatos, CA (US); Mallika SINGH, San Francisco, CA (US); Sophia SOHONI, Santa Clara, CA (US); Zhengping WANG, San Mateo, CA (US); Zhican WANG, San Mateo, CA (US); Caroline E. WELLER, Redwood City, CA (US); Muhammad ALI, Mountain View, CA (US)

(21) Appl. No.: 19/363,949

(22) Filed: Oct. 21, 2025

Related U.S. Application Data
(60) Provisional application No. 63/710,296, filed on Oct. 22, 2024.
(60) Provisional application No. 63/794,157, filed on Apr. 24, 2025.

(51) Int. Cl. A61K 31/5377; A61K 45/06; A61P 35/00; C07K 16/22; C07K 16/28

(52) U.S. Cl. CPC A61K 31/5377; A61K 45/06; A61P 35/00; C07K 16/22; C07K 16/2818; C07K 16/2863

(57) ABSTRACT

The disclosure features methods of treating RAS protein-related disease using Compound A, or a pharmaceutically acceptable salt thereof. The disclosure also features methods of treating RAS protein-related disease including combinations including Compound A, or a pharmaceutically acceptable salt thereof, and additional therapeutic agents.

### FIELD

The present disclosure relates to the treatment of diseases or disorders associated with RAS proteins, particularly those resulting from a RAS G12D mutation. More specifically, the present disclosure provides uses for a RAS(ON) G12D-selective, covalent tri-complex inhibitor, such as Compound A (zoldonrasib, RMC-9805, or RM-036) in treating RAS protein-related disease or disorder, such as a RAS G12D mutant cancer, in subjects in need thereof.

### BACKGROUND

The vast majority of small molecule drugs act by binding a functionally important pocket on a target protein, thereby modulating the activity of that protein. For example, cholesterol lowering drugs known as statins bind the enzyme active site of HMG-CoA reductase, thus preventing the enzyme from engaging with its substrates. The fact that many such drug/target interacting pairs are known may have misled some into believing that a small molecule modulator could be discovered for most, if not all, proteins provided a reasonable amount of time, effort, and resources. This is far from the case. Current estimates are that only about 10% of all human proteins are targetable by small molecules. The other 90% are currently considered refractory or intractable toward the abovementioned small molecule drug discovery. Such targets are commonly referred to as “undruggable.” These undruggable targets include a vast and largely untapped reservoir of medically important human proteins. Thus, there exists a great deal of interest in discovering new molecular modalities capable of modulating the function of such undruggable targets.

It has been well established in literature that RAS proteins (KRAS, HRAS, and NRAS) play an essential role in various human cancers and are therefore appropriate targets for anticancer therapy. Indeed, mutations in RAS proteins account for approximately 30% of all human cancers in the United States, many of which are fatal. Dysregulation of RAS proteins by activating mutations, overexpression or upstream activation is common in human tumors, and activating mutations in RAS are frequently found in human cancer and RASopathies. For example, activating mutations at codon 12 in RAS proteins function by inhibiting both GTPase-activating protein (GAP)-dependent and intrinsic hydrolysis rates of GTP, significantly skewing the population of RAS mutant proteins to the “on” (GTP-bound) state (RAS(ON)), leading to oncogenic MAPK signaling. Notably, RAS exhibits a picomolar affinity for GTP, enabling RAS to be activated even in the presence of low concentrations of this nucleotide. Mutations at codons 13 (e.g., G13C) and 61 (e.g., Q61K) of RAS are also responsible for oncogenic activity in some cancers.

In normal cells, RAS proteins play a critical role in regulating cell growth, differentiation, and survival, acting as molecular switches, relaying signals from cell surface receptors to intracellular pathways that control key cellular processes. Genetic studies have demonstrated that complete deletion of RAS genes is lethal in mouse models and leads to the absence of cellular proliferation in vitro (Drosten et al. Oncogene 33, 2857-2865 (2014); Drosten et al. EMBO J. 29, 1091-1104 (2010)). Furthermore, KRAS conditional knockout in adult bone marrow has been shown to induce significant hematopoietic defects, including splenomegaly, an expanded neutrophil compartment, and reduced B cell number (Zhang et. al., Stem Cells; 34(7):1859-71 (2016)). Targeting the mutant form of RAS, rather than wild-type RAS, has emerged as a strategy to treat RAS mutant cancer due to its specific involvement in oncogenic signaling. Despite extensive drug discovery efforts against RAS during the last several decades, only two agents targeting the KRAS G12C mutant have been approved in the U.S. (sotorasib and adagrasib). However, there are no approved targeted therapies for RAS G12D driven cancers.

Further efforts are needed to uncover additional therapies for RAS protein-related disease and disorders, e.g., cancers harboring a RAS G12D mutation.

### SUMMARY

Provided herein are methods of treating RAS protein-related disorders using Compound A, or a pharmaceutically acceptable salt thereof, which is a RAS inhibitor.

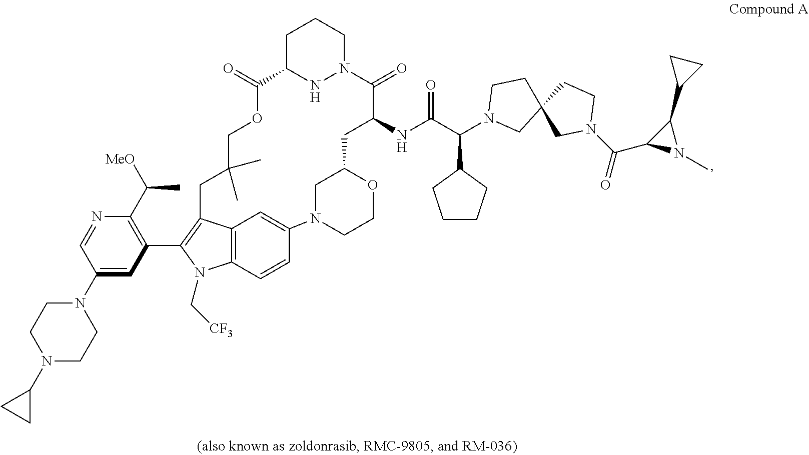

In an aspect, the disclosure features a method of treating a RAS protein-related disorder (e.g., cancer) in a human subject in need thereof. The method includes orally administering a total daily dose of 100 mg to 1400 mg (e.g., 100 mg to 1400 mg, 150 mg to 1400 mg, 200 mg to 1400 mg, 250 mg to 1400 mg, 300 mg to 1400 mg, 350 mg to 1400 mg, 400 mg to 1400 mg, 450 mg to 1400 mg, 500 mg to 1400 mg, 550 mg to 1400 mg, 600 mg to 1400 mg, 650 mg to 1400 mg, 700 mg to 1400 mg, 750 mg to 1400 mg, 800 mg to 1400 mg, 850 mg to 1400 mg, 900 mg to 1400 mg, 950 mg to 1400 mg, 1000 mg to 1400 mg, 1100 mg to 1400 mg, 1200 mg to 1400 mg, 1300 mg to 1400 mg, 100 mg to 1300 mg, 150 mg to 1300 mg, 200 mg to 1300 mg, 250 mg to 1300 mg, 300 mg to 1300 mg, 350 mg to 1300 mg, 400 mg to 1300 mg, 450 mg to 1300 mg, 500 mg to 1300 mg, 550 mg to 1300 mg, 600 mg to 1300 mg, 650 mg to 1300 mg, 700 mg to 1300 mg, 750 mg to 1300 mg, 800 mg to 1300 mg, 850 mg to 1300 mg, 900 mg to 1300 mg, 950 mg to 1300 mg, 1000 mg to 1300 mg, 1100 mg to 1300 mg, 1200 mg to 1300 mg, 100 mg to 1200 mg, 150 mg to 1200 mg, 200 mg to 1200 mg, 250 mg to 1200 mg, 300 mg to 1200 mg, 350 mg to 1200 mg, 400 mg to 1200 mg, 450 mg to 1200 mg, 500 mg to 1200 mg, 550 mg to 1200 mg, 600 mg to 1200 mg, 650 mg to 1200 mg, 700 mg to 1200 mg, 750 mg to 1200 mg, 800 mg to 1200 mg, 850 mg to 1200 mg, 900 mg to 1200 mg, 950 mg to 1200 mg, 1000 mg to 1200 mg, 1100 mg to 1200 mg, 100 mg to 1100 mg, 150 mg to 1100 mg, 200 mg to 1100 mg, 250 mg to 1100 mg, 300 mg to 1100 mg, 350 mg to 1100 mg, 400 mg to 1100 mg, 450 mg to 1100 mg, 500 mg to 1100 mg, 550 mg to 1100 mg, 600 mg to 1100 mg, 650 mg to 1100 mg, 700 mg to 1100 mg, 750 mg to 1100 mg, 800 mg to 1100 mg, 850 mg to 1100 mg, 900 mg to 1100 mg, 950 mg to 1100 mg, 1000 mg to 1100 mg, 100 mg to 1000 mg, 150 mg to 1000 mg, 200 mg to 1000 mg, 250 mg to 1000 mg, 300 mg to 1000 mg, 350 mg to 1000 mg, 400 mg to 1000 mg, 450 mg to 1000 mg, 500 mg to 1000 mg, 550 mg to 1000 mg, 600 mg to 1000 mg, 650 mg to 1000 mg, 700 mg to 1000 mg, 750 mg to 1000 mg, 800 mg to 1000 mg, 850 mg to 1000 mg, 900 mg to 1000 mg, 950 mg to 1000 mg, 100 mg to 950 mg, 150 mg to 950 mg, 200 mg to 950 mg, 250 mg to 950 mg, 300 mg to 950 mg, 350 mg to 950 mg, 400 mg to 950 mg, 450 mg to 950 mg, 500 mg to 950 mg, 550 mg to 950 mg, 600 mg to 950 mg, 650 mg to 950 mg, 700 mg to 950 mg, 750 mg to 950 mg, 800 mg to 950 mg, 850 mg to 950 mg, 900 mg to 950 mg, 100 mg to 900 mg, 150 mg to 900 mg, 200 mg to 900 mg, 250 mg to 900 mg, 300 mg to 900 mg, 350 mg to 900 mg, 400 mg to 900 mg, 450 mg to 900 mg, 500 mg to 900 mg, 550 mg to 900 mg, 600 mg to 900 mg, 650 mg to 900 mg, 700 mg to 900 mg, 750 mg to 900 mg, 800 mg to 900 mg, 850 mg to 900 mg, 100 mg to 850 mg, 150 mg to 850 mg, 200 mg to 850 mg, 250 mg to 850 mg, 300 mg to 850 mg, 350 mg to 850 mg, 400 mg to 850 mg, 450 mg to 850 mg, 500 mg to 850 mg, 550 mg to 850 mg, 600 mg to 850 mg, 650 mg to 850 mg, 700 mg to 850 mg, 750 mg to 850 mg, 800 mg to 850 mg, 100 mg to 800 mg, 150 mg to 800 mg, 200 mg to 800 mg, 250 mg to 800 mg, 300 mg to 800 mg, 350 mg to 800 mg, 400 mg to 800 mg, 450 mg to 800 mg, 500 mg to 800 mg, 550 mg to 800 mg, 600 mg to 800 mg, 650 mg to 800 mg, 700 mg to 800 mg, 750 mg to 800 mg, 100 mg to 750 mg, 150 mg to 750 mg, 200 mg to 750 mg, 250 mg to 750 mg, 300 mg to 750 mg, 350 mg to 750 mg, 400 mg to 750 mg, 450 mg to 750 mg, 500 mg to 750 mg, 550 mg to 750 mg, 600 mg to 750 mg, 650 mg to 750 mg, 700 mg to 750 mg, 100 mg to 700 mg, 150 mg to 700 mg, 200 mg to 700 mg, 250 mg to 700 mg, 300 mg to 700 mg, 350 mg to 700 mg, 400 mg to 700 mg, 450 mg to 700 mg, 500 mg to 700 mg, 550 mg to 700 mg, 600 mg to 700 mg, 650 mg to 700 mg, 100 mg to 650 mg, 150 mg to 650 mg, 200 mg to 650 mg, 250 mg to 650 mg, 300 mg to 650 mg, 350 mg to 650 mg, 400 mg to 650 mg, 450 mg to 650 mg, 500 mg to 650 mg, 550 mg to 650 mg, 600 mg to 650 mg, 100 mg to 600 mg, 150 mg to 600 mg, 200 mg to 600 mg, 250 mg to 600 mg, 300 mg to 600 mg, 350 mg to 600 mg, 400 mg to 600 mg, 450 mg to 600 mg, 500 mg to 600 mg, 550 mg to 600 mg, 100 mg to 550 mg, 150 mg to 550 mg, 200 mg to 550 mg, 250 mg to 550 mg, 300 mg to 550 mg, 350 mg to 550 mg, 400 mg to 550 mg, 450 mg to 550 mg, 500 mg to 550 mg, 100 mg to 500 mg, 150 mg to 500 mg, 200 mg to 500 mg, 250 mg to 500 mg, 300 mg to 500 mg, 350 mg to 500 mg, 400 mg to 500 mg, 450 mg to 500 mg, 100 mg to 450 mg, 150 mg to 450 mg, 200 mg to 450 mg, 250 mg to 450 mg, 300 mg to 450 mg, 350 mg to 450 mg, 400 mg to 450 mg, 100 mg to 400 mg, 150 mg to 400 mg, 200 mg to 400 mg, 250 mg to 400 mg, 300 mg to 400 mg, 350 mg to 400 mg, 100 mg to 350 mg, 150 mg to 350 mg, 200 mg to 350 mg, 250 mg to 350 mg, 300 mg to 350 mg, 100 mg to 300 mg, 150 mg to 300 mg, 200 mg to 300 mg, 250 mg to 300 mg, 100 mg to 250 mg, 150 mg to 250 mg, 200 mg to 250 mg, 100 mg to 200 mg, 150 mg to 200 mg, or 100 mg to 150 mg) Compound A to the subject in need thereof.

In some embodiments, the method includes administering a total daily dose of 100 mg to 1400 mg (e.g., 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, 510 mg, 520 mg, 530 mg, 540 mg, 550 mg, 560 mg, 570 mg, 580 mg, 590 mg, 600 mg, 610 mg, 620 mg, 630 mg, 640 mg, 650 mg, 660 mg, 670 mg, 680 mg, 690 mg, 700 mg, 710 mg, 720 mg, 730 mg, 740 mg, 750 mg, 760 mg, 770 mg, 780 mg, 790 mg, 800 mg, 810 mg, 820 mg, 830 mg, 840 mg, 850 mg, 860 mg, 870 mg, 880 mg, 890 mg, 900 mg, 910 mg, 920 mg, 930 mg, 940 mg, 950 mg, 960 mg, 970 mg, 980 mg, 990 mg, 1000 mg, 1010 mg, 1020 mg, 1030 mg, 1040 mg, 1050 mg, 1060 mg, 1070 mg, 1080 mg, 1090 mg, 1100 mg, 1110 mg, 1120 mg, 1130 mg, 1140 mg, 1150 mg, 1160 mg, 1170 mg, 1180 mg, 1190 mg, 1200 mg, 1250 mg, 1300 mg, 1350 mg, or 1400 mg) Compound A to the subject in need thereof.

In some embodiments, the method includes administering a total daily dose of 600 mg to 1200 mg, 625 mg to 1175 mg, 650 mg to 1150 mg, 675 mg to 1125 mg, 700 mg to 1100 mg, 725 mg to 1075 mg, 750 mg to 1050 mg, 775 mg to 1025 mg, 800 mg to 1000 mg, or 825 mg to 975 mg Compound A to the subject in need thereof.

In some embodiments, the method includes administering 150 mg of Compound A to the subject in need thereof. In some embodiments, the method includes administering 300 mg of Compound A to the subject in need thereof. In some embodiments, the method includes administering 600 mg of Compound A to the subject in need thereof. In some embodiments, the method includes administering 900 mg of Compound A to the subject in need thereof. In some embodiments, the method includes administering 1200 mg of Compound A to the subject in need thereof.

In some embodiments, Compound A is administered to the subject daily. In some embodiments, Compound A is administered to the subject once, twice, or more per day. In some embodiments, Compound A is administered to the subject once per day. In some embodiments, Compound A is administered to the subject twice per day.

In some embodiments, Compound A is administered to the subject twice per day. In some embodiments, the method includes administering 150 mg to 600 mg twice daily (e.g., 200 mg to 600 mg, 250 mg to 600 mg, 300 mg to 600 mg, 350 mg to 600 mg, 400 mg to 600 mg, 450 mg to 600 mg, 500 mg to 600 mg, 550 mg to 600 mg, 200 mg to 550 mg, 250 mg to 550 mg, 300 mg to 550 mg, 350 mg to 550 mg, 400 mg to 550 mg, 450 mg to 500 mg, 200 mg to 500 mg, 350 mg to 500 mg, 400 mg to 500 mg, 450 mg to 500 mg, 200 mg to 450 mg, 250 mg to 450 mg, 300 mg to 450 mg, 350 mg to 450 mg, 400 mg to 450 mg, 200 mg to 400 mg, 250 mg to 400 mg, 300 mg to 400 mg, 350 mg to 400 mg, 200 mg to 350 mg, 250 mg to 350 mg, 300 mg to 350 mg, 200 mg to 300 mg, 250 mg to 300 mg, or 200 mg to 250 mg) Compound A to the subject in need thereof. In some embodiments, the method includes administering 150 mg of Compound A to the subject twice per day. In some embodiments, the method includes administering 300 mg of Compound A to the subject twice per day. In some embodiments, the method includes administering 450 mg of Compound A to the subject twice per day. In some embodiments, the method includes administering 600 mg of Compound A to the subject twice per day.

In some embodiments, the RAS protein-related disorder is a cancer. In some embodiments, the cancer comprises a RAS mutation. In some embodiments, the RAS mutation is at position 12. In some embodiments, the RAS mutation is G12D. In some embodiments, the cancer is locally advanced or metastatic. In some embodiments, the cancer is pancreatic cancer. In some embodiments, the cancer is pancreatic ductal adenocarcinoma. In some embodiments, the cancer is pancreatic adenocarcinoma. In some embodiments, the cancer is lung cancer. In some embodiments, the cancer is non-small cell lung cancer. In some embodiments, the cancer is colorectal cancer. In some embodiments, the RAS protein is KRAS. In some embodiments, the method further comprises administering an additional anticancer therapy. In some embodiments, the additional anticancer therapy is an EGFR inhibitor, a second RAS inhibitor, a SHP2 inhibitor, a SOS1 inhibitor, a Raf inhibitor, a MEK inhibitor, an ERK inhibitor, a PI3K inhibitor, a PTEN inhibitor, an AKT inhibitor, an mTORC1 inhibitor, a BRAF inhibitor, a PD-L1 inhibitor, a PD-1 inhibitor, a CDK4/6 inhibitor, a HER2 inhibitor, or a combination thereof. In some embodiments, the additional anticancer therapy is a RAS^{MULTI} inhibitor or a pan-RAS inhibitor (e.g., a pan-KRAS inhibitor). In some embodiments, the additional anticancer therapy is a RAS^{MULTI}(ON) inhibitor. In some embodiments, the additional therapy is a KRAS^{G12D}(OFF) inhibitor. In some embodiments, the additional therapy is pembrolizumab or a biosimilar thereof. In some embodiments, the additional anticancer therapy is ivonescimab or a biosimilar thereof. In some embodiments, the additional anticancer therapy is cetuximab or a biosimilar thereof. In some embodiments, the additional anticancer therapy is a chemotherapeutic agent.

In another aspect, the invention features a method of avoiding an adverse drug interaction with Compound A in a human subject in need of Compound A, the method comprising administering to the subject a therapeutically effective amount of Compound A, and avoiding co-administration of a cytochrome p450 (CYP) 3A4 inhibitor, wherein said subject is also in need of the CYP3A4 inhibitor.

In another aspect, the invention features a method of avoiding an adverse drug interaction with Compound A in a human subject in need of Compound A, comprising discontinuing administration of a CYP3A4 inhibitor to avoid an adverse drug interaction with Compound A, and administering to the subject a therapeutically effective amount of Compound A. In another aspect, the invention features a method of avoiding an adverse drug interaction with Compound A in a human subject in need of Compound A, comprising discontinuing administration of a CYP3A4 inhibitor to avoid an adverse drug interaction with Compound A, followed by administering to the subject a therapeutically effective amount of Compound A.

In another aspect, the invention features a method of administering Compound A to a human subject with cancer, wherein said subject is also in need of a CYP3A4 inhibitor, comprising administering to the subject a total daily dose between 100 mg to 1400 mg of Compound A while avoiding CYP3A4 inhibitor co-administration, and any one or more of the following:
(a) advising the subject that the CYP3A4 inhibitor should be avoided or discontinued,
(b) advising the subject that co-administration of Compound A with the CYP3A4 inhibitor can alter the therapeutic effect or adverse reaction profile of Compound A,
(c) advising the subject that use of Compound A in subjects being treated with the CYP3A4 inhibitor is contraindicated, or
(d) advising the subject that CYP3A4 inhibitors should be used with caution in subjects receiving Compound A due to the potential for an adverse reaction profile.

In another aspect, the invention features a method of administering Compound A to a human subject with cancer, wherein said subject is also in need of a CYP3A4 inhibitor, comprising administering to the subject a total daily dose between 100 mg to 1400 mg of Compound A while avoiding CYP3A4 inhibitor co-administration, and any one or more of the following:
(a) advising the subject that the CYP3A4 inhibitor should be avoided or discontinued,
(b) advising the subject that co-administration of Compound A with the CYP3A4 inhibitor can alter the therapeutic effect or adverse reaction profile of Compound A,
(c) advising the subject that use of Compound A in subjects being treated with the CYP3A4 inhibitor is contraindicated, or
(d) advising the subject that CYP3A4 inhibitors should be used with caution in subjects receiving Compound A due to the potential for an adverse reaction profile.

In another aspect, the invention features a method of avoiding an adverse drug interaction with Compound A in a human subject in need of Compound A, the method comprising administering to the subject a therapeutically effective amount of Compound A, and avoiding co-administration of a cytochrome p450 (CYP) 3A4 substrate, wherein said subject is also in need of the CYP3A4 substrate.

In another aspect, the invention features a method of avoiding an adverse drug interaction with Compound A in a human subject in need of Compound A, comprising discontinuing administration of a CYP3A4 substrate to avoid an adverse drug interaction with Compound A, and administering to the subject a therapeutically effective amount of Compound A. In another aspect, the invention features a method of avoiding an adverse drug interaction with Compound A in a human subject in need of Compound A, comprising discontinuing administration of a CYP3A4 substrate to avoid an adverse drug interaction with Compound A, followed by administering to the subject a therapeutically effective amount of Compound A.

In another aspect, the invention features a method of administering Compound A to a human subject with cancer, wherein said subject is also in need of a CYP3A4 substrate, comprising administering to the subject a total daily dose between 100 mg to 1400 mg of Compound A while avoiding CYP3A4 substrate co-administration, and any one or more of the following:
(a) advising the subject that the CYP3A4 substrate should be avoided or discontinued,
(b) advising the subject that co-administration of Compound A with the CYP3A4 substrate can alter the therapeutic effect or adverse reaction profile of Compound A,
(c) advising the subject that use of Compound A in subjects being treated with the CYP3A4 substrate is contraindicated, or
(d) advising the subject that CYP3A4 substrates should be used with caution in subjects receiving Compound A due to the potential for an adverse reaction profile.

In another aspect, the invention features a method of avoiding an adverse drug interaction with Compound A in a human subject in need of Compound A, the method comprising administering to the subject a therapeutically effective amount of Compound A, and avoiding co-administration of a cytochrome p450 (CYP) 3A4 inducer, wherein said subject is also in need of the CYP3A4 inducer.

In another aspect, the invention features a method of avoiding an adverse drug interaction with Compound A in a human subject in need of Compound A, comprising discontinuing administration of a CYP3A4 inducer to avoid an adverse drug interaction with Compound A, and administering to the subject a therapeutically effective amount of Compound A. In another aspect, the invention features a method of avoiding an adverse drug interaction with Compound A in a human subject in need of Compound A, comprising discontinuing administration of a CYP3A4 inducer to avoid an adverse drug interaction with Compound A, followed by administering to the subject a therapeutically effective amount of Compound A.

In another aspect, the invention features a method of administering Compound A to a human subject with cancer, wherein said subject is also in need of a CYP3A4 inducer, comprising administering to the subject a total daily dose between 100 mg to 1400 mg of Compound A while avoiding CYP3A4 inducer co-administration, and any one or more of the following:
(a) advising the subject that the CYP3A4 inducer should be avoided or discontinued,
(b) advising the subject that co-administration of Compound A with the CYP3A4 inducer can alter the therapeutic effect or adverse reaction profile of Compound A,
(c) advising the subject that use of Compound A in subjects being treated with the CYP3A4 inducer is contraindicated, or
(d) advising the subject that CYP3A4 inducers should be used with caution in subjects receiving Compound A due to the potential for an adverse reaction profile.

In another aspect, the invention features a method of avoiding an adverse drug interaction with Compound A in a human subject in need of Compound A, the method comprising administering to the subject a therapeutically effective amount of Compound A, and avoiding co-administration of a proton pump inhibitor (PPI), wherein said subject is also in need of the PPI.

In another aspect, the invention features a method of avoiding an adverse drug interaction with Compound A in a human subject in need of Compound A, comprising discontinuing administration of a PPI to avoid an adverse drug interaction with Compound A, and administering to the subject a therapeutically effective amount of Compound A. In another aspect, the invention features a method of avoiding an adverse drug interaction with Compound A in a human subject in need of Compound A, comprising discontinuing administration of a PPI to avoid an adverse drug interaction with Compound A, followed administering to the subject a therapeutically effective amount of Compound A.

In another aspect, the invention features a method of administering Compound A to a human subject with cancer, wherein said subject is also in need of a PPI, comprising administering to the subject a total daily dose between 100 mg to 1400 mg of Compound A while avoiding PPI co-administration, and any one or more of the following:
(a) advising the subject that the PPI should be avoided or discontinued,
(b) advising the subject that co-administration of Compound A with the PPI can alter the therapeutic effect or adverse reaction profile of Compound A,
(c) advising the subject that use of Compound A in subjects being treated with the PPI is contraindicated, or
(d) advising the subject that PPIs should be used with caution in subjects receiving Compound A due to the potential for an adverse reaction profile.

In another aspect, the invention features a method of avoiding an adverse drug interaction with Compound A in a human subject in need of Compound A, the method comprising administering to the subject a therapeutically effective amount of Compound A, and avoiding co-administration of a H-2 receptor antagonist (H2 blocker), wherein said subject is also in need of the H2 blocker.

In another aspect, the invention features a method of avoiding an adverse drug interaction with Compound A in a human subject in need of Compound A, comprising discontinuing administration of a H2 blocker to avoid an adverse drug interaction with Compound A, and administering to the subject a therapeutically effective amount of Compound A. In another aspect, the invention features a method of avoiding an adverse drug interaction with Compound A in a human subject in need of Compound A, comprising discontinuing administration of a H2 blocker to avoid an adverse drug interaction with Compound A, followed administering to the subject a therapeutically effective amount of Compound A.

In another aspect, the invention features a method of administering Compound A to a human subject with cancer, wherein said subject is also in need of a H2 blocker, comprising administering to the subject a total daily dose between 100 mg to 1400 mg of Compound A while avoiding H2 blocker co-administration, and any one or more of the following:
(a) advising the subject that the H2 blocker should be avoided or discontinued,
(b) advising the subject that co-administration of Compound A with the H2 blocker can alter the therapeutic effect or adverse reaction profile of Compound A,
(c) advising the subject that use of Compound A in subjects being treated with the H2 blocker is contraindicated, or
(d) advising the subject that H2 blockers should be used with caution in subjects receiving Compound A due to the potential for an adverse reaction profile.

In another aspect, the invention features a method of avoiding an adverse drug interaction with Compound A in a human subject in need of Compound A, the method comprising administering to the subject a therapeutically effective amount of Compound A, and avoiding co-administration of a disease-modifying antirheumatic drug (DMARD), wherein said subject is also in need of the DMARD.

In another aspect, the invention features a method of avoiding an adverse drug interaction with Compound A in a human subject in need of Compound A, comprising discontinuing administration of a DMARD to avoid an adverse drug interaction with Compound A, and administering to the subject a therapeutically effective amount of Compound A. In another aspect, the invention features a method of avoiding an adverse drug interaction with Compound A in a human subject in need of Compound A, comprising discontinuing administration of a DMARD to avoid an adverse drug interaction with Compound A, followed by administering to the subject a therapeutically effective amount of Compound A.

In another aspect, the invention features a method of administering Compound A to a human subject with cancer, wherein said subject is also in need of a DMARD, comprising administering to the subject a total daily dose between 100 mg to 1400 mg of Compound A while avoiding DMARD co-administration, and any one or more of the following:
(a) advising the subject that the DMARD should be avoided or discontinued,
(b) advising the subject that co-administration of Compound A with the DMARD can alter the therapeutic effect or adverse reaction profile of Compound A,
(c) advising the subject that use of Compound A in subjects being treated with the DMARD is contraindicated, or
(d) advising the subject that DMARDs should be used with caution in subjects receiving Compound A due to the potential for an adverse reaction profile.

In another aspect, the invention features a method of avoiding an adverse drug interaction with Compound A in a human subject in need of Compound A, the method comprising administering to the subject a therapeutically effective amount of Compound A, and avoiding co-administration of an immune suppressive agent, wherein said subject is also in need of the immune suppressive agent.

In another aspect, the invention features a method of avoiding an adverse drug interaction with Compound A in a human subject in need of Compound A, comprising discontinuing administration of an immune suppressive agent to avoid an adverse drug interaction with Compound A, and administering to the subject a therapeutically effective amount of Compound A. In another aspect, the invention features a method of avoiding an adverse drug interaction with Compound A in a human subject in need of Compound A, comprising discontinuing administration of an immune suppressive agent to avoid an adverse drug interaction with Compound A, followed by administering to the subject a therapeutically effective amount of Compound A.

In another aspect, the invention features a method of administering Compound A to a human subject with cancer, wherein said subject is also in need of an immune suppressive agent, comprising administering to the subject a total daily dose between 100 mg to 1400 mg of Compound A while avoiding immune suppressive agent co-administration, and any one or more of the following:
(a) advising the subject that the immune suppressive agent should be avoided or discontinued,
(b) advising the subject that co-administration of Compound A with the immune suppressive agent can alter the therapeutic effect or adverse reaction profile of Compound A,
(c) advising the subject that use of Compound A in subjects being treated with the immune suppressive agent is contraindicated, or
(d) advising the subject that immune suppressive agents should be used with caution in subjects receiving Compound A due to the potential for an adverse reaction profile.

In another aspect, the invention features a method of avoiding an adverse drug interaction with Compound A in a human subject in need of Compound A, the method comprising administering to the subject a therapeutically effective amount of Compound A, and avoiding co-administration of a P-glycoprotein (P-gp) substrate, wherein said subject is also in need of the P-gp substrate.

In another aspect, the invention features a method of avoiding an adverse drug interaction with Compound A in a human subject in need of Compound A, comprising discontinuing administration of a P-gp substrate to avoid an adverse drug interaction with Compound A, and administering to the subject a therapeutically effective amount of Compound A. In another aspect, the invention features a method of avoiding an adverse drug interaction with Compound A in a human subject in need of Compound A, comprising discontinuing administration of a P-gp substrate to avoid an adverse drug interaction with Compound A, followed by administering to the subject a therapeutically effective amount of Compound A.

In another aspect, the invention features a method of administering Compound A to a human subject with cancer, wherein said subject is also in need of a P-gp substrate, comprising administering to the subject a total daily dose between 100 mg to 1400 mg of Compound A while avoiding immune suppressive agent co-administration, and any one or more of the following:
(a) advising the subject that the P-gp substrate should be avoided or discontinued,
(b) advising the subject that co-administration of Compound A with the P-gp substrate can alter the therapeutic effect or adverse reaction profile of Compound A,
(c) advising the subject that use of Compound A in subjects being treated with the P-gp substrate is contraindicated, or
(d) advising the subject that P-gp substrates should be used with caution in subjects receiving Compound A due to the potential for an adverse reaction profile.

In another aspect, the invention features a method of avoiding an adverse drug interaction with Compound A in a human subject in need of Compound A, the method comprising administering to the subject a therapeutically effective amount of Compound A, and avoiding co-administration of a P-glycoprotein (P-gp) inhibitor, wherein said subject is also in need of the P-gp inhibitor.

In another aspect, the invention features a method of avoiding an adverse drug interaction with Compound A in a human subject in need of Compound A, comprising discontinuing administration of a P-gp inhibitor to avoid an adverse drug interaction with Compound A, and administering to the subject a therapeutically effective amount of Compound A. In another aspect, the invention features a method of avoiding an adverse drug interaction with Compound A in a human subject in need of Compound A, comprising discontinuing administration of a P-gp inhibitor to avoid an adverse drug interaction with Compound A, followed by administering to the subject a therapeutically effective amount of Compound A.

In another aspect, the invention features a method of administering Compound A to a human subject with cancer, wherein said subject is also in need of a P-gp inhibitor, comprising administering to the subject a total daily dose between 100 mg to 1400 mg of Compound A while avoiding immune suppressive agent co-administration, and any one or more of the following:
(a) advising the subject that the P-gp inhibitor should be avoided or discontinued,
(b) advising the subject that co-administration of Compound A with the P-gp inhibitor can alter the therapeutic effect or adverse reaction profile of Compound A,
(c) advising the subject that use of Compound A in subjects being treated with the P-gp inhibitor is contraindicated, or
(d) advising the subject that P-gp inhibitors should be used with caution in subjects receiving Compound A due to the potential for an adverse reaction profile.

In another aspect, the invention features a method of avoiding an adverse drug interaction with Compound A in a human subject in need of Compound A, the method comprising administering to the subject a therapeutically effective amount of Compound A, and avoiding co-administration of an organic anion-transporting polypeptide 1B1 or 1B3 (OATP1B1 or OATP1B3) substrate, wherein said subject is also in need of the OATP1B1 or OATP1B3 substrate.

In another aspect, the invention features a method of avoiding an adverse drug interaction with Compound A in a human subject in need of Compound A, comprising discontinuing administration of an OATP1B1 or OATP1B3 substrate to avoid an adverse drug interaction with Compound A, and administering to the subject a therapeutically effective amount of Compound A. In another aspect, the invention features a method of avoiding an adverse drug interaction with Compound A in a human subject in need of Compound A, comprising discontinuing administration of an OATP1B1 or OATP1B3 substrate to avoid an adverse drug interaction with Compound A, followed by administering to the subject a therapeutically effective amount of Compound A.

In another aspect, the invention features a method of administering Compound A to a human subject with cancer, wherein said subject is also in need of a OATP1B1 or OATP1B3 substrate, comprising administering to the subject a total daily dose between 100 mg to 1400 mg of Compound A while avoiding immune suppressive agent co-administration, and any one or more of the following: (a) advising the subject that the OATP1B1 or OATP1B3 substrate should be avoided or discontinued,
(b) advising the subject that co-administration of Compound A with the OATP1B1 or OATP1B3 substrate can alter the therapeutic effect or adverse reaction profile of Compound A, (c) advising the subject that use of Compound A in subjects being treated with the OATP1B1 or OATP1B3 substrate is contraindicated, or
(d) advising the subject that OATP1B1 or OATP1B3 substrates should be used with caution in subjects receiving Compound A due to the potential for an adverse reaction profile.

In another aspect, the invention features a method of avoiding an adverse drug interaction with Compound A in a human subject in need of Compound A, the method comprising administering to the subject a therapeutically effective amount of Compound A, and avoiding co-administration of a breast cancer resistance protein B3 (BCRP) substrate, wherein said subject is also in need of the BCRP substrate.

In another aspect, the invention features a method of avoiding an adverse drug interaction with Compound A in a human subject in need of Compound A, comprising discontinuing administration of a BCRP substrate to avoid an adverse drug interaction with Compound A, and administering to the subject a therapeutically effective amount of Compound A.

In another aspect, the invention features a method of administering Compound A to a human subject with cancer, wherein said subject is also in need of a BCRP substrate, comprising administering to the subject a total daily dose between 100 mg to 1400 mg of Compound A while avoiding immune suppressive agent co-administration, and any one or more of the following:
(a) advising the subject that the BCRP substrate should be avoided or discontinued,
(b) advising the subject that co-administration of Compound A with the BCRP substrate can alter the therapeutic effect or adverse reaction profile of Compound A,
(c) advising the subject that use of Compound A in subjects being treated with the BCRP substrate is contraindicated, or
(d) advising the subject that BCRP substrates should be used with caution in subjects receiving Compound A due to the potential for an adverse reaction profile.

In another aspect, the invention features a method of avoiding an adverse drug interaction with Compound A in a human subject in need of Compound A, the method comprising administering to the subject a therapeutically effective amount of Compound A, and avoiding co-administration of a drug known to prolong QTc interval, wherein said subject is also in need of the drug known to prolong QTc interval.

In another aspect, the invention features a method of avoiding an adverse drug interaction with Compound A in a human subject in need of Compound A, comprising discontinuing administration of a drug known to prolong QTc interval to avoid an adverse drug interaction with Compound A, and administering to the subject a therapeutically effective amount of Compound A. In another aspect, the invention features a method of avoiding an adverse drug interaction with Compound A in a human subject in need of Compound A, comprising discontinuing administration of a drug known to prolong QTc interval to avoid an adverse drug interaction with Compound A, followed by administering to the subject a therapeutically effective amount of Compound A.

In another aspect, the invention features a method of administering Compound A to a human subject with cancer, wherein said subject is also in need of a drug known to prolong QTc interval, comprising administering to the subject a total daily dose between 100 mg to 1400 mg of Compound A while avoiding immune suppressive agent co-administration, and any one or more of the following:
(a) advising the subject that the drug known to prolong QTc interval should be avoided or discontinued,
(b) advising the subject that co-administration of Compound A with the drug known to prolong QTc interval can alter the therapeutic effect or adverse reaction profile of Compound A,
(c) advising the subject that use of Compound A in subjects being treated with the drug known to prolong QTc interval is contraindicated, or
(d) advising the subject that drugs known to prolong QTc interval should be used with caution in subjects receiving Compound A due to the potential for an adverse reaction profile.

In another aspect, the invention features a method of reducing a treatment related adverse event in a human subject in need of Compound A, the method comprising administering to the subject a total daily dose between 100 mg to 1400 mg of Compound A.

In another aspect, the invention features a method of reducing nausea in a human subject in need of Compound A, the method comprising administering to the subject a total daily dose between 100 mg to 1400 mg of Compound A.

In another aspect, the invention features a method of reducing diarrhea in a human subject in need of Compound A, the method comprising administering to the subject a total daily dose between 100 mg to 1400 mg of Compound A.

In another aspect, the invention features a method of reducing vomiting in a human subject in need of Compound A, the method comprising administering to the subject a total daily dose between 100 mg to 1400 mg of Compound A.

In another aspect, the invention features a method of reducing fatigue in a human subject in need of Compound A, the method comprising administering to the subject a total daily dose between 100 mg to 1400 mg of Compound A.

In another aspect, the invention features a method of reducing rash in a human subject in need of Compound A, the method comprising administering to the subject a total daily dose between 100 mg to 1400 mg of Compound A.

In another aspect, the invention features a method of reducing an increase in alanine transaminase in a human subject in need of Compound A, the method comprising administering to the subject a total daily dose between 100 mg to 1400 mg of Compound A.

In another aspect, the invention features a method of reducing an increase in aspartate transferase in a human subject in need of Compound A, the method comprising administering to the subject a total daily dose between 100 mg to 1400 mg of Compound A.

In some embodiments, the method further comprises reducing the dose of Compound A from a total daily dose of 1400 mg to a total daily dose of 1200 mg. In some embodiments, the method further comprises reducing the dose of Compound A from a total daily dose of 1200 mg to a total daily dose of 900 mg. In some embodiments, the method further comprises reducing the dose of Compound A from a total daily dose of 600 mg to a total daily dose of 300 mg. In some embodiments, the subject has cancer. In some embodiments, the cancer comprises a RAS mutation. In some embodiments, the RAS mutation is G12D. In some embodiments, the RAS protein is KRAS. In some embodiments, the cancer is locally advanced or metastatic. In some embodiments, the cancer is pancreatic cancer. In some embodiments, the cancer is pancreatic ductal adenocarcinoma. In some embodiments, the cancer is pancreatic adenocarcinoma. In some embodiments, the cancer is lung cancer. In some embodiments, the cancer is colorectal cancer.

It is specifically contemplated that any limitation discussed with respect to one embodiment of the invention may apply to any other embodiment of the invention. Furthermore, any compound or composition of the invention may be used in any method of the invention, and any method of the invention may be used to produce or to utilize any compound or composition of the invention.

### BRIEF DESCRIPTION OF THE FIGURES

FIG. 1 shows the phase 1 study design in the treatment arm of Compound A monotherapy.

FIG. 2 shows demographics and baseline characteristics for enrolled patients with KRAS G12D tumors (Data cutoff date: Sep. 2, 2024). The patients treated with Compound A include the following tumor types: pancreatic ductal adenocarcinoma, colorectal cancer, non-small cell lung cancer, sinonasal adenocarcinoma, melanoma of the vulva, metastatic small bowel carcinoma, ovarian cancer, uterine adenocarcinoma, high grade mucinous carcinoma peritonei, ampullary cancer, duodenal, signet ring gastric carcinoma, uterine, and appendiceal. ECOG PS is Eastern Cooperative Oncology Group Performance Status.

FIG. 3A and FIG. 3B shows dose-dependent exposure increases and achieves exposure levels comparable to those in pre-clinical models which induced tumor regressions (Data as of Jul. 16, 2024).

FIG. 3A includes steady-state concentrations from Cycle 1 Day 15; the error bars represent standard deviation. FIG. 3B includes steady-state AUC in Cycle 1 Day 15, where each circle represents an individual patient AUC. Note: AUC is area under the curve; BID is twice daily; PDAC is pancreatic ductal adenocarcinoma; PK is pharmacokinetics; QD is once daily; RP2D is recommended phase 2 dose.

FIG. 4 shows treatment related adverse events (TRAEs) for patients receiving Compound A (Data cutoff date: Sep. 2, 2024). The median time on treatment was 2.8 months (range: 0.1-8.9). Note: AE is adverse event; ALT is alanine transaminase; AST is aspartate transferase; SAE is serious adverse event.

FIG. 5 shows treatment related adverse events (TRAEs) for patients receiving 1200 mg Compound A daily dose (Data cutoff date: Sep. 2, 2024). The median time on treatment was 2.8 months (range: 0.2-6.7).

FIG. 6 shows patient demographics and baseline characteristics for the subset of study patients with KRAS G12D PDAC (Data cutoff date: Sep. 2, 2024).

FIG. 7 shows ORR and DCR in patients with KRAS G12D PDAC treated with Compound A (Data cutoff date: Sep. 2, 2024). All treated patients with PDAC who received a first daily dose at least 14 weeks prior to data cutoff date (applies to Waterfall plot and ORR table); 3 additional patients (N=2 at 1200 mg daily; N=1 at <1200 mg daily) are not displayed on the Waterfall plot due to withdrawal of consent or clinical progression; Among patients with a response (confirmed or unconfirmed), 55% of first response occurred after 2 months of Compound A treatment (all dose levels). Note: CR is complete response; DCR is disease control rate; NE is not evaluable; PD is progressive disease; PR is partial response; PRu* is unconfirmed partial response; SD is stable disease; ORR is objective response rate; RECIST is response evaluation criteria in solid tumors.

FIG. 8 shows a case report of a patient with KRAS G12D PDAC treated with 1200 mg QD Compound A.

FIG. 9 shows the demographics and baseline characteristics for patients treated with Compound A as of the data cutoff date Dec. 2, 2024.

FIG. 10A and FIG. 10B graphically depicts Compound A exposure showed dose-dependent increases and achieved levels predicted to induce tumor regressions as of the data cutoff date Dec. 2, 2024.

FIG. 11 shows the treatment related adverse events for patients receiving Compound A as of the data cutoff date Dec. 2, 2024. Median time on treatment was 3.22 months (range: 0.03-10.81).

FIG. 12 shows the treatment related adverse events for patients receiving 1200 mg QD Compound A as of the data cutoff date Dec. 2, 2024.

FIG. 13 shows the demographics and baseline characteristics for patients with NSCLC treated with 1200 mg QD Compound A as of the data cutoff date Dec. 2, 2024.

FIG. 14 shows a waterfall plot, the overall response rate and disease control rate with KRAS G12D NSCLC treated with 1200 mg QD Compound A as of the data cutoff date Dec. 2, 2024.

FIG. 15 shows a swimmer's plot depicting the duration of treatment and responses in patients with KRAS G12D NSCLC treated with 1200 mg QD Compound A as of the data cutoff date Dec. 2, 2024.

FIG. 16 shows ctDNA clearance in patients with KRAS G12D NSCLC treated with 1200 mg QD Compound A as of the data cutoff date Dec. 2, 2024.

FIG. 17 depicts a case report of a 70-year-old patient with KRAS G12D NSCLC.

FIG. 18 depicts a case report of a 36-year-old patient with KRAS G12D NSCLC.

### DETAILED DESCRIPTION

In patients with KRAS-mutant tumors, the G12D mutation is the most frequently occurring KRAS mutation. In the United States, approximately 58,000 new cases of cancer with KRASG12D mutations are observed annually, with the majority of those cases in patients with colorectal cancer (CRC, 39%) and pancreatic ductal adenocarcinoma (PDAC, 39%), followed by non-small cell lung cancer (NSCLC, 14%) and other (8%) cancers. Currently, there is no approved RAS-targeted therapy for patients with KRASG12D mutant cancers, representing an unmet medical need for this population with a typically low response rate to the current standard of care therapies resulting in a poor prognosis.

Targeted therapies directed at RAS mutants or other nodes in the MAPK signaling cascade have historically been associated with significant safety and tolerability challenges, limiting their clinical utility both as monotherapies and in combination regimens.

For instance, KRAS(OFF) G12C inhibitors such as adagrasib have demonstrated notable adverse event profiles. In the KRYSTAL-1 phase II study involving patients with KRAS G12C-mutated colorectal cancer, treatment-related adverse events (TRAEs) of any grade occurred in 93% of patients receiving adagrasib monotherapy, with 34% experiencing grade 3 or 4 TRAEs, including anemia and diarrhea. Similarly, sotorasib, another KRAS(OFF) G12C inhibitor, has been associated with gastrointestinal toxicities and hepatotoxicity, which are significant concerns in monotherapy settings.

MEK inhibitors, targeting downstream components of the MAPK pathway, also present considerable safety concerns. Trametinib, for example, has been associated with adverse events such as rash, diarrhea, and peripheral edema. In the METRIC study, these adverse events were commonly observed and often required dose modifications.

Combination therapies involving RAS or MAPK pathway inhibitors have further exacerbated toxicity issues. For example, combining KRAS(OFF) G12C inhibitors with immune checkpoint inhibitors has led to increased hepatotoxicity, particularly when sotorasib is administered proximal to checkpoint inhibitor therapy. Additionally, combinations of BRAF and MEK inhibitors have been associated with higher risks of cardiovascular adverse events, including pulmonary embolism and decreased left ventricular ejection fraction.

Given this well-documented toxicity landscape, the observed safety and tolerability of Compound A is both surprising and unexpected. Unlike many agents targeting the RAS/MAPK axis, Compound A has demonstrated a low incidence of high-grade TRAEs and minimal impact on liver function or mucosal health in clinical settings. This safety profile not only supports its use as a monotherapy at active doses without the need for frequent dose modification but also suggests that Compound A may serve as a suitable partner in combination therapies, with a reduced risk of overlapping toxicities. These findings represent an unexpected advantage relative to prior RAS- and MAPK-directed compounds and support the methods described herein.

As discussed herein, Applicants have surprisingly discovered, in part, methods of safely and effectively treating a disease or disorder in subjects caused by a RAS G12D mutation.

### Definitions

In this application, unless otherwise clear from context, (i) the term “a” means “one or more”; (ii) the term “or” is used to mean “and/or” unless explicitly indicated to refer to alternatives only or the alternative are mutually exclusive, although the disclosure supports a definition that refers to only alternatives and “and/or”; (iii) the terms “comprising” and “including” are understood to encompass itemized components or steps whether presented by themselves or together with one or more additional components or steps; and (iv) where ranges are provided, endpoints are included.

As used herein, the term “about” is used to indicate that a value includes the standard deviation of error for the device or method being employed to determine the value. In certain embodiments, the term “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

Note that when a range or amount is provided in the disclosure herein, +/−5% of each range endpoint or specific amount is included, unless otherwise indicated. For example, a range of 100 mg to 1400 mg of Compound A is understood to encompass 100 mg (+/−5%) to 1400 mg (+/−5%), e.g., 95 mg to 1470 mg Compound A.

As used herein, the term “administration” refers to the administration of a composition comprising Compound A to a subject or system. Administration also includes administering a prodrug derivative or analog or pharmaceutically acceptable salt to the subject, which can form an equivalent amount of active compound within the subject's body. Administration to an animal subject (e.g., to a human) may be by any appropriate route. For example, in some embodiments, administration may be bronchial (including by bronchial instillation), buccal, enteral, intradermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal or vitreal. In some embodiments, a composition comprising Compound A is administered orally.

As used herein, “biosimilar” means a biological product that is similar to a reference biological product, notwithstanding minor differences in clinically inactive components, and that has no clinically meaningful differences from the reference product in terms of safety, purity, or potency, consistent with applicable regulatory standards (e.g., FDA, EMA). In some embodiments, a biosimilar includes a product licensed under 42 U.S.C. § 262(k), Article 10(4) of Directive 2001/83/EC, or an equivalent pathway.

The term “combination therapy” refers to a method of treatment including administering to a subject at least two active therapeutic agents, as one or more pharmaceutical compositions, as part of a therapeutic regimen. For example, a combination therapy may include administration of a single pharmaceutical composition including at least two therapeutic agents and one or more pharmaceutically acceptable carrier, excipient, diluent, or surfactant. A combination therapy may include administration of two or more pharmaceutical compositions, each composition including one or more therapeutic agent and one or more pharmaceutically acceptable carrier, excipient, diluent, or surfactant. The two or more agents may optionally be administered simultaneously (as a single or as separate compositions) or sequentially (as separate compositions). The therapeutic agents may be administered in an effective amount. The therapeutic agent may be administered in a therapeutically effective amount. In some embodiments, the effective amount of one or more of the therapeutic agents may be lower when used in a combination therapy than the therapeutic amount of the same therapeutic agent when it is used as a monotherapy, e.g., due to an additive or synergistic effect of combining the two or more therapeutics.

As used herein, the term “dosage form” refers to a physically discrete unit of a compound (e.g., Compound A) for administration to a subject. Each unit contains a predetermined quantity of compound. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or compound administered to a particular subject is determined by one or more attending physicians and may involve administration of multiple dosage forms.

As used herein, the term “dosing regimen” refers to a set of unit doses (typically more than one) that are administered individually to a subject, typically separated by periods of time. In some embodiments, a given therapeutic compound (e.g., Compound A) has a recommended dosing regimen, which may involve one or more doses. In some embodiments, a dosing regimen includes a plurality of doses each of which are separated from one another by a time period of the same length; in some embodiments, a dosing regimen includes a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen includes a first dose in a first dose amount, followed by one or more additional doses in a second dose amount different from the first dose amount. In some embodiments, a dosing regimen includes a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

The term “disorder” is used in this disclosure to mean, and is used interchangeably with, the terms disease, condition, or illness, unless otherwise indicated.

The terms “inhibit,” “block,” and “suppress” are used interchangeably and refer to any statistically significant decrease in a biological activity, including full blocking of the activity. As used herein, the term “inhibitor” refers to a compound that prevents a biomolecule, (e.g., a protein, nucleic acid) from completing or initiating a reaction. An inhibitor can inhibit a reaction by competitive, uncompetitive, or non-competitive means, for example. With respect to its binding mechanism, an inhibitor may be an irreversible inhibitor or a reversible inhibitor. Exemplary inhibitors include, but are not limited to, nucleic acids, DNA, RNA, shRNA, siRNA, proteins, protein mimetics, peptides, peptidomimetics, antibodies, small molecules, chemicals, analogs that mimic the binding site of an enzyme, receptor, or other protein. In some embodiments, the inhibitor is a small molecule, e.g., a low molecular weight organic compound, e.g., an organic compound having a molecular weight (MW) of less than 1200 Daltons (Da). In some embodiments, the MW is less than 1100 Da. In some embodiments, the MW is less than 1000 Da. In some embodiments, the MW is less than 900 Da. In some embodiments, the range of the MW of the small molecule is between 800 Da and 1200 Da. Small molecule inhibitors include cyclic and acyclic compounds. Small molecules inhibitors include natural products, derivatives, and analogs thereof. Small molecule inhibitors can include a covalent cross-linking group capable of forming a covalent cross-link, e.g., with an amino acid side-chain of a target protein.

As used herein “patient” or “subject” are used interchangeably and refer to a mammal, for whom diagnosis, prognosis, or therapy is desired. Mammalian subjects include, but are not limited to, humans, domestic animals, farm animals, sports animals, and zoo animals including, for example, humans, non-human primates, dogs, cats, guinea pigs, rabbits, rats, mice, horses, and cattle. In certain embodiments, the subject has been diagnosed with cancer. In certain embodiments, the subject is a human afflicted with a tumor (e.g., cancer) who has been diagnosed with a need for treatment for a tumor (e.g., cancer).

As used herein, the term “pharmaceutical composition” refers to a compound, such as Compound A disclosed herein, or a pharmaceutically acceptable salt thereof, formulated together with a pharmaceutically acceptable excipient.

A “pharmaceutically acceptable excipient,” as used herein, refers to any inactive ingredient (for example, a vehicle capable of suspending or dissolving the active compound) having the properties of being nontoxic and noninflammatory in a subject. Typical excipients include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Excipients include, but are not limited to: butylated optionally substituted hydroxyltoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, optionally substituted hydroxylpropyl cellulose, optionally substituted hydroxylpropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol. Those of ordinary skill in the art are familiar with a variety of agents and materials useful as excipients. See, e.g., Ansel, et al., Ansel's Pharmaceutical Dosage Forms and Drug Delivery Systems. Philadelphia: Lippincott, Williams & Wilkins, 2004; Gennaro, et al., Remington: The Science and Practice of Pharmacy. Philadelphia: Lippincott, Williams & Wilkins, 2000; and Rowe, Handbook of Pharmaceutical Excipients. Chicago, Pharmaceutical Press, 2005. In some embodiments, a composition includes at least two different pharmaceutically acceptable excipients.

The term “pharmaceutically acceptable salt,” as use herein, refers to those salts of the compounds described herein that are, within the scope of sound medical judgment, suitable for use in contact with the tissues of humans and other animals without undue toxicity, irritation, allergic response and the like, and are commensurate with a reasonable benefit/risk ratio. Pharmaceutically acceptable salts are well known in the art. For example, pharmaceutically acceptable salts are described in: Berge et al., J. Pharmaceutical Sciences 66:119, 1977 and in Pharmaceutical Salts: Properties, Selection, and Use, (Eds. P. H. Stahl and C. G. Wermuth), WileyVCH, 2008. The salts can be prepared in situ during the final isolation and purification of the compounds described herein or separately by reacting the free base group with a suitable organic acid.

The terms “RAS pathway” and “RAS/MAPK pathway” are used interchangeably herein to refer to a signal transduction cascade downstream of various cell surface growth factor receptors in which activation of RAS (and its various isoforms and allotypes) is a central event that drives a variety of cellular effector events that determine the proliferation, activation, differentiation, mobilization, and other functional properties of the cell. SHP2 conveys positive signals from growth factor receptors to the RAS activation/deactivation cycle, which is modulated by guanine nucleotide exchange factors (GEFs, such as SOS1) that load GTP onto RAS to produce functionally active GTP-bound RAS as well as GTP-accelerating proteins (GAPs, such as NF1) that facilitate termination of the signals by conversion of GTP to GDP. GTP-bound RAS produced by this cycle conveys essential positive signals to a series of serine/threonine kinases including RAF and MAP kinases, from which emanate additional signals to various cellular effector functions.

The terms “RAS inhibitor” and “inhibitor of [a] RAS” are used interchangeably to refer to any inhibitor that targets, that is, selectively binds to or inhibits a RAS protein.

As used herein, the term “RAS(ON) inhibitor” refers to an inhibitor that targets, that is, selectively binds to or inhibits, the GTP-bound, active state of RAS (e.g., selective over the GDP-bound, inactive state of RAS). Inhibition of the GTP-bound, active state of RAS includes, for example, the inhibition of oncogenic signaling from the GTP-bound, active state of RAS. In some embodiments, the RAS(ON) inhibitor is an inhibitor that selectively binds to and inhibits the GTP-bound, active state of RAS. In certain embodiments, RAS(ON) inhibitors may also bind to or inhibit the GDP-bound, inactive state of RAS (e.g., with a lower affinity or inhibition constant than for the GTP-bound, active state of RAS). In certain embodiments, a RAS(ON) inhibitor useful in the present disclosure may form a high affinity three-component complex, or conjugate, between a synthetic ligand and two intracellular proteins which do not interact under normal physiological conditions: the target protein of interest (e.g., RAS), and a widely expressed cytosolic chaperone (presenter protein) in the cell (e.g., cyclophilin A). More specifically, in some embodiments, the inhibitors of RAS described herein induce a new binding pocket in RAS by driving formation of a high affinity tri-complex, or conjugate, between the RAS protein and the widely expressed cytosolic chaperone, cyclophilin A (CypA). A RAS(ON) inhibitor may be an antibody-drug conjugate. See also doi.org/10.1021/acs.jmedchem.4c02929.

As used herein, the term “RAS(OFF) inhibitor” refers to an inhibitor that targets, that is, selectively binds to or inhibits, the GDP-bound, inactive state of RAS (e.g., selective over the GTP-bound, active state of RAS). RAS(OFF) inhibitors are known in the art and described. Non-limiting examples of RAS(OFF) inhibitors include A2A-03, ABREV01, ADT-007, ABT-200, ADT-030, ADT-1004, BBP-454, BGB-53038, BI-2865, BI-2493, BI 3706674, BRSD-143, ERAS-4, ERAS-254, ERAS-4001, HB-700 (G12X+G13D), HZ-V068, ID12241161, JAB-23400, LY4066434, OC211, PF-07985045, PF-07934040, PF-4040, QTX2024, QTX3034, RSC-1255, SIL204, SYNB021225, YL-17231, and ZG2001. Non-limiting examples of RASG12C(OFF) inhibitors include adagrasib (MRTX849), divarasib (RG6330/GDC-6036), fulzerasib (IBI351/GFH925), garsorasib (D-1553), glecirasib (JAB-21822), olomorasib (LY3537982), opnurasib (JDQ443), sotorasib (AMG 510), ARS-853, ARS-1620, BI-0474, BI 1823911, BPI-421286, D3S-001, ERAS-3490, GEC255, GH35, HBI-2438, HS-10370, JAB-21000, JAB-21822, JMKX001899, JNJ-74699157 (ARS-3248), MK-1084, SK-17, and YL-15293. Non-limiting examples of RASG12D(OFF) inhibitors include AST2169, BPI-501836, DN022150, ERAS-4693, ERAS-5024, GDC-7035 (RG6620), HBW-012-D, HBW-012-E, HBW-012336, HRS-4642, HS-10529, INCB186748, JAB-22000, KD-8, KRB-456, LY3962673, MRTX282, MRTX1133, Q2a, QLC1101, RNK08954, SHR1127, TH-Z827, TH-Z835, TSN1611, and VRTX153, HJ-119, JR-6000, NKT-G12D, FWD-K02, JAB-BX600, EB-TM1, ABSK141, and BPI-2491. Non-limiting examples of RASG12V(OFF) inhibitors include JAB-23000 and QTX3544.

As used herein, the terms “RAS(ON) multi-selective inhibitor,” “RASMULTI inhibitor,” “RASMULTI(ON) inhibitor,” and “RAS(MULTI) inhibitor” refer to a RAS inhibitor of at least three RAS isoforms, including wild-type and/or variants with missense mutations at one of the following positions: 12, 13, 59, 61, or 146. In some embodiments, a RAS(ON) multi-selective inhibitor (e.g., daraxonrasib or RMC-6236) refers to a RAS inhibitor of at least three RAS variants with missense mutations at one of the following positions: 12, 13, and 61. Exemplary RAS(ON) multi-selective inhibitors are described herein.

As used herein, the terms “RAS(ON) mutant-selective inhibitor” refers to a RAS inhibitor selective for a RAS(ON) variant with missense mutation at one of the following positions: 12, 13, or 61. Non-limiting examples of RAS(ON) mutant-selective inhibitors include RAS(ON) G12C-selective inhibitors (e.g., elironrasib or RMC-6291), RAS(ON) G12D-selective inhibitors (e.g., zoldonrasib or RMC-9805 or Compound A), RAS(ON) Q61H-selective inhibitors (e.g., RMC-0708), RAS(ON) G12V-selective inhibitors (e.g. RMC-5127), and RAS(ON) G13D-selective inhibitors. Exemplary RAS(ON) mutant-selective inhibitors are described herein.

A “therapeutic agent” is any substance, e.g., a compound or composition, capable of treating a disease or disorder. In some embodiments, therapeutic agents that are useful in connection with the present disclosure include RAS inhibitors and cancer chemotherapeutics. Many such therapeutic agents are known in the art and are disclosed herein.

The term “therapeutically effective amount” means an amount that is sufficient, when administered to a population suffering from or susceptible to a disease, disorder, or condition in accordance with a therapeutic dosing regimen, to treat the disease, disorder, or condition. In some embodiments, a therapeutically effective amount is one that reduces the incidence or severity of, or delays onset of, one or more symptoms of the disease, disorder, or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine). Those of ordinary skill in the art will appreciate that, in some embodiments, a therapeutically effective amount may be formulated or administered in a single dose. In some embodiments, a therapeutically effective amount may be formulated or administered in a plurality of doses, for example, as part of a dosing regimen.

The term “treatment” (also “treat” or “treating”), in its broadest sense, refers to any administration of a substance (e.g., a combination therapy as disclosed herein) that partially or completely alleviates, ameliorates, relieves, inhibits, delays onset of, reduces severity of, or reduces incidence of one or more symptoms, features, or causes of a particular disease, disorder, or condition. In some embodiments, such treatment may be administered to a subject who is diagnosed with the disease, disorder or condition but does not exhibit signs of the relevant disease, disorder or condition or of a subject who exhibits only early signs of the disease, disorder, or condition. Alternatively, or additionally, in some embodiments, treatment may be administered to a subject who exhibits one or more established signs of the relevant disease, disorder or condition. In some embodiments, treatment may be of a subject who has been diagnosed as suffering from the relevant disease, disorder, or condition. In any treatment method herein, a patient or subject may be in need of such treatment.

### Treatment Methods and Uses

The present disclosure provides, inter alia, the use of a RAS(ON) G12D-selective inhibitor therapy in methods of treating subjects with disease or disorder resulting from a RAS G12D mutations (e.g., RASG12D-mutant solid tumors). In various embodiments, the RAS(ON) G12D-selective inhibitor is Compound A.

Accordingly, a RAS(ON) G12D-selective inhibitor useful according to the present disclosure is a compound such as Compound A

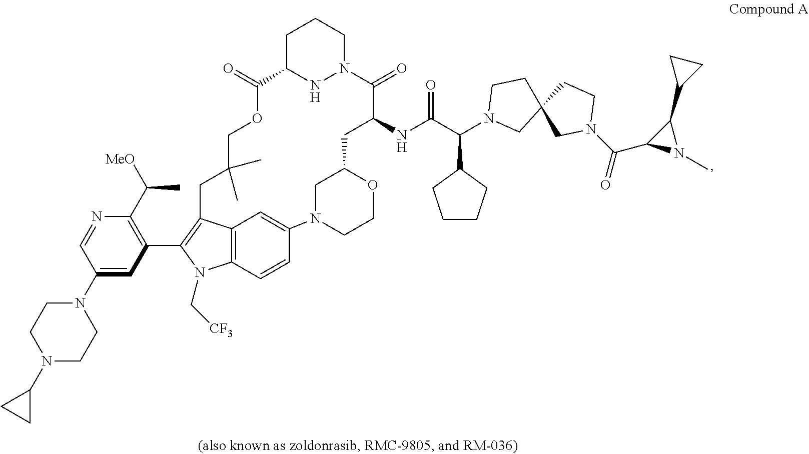

Compound A is RAS inhibitor—more specifically, an oral RAS(ON) G12D-selective, covalent tri-complex inhibitor, that is selective for the active, GTP-bound state, of the canonical RAS isoforms harboring a G12D mutation. Compound A binds to cyclophilin A, which is abundantly expressed in normal tissues and tumors, resulting in a binary complex that covalently binds to RAS(ON) G12D to form a tri-complex, blocking downstream RAS signaling, see e.g., Weller et al., Science, Vol 389, Issue 6758, (2025).

Compound A may exist as a conformational stereoisomer, such as an atropisomer. Pharmaceutically acceptable salts of Compound A are also contemplated, as are solvates, hydrates and polymorphs. Compound A can be prepared as described in WO 2023060253, incorporated herein by reference in its entirety.

Compound A can be present as a pharmaceutically acceptable isotopically labeled version, wherein one or more atoms is replaced by atoms having the same atomic number, but an atomic mass or mass number different from the atomic mass or mass number usually found in nature. Examples of isotopes that can be incorporated into Compound A include isotopes of hydrogen, carbon, nitrogen, oxygen, and fluorine, such as ²H, ³H, ¹¹C, ¹³C, ¹⁴C, ¹³N, ¹⁵N, ¹⁵O, ¹⁷O, and ¹⁸O, respectively. These radio-labeled compounds could be useful to help determine or measure the effectiveness of Compound A, by characterizing, for example, the site or mode of action. Certain isotopically labeled versions of Compound A, for example, those incorporating a radioactive isotope, are useful in drug and/or substrate tissue distribution studies. The radioactive isotopes tritium, i.e., ³H, and carbon-14, i.e., ¹⁴C, are particularly useful for this purpose in view of their ease of incorporation and ready means of detection. Non-limiting examples of such incorporation can be seen in, e.g., WO 2023060253.

Substitution with heavier isotopes such as deuterium, i.e., ²H, may afford certain therapeutic advantages resulting from greater metabolic stability, for example, increased in vivo half-life or reduced dosage requirements. Substitution with positron emitting isotopes, such as ¹¹C, ¹⁵O and ¹³N, can be useful in Positron Emission Topography (PET) studies.

Further provided is a method of treating cancer in a subject in need thereof, the method comprising administering to the subject a therapeutically effective amount of Compound A. The cancer may, for example, be pancreatic cancer (e.g., pancreatic adenocarcinoma or pancreatic ductal adenocarcinoma), colorectal cancer, non-small cell lung cancer, acute myeloid leukemia, multiple myeloma, melanoma, thyroid gland adenocarcinoma, renal cancer, bladder cancer, biliary/gall bladder cancer, gastroesophageal cancer, uterine/endometrial cancer, a myelodysplastic syndrome, head and neck squamous cell carcinoma, or squamous cell lung carcinoma. In some embodiments, the cancer comprises a RAS mutation, such as KRAS G12D. In some embodiments, the cancer comprises a KRAS G12D mutation and one or more additional cancer cells having RAS mutations selected from KRAS G12C, KRAS G12V, KRAS G12S, KRAS G13C, KRAS G13D, KRAS Q61H, KRAS Q61R, KRAS Q61K, or KRAS Q61L, or a combination thereof. In some embodiments, the cancer comprises an additional RAS mutation, such as NRAS G12D, NRAS Q61R, NRAS Q61K, NRAS Q61L, NRAS Q61H, or NRAS Q61P, or a combination thereof. Other RAS mutations are described herein.

Further provided is a method of treating a RAS protein-related disease in a subject in need thereof, the method comprising administering to the subject a therapeutically effective amount of Compound A, or a pharmaceutically acceptable salt thereof.

In some embodiments of any of the methods described herein, the method includes administering a total daily dose between 100 mg to 1400 mg (e.g., 100 mg to 1400 mg, 150 mg to 1400 mg, 200 mg to 1400 mg, 250 mg to 1400 mg, 300 mg to 1400 mg, 350 mg to 1400 mg, 400 mg to 1400 mg, 450 mg to 1400 mg, 500 mg to 1400 mg, 550 mg to 1400 mg, 600 mg to 1400 mg, 650 mg to 1400 mg, 700 mg to 1400 mg, 750 mg to 1400 mg, 800 mg to 1400 mg, 850 mg to 1400 mg, 900 mg to 1400 mg, 950 mg to 1400 mg, 1000 mg to 1400 mg, 1100 mg to 1400 mg, 1200 mg to 1400 mg, 1300 mg to 1400 mg, 100 mg to 1300 mg, 150 mg to 1300 mg, 200 mg to 1300 mg, 250 mg to 1300 mg, 300 mg to 1300 mg, 350 mg to 1300 mg, 400 mg to 1300 mg, 450 mg to 1300 mg, 500 mg to 1300 mg, 550 mg to 1300 mg, 600 mg to 1300 mg, 650 mg to 1300 mg, 700 mg to 1300 mg, 750 mg to 1300 mg, 800 mg to 1300 mg, 850 mg to 1300 mg, 900 mg to 1300 mg, 950 mg to 1300 mg, 1000 mg to 1300 mg, 1100 mg to 1300 mg, 1200 mg to 1300 mg, 100 mg to 1200 mg, 150 mg to 1200 mg, 200 mg to 1200 mg, 250 mg to 1200 mg, 300 mg to 1200 mg, 350 mg to 1200 mg, 400 mg to 1200 mg, 450 mg to 1200 mg, 500 mg to 1200 mg, 550 mg to 1200 mg, 600 mg to 1200 mg, 650 mg to 1200 mg, 700 mg to 1200 mg, 750 mg to 1200 mg, 800 mg to 1200 mg, 850 mg to 1200 mg, 900 mg to 1200 mg, 950 mg to 1200 mg, 1000 mg to 1200 mg, 1100 mg to 1200 mg, 100 mg to 1100 mg, 150 mg to 1100 mg, 200 mg to 1100 mg, 250 mg to 1100 mg, 300 mg to 1100 mg, 350 mg to 1100 mg, 400 mg to 1100 mg, 450 mg to 1100 mg, 500 mg to 1100 mg, 550 mg to 1100 mg, 600 mg to 1100 mg, 650 mg to 1100 mg, 700 mg to 1100 mg, 750 mg to 1100 mg, 800 mg to 1100 mg, 850 mg to 1100 mg, 900 mg to 1100 mg, 950 mg to 1100 mg, 1000 mg to 1100 mg, 100 mg to 1000 mg, 150 mg to 1000 mg, 200 mg to 1000 mg, 250 mg to 1000 mg, 300 mg to 1000 mg, 350 mg to 1000 mg, 400 mg to 1000 mg, 450 mg to 1000 mg, 500 mg to 1000 mg, 550 mg to 1000 mg, 600 mg to 1000 mg, 650 mg to 1000 mg, 700 mg to 1000 mg, 750 mg to 1000 mg, 800 mg to 1000 mg, 850 mg to 1000 mg, 900 mg to 1000 mg, 950 mg to 1000 mg, 100 mg to 950 mg, 150 mg to 950 mg, 200 mg to 950 mg, 250 mg to 950 mg, 300 mg to 950 mg, 350 mg to 950 mg, 400 mg to 950 mg, 450 mg to 950 mg, 500 mg to 950 mg, 550 mg to 950 mg, 600 mg to 950 mg, 650 mg to 950 mg, 700 mg to 950 mg, 750 mg to 950 mg, 800 mg to 950 mg, 850 mg to 950 mg, 900 mg to 950 mg, 100 mg to 900 mg, 150 mg to 900 mg, 200 mg to 900 mg, 250 mg to 900 mg, 300 mg to 900 mg, 350 mg to 900 mg, 400 mg to 900 mg, 450 mg to 900 mg, 500 mg to 900 mg, 550 mg to 900 mg, 600 mg to 900 mg, 650 mg to 900 mg, 700 mg to 900 mg, 750 mg to 900 mg, 800 mg to 900 mg, 850 mg to 900 mg, 100 mg to 850 mg, 150 mg to 850 mg, 200 mg to 850 mg, 250 mg to 850 mg, 300 mg to 850 mg, 350 mg to 850 mg, 400 mg to 850 mg, 450 mg to 850 mg, 500 mg to 850 mg, 550 mg to 850 mg, 600 mg to 850 mg, 650 mg to 850 mg, 700 mg to 850 mg, 750 mg to 850 mg, 800 mg to 850 mg, 100 mg to 800 mg, 150 mg to 800 mg, 200 mg to 800 mg, 250 mg to 800 mg, 300 mg to 800 mg, 350 mg to 800 mg, 400 mg to 800 mg, 450 mg to 800 mg, 500 mg to 800 mg, 550 mg to 800 mg, 600 mg to 800 mg, 650 mg to 800 mg, 700 mg to 800 mg, 750 mg to 800 mg, 100 mg to 750 mg, 150 mg to 750 mg, 200 mg to 750 mg, 250 mg to 750 mg, 300 mg to 750 mg, 350 mg to 750 mg, 400 mg to 750 mg, 450 mg to 750 mg, 500 mg to 750 mg, 550 mg to 750 mg, 600 mg to 750 mg, 650 mg to 750 mg, 700 mg to 750 mg, 100 mg to 700 mg, 150 mg to 700 mg, 200 mg to 700 mg, 250 mg to 700 mg, 300 mg to 700 mg, 350 mg to 700 mg, 400 mg to 700 mg, 450 mg to 700 mg, 500 mg to 700 mg, 550 mg to 700 mg, 600 mg to 700 mg, 650 mg to 700 mg, 100 mg to 650 mg, 150 mg to 650 mg, 200 mg to 650 mg, 250 mg to 650 mg, 300 mg to 650 mg, 350 mg to 650 mg, 400 mg to 650 mg, 450 mg to 650 mg, 500 mg to 650 mg, 550 mg to 650 mg, 600 mg to 650 mg, 100 mg to 600 mg, 150 mg to 600 mg, 200 mg to 600 mg, 250 mg to 600 mg, 300 mg to 600 mg, 350 mg to 600 mg, 400 mg to 600 mg, 450 mg to 600 mg, 500 mg to 600 mg, 550 mg to 600 mg, 100 mg to 550 mg, 150 mg to 550 mg, 200 mg to 550 mg, 250 mg to 550 mg, 300 mg to 550 mg, 350 mg to 550 mg, 400 mg to 550 mg, 450 mg to 550 mg, 500 mg to 550 mg, 100 mg to 500 mg, 150 mg to 500 mg, 200 mg to 500 mg, 250 mg to 500 mg, 300 mg to 500 mg, 350 mg to 500 mg, 400 mg to 500 mg, 450 mg to 500 mg, 100 mg to 450 mg, 150 mg to 450 mg, 200 mg to 450 mg, 250 mg to 450 mg, 300 mg to 450 mg, 350 mg to 450 mg, 400 mg to 450 mg, 100 mg to 400 mg, 150 mg to 400 mg, 200 mg to 400 mg, 250 mg to 400 mg, 300 mg to 400 mg, 350 mg to 400 mg, 100 mg to 350 mg, 150 mg to 350 mg, 200 mg to 350 mg, 250 mg to 350 mg, 300 mg to 350 mg, 100 mg to 300 mg, 150 mg to 300 mg, 200 mg to 300 mg, 250 mg to 300 mg, 100 mg to 250 mg, 150 mg to 250 mg, 200 mg to 250 mg, 100 mg to 200 mg, 150 mg to 200 mg, or 100 mg to 150 mg) of Compound A to a subject in need thereof. In each of the preceding embodiments, the total daily dose can be divided and administered once, twice, or more daily.

In some embodiments, the method includes administering a total daily dose of 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, 510 mg, 520 mg, 530 mg, 540 mg, 550 mg, 560 mg, 570 mg, 580 mg, 590 mg, 600 mg, 610 mg, 620 mg, 630 mg, 640 mg, 650 mg, 660 mg, 670 mg, 680 mg, 690 mg, 700 mg, 710 mg, 720 mg, 730 mg, 740 mg, 750 mg, 760 mg, 770 mg, 780 mg, 790 mg, 800 mg, 810 mg, 820 mg, 830 mg, 840 mg, 850 mg, 860 mg, 870 mg, 880 mg, 890 mg, 900 mg, 910 mg, 920 mg, 930 mg, 940 mg, 950 mg, 960 mg, 970 mg, 980 mg, 990 mg, 1000 mg, 1010 mg, 1020 mg, 1030 mg, 1040 mg, 1050 mg, 1060 mg, 1070 mg, 1080 mg, 1090 mg, 1100 mg, 1110 mg, 1120 mg, 1130 mg, 1140 mg, 1150 mg, 1160 mg, 1170 mg, 1180 mg, 1190 mg, 1200 mg, 1250 mg, 1300 mg, 1350 mg, or 1400 mg of Compound A to the subject in need thereof. In each of the preceding embodiments, the total daily dose can be administered once or twice daily.

In some embodiments, the method includes administering a total daily dose of 150 mg to 1200 mg of Compound A to the subject in need thereof. In some embodiments, the method includes administering a total daily dose of 300 mg to 1200 mg of Compound A to the subject in need thereof. In some embodiments, the method comprises administering a total daily dose of 600 mg to 1200 mg of Compound A to the subject in need thereof. In some embodiments, the method comprises administering a total daily dose of 900 mg to 1200 mg of Compound A to the subject in need thereof.

In some embodiments, the method comprises administering a total daily dose between 100 mg to 1200 mg of Compound A to the subject in need thereof. In some embodiments, the method comprises administering a total daily dose between 150 mg to 1200 mg of Compound A to the subject in need thereof. In some embodiments, the method comprises administering a total daily dose between 200 mg to 1200 mg of Compound A to the subject in need thereof. In some embodiments, the method comprises administering a total daily dose between 250 mg to 1200 mg of Compound A to the subject in need thereof. In some embodiments, the method comprises administering a total daily dose between 300 mg to 1200 mg of Compound A to the subject in need thereof. In some embodiments, the method comprises administering a total daily dose between 350 mg to 1200 mg of Compound A to the subject in need thereof. In some embodiments, the method comprises administering a total daily dose between 400 mg to 1200 mg of Compound A to the subject in need thereof. In some embodiments, the method comprises administering a total daily dose between 450 mg to 1200 mg of Compound A to the subject in need thereof. In some embodiments, the method comprises administering a total daily dose between 500 mg to 1200 mg of Compound A to the subject in need thereof. In some embodiments, the method comprises administering a total daily dose between 550 mg to 1200 mg of Compound A to the subject in need thereof. In some embodiments, the method comprises administering a total daily dose between 600 mg to 1200 mg of Compound A to the subject in need thereof. In some embodiments, the method comprises administering a total daily dose between 650 mg to 1200 mg of Compound A to the subject in need thereof. In some embodiments, the method comprises administering a total daily dose between 700 mg to 1200 mg of Compound A to the subject in need thereof. In some embodiments, the method comprises administering a total daily dose between 750 mg to 1200 mg of Compound A to the subject in need thereof. In some embodiments, the method comprises administering a total daily dose between 800 mg to 1200 mg of Compound A to the subject in need thereof. In some embodiments, the method comprises administering a total daily dose between 850 mg to 1200 mg of Compound A to the subject in need thereof. In some embodiments, the method comprises administering a total daily dose between 900 mg to 1200 mg of Compound A to the subject in need thereof. In some embodiments, the method comprises administering a total daily dose between 950 mg to 1200 mg of Compound A to the subject in need thereof. In some embodiments, the method comprises administering a total daily dose between 1000 mg to 1200 mg of Compound A to the subject in need thereof. In some embodiments, the method comprises administering a total daily dose between 1100 mg to 1200 mg of Compound A to the subject in need thereof.

In some embodiments, the method comprises administering a total daily dose between 100 mg to 1100 mg of Compound A to the subject in need thereof. In some embodiments, the method comprises administering a total daily dose between 150 mg to 1100 mg of Compound A to the subject in need thereof. In some embodiments, the method comprises administering a total daily dose between 200 mg to 1100 mg of Compound A to the subject in need thereof. In some embodiments, the method comprises administering a total daily dose between 250 mg to 1100 mg of Compound A to the subject in need thereof. In some embodiments, the method comprises administering a total daily dose between 300 mg to 1100 mg of Compound A to the subject in need thereof. In some embodiments, the method comprises administering a total daily dose between 350 mg to 1100 mg of Compound A to the subject in need thereof. In some embodiments, the method comprises administering a total daily dose between 400 mg to 1100 mg of Compound A to the subject in need thereof. In some embodiments, the method comprises administering a total daily dose between 450 mg to 1100 mg of Compound A to the subject in need thereof. In some embodiments, the method comprises administering a total daily dose between 500 mg to 1100 mg of Compound A to the subject in need thereof. In some embodiments, the method comprises administering a total daily dose between 550 mg to 1100 mg of Compound A to the subject in need thereof. In some embodiments, the method comprises administering a total daily dose between 600 mg to 1100 mg of Compound A to the subject in need thereof. In some embodiments, the method comprises administering a total daily dose between 650 mg to 1100 mg of Compound A to the subject in need thereof. In some embodiments, the method comprises administering a total daily dose between 700 mg to 1100 mg of Compound A to the subject in need thereof. In some embodiments, the method comprises administering a total daily dose between 750 mg to 1100 mg of Compound A to the subject in need thereof. In some embodiments, the method comprises administering a total daily dose between 800 mg to 1100 mg of Compound A to the subject in need thereof. In some embodiments, the method comprises administering a total daily dose between 850 mg to 1100 mg of Compound A to the subject in need thereof. In some embodiments, the method comprises administering a total daily dose between 900 mg to 1100 mg of Compound A to the subject in need thereof. In some embodiments, the method comprises administering a total daily dose between 950 mg to 1100 mg of Compound A to the subject in need thereof. In some embodiments, the method comprises administering a total daily dose between 1000 mg to 1100 mg of Compound A to the subject in need thereof.

In some embodiments, the method comprises administering a total daily dose between 100 mg to 1000 mg of Compound A to the subject in need thereof. In some embodiments, the method comprises administering a total daily dose between 150 mg to 1000 mg of Compound A to the subject in need thereof. In some embodiments, the method comprises administering a total daily dose between 200 mg to 1000 mg of Compound A to the subject in need thereof. In some embodiments, the method comprises administering a total daily dose between 250 mg to 1000 mg of Compound A to the subject in need thereof. In some embodiments, the method comprises administering a total daily dose between 300 mg to 1000 mg of Compound A to the subject in need thereof. In some embodiments, the method comprises administering a total daily dose between 350 mg to 1000 mg of Compound A to the subject in need thereof. In some embodiments, the method comprises administering a total daily dose between 400 mg to 1000 mg of Compound A to the subject in need thereof. In some embodiments, the method comprises administering a total daily dose between 450 mg to 1000 mg of Compound A to the subject in need thereof. In some embodiments, the method comprises administering a total daily dose between 500 mg to 1000 mg of Compound A to the subject in need thereof. In some embodiments, the method comprises administering a total daily dose between 550 mg to 1000 mg of Compound A to the subject in need thereof. In some embodiments, the method comprises administering a total daily dose between 600 mg to 1000 mg of Compound A to the subject in need thereof. In some embodiments, the method comprises administering a total daily dose between 650 mg to 1000 mg of Compound A to the subject in need thereof. In some embodiments, the method comprises administering a total daily dose between 700 mg to 1000 mg of Compound A to the subject in need thereof. In some embodiments, the method comprises administering a total daily dose between 750 mg to 1000 mg of Compound A to the subject in need thereof. In some embodiments, the method comprises administering a total daily dose between 800 mg to 1000 mg of Compound A to the subject in need thereof. In some embodiments, the method comprises administering a total daily dose between 850 mg to 1000 mg of Compound A to the subject in need thereof. In some embodiments, the method comprises administering a total daily dose between 900 mg to 1000 mg of Compound A to the subject in need thereof. In some embodiments, the method comprises administering a total daily dose between 950 mg to 1000 mg of Compound A to the subject in need thereof.

In some embodiments, the method comprises administering a total daily dose between 100 mg to 950 mg of Compound A to the subject in need thereof. In some embodiments, the method comprises administering a total daily dose between 150 mg to 950 mg of Compound A to the subject in need thereof. In some embodiments, the method comprises administering a total daily dose between 200 mg to 950 mg of Compound A to the subject in need thereof. In some embodiments, the method comprises administering a total daily dose between 250 mg to 950 mg of Compound A to the subject in need thereof. In some embodiments, the method comprises administering a total daily dose between 300 mg to 950 mg of Compound A to the subject in need thereof. In some embodiments, the method comprises administering a total daily dose between 350 mg to 950 mg of Compound A to the subject in need thereof. In some embodiments, the method comprises administering a total daily dose between 400 mg to 950 mg of Compound A to the subject in need thereof. In some embodiments, the method comprises administering a total daily dose between 450 mg to 950 mg of Compound A to the subject in need thereof. In some embodiments, the method comprises administering a total daily dose between 500 mg to 950 mg of Compound A to the subject in need thereof. In some embodiments, the method comprises administering a total daily dose between 550 mg to 950 mg of Compound A to the subject in need thereof. In some embodiments, the method comprises administering a total daily dose between 600 mg to 950 mg of Compound A to the subject in need thereof. In some embodiments, the method comprises administering a total daily dose between 650 mg to 950 mg of Compound A to the subject in need thereof. In some embodiments, the method comprises administering a total daily dose between 700 mg to 950 mg of Compound A to the subject in need thereof. In some embodiments, the method comprises administering a total daily dose between 750 mg to 950 mg of Compound A to the subject in need thereof. In some embodiments, the method comprises administering a total daily dose between 800 mg to 950 mg of Compound A to the subject in need thereof. In some embodiments, the method comprises administering a total daily dose between 850 mg to 950 mg of Compound A to the subject in need thereof. In some embodiments, the method comprises administering a total daily dose between 900 mg to 950 mg of Compound A to the subject in need thereof.

In some embodiments, the method comprises administering a total daily dose between 100 mg to 900 mg of Compound A to the subject in need thereof. In some embodiments, the method comprises administering a total daily dose between 150 mg to 900 mg of Compound A to the subject in need thereof. In some embodiments, the method comprises administering a total daily dose between 200 mg to 900 mg of Compound A to the subject in need thereof. In some embodiments, the method comprises administering a total daily dose between 250 mg to 900 mg of Compound A to the subject in need thereof. In some embodiments, the method comprises administering a total daily dose between 300 mg to 900 mg of Compound A to the subject in need thereof. In some embodiments, the method comprises administering a total daily dose between 350 mg to 900 mg of Compound A to the subject in need thereof. In some embodiments, the method comprises administering a total daily dose between 400 mg to 900 mg of Compound A to the subject in need thereof. In some embodiments, the method comprises administering a total daily dose between 450 mg to 900 mg of Compound A to the subject in need thereof. In some embodiments, the method comprises administering a total daily dose between 500 mg to 900 mg of Compound A to the subject in need thereof. In some embodiments, the method comprises administering a total daily dose between 550 mg to 900 mg of Compound A to the subject in need thereof. In some embodiments, the method comprises administering a total daily dose between 600 mg to 900 mg of Compound A to the subject in need thereof. In some embodiments, the method comprises administering a total daily dose between 650 mg to 900 mg of Compound A to the subject in need thereof. In some embodiments, the method comprises administering a total daily dose between 700 mg to 900 mg of Compound A to the subject in need thereof. In some embodiments, the method comprises administering a total daily dose between 750 mg to 900 mg of Compound A to the subject in need thereof. In some embodiments, the method comprises administering a total daily dose between 800 mg to 900 mg of Compound A to the subject in need thereof. In some embodiments, the method comprises administering a total daily dose between 850 mg to 900 mg of Compound A to the subject in need thereof.

In some embodiments, the method comprises administering a total daily dose between 100 mg to 850 mg of Compound A to the subject in need thereof. In some embodiments, the method comprises administering a total daily dose between 150 mg to 850 mg of Compound A to the subject in need thereof. In some embodiments, the method comprises administering a total daily dose between 200 mg to 850 mg of Compound A to the subject in need thereof. In some embodiments, the method comprises administering a total daily dose between 250 mg to 850 mg of Compound A to the subject in need thereof. In some embodiments, the method comprises administering a total daily dose between 300 mg to 850 mg of Compound A to the subject in need thereof. In some embodiments, the method comprises administering a total daily dose between 350 mg to 850 mg of Compound A to the subject in need thereof. In some embodiments, the method comprises administering a total daily dose between 400 mg to 850 mg of Compound A to the subject in need thereof. In some embodiments, the method comprises administering a total daily dose between 450 mg to 850 mg of Compound A to the subject in need thereof. In some embodiments, the method comprises administering a total daily dose between 500 mg to 850 mg of Compound A to the subject in need thereof. In some embodiments, the method comprises administering a total daily dose between 550 mg to 850 mg of Compound A to the subject in need thereof. In some embodiments, the method comprises administering a total daily dose between 600 mg to 850 mg of Compound A to the subject in need thereof. In some embodiments, the method comprises administering a total daily dose between 650 mg to 850 mg of Compound A to the subject in need thereof. In some embodiments, the method comprises administering a total daily dose between 700 mg to 850 mg of Compound A to the subject in need thereof. In some embodiments, the method comprises administering a total daily dose between 750 mg to 850 mg of Compound A to the subject in need thereof. In some embodiments, the method comprises administering a total daily dose between 800 mg to 850 mg of Compound A to the subject in need thereof.

In some embodiments, the method comprises administering a total daily dose between 100 mg to 800 mg of Compound A to the subject in need thereof. In some embodiments, the method comprises administering a total daily dose between 150 mg to 800 mg of Compound A to the subject in need thereof. In some embodiments, the method comprises administering a total daily dose between 200 mg to 800 mg of Compound A to the subject in need thereof. In some embodiments, the method comprises administering a total daily dose between 250 mg to 800 mg of Compound A to the subject in need thereof. In some embodiments, the method comprises administering a total daily dose between 300 mg to 800 mg of Compound A to the subject in need thereof. In some embodiments, the method comprises administering a total daily dose between 350 mg to 800 mg of Compound A to the subject in need thereof. In some embodiments, the method comprises administering a total daily dose between 400 mg to 800 mg of Compound A to the subject in need thereof. In some embodiments, the method comprises administering a total daily dose between 450 mg to 800 mg of Compound A to the subject in need thereof. In some embodiments, the method comprises administering a total daily dose between 500 mg to 800 mg of Compound A to the subject in need thereof. In some embodiments, the method comprises administering a total daily dose between 550 mg to 800 mg of Compound A to the subject in need thereof. In some embodiments, the method comprises administering a total daily dose between 600 mg to 800 mg of Compound A to the subject in need thereof. In some embodiments, the method comprises administering a total daily dose between 650 mg to 800 mg of Compound A to the subject in need thereof. In some embodiments, the method comprises administering a total daily dose between 700 mg to 800 mg of Compound A to the subject in need thereof. In some embodiments, the method comprises administering a total daily dose between 750 mg to 800 mg of Compound A to the subject in need thereof.

In some embodiments, the method comprises administering a total daily dose between 100 mg to 750 mg of Compound A to the subject in need thereof. In some embodiments, the method comprises administering a total daily dose between 150 mg to 750 mg of Compound A to the subject in need thereof. In some embodiments, the method comprises administering a total daily dose between 200 mg to 750 mg of Compound A to the subject in need thereof. In some embodiments, the method comprises administering a total daily dose between 250 mg to 750 mg of Compound A to the subject in need thereof. In some embodiments, the method comprises administering a total daily dose between 300 mg to 750 mg of Compound A to the subject in need thereof. In some embodiments, the method comprises administering a total daily dose between 350 mg to 750 mg of Compound A to the subject in need thereof. In some embodiments, the method comprises administering a total daily dose between 400 mg to 750 mg of Compound A to the subject in need thereof. In some embodiments, the method comprises administering a total daily dose between 450 mg to 750 mg of Compound A to the subject in need thereof. In some embodiments, the method comprises administering a total daily dose between 500 mg to 750 mg of Compound A to the subject in need thereof. In some embodiments, the method comprises administering a total daily dose between 550 mg to 750 mg of Compound A to the subject in need thereof. In some embodiments, the method comprises administering a total daily dose between 600 mg to 750 mg of Compound A to the subject in need thereof. In some embodiments, the method comprises administering a total daily dose between 650 mg to 750 mg of Compound A to the subject in need thereof. In some embodiments, the method comprises administering a total daily dose between 700 mg to 750 mg of Compound A to the subject in need thereof.

In some embodiments, the method comprises administering a total daily dose between 100 mg to 700 mg of Compound A to the subject in need thereof. In some embodiments, the method comprises administering a total daily dose between 150 mg to 700 mg of Compound A to the subject in need thereof. In some embodiments, the method comprises administering a total daily dose between 200 mg to 700 mg of Compound A to the subject in need thereof. In some embodiments, the method comprises administering a total daily dose between 250 mg to 700 mg of Compound A to the subject in need thereof. In some embodiments, the method comprises administering a total daily dose between 300 mg to 700 mg of Compound A to the subject in need thereof. In some embodiments, the method comprises administering a total daily dose between 350 mg to 700 mg of Compound A to the subject in need thereof. In some embodiments, the method comprises administering a total daily dose between 400 mg to 700 mg of Compound A to the subject in need thereof. In some embodiments, the method comprises administering a total daily dose between 450 mg to 700 mg of Compound A to the subject in need thereof. In some embodiments, the method comprises administering a total daily dose between 500 mg to 700 mg of Compound A to the subject in need thereof. In some embodiments, the method comprises administering a total daily dose between 550 mg to 700 mg of Compound A to the subject in need thereof. In some embodiments, the method comprises administering a total daily dose between 600 mg to 700 mg of Compound A to the subject in need thereof. In some embodiments, the method comprises administering a total daily dose between 650 mg to 700 mg of Compound A to the subject in need thereof.

In some embodiments, the method comprises administering a total daily dose between 100 mg to 650 mg of Compound A to the subject in need thereof. In some embodiments, the method comprises administering a total daily dose between 150 mg to 650 mg of Compound A to the subject in need thereof. In some embodiments, the method comprises administering a total daily dose between 200 mg to 650 mg of Compound A to the subject in need thereof. In some embodiments, the method comprises administering a total daily dose between 250 mg to 650 mg of Compound A to the subject in need thereof. In some embodiments, the method comprises administering a total daily dose between 300 mg to 650 mg of Compound A to the subject in need thereof. In some embodiments, the method comprises administering a total daily dose between 350 mg to 650 mg of Compound A to the subject in need thereof. In some embodiments, the method comprises administering a total daily dose between 400 mg to 650 mg of Compound A to the subject in need thereof. In some embodiments, the method comprises administering a total daily dose between 450 mg to 650 mg of Compound A to the subject in need thereof. In some embodiments, the method comprises administering a total daily dose between 500 mg to 650 mg of Compound A to the subject in need thereof. In some embodiments, the method comprises administering a total daily dose between 550 mg to 650 mg of Compound A to the subject in need thereof. In some embodiments, the method comprises administering a total daily dose between 600 mg to 650 mg of Compound A to the subject in need thereof.

In some embodiments, the method comprises administering a total daily dose between 100 mg to 600 mg of Compound A to the subject in need thereof. In some embodiments, the method comprises administering a total daily dose between 150 mg to 600 mg of Compound A to the subject in need thereof. In some embodiments, the method comprises administering a total daily dose between 200 mg to 600 mg of Compound A to the subject in need thereof. In some embodiments, the method comprises administering a total daily dose between 250 mg to 600 mg of Compound A to the subject in need thereof. In some embodiments, the method comprises administering a total daily dose between 300 mg to 600 mg of Compound A to the subject in need thereof. In some embodiments, the method comprises administering a total daily dose between 350 mg to 600 mg of Compound A to the subject in need thereof. In some embodiments, the method comprises administering a total daily dose between 400 mg to 600 mg of Compound A to the subject in need thereof. In some embodiments, the method comprises administering a total daily dose between 450 mg to 600 mg of Compound A to the subject in need thereof. In some embodiments, the method comprises administering a total daily dose between 500 mg to 600 mg of Compound A to the subject in need thereof. In some embodiments, the method comprises administering a total daily dose between 550 mg to 600 mg of Compound A to the subject in need thereof.

In some embodiments, the method comprises administering a total daily dose between 100 mg to 550 mg of Compound A to the subject in need thereof. In some embodiments, the method comprises administering a total daily dose between 150 mg to 550 mg of Compound A to the subject in need thereof. In some embodiments, the method comprises administering a total daily dose between 200 mg to 550 mg of Compound A to the subject in need thereof. In some embodiments, the method comprises administering a total daily dose between 250 mg to 550 mg of Compound A to the subject in need thereof. In some embodiments, the method comprises administering a total daily dose between 300 mg to 550 mg of Compound A to the subject in need thereof. In some embodiments, the method comprises administering a total daily dose between 350 mg to 550 mg of Compound A to the subject in need thereof. In some embodiments, the method comprises administering a total daily dose between 400 mg to 550 mg of Compound A to the subject in need thereof. In some embodiments, the method comprises administering a total daily dose between 450 mg to 550 mg of Compound A to the subject in need thereof. In some embodiments, the method comprises administering a total daily dose between 500 mg to 550 mg of Compound A to the subject in need thereof.

In some embodiments, the method comprises administering a total daily dose between 100 mg to 500 mg of Compound A to the subject in need thereof. In some embodiments, the method comprises administering a total daily dose between 150 mg to 500 mg of Compound A to the subject in need thereof. In some embodiments, the method comprises administering a total daily dose between 200 mg to 500 mg of Compound A to the subject in need thereof. In some embodiments, the method comprises administering a total daily dose between 250 mg to 500 mg of Compound A to the subject in need thereof. In some embodiments, the method comprises administering a total daily dose between 300 mg to 500 mg of Compound A to the subject in need thereof. In some embodiments, the method comprises administering a total daily dose between 350 mg to 500 mg of Compound A to the subject in need thereof. In some embodiments, the method comprises administering a total daily dose between 400 mg to 500 mg of Compound A to the subject in need thereof. In some embodiments, the method comprises administering a total daily dose between 450 mg to 500 mg of Compound A to the subject in need thereof.

In some embodiments, the method comprises administering a total daily dose between 100 mg to 450 mg of Compound A to the subject in need thereof. In some embodiments, the method comprises administering a total daily dose between 150 mg to 450 mg of Compound A to the subject in need thereof. In some embodiments, the method comprises administering a total daily dose between 200 mg to 450 mg of Compound A to the subject in need thereof. In some embodiments, the method comprises administering a total daily dose between 250 mg to 450 mg of Compound A to the subject in need thereof. In some embodiments, the method comprises administering a total daily dose between 300 mg to 450 mg of Compound A to the subject in need thereof. In some embodiments, the method comprises administering a total daily dose between 350 mg to 450 mg of Compound A to the subject in need thereof. In some embodiments, the method comprises administering a total daily dose between 400 mg to 450 mg of Compound A to the subject in need thereof.

In some embodiments, the method comprises administering a total daily dose between 100 mg to 400 mg of Compound A to the subject in need thereof. In some embodiments, the method comprises administering a total daily dose between 150 mg to 400 mg of Compound A to the subject in need thereof. In some embodiments, the method comprises administering a total daily dose between 200 mg to 400 mg of Compound A to the subject in need thereof. In some embodiments, the method comprises administering a total daily dose between 250 mg to 400 mg of Compound A to the subject in need thereof. In some embodiments, the method comprises administering a total daily dose between 300 mg to 400 mg of Compound A to the subject in need thereof. In some embodiments, the method comprises administering a total daily dose between 350 mg to 400 mg of Compound A to the subject in need thereof.

In some embodiments, the method comprises administering a total daily dose between 100 mg to 350 mg of Compound A to the subject in need thereof. In some embodiments, the method comprises administering a total daily dose between 150 mg to 350 mg of Compound A to the subject in need thereof. In some embodiments, the method comprises administering a total daily dose between 200 mg to 350 mg of Compound A to the subject in need thereof. In some embodiments, the method comprises administering a total daily dose between 250 mg to 350 mg of Compound A to the subject in need thereof. In some embodiments, the method comprises administering a total daily dose between 300 mg to 350 mg of Compound A to the subject in need thereof.

In some embodiments, the method comprises administering a total daily dose between 100 mg to 300 mg of Compound A to the subject in need thereof. In some embodiments, the method comprises administering a total daily dose between 150 mg to 300 mg of Compound A to the subject in need thereof. In some embodiments, the method comprises administering a total daily dose between 200 mg to 300 mg of Compound A to the subject in need thereof. In some embodiments, the method comprises administering a total daily dose between 250 mg to 300 mg of Compound A to the subject in need thereof.

In some embodiments, the method comprises administering a total daily dose between 100 mg to 250 mg of Compound A to the subject in need thereof. In some embodiments, the method comprises administering a total daily dose between 150 mg to 250 mg of Compound A to the subject in need thereof. In some embodiments, the method comprises administering a total daily dose between 200 mg to 250 mg of Compound A to the subject in need thereof.

In some embodiments, the method comprises administering a total daily dose between 100 mg to 200 mg of Compound A to the subject in need thereof. In some embodiments, the method comprises administering a total daily dose between 150 mg to 200 mg of Compound A to the subject in need thereof.

In some embodiments, the method comprises administering a total daily dose between 100 mg to 150 mg of Compound A to the subject in need thereof.

In various embodiments, Compound A is administered daily. In some embodiments, Compound A is administered once, twice, or more daily. In some embodiments, Compound A is administered twice daily. In various embodiments, Compound A is administered in a divided daily dose, such as two, three, four, five, or six times a day.

In some embodiments of the methods disclosed herein, the subject is administered Compound A at a disclosed dose orally at least once daily (QD).

In some embodiments of the methods disclosed herein, the subject is administered Compound A at a disclosed dose orally at least twice daily (BID).

In some embodiments of the method disclosed herein, the subject is administered 300 mg to 600 mg twice daily of Compound A. In some embodiments, the method includes administering 400 mg to 500 mg twice daily of Compound A to the subject in need thereof. In some embodiments, the method includes administering 300 mg twice daily of Compound A to the subject in need thereof. In some embodiments, the method includes administering 450 mg twice daily of Compound A to the subject in need thereof. In some embodiments, the method includes administering 600 mg twice daily of Compound A to the subject in need thereof.

In various embodiments, Compound A is administered with food. In various embodiments, Compound A is administered without food.

In various embodiments, Compound A is administered 1, 2, 3, 4, 5, 6 or 7 times per week. In various embodiments, Compound A is administered 7 days per week. In various embodiments, Compound A is administered 6 days per week. For example, Compound A is administered on days 1, 2, 3, 4, 5, and 6 days of each 7 days. In various embodiments, Compound A is administered 5 days per week. For example, Compound A is administered on days 1, 2, 3, 4, and 5 days of each 7 days. In various embodiments, Compound A is administered 4 days per week. For example, Compound A is administered on days 1, 2, 3, and 4 days of each 7 days. In various embodiments, Compound A is administered 3 days per week. For example, Compound A is administered on days 1, 2, and 3 of each 7 days. In various embodiments, Compound A is administered 2 days per week. For example, Compound A is administered on days 1 and 2 of each 7 days.

In various embodiments, the subject is administered Compound A for at least 1 month, at least 2 months, at least 3 months, at least 4 months, at least 5 months, at least 6 months, at least 7 months, at least 8 months, at least 9 months, at least 10 months, at least 11 months, at least 12 months, at least 15 months, at least 18 months, at least 21 months, or at least 23 months, e.g., for 1 month, 2 months, 3 months, 4 months, 5 months, 6 months, 7 months, 8 months, 9 months, 10 months, 11 months, 12 months, 15 months, 18 months, 21 months, 24 months or longer. In various embodiments, the subject is administered Compound A for at least 1 month. In various embodiments, the subject is administered Compound A for at least 3 months. In various embodiments, the subject is administered Compound A for at least 6 months. In various embodiments, the subject is administered Compound A for at least 8 months. In various embodiments, the subject is administered Compound A for at least 10 months. In various embodiments, the subject is administered Compound A for at least 12 months.

In some embodiments, Compound A is administered in treatment cycles. In some embodiments, the treatment cycle is 7 days, 14 days, 21 days, 28 days, 1 month, 2 months, 3 months, 4 months, 5 months, 6 months, 7 months, 8 months, 9 months, 10 months, 11 months or 1 year. In various embodiments, the subject undergoes 1, 2, 3, 4, or more treatment cycles. In some embodiments, the subject undergoes at least 3 treatment cycles, at least 5 treatment cycles, at least 8 treatment cycles, at least 10 treatment cycles, at least 15 treatment cycles, at least 20 treatment cycles, at least 25 treatment cycles or more.

In some embodiments, the subject is human. In some embodiments, the subject is a male. In some embodiments, the subject is a female. In some embodiments, the subject is at least about any of 18, 20, 25, 30, 35, 40, 45, 50, 55, 60, 65, 70, 75, 80, 85, or 90 years old. In some embodiments, the subject is under about any of 25, 30, 35, 40, 45, 50, 55, 60, 65, 70, 75, 80, 85, or 90 years old. In some embodiments, the subject has one or more of the characteristics of the patients described in the examples of the present disclosure.

In another aspect, the disclosure features a method of administering Compound A to a subject in need thereof, the method including administering to the subject a therapeutically effective amount of Compound A and avoiding co-administration of a cytochrome p450 (CYP) 3A4 inhibitor, wherein said subject is also in need of a CYP3A4 inhibitor. In some embodiments, the methods include administering a Compound A to a subject who has discontinued use or avoids concomitant use of products comprising a CYP3A4 inhibitor. In various embodiments, the subject is in further need of treatment with a CYP3A4 inhibitor. In some embodiments, the subject is not administered a CYP3A4 inhibitor in combination with Compound A. Exemplary CYP3A4 inhibitors include, but are not limited to, boceprevir, clarithromycin, cobicistat, danoprevir and ritonavir, elvitegravir and ritonavir, grapefruit juice, idelalisib, indinavir and ritonavir, itraconazole, ketoconazole, lopinavir and ritonavir, nefazodone, nelfinavir, paritaprevir and ritonavir and (ombitasvir and/or dasabuvir), posaconazole, ritonavir, saquinavir and ritonavir, telaprevir, tipranavir and ritonavir, telithromycin, troleandomycin, voriconazole. In some embodiments, the subject has a RAS G12D mutant cancer.

In another aspect, the disclosure provides methods of administering Compound A to a subject in need thereof, the methods include discontinuing administration of a CYP3A4 inhibitor to avoid an adverse drug interaction with Compound A, and administering to the subject a therapeutically effective amount of Compound A. In some embodiments, the CYP3A4 inhibitor is discontinued within 1 month prior to being administered Compound A. In some embodiments, the CYP3A4 inhibitor is discontinued within 2 weeks prior to being administered Compound A. In some embodiments, the subject is advised that co-administration of Compound A and the CYP3A4 inhibitor can alter the therapeutic effect or adverse reaction profile of Compound A.

In another aspect, the disclosure provides methods of administering Compound A to a subject in need thereof, wherein said subject is also in need of a CYP3A4 inhibitor, the method including administering to the subject a daily dosage of 100 mg to 1400 mg of Compound A while avoiding CYP3A4 inhibitor co-administration, and any one or more of the following:
(a) advising the subject that the CYP3A4 inhibitor should be avoided or discontinued,
(b) advising the subject that co-administration of Compound A with the CYP3A4 inhibitor can alter the therapeutic effect or adverse reaction profile of Compound A,
(c) advising the subject that use of Compound A in subjects being treated with the CYP3A4 inhibitor is contraindicated, or
(d) advising the subject that CYP3A4 inhibitors should be used with caution in subjects receiving Compound A due to the potential for an adverse reaction profile. In some embodiments, the method further includes avoiding administering the CYP3A4 inhibitor. In some embodiments, the method further includes discontinuing administration of the CYP3A4 inhibitor.

In yet another aspect, the disclosure features methods of administering Compound A to a subject in need thereof, the methods include administering to the subject a therapeutically effective amount of Compound A and avoiding co-administration of a cytochrome p450 (CYP) 3A4 substrate, wherein said subject is also in need of the CYP3A4 substrate. Exemplary CYP3A4 substrates include, but are not limited to, alfentanil, alprazolam, aprepitant, atorvastatin, avanafil, budesonide, buspirone, colchicine, conivaptan, darifenacin, darunavir, dasatinib, dipivefrine, dronedarone, ebastine, eletriptan, eliglustat, eplerenone, everolimus, felodipine, ibrutinib, indinavir, isavuconazole, ivabradine, lemborexant, lomitapide, lovastatin, lurasidone, maraviroc, midazolam, mobocertinib, naloxegol, nisoldipine, pimozide, quetiapine, rivaroxaban, saquinavir, simvastatin, sirolimus, tacrolimus, tadalafil, ticagrelor, tipranavir, tolvaptan, triazolam, vardenafil, astemizole, conivaptan, cyclosporine, dronedarone, everolimus, levomethadyl acetate, lomitapide, pimozide, quinidine, sirolimus, tacrolimus, tolvaptan, and venetoclax.

In some embodiments, the methods include administering Compound A to a subject in need thereof, the method including discontinuing administration of a CYP3A4 substrate to avoid an adverse drug interaction with Compound A, and administering to the subject a therapeutically effective amount of Compound A. In some embodiments, the CYP3A4 substrate is discontinued within 1 month prior to being administered Compound A. In some embodiments, the CYP3A4 substrate is discontinued within 2 weeks prior to being administered Compound A. In some embodiments, the subject is advised that co-administration of Compound A and the CYP3A4 substrate can alter the therapeutic effect or adverse reaction profile of Compound A.

In some embodiments, the methods include administering Compound A to a subject in need thereof, wherein said subject is also in need of a CYP3A4 substrate, the method includes administering to the subject 100 mg to 1400 mg Compound A per day while avoiding CYP3A4 substrate co-administration, and any one or more of the following:
(a) advising the subject that CYP3A4 substrate should be avoided or discontinued,
(b) advising the subject that co-administration of Compound A with CYP3A4 substrates can alter the therapeutic effect or adverse reaction profile of Compound A,
(c) advising the subject that use of Compound A in subjects being treated with CYP3A4 substrates is contraindicated, or
(d) advising the subject that CYP3A4 substrates should be used with caution in subjects receiving Compound A due to the potential for an adverse reaction profile. In some embodiments, the method further includes avoiding administering the CYP3A4 substrate. In some embodiments, the method further includes discontinuing administration of the CYP3A4 substrate.

In yet another aspect, the disclosure features methods of administering Compound A to a subject in need thereof, the methods include administering to the subject a therapeutically effective amount of Compound A and avoiding co-administration of a cytochrome p450 (CYP) 3A4 inducer, wherein said subject is also in need of the CYP3A4 inducer. Exemplary CYP3A4 inducers include, but are not limited to, apalutamide, carbamazepine, enzalutamide, mitotane, phenytoin, St. John's wort, and rifampin.

In some embodiments, the methods include administering Compound A to a subject in need thereof, the method includes discontinuing administration of a CYP3A4 inducer to avoid an adverse drug interaction with Compound A, and administering to the subject a therapeutically effective amount of Compound A. In some embodiments, the CYP3A4 inducer is discontinued within 1 month prior to being administered Compound A. In some embodiments, the CYP3A4 inducer is discontinued within 2 weeks prior to being administered Compound A. In some embodiments, the subject is advised that co-administration of Compound A and the CYP3A4 inducer can alter the therapeutic effect or adverse reaction profile of Compound A.

In some embodiments, the methods include administering Compound A to a subject in need thereof, wherein said subject is also in need of a CYP3A4 inducer, the method includes administering to the subject 100 mg to 1400 mg Compound A per day while avoiding CYP3A4 inducer co-administration, and any one or more of the following:
(a) advising the subject that CYP3A4 inducer should be avoided or discontinued,
(b) advising the subject that co-administration of Compound A with CYP3A4 inducer can alter the therapeutic effect or adverse reaction profile Compound A,
(c) advising the subject that use of Compound A in subjects being treated with CYP3A4 inducer is contraindicated, or
(d) advising the subject that CYP3A4 inducers should be used with caution in subjects receiving Compound A due to the potential for an adverse reaction profile. In some embodiments, the method further includes avoiding administering the CYP3A4 inducer. In some embodiments, the method further includes discontinuing administration of the CYP3A4 inducer.

In another aspect, the disclosure features methods of administering Compound A to a subject in need thereof, the methods include administering to the subject a therapeutically effective amount of Compound A and avoiding co-administration of a proton pump inhibitor (PPI), wherein said subject is also in need of the PPI. Exemplary PPIs include, but are not limited to, dexlansoprazole, esomeprazole, lansoprazole, omeprazole, pantoprazole, and rabeprazole.

In some embodiments, the methods include administering Compound A to a subject in need thereof, the method includes discontinuing administration of a PPI to avoid an adverse drug interaction with Compound A, and administering to the subject a therapeutically effective amount of Compound A. In some embodiments, the PPI is discontinued within 1 month prior to being administered Compound A. In some embodiments, the PPI is discontinued within 2 weeks prior to being administered Compound A. In some embodiments, the subject is advised that co-administration of Compound A and the PPI can alter the therapeutic effect or adverse reaction profile of Compound A.

In some embodiments, the methods include administering Compound A to a subject in need thereof, wherein said subject is also in need of a PPI, the method includes administering to the subject 100 mg to 1400 mg Compound A per day while avoiding PPI co-administration, and any one or more of the following:
(a) advising the subject that a PPI should be avoided or discontinued,
(b) advising the subject that co-administration of Compound A with a PPI can alter the therapeutic effect or adverse reaction profile Compound A,
(c) advising the subject that use of Compound A in subjects being treated with a PPI is contraindicated, or
(d) advising the subject that PPIs should be used with caution in subjects receiving Compound A due to the potential for an adverse reaction profile. In some embodiments, the method further includes avoiding administering the PPI. In some embodiments, the method further includes discontinuing administration of the PPI.

In still another aspect, the disclosure features methods of administering Compound A to a subject in need thereof, the methods include administering to the subject a therapeutically effective amount of Compound A and avoiding co-administration of a H-2 receptor antagonists (H2 blockers), wherein said subject is also in need of the H2 blockers. Exemplary H2 blockers include, but are not limited to, cimetidine, famotidine, nizatidine, and ranitidine.

In some embodiments, the methods include administering Compound A to a subject in need thereof, the method includes discontinuing administration of a H2 blocker to avoid an adverse drug interaction with Compound A, and administering to the subject a therapeutically effective amount of Compound A. In some embodiments, the H2 blocker is discontinued within 1 month prior to being administered Compound A. In some embodiments, the H2 blocker is discontinued within 2 weeks prior to being administered Compound A. In some embodiments, the subject is advised that co-administration of Compound A and the H2 blocker can alter the therapeutic effect or adverse reaction profile of Compound A.

In some embodiments, the methods include administering Compound A to a subject in need thereof, wherein said subject is also in need of a H2 blocker, the method includes administering to the subject 100 mg to 1400 mg Compound A per day while avoiding H2 blocker co-administration, and any one or more of the following:
(a) advising the subject that a H2 blocker should be avoided or discontinued,
(b) advising the subject that co-administration of Compound A with a H2 blocker can alter the therapeutic effect or adverse reaction profile Compound A,
(c) advising the subject that use of Compound A in subjects being treated with a H2 blocker is contraindicated, or
(d) advising the subject that H2 blockers should be used with caution in subjects receiving Compound A due to the potential for an adverse reaction profile. In some embodiments, the method further includes avoiding administering the H2 blocker. In some embodiments, the method further includes discontinuing administration of the H2 blocker.

In another aspect, the disclosure features methods of administering Compound A to a subject in need thereof, the methods include administering to the subject a therapeutically effective amount of Compound A and avoiding co-administration of disease-modifying antirheumaic drugs (DMARDS), wherein said subject is also in need of the DMARDS. Exemplary DMARDS include, but are not limited to, abatacept, apremilast, azathioprine, belimumab, ciclosporin, hydroxychloroquine, ixekizumab, leflunomide, methotrexate, mycophenolate, rituximab, sarilumab, secukinumab, sulfasalazine, tocilizumab, and ustekinumab.

In some embodiments, the methods include administering Compound A to a subject in need thereof, the method includes discontinuing administration of a DMARD to avoid an adverse drug interaction with Compound A, and administering to the subject a therapeutically effective amount of Compound A. In some embodiments, the DMARD is discontinued within 1 month prior to being administered Compound A. In some embodiments, the DMARD is discontinued within 2 weeks prior to being administered Compound A. In some embodiments, the subject is advised that co-administration of Compound A and the DMARD can alter the therapeutic effect or adverse reaction profile of Compound A.

In some embodiments, the methods include administering Compound A to a subject in need thereof, wherein said subject is also in need of a DMARD, the method includes administering to the subject 100 mg to 1400 mg Compound A per day while avoiding DMARD co-administration, and any one or more of the following:
(a) advising the subject that a DMARD should be avoided or discontinued,
(b) advising the subject that co-administration of Compound A with a DMARD can alter the therapeutic effect or adverse reaction profile Compound A,
(c) advising the subject that use of Compound A in subjects being treated with a DMARD is contraindicated, or
(d) advising the subject that DMARDS should be used with caution in subjects receiving Compound A due to the potential for an adverse reaction profile. In some embodiments, the method further includes avoiding administering the DMARD. In some embodiments, the method further includes discontinuing administration of the DMARD.

In yet another aspect, the disclosure features methods of administering Compound A to a subject in need thereof, the methods include administering to the subject a therapeutically effective amount of Compound A and avoiding co-administration of an immunosuppressive agent, wherein said subject is also in need of the immunosuppressive agent. Exemplary immunosuppressive agents include, but are not limited to, adalimumab, anakinra, certolizumab, cyclosporine, etanercept, everolimus, golimumab, infliximab, natalizumab, sirolimus, tacrolimus, tofacitinib, and vedolizumab.

In some embodiments, the methods include administering Compound A to a subject in need thereof comprising discontinuing administration of an immunosuppressive agent to avoid an adverse drug interaction with Compound A, and administering to the subject a therapeutically effective amount of Compound A. In some embodiments, the immunosuppressive agent is discontinued within 1 month prior to being administered Compound A. In some embodiments, the immunosuppressive agent is discontinued within 2 weeks prior to being administered Compound A. In some embodiments, the subject is advised that co-administration of Compound A and the immunosuppressive agent can alter the therapeutic effect or adverse reaction profile of Compound A.

In some embodiments, the methods include administering Compound A to a subject in need thereof, wherein said subject is also in need of an immunosuppressive agent, the method includes administering to the subject 100 mg to 1400 mg Compound A per day while avoiding immunosuppressive agent co-administration, and any one or more of the following:
(a) advising the subject that an immunosuppressive agent should be avoided or discontinued,
(b) advising the subject that co-administration of Compound A with an immunosuppressive agent can alter the therapeutic effect or adverse reaction profile Compound A,
(c) advising the subject that use of Compound A in subjects being treated with an immunosuppressive agent is contraindicated, or
(d) advising the subject that immunosuppressive agents should be used with caution in subjects receiving Compound A due to the potential for an adverse reaction profile. In some embodiments, the method further includes avoiding administering the immunosuppressive agent. In some embodiments, the method further includes discontinuing administration of the immunosuppressive agent.

In another aspect, the disclosures provides methods of administering Compound A to a subject in need thereof, the method includes administering to the subject a therapeutically effective amount of Compound A, and avoiding co-administration of a P-glycoprotein (P-gp) substrate, wherein said subject is also in need of P-gp substrate. Exemplary P-gp substrates include, but are not limited to, dabigatran, etexilate, and fexofenadine.

In some embodiments, the methods include administering Compound A to a subject in need thereof comprising discontinuing administration of a P-gp substrate to avoid an adverse drug interaction with Compound A, and administering to the subject a therapeutically effective amount of Compound A. In some embodiments, the P-gp substrate is discontinued within 1 month prior to being administered Compound A. In some embodiments, the P-gp substrate is discontinued within 2 weeks prior to being administered Compound A. In some embodiments, the subject is advised that co-administration of Compound A and the P-gp substrate can alter the therapeutic effect or adverse reaction profile of Compound A.

In some embodiments, the methods include administering Compound A to a subject in need thereof, wherein said subject is also in need of a P-gp substrate, the method includes administering to the subject 100 mg to 1400 mg Compound A per day while avoiding immunosuppressive agent co-administration, and any one or more of the following:
(a) advising the subject that a P-gp substrate should be avoided or discontinued,
(b) advising the subject that co-administration of Compound A with a P-gp substrate can alter the therapeutic effect or adverse reaction profile Compound A,
(c) advising the subject that use of Compound A in subjects being treated with a P-gp substrate is contraindicated, or
(d) advising the subject that P-gp substrates should be used with caution in subjects receiving Compound A due to the potential for an adverse reaction profile. In some embodiments, the method further includes avoiding administering the P-gp substrate. In some embodiments, the method further includes discontinuing administration of the P-gp substrate.

In still another aspect, the disclosures provides methods of administering Compound A to a subject in need thereof, the method includes administering to the subject a therapeutically effective amount of Compound A, and avoiding co-administration of a P-glycoprotein (P-gp) inhibitor, wherein said subject is also in need of P-gp inhibitor. Exemplary P-gp inhibitors include, but are not limited to, amiodarone, carvedilol, clarithromycin, dronedarone, gleceprevir and pibrentavir, indinavir, indinavir and ritonavir, itraconazole, lapatinib, lopinavir and ritonavir, propafenone, ranolazine, ritonavir, saquinavir and ritonavir, telaprevir, tipranavir and ritonavir, valspodar, and verapamil.

In some embodiments, the methods include administering Compound A to a subject in need thereof comprising discontinuing administration of a P-gp inhibitor to avoid an adverse drug interaction with Compound A, and administering to the subject a therapeutically effective amount of Compound A. In some embodiments, the P-gp inhibitor is discontinued within 1 month prior to being administered Compound A. In some embodiments, the P-gp inhibitor is discontinued within 2 weeks prior to being administered Compound A. In some embodiments, the subject is advised that co-administration of Compound A and the P-gp inhibitor can alter the therapeutic effect or adverse reaction profile of Compound A.

In some embodiments, the methods include administering Compound A to a subject in need thereof, wherein said subject is also in need of a P-gp inhibitor, the method includes administering to the subject 100 mg to 1400 mg Compound A per day while avoiding immunosuppressive agent co-administration, and any one or more of the following:
(a) advising the subject that a P-gp inhibitor should be avoided or discontinued,
(b) advising the subject that co-administration of Compound A with a P-gp inhibitor can alter the therapeutic effect or adverse reaction profile Compound A,
(c) advising the subject that use of Compound A in subjects being treated with a P-gp inhibitor is contraindicated, or
(d) advising the subject that P-gp inhibitors should be used with caution in subjects receiving Compound A due to the potential for an adverse reaction profile. In some embodiments, the method further includes avoiding administering the P-gp inhibitor. In some embodiments, the method further includes discontinuing administration of the P-gp inhibitor.

In another aspect, the disclosures provides methods of administering Compound A to a subject in need thereof, the method includes administering to the subject a therapeutically effective amount of Compound A, and avoiding co-administration of an organic anion-transporting polypeptide 1B1 or 1B3 (OATP1B1 or OATP1B3) substrate, wherein said subject is also in need of a OATP1B1 or OATP1B3 substrate. Exemplary OATP1B1 or OATP1B3 substrates include, but are not limited to, asunaprevir, atorvastatin, bosentan, danoprevir, fexofenadine, glyburide, nateglinide, pitavastatin, pravastatin, repaglinide, rosuvastatin, and simvastatin acid.

In some embodiments, the methods include administering Compound A to a subject in need thereof comprising discontinuing administration of a OATP1B1 or OATP1B3 substrate to avoid an adverse drug interaction with Compound A, and administering to the subject a therapeutically effective amount of Compound A. In some embodiments, the OATP1B1 or OATP1B3 substrate is discontinued within 1 month prior to being administered Compound A. In some embodiments, the OATP1B1 or OATP1B3 substrate is discontinued within 2 weeks prior to being administered Compound A. In some embodiments, the subject is advised that co-administration of Compound A and the OATP1B1 or OATP1B3 substrate can alter the therapeutic effect or adverse reaction profile of Compound A.

In some embodiments, the methods include administering Compound A to a subject in need thereof, wherein said subject is also in need of a OATP1B1 or OATP1B3 substrate, the method includes administering to the subject 100 mg to 1400 mg Compound A per day while avoiding immunosuppressive agent co-administration, and any one or more of the following:
(a) advising the subject that a OATP1B1 or OATP1B3 substrate should be avoided or discontinued,
(b) advising the subject that co-administration of Compound A with a OATP1B1 or OATP1B3 substrate can alter the therapeutic effect or adverse reaction profile Compound A,
(c) advising the subject that use of Compound A in subjects being treated with a OATP1B1 or OATP1B3 substrate is contraindicated, or
(d) advising the subject that OATP1B1 or OATP1B3 substrates should be used with caution in subjects receiving Compound A due to the potential for an adverse reaction profile. In some embodiments, the method further includes avoiding administering the OATP1B1 or OATP1B3 substrate.

In some embodiments, the method further includes discontinuing administration of the OATP1B1 or OATP1B3 substrate.

In another aspect, the disclosures provides methods of administering Compound A to a subject in need thereof, the method includes administering to the subject a therapeutically effective amount of Compound A, and avoiding co-administration of a breast cancer resistance protein B3 (BCRP) substrate, wherein said subject is also in need of a BCRP substrate. Exemplary BCRP substrates include, but are not limited to, rosuvastatin, simvastatin, and sulfasalazine.

In some embodiments, the methods include administering Compound A to a subject in need thereof comprising discontinuing administration of a BCRP substrate to avoid an adverse drug interaction with Compound A, and administering to the subject a therapeutically effective amount of Compound A. In some embodiments, the BCRP substrate is discontinued within 1 month prior to being administered Compound A. In some embodiments, the BCRP substrate is discontinued within 2 weeks prior to being administered Compound A. In some embodiments, the subject is advised that co-administration of Compound A and the BCRP substrate can alter the therapeutic effect or adverse reaction profile of Compound A.

In some embodiments, the methods include administering Compound A to a subject in need thereof, wherein said subject is also in need of a BCRP substrate, the method includes administering to the subject 100 mg to 1400 mg Compound A per day while avoiding immunosuppressive agent co-administration, and any one or more of the following:
(a) advising the subject that a BCRP substrate should be avoided or discontinued,
(b) advising the subject that co-administration of Compound A with a BCRP substrate can alter the therapeutic effect or adverse reaction profile Compound A,
(c) advising the subject that use of Compound A in subjects being treated with a BCRP substrate is contraindicated, or
(d) advising the subject that BCRP substrates should be used with caution in subjects receiving Compound A due to the potential for an adverse reaction profile. In some embodiments, the method further includes avoiding administering the BCRP substrate. In some embodiments, the method further includes discontinuing administration of the BCRP substrate.

In another aspect, the disclosures provides methods of administering Compound A to a subject in need thereof, the method includes administering to the subject a therapeutically effective amount of Compound A, and avoiding co-administration of a drug known to prolong QTc interval, wherein said subject is also in need of a drug known to prolong QTc interval. Exemplary drugs known to prolong QTc interval include, but are not limited to, amiodarone, anagrelide, arsenic trioxide, azithromycin, chloroquine, chlorpromazine, cilostazol, ciprofloxacin, citalopram, disopyramide, dofetilide, donepezil, dronedarone, droperidol, erythromycin, escitalopram, flecainide, fluconazole, haloperidol, ibutilide, levofloxacin, methadone, moxifloxacin, ondansetron, oxaliplatin, pentamidine, pimozide, procainamide, propofol, quinidine, sevoflurane, sotalol, thioridazine, and vandetanib.

In some embodiments, the methods include administering Compound A to a subject in need thereof comprising discontinuing administration of a drug known to prolong QTc interval to avoid an adverse drug interaction with Compound A, and administering to the subject a therapeutically effective amount of Compound A. In some embodiments, the drug known to prolong QTc interval is discontinued within 1 month prior to being administered Compound A. In some embodiments, the drug known to prolong QTc interval is discontinued within 2 weeks prior to being administered Compound A. In some embodiments, the subject is advised that co-administration of Compound A and the drug known to prolong QTc interval can alter the therapeutic effect or adverse reaction profile of Compound A.

In some embodiments, the methods include administering Compound A to a subject in need thereof, wherein said subject is also in need of a drug known to prolong QTc interval, the method includes administering to the subject 100 mg to 1400 mg Compound A per day while avoiding immunosuppressive agent co-administration, and any one or more of the following:
(a) advising the subject that a drug known to prolong QTc interval should be avoided or discontinued,
(b) advising the subject that co-administration of Compound A with a drug known to prolong QTc interval can alter the therapeutic effect or adverse reaction profile Compound A,
(c) advising the subject that use of Compound A in subjects being treated with a drug known to prolong QTc interval is contraindicated, or
(d) advising the subject that drugs known to prolong QTc interval should be used with caution in subjects receiving Compound A due to the potential for an adverse reaction profile. In some embodiments, the method further includes avoiding administering the drug known to prolong QTc interval. In some embodiments, the method further includes discontinuing administration of the drug known to prolong QTc interval.

In various embodiments, the disclosure provides a method of treating a subject in need of a therapy comprising Compound A, the method generally comprising administering Compound A at an initial dose and, upon the occurrence of an adverse event, administering a reduced dose of Compound A. The adverse event may be any treatment-related adverse event, intolerance, or clinical finding warranting dose modification. In some cases, the adverse event prompting dose reduction may include but is not limited to fatigue, gastrointestinal toxicity, rash, or laboratory abnormalities such as elevated liver enzymes. The dose reduction maintains therapeutic exposure while minimizing further toxicity. The dose reduction may occur after temporary interruption of treatment with Compound A or without interruption, depending on the severity of the adverse event. Dose reductions may be implemented sequentially or as a single step, and may occur with either once daily (QD) or twice daily (BID) dosing regimens. The reduced dose may be maintained until the adverse event resolves or stabilizes, and the subject may optionally be re-escalated to the initial dose or further reduced in dose as clinically indicated.

In some embodiments, the method comprises administering Compound A to the subject at a dose of about 1200 mg (QD or BID) and, upon observation of an adverse event, administering to the subject Compound A subsequent dose of about 900 mg (QD or BID). In another embodiment, the method comprises administering Compound A to the subject at a dose of about 900 mg (QD or BID) and, upon occurrence of an adverse event, administering to the subject Compound A at a subsequent dose of about 600 mg (QD or BID). In another embodiment, the method comprises administering Compound A to the subject at a dose of about 600 mg (QD or BID) and, upon occurrence of an adverse event, administering to the subject Compound A at a subsequent dose of about 300 mg (QD or BID).

In some embodiments, the subject is monitored for the occurrence, severity, and resolution of adverse events throughout the course of treatment. Monitoring may include clinical assessment, physical examination, laboratory testing, or patient-reported outcomes.

In some embodiments, the method further comprises re-initiating treatment at the next lower dose following temporary discontinuation, or maintaining the reduced dose for the remainder of therapy. The dose-reduction sequence may proceed stepwise through one or more levels, for example from about 1200 mg total daily dose, to about 900 mg total daily dose, to about 600 mg total daily, dose to about 300 mg total daily dose, as necessary to maintain tolerability while preserving clinical efficacy.

In another aspect, the methods of the present disclosure include a method of reducing a treatment related adverse event in a subject in need of treatment with Compound A, the method comprising administering to the subject an amount of Compound A as disclosed herein. In some embodiments, the methods include reducing nausea in a subject in need of treatment of Compound A comprising administering between 100 mg to 1400 mg of Compound A daily. In some embodiments, the methods include reducing diarrhea in a subject in need of treatment of Compound A comprising administering between 100 mg to 1400 mg of Compound A daily. In some embodiments, the methods include reducing vomiting in a subject in need of treatment of Compound A comprising administering between 100 mg to 1400 mg of Compound A daily. In some embodiments, the methods include reducing fatigue in a subject in need of treatment of Compound A comprising administering between 100 mg to 1400 mg of Compound A daily. In some embodiments, the methods include reducing rash in a subject in need of treatment of Compound A comprising administering between 100 mg to 1400 mg of Compound A daily. In some embodiments, the methods include reducing increased alanine transaminase in a subject in need of treatment of Compound A comprising administering between 100 mg to 1400 mg of Compound A daily. In some embodiments, the methods include reducing increased aspartate transferase in a subject in need of treatment of Compound A comprising administering between 100 mg to 1400 mg of Compound A daily. In some embodiments, the methods include reducing stomatitis in a subject in need of treatment of Compound A comprising administering between 100 mg to 1400 mg of Compound A daily. In some embodiments, the methods include reducing mucositis in a subject in need of treatment of Compound A comprising administering between 100 mg to 1400 mg of Compound A daily.

In some embodiments, a method of reducing a treatment related adverse event in a subject includes reducing the dose of Compound A administered to a subject in need thereof from 1400 mg of Compound A daily to 1200 mg of Compound A daily. In some embodiments, a method of reducing a treatment related adverse event in a subject includes reducing the dose of Compound A administered to a subject in need thereof from 1200 mg of Compound A daily to 900 mg of Compound A daily. In some embodiments, a method of reducing a treatment related adverse event in a subject includes reducing the dose of Compound A administered to a subject in need thereof from 900 mg of Compound A daily to 600 mg of Compound A daily. In some embodiments, a method of reducing a treatment related adverse event in a subject includes reducing the dose of Compound A administered to a subject in need thereof from 600 mg of Compound A daily to 300 mg of Compound A daily.

In some embodiments, the disclosure provides a method of treating a disease that is characterized by aberrant RAS activity due to a RAS G12D mutation. In some embodiments, the disease is a RASopathy. In some embodiments, the disease is a cancer.

In some embodiments, the disclosure provides a method of treating a disease or disorder that is characterized by aberrant RAS activity due to a RAS mutant. In some embodiments, the disease or disorder is a RASopathy. In some embodiments, the disease or disorder is a cancer.

Accordingly, also provided is a method of treating a RASopathy in a subject in need thereof, the method comprising administering to the subject a therapeutically effective amount of Compound A or a pharmaceutical composition comprising such a compound. A RASopathy is a group of genetic disorders that are caused by mutations in genes involved in the RAS/MAPK signaling pathway. RASopathies are characterized by a range of clinical features and can affect multiple organ systems, including the cardiovascular, musculoskeletal, neurological, and dermatological systems.

In some embodiments, the methods include treating a RASopathy selected from Noonan syndrome, Costello syndrome, cardiofaciocutaneous syndrome, neurofibromatosis type 1, and Legius syndrome. While each RASopathy has unique features, they all share certain similarities, such as facial dysmorphisms, cardiac abnormalities, developmental delays, and an increased risk of certain cancers.

RASopathies are typically diagnosed through a combination of clinical evaluation, genetic testing, and imaging studies. Treatment and management of RASopathies depend on the specific type and severity of the disorder, but may include medication, surgery, and supportive therapies such as physical and occupational therapy.

Exemplary RAS related non-cancerous indications are summarized in Table 1.

TABLE 1
Exemplary RAS related Non-cancerous Indications

| Disease or disorder | | References |
|---|---|---|
| Immune disease | Autoimmune disease | Journal of Clinical Immunology vol. 35, pp. 454-458 |
| | | (2015) |
| | Rheumatoid arthritis | The Open Rheumatoid Journal vol. 6, pp. 259-272 |
| | | (2012) |
| | RAS-related autoimmune | PNAS vol. 104, pp. 8953-8958 (2007) |
| | lymphoproliferative disorders | Blood vol. 117, pp. 2887-2890 (2011) |
| Infection | Influenza | Cancer Research vol. 61, pp. 8188-8193 (2001) |
| | | PloS ONE vol. 6, el6324 (2011) |
| | | Seikagaku: The Journal of the Japanese |
| | | Biochemical Society vol. 87, Issue 1 |
| | EBV infection | Oncogene vol. 23, pp. 8619-8628 (2004) |
| | HIV infection | Journal of Biological Chemistry vol. 275, pp. 16513- |
| | | 16517 (2000) |
| Neurologic | Alzheimer's disease | Biochimica et Biophysica Acta vol. 1802, pp. 396- |
| disease | | 405 (2010) |
| | | Neurobiology of Disease vol. 43, pp. 38-45 (2011) |
| | Parkinson's disease | Biochimica et Biophysica Acta vol. 1802, pp. 396- |
| | | 405 (2010) |
| | ALS | Biochimica et Biophysica Acta vol. 1802, pp. 396- |
| | | 405 (2010) |
| RAS/MAPK | Noonan Syndrome | Human Molecular Genetics vol. 15, pp. R220-R226 |
| syndrome | | (2006) |
| | Costello syndrome | Genetics in Medicine vol. 14, pp. 285-292 (2012) |
| | CFC syndrome | Human Mutation vol. 29, pp. 992-1006 (2008) |
| Other diseases | Cirrhosis/Chronic hepatitis | Gastroenterologia Japonica vol. 24, pp. 270-276 |
| or disorders | | (1989) |
| | Memory impairment | Nature Communications vol. 7, 12926 (2016) |

Further provided is a method of treating cancer in a subject in need thereof, the method comprising administering to the subject an amount of Compound A as disclosed herein or a pharmaceutical composition comprising such a compound. In some embodiments, the cancer is colorectal cancer, non-small cell lung cancer, small-cell lung cancer, pancreatic cancer (e.g., pancreatic adenocarcinoma or pancreatic ductal adenocarcinoma), appendiceal cancer, melanoma, acute myeloid leukemia, small bowel cancer, ampullary cancer, germ cell cancer, cervical cancer, cancer of unknown primary origin, endometrial cancer, esophagogastric cancer, GI neuroendocrine cancer, ovarian cancer, sex cord stromal tumor cancer, hepatobiliary cancer, or bladder cancer. In some embodiments, the cancer is appendiceal, endometrial or melanoma. Also provided is a method of treating a RAS protein-related disorder in a subject in need thereof, the method comprising administering to the subject a therapeutically effective amount of a compound of the present invention, or a pharmaceutically acceptable salt thereof, or a pharmaceutical composition comprising such a compound or salt.

As used herein, the terms “cancer” or “tumor” refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist isolated within an animal, or can be non-tumorigenic, such as a leukemia cell. Cancers include, but are not limited to, B cell malignancies, for example, multiple myeloma, the heavy chain diseases, such as, for example, alpha chain disease, gamma chain disease, and mu chain disease, benign monoclonal gammopathy, and immunocytic amyloidosis, skin cancer, breast cancer, lung cancer, bronchus cancer, colorectal cancer, prostate cancer, pancreatic cancer (e.g., pancreatic adenocarcinoma or pancreatic ductal adenocarcinoma), stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, and the like. Other non-limiting examples of types of cancers applicable to the methods encompassed by the present disclosure include human sarcomas and carcinomas, for example, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer (e.g., pancreatic adenocarcinoma or pancreatic ductal adenocarcinoma), breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, for example, acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, the cancer is an epithelial cancer such as, but not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer (e.g., pancreatic adenocarcinoma or pancreatic ductal adenocarcinoma), prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (for example, serous ovarian carcinoma), or breast carcinoma.

In some embodiments, Compound A, pharmaceutical compositions comprising Compound A or salt thereof, and methods provided herein may be used for the treatment of a wide variety of cancers including tumors such as lung, prostate, breast, brain, skin, cervical carcinomas, testicular carcinomas, etc. More particularly, cancers that may be treated and methods of the disclosure include, but are not limited to tumor types such as astrocytic, breast, cervical, colorectal, endometrial, esophageal, gastric, head and neck, hepatocellular, laryngeal, lung, oral, ovarian, prostate and thyroid carcinomas and sarcomas. Other cancers include, for example:
Cardiac, for example: sarcoma (angiosarcoma, fibrosarcoma, rhabdomyosarcoma, liposarcoma), myxoma, rhabdomyoma, fibroma, lipoma and teratoma;
Lung, for example: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma;
Gastrointestinal, for example: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma);
Genitourinary tract, for example: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma);
Liver, for example: hepatoma (hepatocellular carcinoma), cholangiocarcinoma, hepatoblastoma, angiosarcoma, hepatocellular adenoma, hemangioma;
Biliary tract, for example: gall bladder carcinoma, ampullary carcinoma, cholangiocarcinoma;
Bone, for example: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors;
Nervous system, for example: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, neurofibromatosis type 1, meningioma, glioma, sarcoma);
Gynecological, for example: uterus (endometrial carcinoma, uterine carcinoma, uterine corpus endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma);
Hematologic, for example: blood (myeloid leukemia (acute and chronic), acute lymphoblastic leukemia, chronic lymphocytic leukemia, myeloproliferative diseases (e.g., myelofibrosis and myeloproliferative neoplasms, multiple myeloma, myelodysplastic syndrome), Hodgkin's disease, non-Hodgkin's lymphoma (malignant lymphoma);
Skin, for example: malignant melanoma, basal cell carcinoma, squamous cell carcinoma, Kaposi's sarcoma, moles dysplastic nevi, lipoma, angioma, dermatofibroma, keloids, psoriasis; and Adrenal glands, for example: neuroblastoma.

In some embodiments, the cancer comprises a KRAS G12D mutation. In some embodiments, a mutation is a G12D mutation, and one or more mutations selected from:
(a) the following KRAS mutants: G12C, G12V, G13D, G12R, G12A, Q61H, G12S, A146T, G13C, Q61L, Q61R, K117N, A146V, G12F, Q61K, L19F, Q22K, V141, A59T, A146P, G13R, G12L, or G13V, and combinations thereof;
(b) the following HRAS mutants: Q61R, G13R, Q61K, G12S, Q61L, G12D, G13V, G13D, G12C, K117N, A59T, G12V, G13C, Q61H, G13S, A18V, D119N, G13N, A146T, A66T, G12A, A146V, G12N, or G12R, and combinations thereof; and
(c) the following NRAS mutants: Q61R, Q61K, G12D, Q61L, Q61H, G13R, G13D, G12S, G12C, G12V, G12A, G13V, G12R, P185S, G13C, A146T, G60E, Q61P, A59D, E132K, E49K, T501, A146V, or A59T, and combinations thereof;
   or a combination of any of the foregoing. In some embodiments, the cancer comprises at least two RAS mutations, a G12D mutation and at least one mutation selected from the group consisting of G13C, G13D, G13S, G13V, Q61H, Q61K, Q61L, or a combination thereof. In some embodiments, the cancer is non-small cell lung cancer and the RAS mutation comprises a KRAS mutation, such as KRAS G12D. In some embodiments, the cancer is colorectal cancer and the RAS mutation comprises a KRAS mutation, such as KRAS G12D. In some embodiments, the cancer is pancreatic cancer and the RAS mutation comprises a KRAS G12D mutation. In some embodiments, the pancreatic cancer is pancreatic adenocarcinoma. In some embodiments, the pancreatic cancer is pancreatic ductal adenocarcinoma. In some embodiments, the cancer is colorectal cancer.

In some embodiments, the cancer comprises an NRAS G12D mutation. In some embodiments, the cancer comprises an HRAS G12D mutation. In some embodiments, the cancer comprises a NRAS G12D mutation and a KRAS G12D mutation.

Methods of detecting RAS mutations are known in the art. Such means include, but are not limited to direct sequencing, and utilization of a high-sensitivity diagnostic assay (with CE-IVD mark), e.g., as described in Domagala, et al., Pol J Pathol 3: 145-164 (2012), incorporated herein by reference in its entirety, including TheraScreen PCR; AmoyDx; PNACIamp; RealQuality; EntroGen; LightMix; StripAssay; Hybcell plexA; Devyser; Surveyor; Cobas; and TheraScreen Pyro. See, also, e.g., WO 2020/106640.

In some embodiments, the cancer is non-small cell lung cancer and the RAS mutation comprises a KRAS G12D mutation and a KRAS G12C, KRAS G12V or KRAS G12C mutation. In some embodiments, the cancer is colorectal cancer and the RAS mutation comprises a KRAS G12D mutation, and a KRAS G12C, or KRAS G12V mutation. In some embodiments, the cancer is pancreatic cancer and the RAS mutation comprises a KRAS mutation, such as KRAS G12D. In some embodiments, the pancreatic cancer is pancreatic adenocarcinoma. In some embodiments, the pancreatic cancer is pancreatic ductal adenocarcinoma. In some embodiments, the cancer is melanoma. In some embodiments, the cancer is non-small cell lung cancer.

In some embodiments, the subject being treated by Compound A in the disclosed methods is one who has undergone at least one or more prior systemic cancer therapies (e.g., Compound A is a second- or third-line therapy). In some embodiments, the subject being treated by Compound A in the disclosed methods is one who has disease progression following at least one prior systemic cancer therapy (i.e., Compound A is a second line therapy). In some embodiments, the subject being treated by Compound A in the disclosed methods is one who has disease progression following at least two prior systemic cancer therapies (i.e., Compound A is a third line therapy). Prior systemic cancer therapies can be any therapy approved by a regulatory authority (e.g., the FDA or EMA) as treatment given type and stage of cancer. In some cases, the prior systemic cancer therapy is a cancer therapy not yet approved by a regulatory′ authority but undergoing clinical trials. If a subject has had a prior systemic cancer therapy, in some cases, the subject has not undergone any systemic cancer therapy for at least one month, at least two months, at least three months, at least four months, at least five months, or at least six months prior to starting therapy as disclosed herein with Compound A.

In various embodiments, the disclosure provides a method of treating cancer in a subject comprising administering to the subject a composition comprising Compound A in an amount disclosed herein or combination of compounds described herein, wherein the subject has one or more tumors that are resistant or unresponsive to treatment. In various embodiments, the subject has one or more tumors that are resistant or unresponsive to one or more treatments selected from the group consisting of surgery, radiation, chemotherapy, biologic agents, small molecules, cell-based therapy, hormone therapy, and immunotherapy. In various embodiments, treatment is a standard of care therapy, first-line therapy, second-line therapy, or third-line therapy. In various embodiments, the subject has one or more tumors that have progressed during one or more treatments, wherein the treatments are standard of care therapy, first-line therapy, second-line therapy, or third-line therapy.

First-line therapy is defined as a treatment that is administered to a subject suffering from cancer who has not received any prior treatment. Second-line therapy is defined as treatment that is administered to a subject suffering from cancer who has received prior first-line therapy but experienced disease progression during first-line treatment. Third-line therapy is defined as treatment that is administered to a subject suffering from cancer who has received prior first and second-line treatment but has experienced disease progression during second-line treatment. Each particular type of cancer has a first-line, second-line, and third-line therapy. The first-, second-, and third-line therapies for types of cancer are known in the art. In addition, FDA approved drug labels will indicate if a particular drug is approved as a first-, second-, or third-line therapy.

In various embodiments, the disclosure provides a method of treating cancer in a subject comprising administering to the subject a composition comprising Compound A in an amount disclosed herein or combination of compounds described herein, wherein the subject cannot tolerate standard of care therapy, first-line therapy, second-line therapy, or third-line therapy. In various embodiments, the disclosure provides a method of treating cancer in a subject comprising administering to the subject Compound A or combination therapy including Compound A, wherein the subject has experienced tumor recurrence after surgical resection of the primary tumor. In various embodiments, the disclosure provides a method of treating cancer in a subject comprising administering to the subject a composition comprising Compound A in an amount disclosed herein or combination of compounds described herein, wherein the subject has a tumor that cannot be surgically removed. In various embodiments, the disclosure provides a method of treating cancer in a subject comprising administering to the subject a composition comprising Compound A in an amount disclosed herein or combination of compounds described herein, wherein the subject has no treatment options available.

In some embodiments, the cancer includes a mutation in RAS and the cancer is resistant to treatment with a RAS(OFF) inhibitor. In some embodiments, the cancer is resistant to a KRASG12C(OFF) inhibitor, a KRASG12D(OFF) inhibitor, a KRASG12V(OFF) inhibitor or a pan-KRAS inhibitor. As used herein, the term “resistant to treatment” refers to a treatment of a disorder with a therapeutic agent, where the therapeutic agent is ineffective or where the therapeutic agent was previously effective and has become less effective over time. Resistance to treatment includes acquired and/or adaptive resistance to treatment, which refers to a decrease in the efficacy of a treatment over a period of time where the subject is being administered the therapeutic agent. Acquired resistance to treatment may result from the acquisition of a mutation in a target protein that renders the treatment ineffective or less effective. Accordingly, resistance to treatment may persist even after cessation of administration of the therapeutic agent. In particular, a cancer may become resistant to treatment with a RAS(OFF) inhibitor that decreases the efficacy of the RAS(OFF) inhibitor. Measurement of a decrease in the efficacy of the treatment will depend on the disorder being treated, and such methods are known to those of skill in the art. For example, efficacy of a cancer treatment may be measured by the progression of the disease. An effective treatment may slow or halt the progression of the disease. A cancer that is resistant to treatment with a therapeutic agent, e.g., a RAS(OFF) inhibitor, may fail to slow or halt the progression of the disease.

In some embodiments, dosages of Compound A may optionally be administered to a subject with food, such as consuming a standardized high-fat, high calorie meal, or in a fasting state (no food or liquids, except for water for >10 hours). In one embodiment, the dose of Compound A is administered with or without food.

A subject undergoing a therapy is monitored for adverse events (AE) during the course of the therapy. A treatment related AE is an AE that is related to the treatment drug. A treatment emergent AE is one that a subject develops undergoing the treatment that was not present prior to start of therapy. In some cases, the treatment emergent AE is not or suspected not to be related to the treatment itself. AEs are characterized as one of five grades—grade I is a mild AE; grade 2 is a moderate AE; grade 3 is a severe AE; grade 4 is a life-threatening or disabling AE; and grade 5 is death related to AE. In some cases, the subject does not exhibit any grade 3 AE that is treatment related. In some cases, the subject does not exhibit any grade 3 AE. In some cases, the subject does not exhibit any grade 4 AE that is treatment related. In some cases, the subject does not exhibit any grade 4 AE. In various cases, the subject does not exhibit a grade 3 or grade 4 AE that is treatment related after administration of Compound A for at least one month, or at least three months. In some aspects, a treatment described herein (e.g., administering Compound A to a subject in need thereof) provides no grade 4 treatment-related AEs. In some embodiments, a treatment described herein provide no grade 5 treatment-related AEs.

In various cases, the subject being treated with Compound A in the methods disclosed herein, does not exhibit any dose limiting toxicities (DLT) at the dose administered. A DLT is any AE meeting the criteria listed below occurring during the first treatment cycle of Compound A (day 1 through day 21) where relationship to the drug cannot be ruled out.

In some aspects, a treatment described herein provides a favorable safety and tolerability profile, such that dose modifications resulting from treatment-related adverse events occur at a frequency that maintains a favorable dose intensity. As used herein, the term “dose modification” refers to any change in the planned dosing regimen (e.g., a dosing regimen as described herein) of the therapeutic agent (e.g., Compound A), including but not limited to dose reduction or temporary dose interruption. A “treatment-related adverse event” (TRAE) refers to an adverse event that is reasonably attributable to administration of the therapeutic agent, as determined by clinical assessment by a doctor. Unless otherwise specified, the frequency of dose modifications due to TRAEs is expressed as the percentage of patients in a population receiving a treatment regimen (e.g., administration of Compound) who experienced at least one such modification. In some embodiments, the frequency of dose modifications due to TRAEs is about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, or about 10%-15%, 10-20%, 20-65%, 25-70%, 30-75%, or 35-65%. In some embodiments, the frequency of dose interruptions due to TRAEs is about 5-20%, or 7-15%, and the frequency of dose reductions due to TRAEs is about 1-5% or 5-10%. In certain embodiments, the frequency of dose modifications due to TRAEs is about 15% or less.

In some aspects, a treatment described herein maintains a favorable dose intensity, such that patients receive a therapeutically effective proportion of the planned cumulative dose despite dose modifications resulting from treatment-related adverse events. As used herein, the term “mean dose intensity” refers to the ratio of the actual cumulative dose of a therapeutic agent (e.g., administration of Compound) administered to a patient relative to the planned cumulative dose over a defined treatment period (e.g., one or more 21-day dosing cycles), expressed as a percentage. Mean dose intensity therefore reflects the extent to which patients are able to remain on the planned regimen despite dose modifications, interruptions, or reductions. A higher mean dose intensity is indicative of improved treatment tolerability and maintenance of therapeutic exposure. In certain embodiments, the mean dose intensity of a treatment described herein is about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or about 60% to about 90%, about 60-65%, about 65-70%, about 70-75%, about 75-80%, about 80-85%, about 85-90%, about 90-95%, about 95-100% or about 80-90%. In certain embodiments, the mean dose intensity is about 95% or more.

In various cases, the subject of the disclosed methods exhibits a response to the therapy. In some cases, the subject exhibits at least a stable disease (SD) due to administration of Compound A. In some cases, the subject exhibits at least a partial response (PR) due to administration of Compound A. The response of a subject is assessed by the criteria as defined by RECIST 1.1, e.g., as discussed in Eisenhauer et al., Eur J Cancer, 45:228-247 (2009). A complete response (CR) is disappearance of all target lesions and any pathological lymph nodes have a reduction in short axis to less than 10 mm. A partial response (PR) is at least a 30% decrease in the sum of diameters of target lesions, taking as reference the baseline sum diameters. A progressive disease is at least a 20% increase in the sum of diameters of target lesions, taking as reference the smallest sum on study (including the baseline sum if that is the smallest on study), and there must be an absolute increase of at least 5 mm in addition to the relative increase of 20%. A stable disease is neither sufficient shrinkage to qualify for PR nor sufficient increase to qualify for PD. A controlled disease state is when a patient may alternate between exhibiting a stable disease and a partial response. The tumor size can be measured by radiographic scan.

Response rates or results for subjects administered Compound A in the methods disclosed herein can be measured in various ways, after the subject has been taking Compound A for a suitable length of time, as is known to those of skill in the art.

The subject can respond to the therapy as measured by at least a stable disease (SD), as determined by Response Evaluation Criteria in Solid Tumors (RECIST) 1.1 protocol (Eisenhauer, et al., 2009). RECIST v1.1 is discussed in detail in the examples below. An at least stable disease is one that is a stable disease, has shown a partial response (PR) or has shown a complete response (CR) (i.e., “at least SD”=SD+PR+CR, often referred to as disease control). In various embodiments, the stable disease has neither sufficient shrinkage to qualify for partial response (PR) nor sufficient increase to qualify for progressive disease (PD). In various embodiments, the patient exhibits at least a partial response (i.e., “at least PR”=PR+CR, often referred to as objective response).

Response can be measured by one or more of decrease in tumor size, suppression or decrease of tumor growth, decrease in target or tumor lesions, delayed time to progression, no new tumor or lesion, a decrease in new tumor formation, an increase in survival or progression-free survival (PFS), and no metastases. In various embodiments, the progression of a patient's disease can be assessed by measuring tumor size, tumor lesions, or formation of new tumors or lesions, by assessing the patient using a computerized tomography (CT) scan, a positron emission tomography (PET) scan, a magnetic resonance imaging (MRI) scan, an X-ray, ultrasound, or some combination thereof.

Several criteria and definitions published in the literature can be used to determine the effect of one or more treatments on tumors in a subject suffering from cancer. Based on these criteria, tumors are defined as “responsive,” “stable,” or “progressive” when they improve, remain the same, or worsen during treatment, respectively. The amount of a tumor in an individual is the “tumor burden” which can be measured as the number, volume, and/or weight of the tumor.

Examples of the commonly used criteria published in the literature include Response Evaluation Criteria in Solid Tumors (RECIST), Modified Response Evaluation Criteria in Solid Tumors (mRECIST), PET Response Criteria in Solid Tumors (PERCIST), Choi Criteria, Lugano Response Criteria, European Association for the Study of the Liver (EASL) Criteria, Response Evaluation Criteria in the Cancer of the Liver (RECICL), and WHO Criteria in Tumor Response.

As used herein, “progression free survival” or “PFS” is the time from treatment to the date of the first confirmed disease progression per RECIST 1.1 criteria. In various embodiments, the patient exhibits a PFS of at least 1 month. In various embodiments, the patient exhibits a PFS of at least 3 months. In some embodiments, the patient exhibits a PFS of at least 6 months.

“RECIST” shall mean an acronym that stands for “Response Evaluation Criteria in Solid Tumors” and is a set of published rules that define when cancer patients improve (“respond”), stay the same (“stable”) or worsen (“progression”) during treatments. Response as defined by RECIST criteria have been published, for example, a Journal of the National Cancer Institute, Vol. 92, No. 3, Feb. 2, 2000 and RECIST criteria can include other similar published definitions and rule sets. One skilled in the art would understand definitions that go with RECIST criteria, as used herein, such as “Partial Response (PR),” “Complete Response (CR),” “Stable Disease (SD)” and “Progressive Disease (PD).”

As used herein, “survival” refers to the subject remaining alive, and includes overall survival as well as progression free survival.

As used herein, “reducing the tumor,” means reducing the size, volume, or weight of the tumor, reducing the number of metastases, reducing the size or weight of a metastasis, or combinations thereof. In certain embodiments, a metastasis is cutaneous or subcutaneous. Thus, in certain embodiments, administration of the immune checkpoint inhibitor reduces the size or volume of the tumor by at least about 10%, at least about 20%, at least about 30%, at least about 40%, at least about 50%, at least about 60%, at least about 70%, at least about 75%, at least about 80%, at least about 90%, at least about 95%, at least about 98% or at least about 99%, for example, relative to a control drug in a subject of the same genotype. In certain embodiments, administration of Compound A or combination therapy comprising the same, reduces the weight of the tumor by at least about 10%, at least about 20%, at least about 30%, at least about 40%, at least about 50%, at least about 60%, at least about 70%, at least about 75%, at least about 80%, at least about 90%, at least about 95%, at least about 98% or at least about 99%, for example, relative to a control drug in a subject of the same genotype. In certain embodiments, administration of the Compound A or combination therapy comprising the same, reduces the size or volume of a metastasis by at least about 10%, at least about 20%, at least about 30%, at least about 40%, at least about 50%, at least about 60%, at least about 70%, at least about 75%, at least about 80%, at least about 90%, at least about 95%, at least about 98% or at least about 99%, for example, relative to a control drug in a subject of the same genotype. In certain embodiments, administration of the RAS(ON) inhibitor therapy or combination therapy comprising the same, reduces the number of metastases by at least about 10%, at least about 20%, at least about 30%, at least about 40%, at least about 50%, at least about 60%, at least about 70%, at least about 75%, at least about 80%, at least about 90%, at least about 95%, at least about 98% or at least about 99% for example, relative to a control drug in a subject of the same genotype. In certain embodiments, combinations of these effects are achieved.

In some embodiments, a biological sample obtained from the subject is used to determine response to treatment with Compound A. As used herein, the term “biological sample” refers to any sample obtained from a subject. A biological sample can be obtained from a subject prior to or subsequent to a diagnosis, at one or more time points prior to or following treatment or therapy, at one or more time points during which there is no treatment or therapy or can be collected from a healthy subject. The biological sample can be a tissue sample or a fluid sample. In certain embodiments, the biological sample includes a tissue sample, a biopsy sample, a tumor aspirate, a bone marrow aspirate or a blood sample (or a fraction thereof, such as blood or serum). In certain embodiments, the biological sample includes a tumor cell or cancer cell, for example a circulating tumor cell present in a fluid sample, for example, blood or a fraction thereof. In certain embodiments, the biological sample includes a cell free nucleic acid present in a fluid sample, for example, blood or a fraction thereof. In one embodiment, the biological sample comprises a cell lysate (or lysate fraction) or cell extract; or a solution containing one or more molecules derived from a cell or cellular material (for example a polypeptide or nucleic acid). The cell lysate can include proteins, nuclear and/or mitochondrial fractions. In certain embodiments, the cell lysate includes a cytosolic fraction. In certain embodiments, the cell lysate includes a nuclear/mitochondrial fraction and a cytosolic fraction.

The source of a biological sample can be solid tissue as from a fresh, frozen and/or preserved organ, tissue sample, biopsy, or aspirate; blood or any blood constituents; bodily fluids such as cerebral spinal fluid, amniotic fluid, peritoneal fluid or interstitial fluid; or cells from any time in gestation or development of the subject. The biological sample can contain compounds that are not naturally intermixed with the tissue in nature such as preservatives, anticoagulants, buffers, fixatives, nutrients, antibiotics or the like. The biological sample can be preserved as a frozen sample or as formaldehyde- or paraformaldehyde-fixed paraffin-embedded (FFPE) tissue preparation. For example, the sample can be embedded in a matrix, for example, an FFPE block or a frozen sample. However, other tissue and sample types are amenable for use herein. In one embodiment, the other tissue and sample types can be fresh frozen tissue, wash fluids, or cell pellets, or the like. A biological sample can be a tumor sample, which contains nucleic acid molecules from a tumor or cancer. A biological sample that is a tumor sample can be DNA, for example, genomic DNA, or cDNA derived from RNA. In one embodiment, the tumor nucleic acid sample is purified or isolated (for example, it is removed from its natural state). In one embodiment, the sample is a tissue (for example, a tumor biopsy), a CTC or cell free nucleic acid.

In certain embodiments, a tumor sample is isolated from a human subject. In certain embodiments, the analysis is performed on a tumor biopsy embedded in paraffin wax. In one embodiment, the sample can be a fresh frozen tissue sample. In certain embodiments, the sample is a bodily fluid obtained from the subject. The bodily fluid can be blood or fractions thereof (specifically, serum, plasma), urine, saliva, sputum or cerebrospinal fluid (CSF). The sample can contain cellular as well as extracellular sources of nucleic acid. The extracellular sources can be cell-free nucleic acids and/or exosomes. The methods described herein, including the RT-PCR methods, are sensitive, precise and have multi-analyte capability for use with paraffin embedded samples. See, for example, Cronin et al., Am. J Pathol. 164(1):35-42 (2004).

Additional means for assessing response are described in detail in the examples below and can generally be applied to the methods disclosed herein.

In various embodiments, the disclosure provides a method of treating cancer in a subject comprising administering to the subject Compound A in an amount described herein. Accordingly, one embodiment of the present disclosure provides a method treating a subject in need thereof by administering a pharmaceutical composition containing Compound A in an amount described herein, and a pharmaceutically acceptable excipient, as well as methods of using Compound A to prepare such compositions.

In some embodiments, pharmaceutical compositions may be specially formulated for administration in solid or liquid form, including those adapted for the following: oral administration, for example, drenches (aqueous or nonaqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

For use as treatment of subjects, Compound A can be formulated as pharmaceutical compositions. Depending on the subject to be treated, the mode of administration, and the type of treatment desired, e.g., prevention, prophylaxis, or therapy, Compound A is formulated in ways consonant with these parameters. A summary of such techniques may be found in Remington: The Science and Practice of Pharmacy, 21st Edition, Lippincott Williams & Wilkins, (2005); and Encyclopedia of Pharmaceutical Technology, eds. J. Swarbrick and J. C. Boylan, 1988 1999, Marcel Dekker, New York, each of which is incorporated herein by reference.

Compositions can be prepared according to conventional mixing, granulating or coating methods, respectively, and the present pharmaceutical compositions can contain from about 0.1% to about 99%, from about 5% to about 90%, or from about 1% to about 20% of Compound A, by weight or volume. In some embodiments, Compound A may be present in amounts totaling 1 95% by weight of the total weight of a composition, such as a pharmaceutical composition.

The composition may be provided in a dosage form that is suitable for intraarticular, oral, parenteral (e.g., intravenous, intramuscular), rectal, cutaneous, subcutaneous, topical, transdermal, sublingual, nasal, vaginal, intravesicular, intraurethral, intrathecal, epidural, aural, or ocular administration, or by injection, inhalation, or direct contact with the nasal, genitourinary, reproductive or oral mucosa. Thus, the pharmaceutical composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, preparations suitable for iontophoretic delivery, or aerosols. The compositions may be formulated according to conventional pharmaceutical practice.

Formulations may be prepared in a manner suitable for systemic administration or topical or local administration. Systemic formulations include those designed for injection (e.g., intramuscular, intravenous or subcutaneous injection) or may be prepared for transdermal, transmucosal, or oral administration. A formulation will generally include a diluent as well as, in some cases, adjuvants, buffers, preservatives and the like. Compounds, or a pharmaceutically acceptable salt thereof, can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised. See, for example, U.S. Pat. No. 5,624,677.

Systemic administration may also include relatively noninvasive methods such as the use of suppositories, transdermal patches, transmucosal delivery and intranasal administration. Oral administration is also suitable for compounds of the invention, or a pharmaceutically acceptable salt thereof. Suitable forms include syrups, capsules, and tablets, as is understood in the art. In one embodiment the therapeutically effective amount of Compound A is administered orally in the form of a tablet or multiple tablets.

Compound A, as described herein, may be formulated in a variety of ways that are known in the art. For example, the first and second agents of the combination therapy may be formulated together or separately. Other modalities of combination therapy are described herein.

The individually or separately formulated agents can be packaged together as a kit. Non limiting examples include, but are not limited to, kits that contain, e.g., two pills, a pill and a powder, a suppository and a liquid in a vial, two topical creams, etc. The kit can include optional components that aid in the administration of the unit dose to subjects, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one subject, multiple uses for a particular subject (at a constant dose or in which the individual compounds, or a pharmaceutically acceptable salt thereof, may vary in potency as therapy progresses); or the kit may contain multiple doses suitable for administration to multiple subjects (“bulk packaging”). The kit components may be assembled in cartons, blister packs, bottles, tubes, and the like.

Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, optionally substituted hydroxylpropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Two or more compounds may be mixed together in a tablet, capsule, or other vehicle, or may be partitioned. In one example, the first compound is contained on the inside of the tablet, and the second compound is on the outside, such that a substantial portion of the second compound is released prior to the release of the first compound.

Formulations for oral use may also be provided as chewable tablets, or as hard gelatin capsules wherein Compound A is mixed with an inert solid diluent (e.g., potato starch, lactose, microcrystalline cellulose, calcium carbonate, calcium phosphate or kaolin), or as soft gelatin capsules wherein Compound A is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Dissolution or diffusion-controlled release can be achieved by appropriate coating of a tablet, capsule, pellet, or granulate formulation of compounds, or by incorporating Compound A into an appropriate matrix. A controlled release coating may include one or more of the coating substances mentioned above or, e.g., shellac, beeswax, glycowax, castor wax, carnauba wax, stearyl alcohol, glyceryl monostearate, glyceryl distearate, glycerol palmitostearate, ethylcellulose, acrylic resins, dl polylactic acid, cellulose acetate butyrate, polyvinyl chloride, polyvinyl acetate, vinyl pyrrolidone, polyethylene, polymethacrylate, methylmethacrylate, 2 optionally substituted hydroxylmethacrylate, methacrylate hydrogels, 1,3 butylene glycol, ethylene glycol methacrylate, or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate methyl methacrylate, polyvinyl chloride, polyethylene, or halogenated fluorocarbon.

The liquid forms in which Compound A, or a composition thereof, can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

In some embodiments, the pharmaceutical composition may further comprise an additional compound having antiproliferative activity. Depending on the mode of administration, compounds, or a pharmaceutically acceptable salt thereof, will be formulated into suitable compositions to permit facile delivery. Each compound, or a pharmaceutically acceptable salt thereof, of a combination therapy may be formulated in a variety of ways that are known in the art. For example, the first and second agents of the combination therapy may be formulated together or separately. Desirably, the first and second agents are formulated together for the simultaneous or near simultaneous administration of the agents.

It will be appreciated that Compound A and pharmaceutical compositions thereof can be formulated and employed in combination therapies, that is, Compound A and pharmaceutical compositions thereof can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics or procedures and the desired therapeutic effect to be achieved. It will also be appreciated that the therapies employed may achieve a desired effect for the same disorder, or they may achieve different effects (e.g., control of any adverse effects).

Administration of each drug in a combination therapy, as described herein, can, independently, be one to four times daily for one day to one year, and may even be for the life of the subject. Chronic, long-term administration may be indicated.

In some embodiments, the disclosure provides a method of treating a disease or disorder that is characterized by aberrant RAS activity due to a RAS G12D mutation, such as a KRAS G12D mutation. In some embodiments, the disease or disorder is a cancer.

### Combination Therapy

The methods of the disclosure may include a compound of the present disclosure used alone or in combination with one or more additional therapies (e.g., non-drug treatments or therapeutic agents). The dosages of one or more of the additional therapies (e.g., non-drug treatments or therapeutic agents) may be reduced from standard dosages when administered alone. For example, doses may be determined empirically from drug combinations and permutations or may be deduced by isobolographic analysis (e.g., Black et al., Neurology 65:S3-S6 (2005)).

A compound of the present invention may be administered before, after, or concurrently with one or more of such additional therapies. When combined, dosages of a compound of the invention and dosages of the one or more additional therapies (e.g., non-drug treatment or therapeutic agent) provide a therapeutic effect (e.g., synergistic or additive therapeutic effect). A compound of the present disclosure and an additional therapy, such as an anti-cancer agent, may be administered together, such as in a unitary pharmaceutical composition, or separately and, when administered separately, this may occur simultaneously or sequentially. Such sequential administration may be close or remote in time.

In certain embodiments, compositions of the disclosure comprise a compound of the present disclosure and one additional therapeutic agent. In certain embodiments, compositions of the disclosure comprise a compound of the present invention and two additional therapeutic agents. In certain embodiments, compositions of the disclosure comprise a compound of the present invention and three additional therapeutic agents. In certain embodiments, compositions of the disclosure comprise a compound of the present invention and four or more additional therapeutic agents.

Also provided are pharmaceutical compositions including the combinations, or a pharmaceutically acceptable salt thereof, and a pharmaceutically acceptable excipient. Compositions comprising a combination of therapeutic agents may be used in methods of modulating RAS (e.g., in a subject or in a cell) and in methods of treating RAS related diseases and disorders (e.g., cancer), as described herein. The present disclosure provides, inter alia, compositions, methods, and kits for treating or preventing a RAS related disease or disorder.

Exemplary agents that may be used in combination with a compound of the present disclosure are described below. All references herein are incorporated by reference for the agents described, including compound or molecular structures disclosed therein, whether explicitly stated as such or not.

a. RAS(ON) Inhibitors

Compositions of the present disclosure may include one or more RAS(ON) inhibitor compounds. RAS(ON) inhibitors disclosed herein may be administered or formulated in combination with an additional therapeutic agent described herein. In some embodiments, the present disclosure provides non-covalent binding of RAS by a RAS(ON) multi-selective inhibitor.

One or more RAS(ON) multi-selective inhibitors useful according to the present disclosure can be found in any of the following patent applications: WO 2025209533, WO 2025201453, WO 2025162395, WO 2025119392, WO2025087431, WO 2025051241, WO 2025045233, WO 2024249299, WO 2024222864, WO 2024206858, WO 2024169914, WO 2024153208, WO 2024149214, WO 2024104364, WO 2024067857, WO 2024060966, WO 2024017859, WO 2024008834, WO 2023240263, WO 2023025832, WO 2022060836, WO 2021091956, CN 120441594, CN 119350371, CN 117903169, CN 117720556, CN 117720555, CN 117720554, CN 117534687, CN 117534685, CN 117534684, each of which is incorporated herein by reference in its entirety, including the compound structures disclosed therein.

In some embodiments, the RAS(ON) inhibitor is a RAS(ON) multi-selective inhibitor (e.g., daraxonrasib (RMC-6236), AN9025, BPI-572270, compound 6A of WO 2024/067857, ERAS-0015, GFH276, GFH547, HJ-099, RMC-7977, RCZY-680, RCZY-690, RG6505, AUBE00.

In some embodiments, the RAS(ON) multi-selective inhibitor is daraxonrasib (RMC-6236):

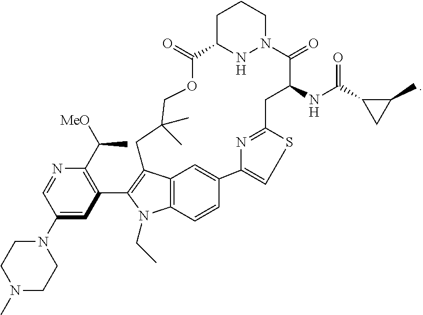

In some embodiments, the RAS(ON) multi-selective inhibitor is compound 6A of WO 2024067857:

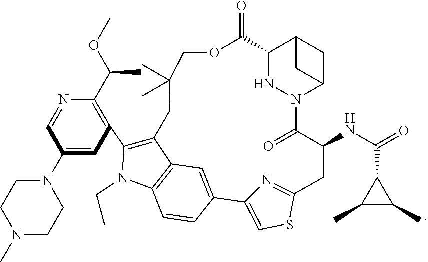

The RAS(ON) multi-selective compounds useful according to the present disclosure exhibit inhibitory activities across a variety of RAS mutants. In some embodiments, a RAS(ON) multi-selective compound inhibits wild type RAS. In some embodiments, a RAS(ON) multi-selective compound inhibits wild type KRAS. In some embodiments, a RAS(ON) multi-selective compound inhibits a RAS mutant with one or more mutations at G12X, G13X, and/or Q61X, wherein X represents any naturally occurring amino acid residue. In certain instances, X is A, C, D, V, S, R, H, K, or L amino acid residue.

In certain embodiments, a RAS(ON) multi-selective compound inhibits a RAS mutant with one or more mutations at G12X, wherein X represents any naturally occurring amino acid residue. In certain instances, X is A, C, D, V, S or R amino acid residue.

In other embodiments, a RAS(ON) multi-selective compound inhibits a RAS mutant with one or more mutations at G13X, wherein X is any naturally occurring amino acid residue. In certain instances, X is A, C, D, V, S or R amino acid residue.

In other embodiments, a RAS(ON) multi-selective compound inhibits a RAS mutant with one or more mutations at Q61X, wherein X is any naturally occurring amino acid residue. In certain instances, X is A, C, D, V, S, R, H, K, or L amino acid residue. In other instances, X is H, K, R, or L amino acid residue.

A variety of RAS proteins may be inhibited by a RAS(ON) multi-selective compound (e.g., KRAS, NRAS, HRAS, and mutants thereof at positions 12, 13 and 61, such as G12A, G12C, G12D, G12V, G12S, G12R, G13C, G13D, Q61H, Q61K, Q61R and Q61L, and others described herein, or a combination thereof). In some embodiments, a RAS(ON) multi-selective compound inhibits a G12A, G12C, G12D, G12R, G12S, G12V, or Q61H mutant of RAS, or a combination thereof.

Compositions and methods described herein may include one or more RAS(ON) mutant-selective inhibitors. Numerous RAS(ON) mutant-selective inhibitors have been disclosed.

In some embodiments, the RAS(ON) mutant-selective inhibitor is a RAS(ON) G12C-selective inhibitor. In some embodiments, the RAS(ON) mutant-selective inhibitor is a RAS(ON) G12D-selective inhibitor. In some embodiments, the RAS(ON) mutant-selective inhibitor is a RAS(ON) G13C-selective inhibitor. In some embodiments, the RAS(ON) mutant-selective inhibitor is a RAS(ON) Q61H-selective inhibitor. In some embodiments, the RAS(ON) mutant-selective inhibitor is a RAS(ON) G12V-selective inhibitor. In some embodiments, the RAS(ON) mutant-selective inhibitor is a RAS(ON) G13D-selective inhibitor. In some embodiments, the RAS(ON) mutant-selective inhibitor is a RAS(ON) G12R-selective inhibitor.

RAS(ON) mutant-selective inhibitors useful in combinations according to the methods of the present disclosure can be found in any one of the following patent applications: WO 2025104149, WO 2025093625, WO 2025080946, WO 2024249299, WO 2024211663, WO 2024211712, WO 2024208934, WO 2024149819, WO 2024008610, WO 2024102421, WO 2023240263, WO 2023133543, WO 2023015559, WO 2023086341, WO 2023208005, WO 2023232776, WO 2023086341, WO 2023060253, WO 2023015559, WO 2022235870, WO 2022235864, WO 2021091967, WO 2021091982, WO 2021108683, WO 2020132597, each of which is incorporated herein by reference in its entirety, including the compound structures disclosed therein.

In some embodiments, the RAS(ON) mutant-selective inhibitor useful according to the present disclosure is a G12D-selective inhibitor, such as zoldonrasib (RMC-9805) or RMC-9945.

In some embodiments, the RAS(ON) mutant-selective inhibitor is a G12C-selective inhibitor, such as elironrasib (RMC-6291) or RMC-4998.

In some embodiments, the RAS(ON) mutant-selective inhibitor is a G12V-selective inhibitor, such as RMC-5127.

In some embodiments, the RAS(ON) mutant-selective inhibitor is a G13C-selective inhibitor, such as RMC-8839. In some embodiments, the RAS(ON) mutant-selective inhibitor is a Q61H-selective inhibitor, such as RMC-0708. In some embodiments, the RAS(ON) mutant-selective inhibitor is a G12R-selective inhibitor, such as RMC-8264.

The RAS(ON) inhibitor compounds described herein may be made from commercially available starting materials or synthesized using known organic, inorganic, or enzymatic processes. By way of example, the RAS(ON) compounds can be synthesized using the methods described in WO 2022060836, WO 2021091956, or WO 2021091982, or any of the other RAS(ON) references cited herein, together with synthetic methods known in the art of synthetic organic chemistry, or variations thereon as appreciated by those skilled in the art.

A RAS(ON) inhibitor may be an antibody-drug conjugate, such as WO 2025051241 and WO 2024189481. See also doi.org/10.1021/acs.jmedchem.4c02929. RAS(OFF) inhibitors are also known, such as WO 2025171055.

In some embodiments, the combination therapy comprising a compound of the present disclosure may include one or more RAS(ON) inhibitors, for example, a compound of the present disclosure plus one or more RAS(ON) multi-selective inhibitors and/or one or more RAS(ON) mutant-selective inhibitors.

### b) RAS/MAPK Inhibitors

Compositions and methods described herein may include a compound of the present disclosure in combination with one or more RAS/MAPK pathway inhibitors. The RAS/MAPK pathway is a signal transduction cascade downstream of various cell surface growth factor receptors in which activation of RAS (and its various isoforms and allotypes) is a central event that drives a variety of cellular effector events that determine the proliferation, activation, differentiation, mobilization, and other functional properties of the cell. SHP2 conveys positive signals from growth factor receptors to the RAS activation/deactivation cycle, which is modulated by guanine nucleotide exchange factors (GEFs, such as SOS1) that load GTP onto RAS to produce functionally active GTP-bound RAS as well as GTP-accelerating proteins (GAPs, such as NF1) that facilitate termination of the signals by conversion of GTP to GDP. GTP-bound RAS produced by this cycle conveys essential positive signals to a series of serine/threonine kinases including RAF and MAP kinases, from which emanate additional signals to various cellular effector functions. In some embodiments, a therapeutic agent that may be combined with a RAS(ON) inhibitor is an inhibitor of the MAP kinase (MAPK) pathway (or “MAPK pathway inhibitor”). MAPK pathway inhibitors include, but are not limited to, one or more MAPK pathway inhibitors described in Cancers (Basel) 2015 September; 7(3): 1758-1784. For example, the MAPK inhibitor may be selected from one or more of trametinib, binimetinib, selumetinib, cobimetinib, LErafAON (NeoPharm), ISIS 5132; vemurafenib, pimasertib, TAK733, RO4987655 (CH4987655); CI-1040; PD-0325901; CH5126766; MAP855; AZD6244; refametinib (RDEA 119/BAY 86-9766); GDC-0973/XL581; AZD8330 (ARRY-424704/ARRY-704); RO5126766 (Roche, described in PLoS One. 2014 Nov. 25; 9(11)); and GSK1120212 (or JTP-74057, described in Clin Cancer Res. 2011 Mar. 1; 17(5):989-1000). The MAPK pathway inhibitor may be PLX8394, LXH254, GDC-5573, or LY3009120. A MAPK pathway inhibitor may be a PI3Kα:RAS breaker, such as BBO-10203.

i. RAS(OFF) Inhibitors, RAS(OFF) Degraders and Other RAS Inhibitor Types

Compositions and methods described herein may include a compound of the present disclosure in combination with one or more RAS(OFF) inhibitors. Numerous mutant-selective and pan-KRAS inhibitors have been disclosed and are known in the art. A RAS(OFF) inhibitor may be administered or formulated in combination with a RAS(ON) inhibitor described herein. RAS(OFF) inhibitors are designed to inhibit RAS activity by targeting different regions of the RAS protein in its inactive state (GDP bound state), preventing its activation and downstream signaling.

In some embodiments, a RAS(OFF) inhibitor is a KRAS(OFF) inhibitor that has a molecular weight of under 700 Da. In some embodiments, the KRAS(OFF) inhibitor is specific for a KRAS^{G12C} mutation. KRAS^{G12C}(OFF) inhibitors use a covalent binding group that allows them to selectively target the KRAS^{G12C} mutant protein, and many such inhibitors comprise a pyrimidine core. KRAS^{G12C}(OFF) inhibitors all target the same cysteine residue in the KRAS^{G12C} mutant protein, leading to a conformational change that locks the protein in an inactive state. KRAS^{G12C}(OFF) inhibitors include, but are not limited to, adagrasib (MRTX849), divarasib (RG6330/GDC-6036), fulzerasib (IB1351/GFH925), garsorasib (D-1553), glecirasib (JAB-21822), olomorasib (LY3537982), opnurasib (JDQ443), sotorasib (AMG 510), ARS-853, ARS-1620, BI-0474, BI 1823911, BPI-421286, D3S-001, ERAS-3490, GEC255, GH35, HBI-2438, HS-10370, JAB-21000, JAB-21822, JMKX001899, JNJ-74699157 (ARS-3248), MK-1084, SK-17, and YL-15293, HRS-7058. In some embodiments, the KRAS(OFF) inhibitor is selected from AMG510 and MRTX849. In some embodiments, the KRAS(OFF) inhibitor is AMG510. In some embodiments, the KRAS(OFF) inhibitor is MRTX849. In some embodiments, the KRAS(OFF) inhibitor is GDC-6036. A RAS(OFF) inhibitor may be an antibody-drug conjugate. See also doi.org/10.1021/acs.jmedchem.4c02929.

In some embodiments, a KRAS(OFF) inhibitor is specific for a KRAS^{G12D} mutation. Non-limiting examples of KRAS^{G12D}(OFF) inhibitors include AST2169, BPI-501836, DN022150, ERAS-4693, ERAS-5024, GDC-7035 (RG6620), HBW-012-D, HBW-012-E, HBW-012336, HRS-4642, HS-10529, INCB186748, JAB-22000, KD-8, KRB-456, LY3962673, MRTX282, MRTX1133, Q2a, QLC1101, RNK08954, SHR1127, TH-Z827, TH-Z835, TSN1611, VRTX153, HJ-119, JR-6000, NKT-G12D, FWD-K02, JAB-BX600, EB-TM1, ABSK141, BPI-2491, HRS-6093, and HRS-7172.

In some embodiments, the small molecule RAS(OFF) inhibitor is specific for a KRAS^{G12V} mutation (e.g., JAB-23000, QTX3544). In some embodiments, the small molecule RAS(OFF) inhibitor is specific for a KRAS^{G13D} mutation.

In some embodiments, reference to the term RAS(OFF) inhibitor includes any such RAS(OFF) inhibitor disclosed in any one of the following patent applications: WO 2025209490, WO 2025202022, WO 2025201480, WO 2025199170, WO 2025194340, WO 2025194134, WO 2025194057, WO 2025194054, WO 2025190342, WO 2025188668, WO 2025179058, WO 2025170938, WO 2025168072, WO 2025167948, WO 2025165972, WO 2025162091, WO 2025157289, WO 2025157260, WO 2025157246, WO 2025157162, WO 2025153038, WO 2025151738, WO 2025151594, WO 2025148979, WO 2025146194, WO 2025136346, WO 2025132549, WO 2025130912, WO 2025129002, WO 2025124415, WO 2025123318, WO 2025123007, WO 2025122619, WO 2025117828, WO 2025111586, WO 2025111582, WO 2025108443, WO 2025106905, WO 2025106901, WO 2025101776, WO 2025096984, WO 2025096957, WO 2025096738, WO 2025092986, WO 2025092798, WO 2025085748, WO 2025085580, WO 2025080653, WO 2025077770, WO 2025077663, WO 2025076523, WO 2025072649, WO 2025072457, WO 2025072451, WO 2025067459, WO 2025067453, WO 2025064848, WO 2025064542, WO 2025061125, WO 2025059366, WO 2025059040, WO 2025054530, WO 2025054347, WO 2025054270, WO 2025053850, WO 2025051242, WO 2025045141, WO 2025049641, WO 2025049619, WO 2025049402, WO 2025049274, WO 2025040767, WO 2025038936, WO 2025036475, WO 2025036470, WO 2025034883, WO 2025034849, WO 2025026903, WO 2025019688, WO 2025018418, WO 2025016899, WO 2025016432, WO 2025011443, WO 2025010415, WO 2025007000, WO 2025006967, WO 2025006962, WO 2025006720, WO 2025006704, WO 2025002430, WO 2025002302, WO 2024259169, WO 2024254404, WO 2024254334, WO 2024255795, WO 2024246099, WO 2024243025, WO 2024238633, WO 2024238343, WO 2024236452, WO 2024235286, WO 2024235225, WO 2024233776, WO 2024230734, WO 2024230707, WO 2024229447, WO 2024229444, WO 2024229442, WO 2024229317, WO 2024227091, WO 2024220645, WO 2024220532, WO 2024218686, WO 2024215862, WO 2024215754, WO 2024213979, WO 2024213122, WO 2024208305, WO 2024209339, WO 2024206766, WO 2024206747, WO 2024197503, WO 2024193698, WO 2024192424, WO 2024179546, WO 2024178313, WO 2024178304, WO 2024173842, WO 2024167922, WO 2024160225, WO 2024159471, WO 2024159470, WO 2024158778, WO 2024158242, WO 2024153119, WO 2024153116, WO 2024138486, WO 2024138206, WO 2024138052, WO 2024131829, WO 2024125642, WO 2024125600, WO 2024123913, WO 2024123102, WO 2024120433, WO 2024120419, WO 2024119277, WO 2024118926, WO 2024109233, WO 2024112654, WO 2024104453, WO 2024104425, WO 2024107686, WO 2024104453, WO 2024103010, WO 2024097559, WO 2024091409, WO 2024088273, WO 2024085661, WO 2024083258, WO 2024083256, WO 2024083246, WO 2024083168, WO 2024078555, WO 2024076674, WO 2024076672, WO 2024076670, WO 2024067714, WO 2024067575, WO 2024064335, WO 2024063578, WO 2024063576, WO 2024061370, WO 2024061333, WO 2024061267, WO 2024056063, WO 2024055112, WO 2024054926, WO 2024054647, WO 2024054625, WO 2024051763, WO 2024051721, WO 2024050742, WO 2024050640, WO 2024046406, WO 2024046370, WO 2024045066, WO 2024044667, WO 2024044649, WO 2024044334, WO 2024041621, WO 2024041606, WO 2024041589, WO 2024041573, WO 2024040131, WO 2024040109, WO 2024040080, WO 2024036270, WO 2024034657, WO 2024034593, WO 2024034591, WO 2024034123, WO 2024032747, WO 2024032704, WO 2024032703, WO 2024032702, WO 2024031088, WO 2024030647, WO 2024030633, WO 2024029613, WO 2024022507, WO 2024022444, WO 2024020159, WO 2024019103, WO 2024017859, WO 2024017392, WO 2024015731, WO 2024015262, WO 2024012456, WO 2024009191, WO 2024008179, WO 2024008178, WO 2024008068, WO 2024006445, WO 2024006424, WO 2024002373, WO 2023287896, WO 2023287730, WO 2023284881, WO 2023284730, WO 2023284537, WO 2023283933, WO 2023283213, WO 2023280280, WO 2023280136, WO 2023280026, WO 2023278600, WO 2023274383, WO 2023327324, WO 2023246914, WO 2023246903, WO 2023246777, WO 2023244713, WO 2023244615, WO 2023244604, WO 2023244600, WO 2023244599, WO 2023230190, WO 2023226630, WO 2023225302, WO 2023225252, WO 2023220421, WO 2023219941, WO 2023217148, WO 2023215802, WO 2023215801, WO 2023213269, WO 2023212548, WO 2023208005, WO 2023205719, WO 2023199180, WO 2023198191, WO 2023197984, WO 2023190748, WO 2023185864, WO 2023183755, WO 2023183585, WO 2023179703, WO 2023179629, WO 2023173017, WO 2023173016, WO 2023173014, WO 2023172737, WO 2023171781, WO 2023159087, WO 2023159086, WO 2023154766, WO 2023152255, WO 2023151674, WO 2023151621, WO 2023150394, WO 2023150284, WO 2023143623, WO 2023143605, WO 2023143352, WO 2023143352, WO 2023143312, WO 2023141570, WO 2023141300, WO 2023138662, WO 2023138601, WO 2023138589, WO 2023138524, WO 2023133183, WO 2023133181, WO 2023130012, WO 2023125989, WO 2023125627, WO 2023122662, WO 2023122154, WO 2023120742, WO 2023119677, WO 2023117681, WO 2023116934, WO 2023116895, WO 2023114733, WO 2023105491, WO 2023104018, WO 2023103906, WO 2023103523, WO 2023101928, WO 2023099624, WO 2023099624, WO 2023099620, WO 2023099612, WO 2023099608, WO 2023099592, WO 2023098832, WO 2023098425, WO 2023097227, WO 2023081840, WO 2023081476, WO 2023078424, WO 2023077441, WO 2023072297, WO 2023072188, WO 2023066371, WO 2023064857, WO 2023061463, WO 2023061294, WO 2023057985, WO 2023056951, WO 2023056421, WO 2023051586, WO 2023049697, WO 2023046135, WO 2023045960, WO 2023041059, WO 2023041059, WO 2023040989, WO 2023040513, WO 2023039240, WO 2023039020, WO 2023036282, WO 2023034290, WO 2023030517, WO 2023030495, WO 2023030385, WO 2023030495, WO 2023030517, WO 2023030685, WO 2023030687, WO 2023034290, WO 2023036282, WO 2023039240, WO 203020347, WO 2023025116, WO 2023287896, WO 2023287730, WO 2023284881, WO 2023284730, WO 2023284537, WO 2023283933, WO 2023283213, WO 2023280280, WO 2023280136, WO 2023280026, WO 2023278600, WO 2023274383, WO 2023327324, WO 2023040989, WO 2023039240, WO 2023039020, WO 2023036282, WO 2023034290, WO 2023030517, WO 2023030495, WO 2023030385, WO 2023025116, WO 2023020523, WO 2023020521, WO 2023020519, WO 2023020518, WO 2023020347, WO 2023018812, WO 2023018810, WO 2023018809, WO 2023018699, WO 2023014979, WO 2023014006, WO 2023004102, WO 2023003417, WO 2023001141, WO 2023001123, WO 2022271658, WO 2022269508, WO 2022266167, WO 2022266069, WO 2022266015, WO 2022265974, WO 2022261154, WO 2022261154, WO 2022251576, WO 2022251296, WO 2022237815, WO 2022232332, WO 2022232331, WO 2022232320, WO 2022232318, WO 2022223037, WO 2022221739, WO 2022221528, WO 2022221386, WO 2022216762 (e.g., Compound 44 or Compound 66a), WO 2022212894, WO 2022192794, WO 2022192790, WO 2022188729, WO 2022187411, WO 2022184178, WO 2022173870, WO 2022173678, WO 2022135346, WO 2022133731, WO 2022133038, WO 2022133345, WO 2022132200, WO 2022119748, WO 2022109485, WO 2022109487, WO 2022066805, WO 2022002102, WO 2022002018, WO 2021259331, WO 2021257828, WO 2021252339, WO 2021248095, WO 2021248090, WO 2021248083, WO 2021248082, WO 2021248079, WO 2021248055, WO 2021245051, WO 2021244603, WO 2021239058, WO 2021231526, WO 2021228161, WO 2021219090, WO 2021219090, WO 2021219072, WO 2021218939, WO 2021217019, WO 2021216770, WO 2021215545, WO 2021215544, WO 2021211864, WO 2021190467, WO 2021185233, WO 2021180181, WO 2021175199, 2021173923, WO 2021169990, WO 2021169963, WO 2021168193, WO 2021158071, WO 2021155716, WO 2021152149, WO 2021150613, WO 2021147967, WO 2021147965, WO 2021143693, WO 2021142252, WO 2021141628, WO 2021139748, WO 2021139678, WO 2021129824, WO 2021129820, WO 2021127404, WO 2021126816, WO 2021126799, WO 2021124222, WO 2021121371, WO 2021121367, WO 2021121330, WO 2021113595, WO 2021107160, WO 2021106231, WO 2021088458, WO 2021086833, WO 2021085653, WO 2021081212, WO 2021058018, WO 2021057832, WO 2021055728, WO 2021031952, WO 2021027911, WO 2021023247, WO 2020259513, WO 2020259432, WO 2020234103, WO 2020233592, WO 2020216190, WO 2020178282, WO 2020146613, WO 2020118066, WO 2020113071, WO 2020106647, WO 2020102730, WO 2020101736, WO 2020097537, WO 2020086739, WO 2020081282, WO 2020050890, WO 2020047192, WO 2020035031, WO 2020028706, WO 2019241157, WO 2019232419, WO 2019217691, WO 2019217307, WO 2019215203, WO 2019213526, WO 2019213516, WO 2019155399, WO 2019150305, WO 2019110751, WO 2019099524, WO 2019051291, WO 2018218070, WO 2018218071, WO 2018218069, WO 2018217651, WO 2018206539, WO 2018143315, WO 2018140600, WO 2018140599, WO 2018140598, WO 2018140514, WO 2018140513, WO 2018140512, WO 2018119183, WO 2018112420, WO 2018068017, WO 2018064510, WO 2017201161, WO 2017172979, WO 2017100546, WO 2017087528, WO 2017058807, WO 2017058805, WO 2017058728, WO 2017058902, WO 2017058792, WO 2017058768, WO 2017058915, WO 2017015562, WO 2016168540, WO 2016164675, WO 2016049568, WO 2016049524, WO 2015054572, WO 2014152588, WO 2014143659, WO 2013155223, KR 20250100539, KR102819454, KR 20240159370, KR 20240101190, KR 20240101189, KR 20240041720, KR 20240041719, JP 2025100453, CN 120647614, CN 120607543, CN 120590414, CN 120574243, CN 120574242, CN 120535536, CN 120535527, CN 120441596, CN 120230123, CN 119607214, CN 119930639, CN 119909188, CN 119751476, CN 119733053, CN 119684316, CN 119684315, CN 119684314, CN 119661556, CN 119661555, CN 119661539, CN 119606974, CN 119528902, CN 119528810, CN 119504612, CN 119490514, CN 119490512, CN 119462648, CN 119371353, CN 119350242, CN 119264124, CN 119241566, CN 119060049, CN 119060066, CN 119019382, CN 118994158, CN 118994031, CN 118806919, CN 118791505, CN 118772176, CN 118754899, CN 118745175, CN 118666870, CN118666869, CN 118580238, CN 118307563, CN 118221700, CN 118221699, CN 118221698, CN 118221685, CN 118126064, CN 118078802, CN 118078801, CN 118005656, CN 117986263, CN 117986263, CN 117946135, CN 117924327, CN 117903117, CN 117800990, CN 117800989, CN 117800976, CN 117736226, CN 117683051, CN 117645627, CN 117624194, CN 117624190, CN 117586280, CN 117486901, CN 117466917, CN 117462688, CN 117362315, CN 117327102, CN 117327094, CN 117327074, CN 117285590, CN 117263959, CN 117247382, CN 117186095, CN 117164605, CN 116969977, CN 116925075, CN 116891489, CN 116731045, CN 116731044, CN 116554208, CN 116514846, CN 116478184, CN 116478141, CN 116410145, CN 116375742, CN 116354988, CN 116332948, CN 116332938, CN 116327956, CN 116262759, CN 116217592, CN 116199703, CN 116162099, CN 116143806, CN 116143805, CN 116120315, CN 116102559, CN 115960105, CN 115894520, CN 115872979, CN 115850267, CN 115785199, CN 115785124, CN 115724842, CN 115724842, CN 115721720, CN 115716840, CN 115703775, CN 115611923, CN 115611898, CN 115583937, CN 115572278, CN 115557949, CN 115521312, CN 115504976, CN 115490709, CN 115466272, CN 115433183, CN 115433179, CN 115403575, CN 115385938, CN 115385937, CN 115385912, CN 115381786, CN 115368383, CN 115368382, CN 115368381, CN 115353506, CN 115322158, CN 115304623, CN 115304602, CN 115197245, CN 115181106, CN 114989195, CN 114989166, CN 114989147, CN 114920741, CN 114920739, CN 114907387, CN 114874234, CN 114874201, CN 114716436, CN 114716435, CN 114685532, CN 114685460, CN 114591319, CN 114539293, CN 114539286, CN 114539246, CN 114437107, CN 114437084, CN 114409653, CN 114380827, CN 114195804, CN 114195788, CN 114437107, CN 114409653, CN 114380827, CN 114195804, CN 114057776, CN 114057744, CN 114057743, CN 113999226, CN 113980032, CN 113980014, CN 113960193, CN 113929676, CN 113754653, CN 113683616, CN 113563323, CN 113527299, CN 113527294, CN 113527293, CN 113493440, CN 113429405, CN 113321654, CN 113248521, CN 113087700, CN 113024544, CN 113004269, CN 112920183, CN 112778284, CN 112390818, CN 112390788, CN 112300196, CN 112300194, CN 112300173, CN 112225734, CN 112142735, CN 112110918, CN 112094269, CN 112047937, CN 109574871, U.S. Ser. No. 12/331,063, US 2025115603, US 2025114346, US 2025114339, US 20240358702, US 2024270736, EP 4574151, or EP 4389751, each of which is incorporated herein by reference in its entirety, including the RAS compound structures disclosed therein which are specifically incorporated herein by reference.

In some embodiments, reference to the term RAS(OFF) inhibitor refers to a pan-KRAS inhibitor, such as selected from one disclosed in any of the following: WO 2025194057, WO 2025194054, WO 2025165972, WO 2025153038, WO 2025151594, WO 2025136346, WO 2025130912, WO 2025129002, WO 2025123007, WO 2025117828, WO 2025106905, WO 2025106901, WO 2025101776, WO 2025096738, WO 2025092798, WO 2025085748, WO 2025077770, WO 2025077663, WO 2025076523, WO 2025064848, WO 2025059366, WO 2025059040, WO 2025049641, WO 2025049619, WO 2025049402, WO 2025045141, WO 2025038936, WO 2025026903, WO 2025016899, WO 2025007000, WO 2025006967, WO 2025006962, WO 2025006720, WO 2025006704, WO 2024255795, WO 2024254404, WO 2024246099, WO 2024238633, WO 2024238343, WO 2024236452, WO 2024235286, WO 2024235225, WO 2024230734, WO 2024220645, WO 2024220532, WO 2024218686, WO 2024215754, WO 2024213979, WO 2024213122, WO 2024209339, WO 2024206766, WO 2024206747, WO 2024192424, WO 2024178313, WO 2024178304, WO 2024173842, WO2024153180, WO 2024119277, WO 2024120433, WO 2024115890, WO 2024112654, WO 2024104453, WO 2024104425, WO 2024107686, WO 2024104453, WO 2024103010, WO 2024085661, WO 2024083246, WO 2024083168, WO 2024067575, WO 2024064335, WO 2024063578, WO 2024063576, WO 2024051852, WO 2024051763, WO 2024046370, WO 2024044667, WO 2024041621, WO 2024041606, WO 2024041589, WO 2024040131, WO 2024040109, WO 2024032747, WO 2024032704, WO 2024032703, WO 2024032702, WO 2024031088, WO 2024030647, WO 2024030633, WO 2024015262, WO 2024009191, WO 2024008068, WO 2024002373, WO 2023287896, WO 2023274324, WO 2023246914 (e.g., compound 14), WO 2023246777, WO 2023230190, WO 2023215802, WO 2023215801, WO 2023197984, WO 2023190748, WO 2023183585, WO 2023179703, WO 2023173017, WO 2023173016, WO 2023173014, WO 2023172737, WO 2023154766, WO 2023143352, WO 2023143312, WO 2023138589, WO 2023133183, WO 2023122662, WO 2023114733, WO 2023099624, WO 2023099623, WO 2023099612, WO 2023099608, WO 2023099592, WO 2023097227, WO 2023064857, WO 2023056421, WO 2023049697, WO 2023046135, WO 2023039240, WO 2023034290, WO 2023020523, WO 2023020521, WO 2023020519, WO 2023020518, WO 2023001123, WO 2022271823, WO 2022261210, WO 2022258974, WO 2022256459, WO 2022250170, WO 2022248885, WO 2022228543, WO 2022216762, WO 2022072783, WO 2016161361, KR 20250100539, KR 20240101190, KR 20240101189, KR 20240041720, KR 20240041719, JP 2025100453, CN 120607543, CN 120535536, CN 120441596, CN 120365289, CN 119751476, CN 119661539, CN 119371353, CN 119019382, CN 118791505, CN 118221700, CN 118126064, CN 117924327, CN 117946135, CN 117800990, CN 117800989, CN 117683051, CN 117486901, CN 117263959, CN 116969977, CN 116332948, or U.S. Ser. No. 12/331,063, each of which is incorporated herein by reference in its entirety, including the compound structures disclosed therein. In some embodiments, combination comprising a pan-KRAS inhibitor therapy comprises ERAS-4001. In some embodiments, the pan-KRAS inhibitor is a pan-KRAS inhibitor in a patent application filed in the name of Medshine Discovery, Inc. In some embodiments, a combination comprising a pan-KRAS inhibitor therapy includes A2A-03, ABREV01, ADT-007, ABT-200, ADT-030, ADT-1004, BBP-454, BGB-53038, BI-2865, BI-2493, BI 3706674, BRSD-143, ERAS-4, ERAS-254, ERAS-4001, HB-700 (G12X+G13D), HZ-V068, ID12241161, JAB-23400, LY4066434, OC211, PF-07985045, PF-07934040, PF-4040, QTX2024, QTX3034, RSC-1255, SIL204, SYNB021225, YL-17231, ZG2001, WEF-001.

In some embodiments, a RAS inhibitor binds to the OFF form as well as the ON form. Non-limiting examples of such inhibitors include, e.g., pan-KRAS: ALTA3263, AMG 410, BBO-11818, HBW-012462, HBW-016-K, HEC211909, JAB-23E73, JAB-23425, JAB-23E73; or a compound disclosed in Zheng, Q.; Shen, T.; Pampel, J.; Shokat, K. M.; Distal Covalent Targeting Suppresses Signaling of Oncogenic KRas(G13C) in Cancer Cells. ACS Chem. Biol. 2025, 20, 7, 1696-1706; G12C: BBO-8520, FMC-376; G12D: AZD0022, GFH375 (VS-7375), INCB161734, QTX3046, TSN1611, TH-Z835, TLN-372.

In some embodiments, a RAS inhibitor binds to the ON form of RAS but is not a tri-complex inhibitor, such as pan-KRAS inhibitors JTX-102 and JTX-105. Pan-KRAS(ON) inhibitors with a high selectivity for the ON form may be found, for example, in WO 2025193878.

In any embodiment employing a RAS(OFF) inhibitor herein, a RAS(OFF) degrader targeting the OFF state of RAS may be employed. These degraders are known in the art, such as ASP3082 (G12D) and ASP5834 (pan-KRAS). RAS degraders may be found, for example, in one or more of the following applications: WO 2025190158, WO 2025169901, WO 2025168124, WO 2025168051, WO 2025162250, WO 2025159142, WO 2025151765, WO 2025125630, WO 2025108479, WO 2025107579, WO 2025103476, WO 2025096855, WO 2025085815, WO 2025083472, WO 2025078984, WO 2025076044, WO 2025058008, WO 2025053850, WO 2025024732, WO 2025019823, WO 2025006783, WO 2025006753, WO 2024263586, WO 2024261257, WO 2024261256, WO 2024241248, WO 2024233838, WO 2024199266, WO 2024188281, WO 2024/59164, WO 2024152247, WO 2024149214, WO 2024131777, WO 2024120424, WO 2024119278, WO 2024118966, WO 2024118960, WO 2024083258, WO 2024083256, WO 2024055112, WO 2024054625, WO 2024050742, WO 2024044334, WO 2024040080, WO 2024034657, WO 2024034593, WO 2024034591, WO 2024034123, WO 2024029613, WO 2024020159, WO 2024019103, WO 2024017392, WO 2023215906, WO 2023185864, WO 2023171781, WO 2023141570, WO 2023138524, WO 2023130012, WO 2023116934, WO 2023099620, WO 2023081476, WO 2023077441, WO 2022260482, CN 120647627, CN 120535501, CN 120463820, CN 120441554, CN 120365263, CN 119219669, CN 119161349, CN 118955610, CN 118772249, CN 118725012, CN 118496502, CN 118496300, CN 118126040, CN 115785199, or US 2025213706, each of which is incorporated herein by reference in its entirety. Non-limiting examples of RAS degraders include: ASP3082 (KRAS G12D); ASP4396 (KRAS G12D); BPI-585725 (G12X and WT), LT-010366 (G12D); PT0253 (G12D), RD0255359 (KRAS G12C/DV); RP03707 (G12D), JR-9000, 356A, SH1718, IPS-06061, HDB-82.

In some embodiments, the RAS(OFF) inhibitor is a peptide-based inhibitor. Peptide-based RAS(OFF) inhibitors have been developed that target specific regions of the RAS protein, such as the Switch II region or the RAS-effector interface. Non-limiting examples include the K-Ras-binding peptide (Krpep-2d), the Ras inhibitory peptide (RasIn) and LUNA18 (NCT05012618). Peptide-based RAS(OFF) inhibitors are a class of compounds that target the RAS protein by disrupting its interaction with its downstream effectors or other signaling proteins. These inhibitors are typically designed to mimic the binding motifs of RAS-interacting proteins or other RAS effectors, such as RAF or PI3K. By binding to RAS at the same site as these effectors, peptide-based inhibitors can effectively compete with these proteins and prevent the activation of downstream signaling pathways. See, e.g., WO 2025162428, WO 2025127968, WO 2025018418, WO 2024219480, WO 2024219446, WO 2024176153, WO 2024101402, WO 2024101386, WO 2023214576, WO 2023140329, WO 2022234853, WO 2022234852, WO 2022234851, WO 2022234639 and CN 120040551, each of which is incorporated herein by reference in its entirety.

Peptide-based RAS(OFF) inhibitors can be further classified into two main categories: those that target the RAS-effector interface, and those that target other regions of the RAS protein. Peptide-based inhibitors that target the RAS-effector interface are designed to bind to the switch regions of RAS that are critical for its interaction with downstream effectors, such as RAF or PI3K. These inhibitors typically contain amino acid residues that are similar to those found in the binding motifs of RAS-interacting proteins or effectors and are often designed to form hydrogen bonds or other interactions with key residues on the surface of RAS.

Peptide-based RAS(OFF) inhibitors that target other regions of the RAS protein are typically designed to disrupt other interactions that are critical for the activation or signaling of RAS. For example, some peptide-based inhibitors are designed to bind to the hypervariable region of RAS, which is thought to play a role in membrane localization and anchoring of the protein. By binding to this region, peptide-based inhibitors can prevent the proper localization of RAS to the plasma membrane, which is necessary for its activation and signaling.

Several common motifs have been identified as important for the binding of RAS-interacting proteins and effectors and are often used in the design of peptide-based inhibitors. One example is the RAF-binding domain (RBD), which is found in many RAS-interacting proteins and is important for the interaction of RAS with downstream effectors such as RAF. The RBD contains a conserved amino acid sequence (Arg-Xaa-Arg) that is critical for binding to RAS, and this motif has been incorporated into several peptide-based inhibitors designed to disrupt the RAS-RAF interaction. Another example is the RAS-binding domain (RBD) of PI3K, which is important for the interaction of RAS with this downstream effector. The RBD of PI13K contains several conserved amino acid residues (such as Arg-Arg-Trp) that are critical for binding to RAS, and these motifs have been used in the design of peptide-based inhibitors that target the RAS-PI3K interaction. Other common motifs used in peptide-based RAS(OFF) inhibitors include the Ras-binding domain (RBD) of other RAS-interacting proteins such as RaIGDS and SOS, as well as sequences that mimic the structure of the switch regions of RAS itself. These motifs are typically used to optimize the binding affinity and selectivity of the inhibitor for the desired target protein or interaction.

In some embodiments, the RAS(OFF) inhibitor is an antibody or antigenic binding peptide specific for RAS(OFF). Antibodies have been developed that bind to specific regions of the RAS protein, such as the Switch II region or the RAS-effector interface. For example, some antibodies have been developed that target the switch regions of RAS proteins, which are critical for the activation of these proteins and their interaction with downstream effectors. Binding of these antibodies to the switch regions can prevent the conformational changes required for RAS activation and downstream signaling. Another approach involves the use of antibodies that target RAS-interacting proteins or downstream effectors, such as RAF or PI13K. Binding of these antibodies to their target proteins can disrupt the RAS-dependent signaling pathways and inhibit the growth and survival of cancer cells. Additionally, some antibodies have been developed that can induce the internalization and degradation of RAS proteins, leading to their depletion and inhibition of downstream signaling. For example, some antibodies have been developed that recognize the unique structure of mutant RAS proteins and target them for degradation via the ubiquitin-proteasome pathway. Non-limiting examples of KRAS(OFF)-specific inhibitory antibodies include anti-p21ser, and K27 (DARPin) (see, e.g., Khan et al, Biochim Biophys Acta Mol Cell Res. 2020 February; 1867(2):118570). See also WO 2024136608 and WO 2024111590, each of which is incorporated herein by reference in its entirety.

Antibody-drug conjugates may also be constructed using RAS inhibitors (e.g., RAS(OFF) inhibitors), such as WO 2025212580 and WO 2024189481, which are incorporated herein by reference in their entirety, including the compound structures disclosed therein.

Vaccines may also be used in combination with compounds of the present invention. Non-limiting examples include: AFNT-111 (KRAS G12V), AFNT-211 (KRAS G12V), AFNT-212 (KRAS G12D), ELI-002 (KRAS G12/13X), HB-700, NT-112 (KRAS G12D), and TG01 (pan-KRAS).

Other RAS modalities useful in combination with compounds of the present invention include: ADGN-123, ADGN-121 (gene editing peptide-RNA nanoparticles G12D); ADT-030 (Ras/B-catenin inhibitor); BBO-10203 (PI3Kα:RAS breaker); BI 1701963 (Pan-KRAS:SOS1); mRNA-5671 (nucleic acid) and R07673396 (RAS inhibitor), AZD0240 (TCR-T cell product targeting G12D), MDG2021 (TCR-T cell product targeting G12D), ADGN-121 (peptide-sgRNA nanoparticles), BION-302 (antibody based) LIB111 (antibody based), SIL-204 (ASO/siRNA-based).

ii. SOS1 Inhibitors

In some embodiments, compositions and methods described herein may include a compound of the present disclosure in combination with one or more SOS1 inhibitors. A SOS1 inhibitor may be administered or formulated in combination with a compound of the present disclosure and/or any additional therapeutic agent described herein. In some embodiments, a SOS1 inhibitor is one or more of RMC-5845, RMC-4948, RMC-0331, BI-1701963, BI-1918455, BI-3406, SDR5, MRTX-0902, ZG2001, and BAY-293. In some embodiments, reference to the term SOS1 inhibitor includes any such SOS1 inhibitor disclosed in any one of the following patent applications: WO 2025070947, WO 2025067316, WO 2025062157, WO 2025059046, WO 2025038785, WO 2025003694, WO 2025000265, WO 2024255827, WO 2024172632, WO 2024172631, WO 2024119028, WO 2024102952, WO 2024083257, WO 2024083255, WO 2024079252, WO 2024075070, WO 2024067744, WO 2024035921, WO 2024027762, WO 2024008185, WO 2023250165, WO 2023215257, WO 2023215256, WO 2023180345, WO 2023109929, WO 2023059597, WO 2023041049, WO 2023029833, WO 2023022497, WO 2022184116, WO 2022171184, WO 2022170952, WO 2022170917, WO 2022170802, WO 2022161461, WO 2022157629, WO 2022139304, WO 2022121813, WO 2022028506, WO 2021228028, WO 2019122129, KR20240128541, CN 119431234, CN 119039237, CN 119039234, CN 118812510, CN 117800922, CN117143175, CN 117143176, CN 116462669, CN 116444447, CN 115806560, CN 115677702, CN 115215847, CN 115028644, CN 114685488, and CN 111393519 each of which is incorporated herein by reference in its entirety, including the compound structures disclosed therein which are specifically incorporated herein by reference.

iii. SHP Inhibitors

In some embodiments, compositions and methods described herein may include a compound of the present disclosure in combination with one or more SHP inhibitors. A SHP inhibitor may be administered or formulated in combination with a compound of the present disclosure and/or any additional therapeutic agent described herein. In some embodiments, the SHP inhibitor is an inhibitor of SHP1. In some embodiments, the SHP inhibitor is an inhibitor of SHP2. In some embodiments, the SHP1 inhibitor is SB8091 or SB6299 aka DA-4511. In some embodiments, a SHP2 inhibitor is one or more of SHP099, TNO155, RMC-4550, RMC-4630, JAB-3068, JAB-3312, RLY-1971, ERAS-601, SH3809, PF-07284892, ARRY-558, or BBP-398. In some embodiments, reference to the term SHP2 inhibitor includes any such SHP2 inhibitor disclosed in any one of the following patent applications: WO 2025075693, WO 2025019666, WO 2025011568, WO 2025011480, WO 2024258652, WO 2024193439, WO 2024175081, WO 2024147703, WO 2024125603, WO 2023282702, WO 2023280283, WO 2023280237, WO 2023018155, WO 2023011513, WO 2022271966, WO 2022271964, WO 2022271911, WO 2022259157, WO 2022242767, WO 2022241975, WO 2022237676, WO 2022237367, WO 2022237178, WO 2022235822, WO 20222084008, WO 2022135568, WO 2022063190, WO 2022043865, WO 2022042331, WO 2022033430, WO 2022017444, WO 2022007869, WO 2021259077, WO 2021249449, WO 2021249057, WO 2021244659, WO 2021218755, WO 2021176072, WO 2021171261, WO 2021149817, WO 2021148010, WO 2021147879, WO 2021143823, WO 2021143701, WO 2021143680, WO 2021281752, WO 2021121397, WO 2021119525, WO 2021115286, WO 2021110796, WO 2021088945, WO 2021073439, WO 2021061706, WO 2021061515, WO 2021043077, WO 2021033153, WO 2021028362, WO 2021033153, WO 2021028362, WO 2021018287, WO 2020259679, WO 2020249079, WO 2020210384, WO 2020201991, WO 2020181283, WO 2020177653, WO 2020165734, WO 2020165733, WO 2020165732, WO 2020156243, WO 2020156242, WO 2020108590, WO 2020104635, WO 2020094104, WO 2020094018, WO 2020081848, WO 2020073949, WO 2020073945, WO 2020072656, WO 2020065453, WO 2020065452, WO 2020063760, WO 2020061103, WO 2020061101, WO 2020033828, WO 2020033286, WO 2020022323, WO 2019233810, WO 2019213318, WO 2019183367, WO 2019183364, WO 2019182960, WO 2019167000, WO 2019165073, WO 2019158019, WO 2019152454, WO 2019051469, WO 2019051084, WO 2018218133, WO 2018172984, WO 2018160731, WO 2018136265, WO 2018136264, WO 2018130928, WO 2018129402, WO 2018081091, WO 2018057884, WO 2018013597, WO 2017216706, WO 2017211303, WO 2017210134, WO 2017156397, WO 2017100279, WO 2017079723, WO 2017078499, WO 2016203406, WO 2016203405, WO 2016203404, WO 2016196591, WO 2016191328, WO 2015107495, WO 2015107494, WO 2015107493, WO 2014176488, WO 2014113584, CN 116332908, CN 119264153, CN 117069698, CN 117143107, CN 115677661, CN 115677660, CN 115611869, CN 115521305, CN 115490697, CN 115466273, CN 115394612, CN 115304613, CN 115304612, CN 115300513, CN 115197225, CN 114957162, CN 114920759, CN 114716448, CN 114671879, CN 114539223, CN 114524772, CN 114213417, CN 114195799, CN 114163457, CN 113896710, CN 113248521, CN 113248449, CN 113135924, CN 113024508, CN 112920131, CN 112823796, CN 112409334, CN 112402385, CN 112174935, 111848599, CN 111704611, CN 111393459, CN 111265529, CN 110143949, CN 108113848, U.S. Ser. No. 11/179,397, U.S. Ser. No. 11/044,675, U.S. Ser. No. 11/034,705, U.S. Ser. No. 11/033,547, U.S. Ser. No. 11/001,561, U.S. Ser. No. 10/988,466, U.S. Ser. No. 10/954,243, U.S. Ser. No. 10/934,302, or U.S. Ser. No. 10/858,359, each of which is incorporated herein by reference in its entirety, including the compound structures disclosed therein which are specifically incorporated herein by reference.

iv. MEK Inhibitors

In some embodiments, compositions and methods described herein may include a compound of the present disclosure in combination with one or more MEK inhibitors. A MEK inhibitor may be administered or formulated in combination with a compound of the present disclosure and/or any additional therapeutic agent described herein. In some embodiments, a MEK inhibitor is one or more of pimasertib, IMM-1-104, selumetinib, cobimetinib (COTELLIC®), trametinib (MEKINIST®), atebimetinib and binimetinib (MEKTOVI®). In some embodiments, a MEK inhibitor targets a MEK mutation that is a Class I MEK1 mutation selected from D67N; P124L; P124S; and L177V. In some embodiments, the MEK mutation is a Class II MEK1 mutation selected from AE51-Q58; ΔF53-Q58; E203K; L177M; C121S; F53L; K57E; Q56P; and K57N. In some embodiments, reference to the term MEK inhibitor includes any such MEK inhibitor disclosed in any one of the following patent applications: WO 2022221866, WO 2022125941, WO 2022208391, WO 2022015736, WO 2022177557, WO 2021018866, WO 2021069486, WO 2021142144, WO 2021168283, WO 2021234097, WO 2019076947, WO 2018233696, WO 2016188472, WO 2014063024, WO 2013019906, WO 2011047238, WO 2007044515, US 2023032403, and CN 115813930, each of which is incorporated herein by reference in its entirety, including the compound structures disclosed therein which are specifically incorporated herein by reference.

v. RAF Inhibitors In some embodiments, compositions and methods described herein may include a compound of the present disclosure in combination with one or more RAF inhibitors. A RAF inhibitor may be administered or formulated in combination with a compound of the present disclosure and/or any additional therapeutic agent described herein. In some embodiments, a RAF inhibitor is VS-6766 or BTDX-4933. In some embodiments, a RAF inhibitor is a BRAF inhibitor. BRAF inhibitors that may be used in combination with a compound of the present disclosure include, for example, VS-6766, IK-595, vemurafenib, dabrafenib, and encorafenib. BRAF may comprise a Class 3 BRAF mutation. In some embodiments, the Class 3 BRAF mutation is selected from one or more of the following amino acid substitutions in human BRAF: D287H; P367R; V459L; G466V; G466E; G466A; S467L; G469E; N581S; N5811; D594N; D594G; D594A; D594H; F595L; G596D; G596R and A762E. In some embodiments, reference to the term RAF inhibitor includes any such RAF inhibitor disclosed in any one of the following patent applications: WO 2023076991, WO 2022226626, WO 2022226261, WO 2019084459, WO 2018203219, WO 201851306, WO 2017212442, WO 2015075483, WO 2013134243, WO 2013134298, WO 2011047238, WO 2011025965, WO 2011025947, WO 2011025951, WO 2011025940, WO 2011025938, WO 2010065893, WO 2009016460, WO 2009130015, WO 2009111278, WO 2009111279, WO 2008028141, and WO 2006024834, each of which is incorporated herein by reference in its entirety, including the compound structures disclosed therein which are specifically incorporated herein by reference.
vi. ERK Inhibitors

In some embodiments, compositions and methods described herein may include a compound of the present disclosure in combination with one or more ERK inhibitors. An ERK inhibitor may be administered or formulated in combination with a compound of the present disclosure and/or any additional therapeutic agent described herein. In some embodiments, an ERK inhibitor is an ERK1/2 inhibitor, such as ERAS-007. In some embodiments, an ERK inhibitor is an ERK 5 inhibitor. In some embodiments, an ERK inhibitor is one or more of ASTX-029 or 1-75. In some embodiments, reference to the term ERK inhibitor includes any such ERK inhibitor disclosed in any one of the following patent applications: WO 2023076305, WO 2022259222, WO 2022221547, WO 2021110169, WO 2021110168, WO 2021252316, WO 2020102686, WO 2020228817, WO 2020107987, WO 2019233456, WO 2019233457, WO 2016025561, WO 2016192063, WO 2016106029, WO 2016106009, WO 2015051341, WO 2014124230, WO 2014052563, WO 2011041152, WO 200910550, WO 2008153858, CN114315837, CN 115057860, CN 107973783, each of which is incorporated herein by reference in its entirety, including the compound structures disclosed therein which are specifically incorporated herein by reference.

vii. MAPK Inhibitors

In some embodiments, compositions and methods described herein may include a compound of the present disclosure in combination with one or more Mitogen-Activated Protein Kinase (MAPK) inhibitors. A MAPK inhibitor may be administered or formulated in combination with a compound of the present disclosure and/or any additional therapeutic agent described herein. In some embodiments, a MAPK inhibitor is a p38MAPK inhibitor or a MAP3K8 inhibitor. In some embodiments, the MAPK inhibitor is one or more of Tilpisertib (GS-4875) and neflamapidmod (VX-745). In some embodiments, reference to the term MAPK inhibitor includes any such MAPK inhibitor disclosed in any one of the following patent applications: WO 2016029263, CN 114767674, CN 115850179, and CN 1743006, each of which is incorporated herein by reference in its entirety, including the compound structures disclosed therein which are specifically incorporated herein by reference.

In some embodiments, a therapeutic agent that may be combined with a compound of the present disclosure is an inhibitor of MAP2K4. A non-limiting example of a MAP2K4 inhibitor useful according to the disclosure is HRX-0233.

### c) Kinase Inhibitors

Compositions and methods described herein may include a compound of the present disclosure in combination with one or more kinase inhibitors. Tyrosine kinases and serine/threonine kinases play a crucial role in various cellular processes such as cell signaling, growth, and differentiation. Kinase inhibitors known in the art have been developed as a treatment for various types of cancer in addition to therapies for conditions such as neurodegenerative diseases, autoimmune disorders, and inflammation.

i. PKA Inhibitors

In some embodiments, compositions and methods described herein may include one or more Protein Kinase A (PKA) inhibitors. A PKA inhibitor may be administered or formulated in combination with a compound of the present disclosure and/or any additional therapeutic agent described herein. In some embodiments, a PKA inhibitor is H89. In some embodiments, reference to the term PKA inhibitor includes any such PKA inhibitor disclosed in any one of the following patent applications: CN 106620678 and CN 114632155, each of which is incorporated herein by reference in its entirety, including the compound structures disclosed therein which are specifically incorporated herein by reference.

ii. FAK Inhibitors

In some embodiments, compositions and methods described herein may include a compound of the present disclosure in combination with one or more Focal Adhesion Kinase (FAK) inhibitors. A FAK inhibitor may be administered or formulated in combination with a compound of the present disclosure and/or any additional therapeutic agent described herein. In some embodiments, a FAK inhibitor is one or more of B1853520, defactinib, GSK2256098, PF-00562271, and VS-4718. In some embodiments, reference to the term FAK inhibitor includes any such FAK inhibitor disclosed in any one of the following patent applications: WO 2022152315, WO 2021098679, WO 2020135442, WO 2020191448, WO 2012022408, WO 2013134353, WO 2012110774, WO 2010062578, CN 111072571, and KR 101691536, each of which is incorporated herein by reference in its entirety, including the compound structures disclosed therein which are specifically incorporated herein by reference.

iii. ROCK Inhibitors

In some embodiments, compositions and methods described herein may include a compound of the present disclosure in combination with one or more Rho-associated, coiled-coil containing protein kinase (ROCK) inhibitors. A ROCK inhibitor may be administered or formulated in combination with a compound of the present disclosure and/or any additional therapeutic agent described herein. In some embodiments, a ROCK inhibitor is GSK269962A. In some embodiments, reference to the term ROCK inhibitor includes any such ROCK inhibitor disclosed in any one of the following patent applications: WO 2023051753, WO 2022237892, WO 2022012409, WO 2021093795, WO 2021214200, WO 2020177292, WO 202011751, WO 2019014304, WO 2019179525, WO 2019089868, WO 2019014300, WO 2018108156, WO 2018009627, WO 2018009625, WO 2018009622, WO 2017123860, WO 2017205709, WO 2016112236, WO 2014068035, WO 2013030367, WO 2012146724, WO 2012067965, WO 2011107608, CN 108129453, CN 108191821, CN 110917352, CN 108558823, CN108047193, CN107973777, CN108047197, CN108129448, CN 115869304, and GB202214708, each of which is incorporated herein by reference in its entirety, including the compound structures disclosed therein which are specifically incorporated herein by reference.

iv. MSK1 Inhibitors

In some embodiments, compositions and methods described herein may include a compound of the present disclosure in combination with one or more Mitogen- and stress-activated kinase (MSK1) inhibitors. A MSK1 inhibitor may be administered or formulated in combination with a compound of the present disclosure and/or any additional therapeutic agent described herein. In some embodiments, a MSK1 inhibitor is one or more of SB-747651A, SB 747651A, Ro 320432, CGP 57380, GSK2830371, SR1664, LY-3214996, PFI-4, MSC-2363318A, and AS601245.

v. RSK Inhibitors

In some embodiments, compositions and methods described herein may include a compound of the present disclosure in combination with one or more ribosomal S6 kinase (RSK) inhibitors. A RSK1 inhibitor may be administered or formulated in combination with a compound of the present disclosure and/or any additional therapeutic agent described herein. In some embodiments, a RSK inhibitor is one or more of BI-D1870, LJH685, SL0101-1, FMK, BRD7389, BIX 02565, LJI308, LJI308-S, LJI308-1, and LJH685-S. In some embodiments, a RSK inhibitor is PMD-026. In some embodiments, reference to the term RSK inhibitor includes any such RSK inhibitor disclosed in any one of the following patent applications: WO 2021249558, WO 2020165646, WO 2017141116, and CN 113801139, each of which is incorporated herein by reference in its entirety, including the compound structures disclosed therein which are specifically incorporated herein by reference.

vi. ALK Inhibitors

In some embodiments, compositions and methods described herein may include a compound of the present disclosure in combination with one or more Anaplastic Lymphoma Kinase (ALK) inhibitors. An ALK inhibitor may be administered or formulated in combination with a compound of the present disclosure and/or any additional therapeutic agent described herein. In some embodiments, an ALK inhibitor is one or more of Crizotinib (Xalkori), Ceritinib (Zykadia), Alectinib (Alecensa), Brigatinib (Alunbrig), Lorlatinib (Lorbrena), Ensartinib (X-396), TAE684, ASP3026, TPX-0131, LDK378 (Ceritinib analog), CEP-37440; 4SC-203, TL-398, PLB1003, TSR-011, CT-707, TPX-0005, and AP26113. Additional examples of ALK kinase inhibitors are described in examples 3-39 of WO05016894. In some embodiments, reference to the term ALK inhibitor includes any such ALK inhibitor disclosed in any one of the following patent applications: WO 2019142095, WO 2019179482, WO 2018130928, WO 2018127184, WO 2017101803, WO 2016192132, WO 2014100431, WO 2012082972, CN 111138492, CN 110526914, CN 109836415, CN 105801603, CN107987056, and CN 105878248, each of which is incorporated herein by reference in its entirety, including the compound structures disclosed therein which are specifically incorporated herein by reference.

### d) Receptor Tyrosine Kinase Inhibitors

Compositions and methods described herein may include a compound of the present disclosure in combination with one or more receptor tyrosine kinase inhibitors. A receptor tyrosine kinase (RTK) inhibitor is a type of molecule (e.g., small molecule, antibody, and nucleic acid) that binds to and blocks the activity of receptor tyrosine kinases or their ligands. RTKs are proteins found on the surface of cells that play a critical role in cell signaling and growth and have been developed as therapeutics for a range of diseases, including cancer, diabetes, and autoimmune disorders. In some embodiments, a therapeutic agent may be a pan-RTK inhibitor, such as afatinib.

i. EGFR Inhibitors

In some embodiments, compositions and methods described herein may include a compound of the present disclosure in combination with one or more EGFR inhibitors. An EGFR inhibitor may be administered or formulated in combination with a compound of the present disclosure and/or any additional therapeutic agent described herein. EGFR inhibitors include, but are not limited to, small molecule antagonists, antibody inhibitors, or specific antisense nucleotide or siRNA. Useful antibody inhibitors of EGFR include cetuximab (ERBITUX®), panitumumab (VECTIBIX®), zalutumumab, nimotuzumab, and matuzumab. Further antibody-based EGFR inhibitors include any anti-EGFR antibody or antibody fragment that can partially or completely block EGFR activation by its natural ligand. In some embodiments, the antibody-based EGFR inhibitor is a bispecific antibody. In some embodiments, the bispecific is selective for EGFR and MET. In some embodiments, the bispecific is amivantamab. Non-limiting examples of antibody-based EGFR inhibitors include those described in Modjtahedi et al., Br. J. Cancer 1993, 67:247-253; Teramoto et al., Cancer 1996, 77:639-645; Goldstein et al., Clin. Cancer Res. 1995, 1:1311-1318; Huang et al., 1999, Cancer Res. 15:59(8):1935-40; and Yang et al., Cancer Res.1999, 59:1236-1243. The EGFR inhibitor can be monoclonal antibody Mab E7.6.3 (Yang, 1999 supra), or Mab C225 (ATCC Accession No. HB-8508), or an antibody or antibody fragment having the binding specificity thereof.

Small molecule antagonists of EGFR include gefitinib (IRESSA®), Lazertinib, erlotinib (TARCEVA®), and lapatinib (TYKERB®). See, e.g., Yan et al., Pharmacogenetics and Pharmacogenomics In Oncology Therapeutic Antibody Development, BioTechniques 2005, 39(4):565-8; and Paez et al., EGFR Mutations In Lung Cancer Correlation With Clinical Response To Gefitinib Therapy, Science 2004, 304(5676):1497-500. In some embodiments, the EGFR inhibitor is osimertinib (TAGRISSO®). In some embodiments, an EGFR inhibitor is one or more of cetuximab, gefitinib (IRESSA®), erlotinib (TARCEVA®), and afatinib (GILOTRIF®). Additional non-limiting examples of small molecule EGFR inhibitors include any of the EGFR inhibitors described in Traxler et al., Exp. Opin. Ther. Patents 1998, 8(12):1599-1625. An EGFR inhibitor may be ERAS-801. In some embodiments, an EGFR inhibitor is an ERBB inhibitor. In humans, the ERBB family contains HER1 (EGFR, ERBB1), HER2 (NEU, ERBB2), HER3 (ERBB3), and HER (ERBB4). In some embodiments, the EGFR inhibitor may be bosutinib, crizotinib, dasatinib, erlotinib, gefitinib, lapatinib, pazopanib, ruxolitinib, sunitinib, vemurafenib, abrocitinib, asciminib, futibatinib, ibrutinib, imatinib, pacritinib, or sorafenib. In some embodiments, reference to the term EGFR inhibitor includes any such EGFR inhibitor disclosed in any one of the following patent applications: WO 2023041071, WO 2023049312, WO 2023020600, WO 2023284747, WO 2022206797, WO 2022258977, WO 2022033416, WO 2022033410, WO 2022105908, WO 2022100641, WO 2022014639, WO 2022007841, WO 2021018009, WO 2021057882, WO 2021252661, WO 2021018003, WO 2021073498, WO 2021238827, WO 2020254547, WO 2020216371, WO 2020147838, WO 2020207483, WO 2020254572, WO 2020001350, WO 2021001351, WO 2019164948, WO 2019218958, WO 2019046775, WO 2019015655, WO 2018121758, WO 2018218963, WO 2017220007, WO 2017205459, WO 2017161937, WO 2016192609, WO 199633980, WO 199630347, WO 199730034, WO 199730044, WO 199738994, WO 199749688, WO 199802434, WO 199738983, WO 199519774, WO 199519970, WO 199713771, WO 199802437, WO 199802438, WO 199732881, WO 199833798, WO 199732880, WO 199732880, WO 199702266, WO 199727199, WO 199807726, WO 1997/34895, WO 199631510, WO 199814449, WO 199814450, WO 199814451, WO 199509847, WO 199719065, WO 199817662, WO 199935146, WO 199935132, WO 199907701, WO 199220642, DE 19629652, EP 682027, EP 837063, EP 0787772, EP 0520722, EP 0566226, CN 115960018, CN 110283162, CN 114044774, CN111973601, CN 111973602, and CN113896744, each of which is incorporated herein by reference in its entirety, including the compound structures disclosed therein which are specifically incorporated herein by reference.

ii. HER2 Inhibitors

In some embodiments, compositions and methods described herein may include a compound of the present disclosure in combination with one or more HER2 inhibitors. A HER2 inhibitor may be administered or formulated in combination with a compound of the present disclosure and/or any additional therapeutic agent described herein. In some embodiments, an HER2 inhibitor is one or more of tucatinib, rastuzumab (HERCEPTIN™), pertuzumab (PERJETA™), lapatinib (TYKERB™), ado-trastuzumab emtansine (KADCYLA™), and neratinib (NERLYNX™). Non-limiting examples of HER2 inhibitors include monoclonal antibodies such as trastuzumab (HERCEPTIN®) and pertuzumab (PERJETA®); small molecule tyrosine kinase inhibitors such as gefitinib (IRESSA®), erlotinib (TARCEVA®), pilitinib, CP-654577, CP-724714, canertinib (CI 1033), HKI-272, lapatinib (GW-572016; TYKERB®), PKI-166, AEE788, BMS-599626, HKI-357, BIBW 2992, ARRY-334543, and JNJ-26483327. In some embodiments, reference to the term HER2 inhibitor includes any such HER2 inhibitor disclosed in any one of the following patent applications: WO 2021156178, WO 2021156180, WO 2021213800, WO 2021088987, WO 2013561183, and WO 2013056108, each of which is incorporated herein by reference in its entirety, including the compound structures disclosed therein which are specifically incorporated herein by reference.

iii. MET Inhibitors

In some embodiments, compositions and methods described herein may include a compound of the present disclosure in combination with one or more MET inhibitors. A MET inhibitor may be administered or formulated in combination with a compound of the present disclosure and/or any additional therapeutic agent described herein. In some embodiments, the MET inhibitor is an anti-MET antibody-drug-conjugate such as MYTX-011, TR1801-ADC, ABBV-399, and SHR-A1403. In some embodiments, a MET inhibitor is one or more of Crizotinib (XALKORI™), Cabozantinib (Cometriq, CABOMETYX™), Capmatinib (TABRECTA™), Tepotinib (TEPMETKO™), Savolitinib (VOLITINIB™) Onartuzumab (METMAB™), Foretinib (GSK1363089), MGCD-265 (Amuvatinib), SU11274, and SU5416. In some embodiments, reference to the term MET inhibitor includes any such MET inhibitor disclosed in any one of the following patent applications: WO 2022226168, WO 2021222045, WO 2020047184, WO 2020015744, WO 2020244654, WO 2020156453, WO 2019206268, WO 2018077227, WO 2017012539, WO 2016015653, WO 2016012963, WO 2012015677, WO 2011162835, WO 2010089507, WO 2009091374, WO 2009056692, WO 2008051547, WO 2007130468, US 2012237524, CN 103497177, CN 107311983, CN 107382968, CN 110218191, and TW201331206, each of which is incorporated herein by reference in its entirety, including the compound structures disclosed therein which are specifically incorporated herein by reference.

iv. AXL Inhibitors

In some embodiments, compositions and methods described herein may include a compound of the present disclosure in combination with one or more AXL inhibitors. An AXL inhibitor may be administered or formulated in combination with a compound of the present disclosure and/or any additional therapeutic agent described herein. AXL is a receptor tyrosine kinase that belongs to the TAM family of receptors, which also includes TYRO3 and MERTK. In some embodiments, an AXL inhibitor is one or more of bemcentib, BGB324, R428, SGI-7079, TP-0903, BMS-777607, UNC2025, and TP-0903. In some embodiments, reference to the term AXL inhibitor includes any such AXL inhibitor disclosed in any one of the following patent applications: WO 2023045816, WO 2022237843, WO 2022246179, WO 2021012717, WO 2021088787, WO 2021067772, WO 2021239133, WO 2021204713, WO 2020238802, WO 2019039525, WO 2019101178, WO 2019074116, WO 2017146236, WO 2016097918, WO 2015012298, WO 2010005876, WO 2010083465, CN 115073367, and JP 2022171109, each of which is incorporated herein by reference in its entirety, including the compound structures disclosed therein which are specifically incorporated herein by reference.

### v) IGFR Inhibitors

In some embodiments, compositions and methods described herein may include a compound of the present disclosure in combination with one or more insulin-like growth factor receptor 1 (IGF-1R) inhibitors. An IGFR inhibitor may be administered or formulated in combination with a compound of the present disclosure and/or any additional therapeutic agent described herein. IGFR inhibitors have been developed to target the IGFR receptor, which plays a critical role in cancer progression and metastasis. In some embodiments, an IGFR inhibitor is one or more of linsitinib, AXL1717, OSI-906 (Linsitinib), BMS-754807, BI 836845, AZ12253801, PQIP (Pyrrolo[1,2-a]quinoxaline), and NVP-AEW541. In some embodiments, reference to the term IGFR inhibitor includes any such IGFR inhibitor disclosed in any one of the following patent applications: WO 2022115946, WO 2022217923, WO 2021203861, WO 2021246413, WO 2020116398, WO 2019046600, WO 2018195250, WO 2018221521, WO 2018204872, WO 2017072196, WO 2016173682, WO 2015162291, WO 2015162292, WO 2010066868, WO 2006069202, and CN 112125916, each of which is incorporated herein by reference in its entirety, including the compound structures disclosed therein which are specifically incorporated herein by reference.

vi. RET Inhibitors

In some embodiments, compositions and methods described herein may include a compound of the present disclosure in combination with one or more Rearranged during transfection (RET) inhibitors. An RET inhibitor may be administered or formulated in combination with a compound of the present disclosure and/or any additional therapeutic agent described herein. RET plays a critical role in various cellular processes, including cell growth, differentiation, survival, and migration. RET is activated by binding of its ligands, such as glial cell line-derived neurotrophic factor (GDNF) family ligands, which leads to the activation of downstream signaling pathways that promote these cellular processes. In some embodiments, a RET inhibitor is one or more of pralsetinib, selpercatinib (LOXO-292), BLU-667, RXDX-105, TPX-0046, GSK3179106, molidustat (BAY 85-3934), and RPI-1 (Retrophin). In some embodiments, reference to the term RET inhibitor includes any such RET inhibitor disclosed in any one of the following patent applications: WO 2021211380, WO 2021057963, WO 2021043209, WO 2021222017, WO 2020035065, WO 2020114487, WO 2020200314, WO 2020200316, WO 2020114494, WO 2018071447, WO 2018213329, WO 2017079140, WO 2014050781, CN 113943285, CN 113683610, CN 113683611, CN 113620944, CN 113620945, CN 113527291, CN 113527292, CN 113527290, CN 113135896, CN 111057075, CN111233899, and CN111362923, each of which is incorporated herein by reference in its entirety, including the compound structures disclosed therein which are specifically incorporated herein by reference.

vii. ROS1 Inhibitors

In some embodiments, compositions and methods described herein may include a compound of the present disclosure in combination with one or more c-ros oncogene 1 (ROS1) inhibitors. A ROS1 inhibitor may be administered or formulated in combination with a compound of the present disclosure and/or any additional therapeutic agent described herein. ROS1 is a receptor tyrosine kinase that belongs to the insulin receptor family and plays a role in various cellular processes, including cell growth, differentiation, survival, and migration. In some embodiments, a ROS1 inhibitor is one or more of taletrectinib, DS-6051b, TPX-0131, GZD824, and PF-06463922. In some embodiments, reference to the term ROS1 inhibitor includes any such ROS1 inhibitor disclosed in any one of the following patent applications: WO 2021098703, WO 2020024825, and US 2017079972, each of which is incorporated herein by reference in its entirety, including the compound structures disclosed therein which are specifically incorporated herein by reference.

viii. PDGFR Inhibitors

In some embodiments, compositions and methods described herein may include a compound of the present disclosure in combination with one or more platelet-derived growth factor receptor (PDGFR) inhibitors. A PDGFR inhibitor may be administered or formulated in combination with a compound of the present disclosure and/or any additional therapeutic agent described herein. PDGFR is a family of receptor tyrosine kinases that consists of two members, PDGFRa and PDGFRP. They are activated by binding to their ligands, such as platelet-derived growth factor (PDGF), which leads to the activation of downstream signaling pathways that promote cell growth, proliferation, and survival. In some embodiments, a PDGFR inhibitor is one or more of CP-673451, imatinib, nintedanib (OFEV™), sunitinib (SUTENT™), pazopanib (VOTRIENT™), regorafenib (STIVARGA™), and dasatinib (SPRYCEL™)

ix. FGF Inhibitors

In some embodiments, compositions and methods described herein may include a compound of the present disclosure in combination with fibroblast growth factor (FGF) inhibitors. An FGF inhibitor may be administered or formulated in combination with a compound of the present disclosure and/or any additional therapeutic agent described herein. FGFRs are a family of receptor tyrosine kinases that consists of four members, FGFR1-4. FGFRs are activated by binding to their ligands, fibroblast growth factors (FGFs), which leads to the activation of downstream signaling pathways that promote cell growth, differentiation, and survival. In some embodiments, the FGFR inhibitor is an inhibitor of FGFR2. In some embodiments, the FGFR inhibitor is an inhibitor of FGFR4. In some embodiments, an FGFR inhibitor is one or more of futibatinib (TAK-659), erdafitinib (BALVERSA™), infigratinib (TRUSELTIQ™), Debio 1347, and rogaratinib (BAY 1163877). In some embodiments, reference to the term FGFR inhibitor includes any such FGFR inhibitor disclosed in any one of the following patent applications: WO 2022033472, WO 2022152274, WO 2022166469, WO 2022206939, WO 2021037219, WO 2021089005, WO 2021113462, WO 2020185532, WO 2019213544, WO 2020164603, WO 2019154364, WO 2019034076, WO 2019213506, WO 2019223766, WO 2018028438, WO 2018153373, WO 2018121650, WO 2018010514, WO 2017028816, WO 2017118438, WO 2016134320, WO 2015008844, WO 2014172644, WO 2014007951, WO 2013179033, WO 2013087578, WO 2012047699, CN 105906630, CN 115869315, CN 115141176, CN 115043832, and CN 115028634, each of which is incorporated herein by reference in its entirety. In some embodiments, the FGF pathway inhibitor targets an FGF ligand. Such FGF pathway inhibitors include FGF ligand traps and antibodies. Non-limiting examples include, FP-1039, an FGF ligand trap consisting of the extracellular domain of FGFR1 fused to the Fc portion of human IgG1, designed to sequester FGF ligands and inhibit FGF signaling, and MFGR1877S, a monoclonal antibody targeting FGF ligands, designed to block FGF-mediated signaling, including the compound structures disclosed therein which are specifically incorporated herein by reference.

x. VEGF Inhibitors

In some embodiments, compositions and methods described herein may include a compound of the present disclosure in combination with one or more vascular endothelial growth factor (VEGF) signaling inhibitors. VEGF (vascular endothelial growth factor) signaling inhibitors are a class of drugs that target the signaling pathway mediated by VEGF and its receptors. VEGF plays a critical role in angiogenesis, the process of forming new blood vessels from existing ones, and it is overexpressed in many types of cancer, making it an attractive target for cancer therapy. A VEGF inhibitor may be administered or formulated in combination with a compound of the present disclosure and/or any additional therapeutic agent described herein. In some embodiments, the VEGF inhibitor is an antibody or antigen binding regions that specifically bind VEGF (e.g., bevacizumab), or soluble VEGF receptors or a ligand binding region thereof) such as VEGF-TRAP™, and anti-VEGF receptor agents (e.g., antibodies or antigen binding regions that specifically bind thereto). In some embodiments, the VEGF inhibitor is one or more of bevacizumab, aflibercept, ramucirumab, sorafenib, sunitinib, and pazopanib.

### e) PI13K/mTOR Pathway Inhibitors

Compositions and methods described herein may include a compound of the present disclosure in combination with one or more inhibitors of the PI3K-AKT-TOR signaling pathway. The PI3K-AKT-mTOR signaling pathway is a critical intracellular pathway that regulates a wide range of cellular processes including cell growth, proliferation, metabolism, and survival. The pathway is initiated when growth factors, such as insulin or IGF-1, bind to cell surface receptors and activate phosphoinositide 3-kinase (PI3K). Activated PI3K then phosphorylates phosphatidylinositol 4,5-bisphosphate (PIP2) to produce phosphatidylinositol 3,4,5-trisphosphate (PIP3), which in turn activates AKT. Activated AKT then phosphorylates a variety of downstream targets including the tuberous sclerosis complex (TSC1/TSC2), leading to the activation of mTOR (mammalian target of rapamycin) complex 1 (mTORC1). Activated mTORC1 promotes protein synthesis and cell growth by phosphorylating key regulators of translation initiation such as S6 kinase (S6K) and eukaryotic initiation factor 4E-binding protein 1 (4E-BP1).

i. PI3K Inhibitors

In some embodiments, compositions and methods described herein may include a compound of the present disclosure in combination with one or more PI3K inhibitors. A PI3K inhibitor may be administered or formulated in combination with a compound of the present disclosure and/or any additional therapeutic agent described herein. A PI3K inhibitor useful in a combination of the disclosure may be a PI3Kα:RAS breaker, such as BBO-10203. PI3K inhibitors include, but are not limited to, wortmannin; 17-hydroxywortmannin analogs described in WO 2006044453; 4-[2-(1H-Indazol-4-yl)-6-[[4-(methylsulfonyl)piperazin-1-yl]methyl]thieno[3,2-d]pyrimidin-4-yl]morpholine (also known as pictilisib or GDC-0941 and described in WO 2009036082 and WO 2009055730); 2-methyl-2-[4-[3-methyl-2-oxo-8-(quinolin-3-yl)-2,3-dihydroimidazo[4,5-c]quinolin-1-yl]phenyl]propionitrile (also known as BEZ 235 or NVP-BEZ 235, and described in WO 2006122806); (S)-I-(4-((2-(2-aminopyrimidin-5-yl)-7-methyl-4-morpholinothieno[3,2-d]pyrimidin-6-yl)methyl)piperazin-1-yl)-2-hydroxypropan-1-one (described in WO 2008070740); LY294002 (2-(4-morpholinyl)-8-phenyl-4H-1-benzopyran-4-one (available from Axon Medchem); PI 103 hydrochloride (3-[4-(4-morpholinylpyrido-[3′,2′:4,5]furo[3,2-d]pyrimidin-2-yl]phenol hydrochloride (available from Axon Medchem); PIK 75 (2-methyl-5-nitro-2-[(6-bromoimidazo[1,2-a]pyridin-3-yl)methylene]-1-methylhydrazide-benzenesulfonic acid, monohydrochloride) (available from Axon Medchem); PIK 90 (N-(7,8-dimethoxy-2,3-dihydro-imidazo[1,2-c]quinazolin-5-yl)-nicotinamide (available from Axon Medchem); AS-252424 (5-[I-[5-(4-fluoro-2-hydroxy-phenyl)-furan-2-yl]-meth-(Z)-ylidene]-thiazolidine-2,4-dione (available from Axon Medchem); TGX-221 (7-methyl-2-(4-morpholinyl)-9-[1-(phenylamino)ethyl]-4H-pyrido-[1,2-a]pyrirnidin-4-one (available from Axon Medchem); XL-765; and XL-147. Other PI3K inhibitors include demethoxyviridin, perifosine, CAL101, PX-866, BEZ235, SF1126, INK1117, IPI-145, BKM120, XL147, XL765, Palomid 529, GSK1059615, ZSTK474, PWT33597, IC87114, TGI 00-115, CAL263, PI-103, GNE-477, CUDC-907, and AEZS-136. In some embodiments, the PI3K inhibitor is alpelisib or copanlisib. In some embodiments, reference to the term PI3K inhibitor includes any such PI3K inhibitor disclosed in any one of the following patent applications WO 2025072451 A1, WO 2025061125 A1, WO 2025051235 A1, WO 2025045106 A1, WO 2025040167 A1, WO 2025036439 A1, WO 2025038698 A1, WO 2025038395 A1, WO 2025034858 A1, WO 2025034849 A1, WO 2025029683 A1, WO 2025016314 A1, WO 2025003330 A1, WO 2025007074 A1, WO 2025002179 A1, WO 2024260464 A1, WO 2024229121 A1, WO 2024222894 A1, WO 2024215799 A1, WO 2024192309 A1, WO 2024183806 A1, WO 2024182404 A1, WO 2024182447 A1, each of which is incorporated herein by reference in its entirety.

ii. AKT Inhibitors

In some embodiments, compositions and methods described herein may include a compound of the present disclosure in combination with one or more AKT inhibitors. An AKT inhibitor may be administered or formulated in combination with a compound of the present disclosure and/or any additional therapeutic agent described herein. AKT inhibitors include, but are not limited to, ipatasertib, GSK-2141795, Akt-1-1 (inhibits Aktl) (Barnett et al., Biochem. J. 2005, 385(Pt. 2): 399-408); Akt-1-1,2 (inhibits Akl and 2) (Barnett et al., Biochem. J. 2005, 385(Pt. 2): 399-408); API-59CJ-Ome (e.g., Jin et al., Br. J. Cancer 2004, 91:1808-12); 1-H-imidazo[4,5-c]pyridinyl compounds (e.g., WO 05/011700); indole-3-carbinol and derivatives thereof (e.g., U.S. Pat. No. 6,656,963; Sarkar and Li J Nutr. 2004, 134(12 Suppl):3493S-3498S); perifosine (e.g., interferes with Akt membrane localization; Dasmahapatra et al. Clin. Cancer Res. 2004, 10(15):5242-52); phosphatidylinositol ether lipid analogues (e.g., Gills and Dennis Expert. Opin. Investig. Drugs 2004, 13:787-97); and triciribine (TCN or API-2 or NCI identifier: NSC 154020; Yang et al., Cancer Res. 2004, 64:4394-9). The PI3K/AKT inhibitor may include, but is not limited to, one or more PI3K/AKT inhibitors described in Cancers (Basel) 2015 September; 7(3): 1758-1784. For example, the PI3K/AKT inhibitor may be selected from one or more of NVP-BEZ235; BGT226; XL765/SAR245409; SF1126; GDC-0980; PI-103; PF-04691502; PKI-587; and GSK2126458.

iii. mTOR Inhibitors

In some embodiments, compositions and methods described herein may include a compound of the present disclosure in combination with one or more mTOR inhibitors. A mTOR inhibitor may be administered or formulated in combination with a compound of the present disclosure and/or any additional therapeutic agent described herein. mTOR inhibitors include, but are not limited to, ATP-competitive mTORC1/mTORC2 inhibitors, e.g., PI-103, PP242, PP30; Torin 1; FKBP12 enhancers; 4H-1-benzopyran-4-one derivatives; and rapamycin (also known as sirolimus) and derivatives thereof, including: temsirolimus (Torisel®); everolimus (Afinitor®; WO 199409010); ridaforolimus (also known as deforolimus or AP23573); rapalogs, e.g., as disclosed in WO 199802441 and WO 200114387, e.g. AP23464 and AP23841; 40-(2-hydroxyethyl)rapamycin; 40-[3-hydroxy(hydroxymethyl)methylpropanoate]-rapamycin (also known as CC1779); 40-epi-(tetrazolyt)-rapamycin (also called ABT578); 32-deoxorapamycin; 16-pentynyloxy-32(S)-dihydrorapanycin; derivatives disclosed in WO 2005005434; derivatives disclosed in U.S. Pat. Nos. 5,258,389, 5,118,677, 5,118,678, 5,100,883, 5,151,413, 5,120,842, and 5,256,790, and in WO 1994090101, WO 199205179, WO 1993111130, WO 199402136, WO 199402485, WO 199514023, WO 199402136, WO 199516691, WO 199641807, WO 199641807, and WO 2018204416; and phosphorus-containing rapamycin derivatives (e.g., WO 2005016252). In some embodiments, the mTOR inhibitor is a bisteric inhibitor (see, e.g., WO 2018204416, WO 2019212990 and WO 2019212991), such as RMC-5552.

iv. MNK Inhibitors

In some embodiments, compositions and methods described herein may include a compound of the present disclosure in combination with one or more mitogen-activated protein kinase-interacting kinase (MNK) inhibitors. A MNK inhibitor may be administered or formulated in combination with a compound of the present disclosure and/or any additional therapeutic agent described herein. MNK proteins are activated downstream of the mitogen-activated protein kinase (MAPK) signaling pathway, which plays a critical role in the regulation of cellular proliferation, differentiation, and survival. MNKs phosphorylate eIF4E, a key component of the eukaryotic translation initiation complex, which enhances the translation of specific mRNAs, including those encoding proteins involved in cell cycle regulation and oncogenesis. In some embodiments, a MNK inhibitor is one or more tomivosertib (eFT508), CGP57380, and SEL201. In some embodiments, reference to the term MNK inhibitor includes any such MNK inhibitor disclosed in any one of the following patent applications: WO 2021098691, WO 2020108619, WO 2020086713, WO 2018152117, WO 2018228275, WO 2015200481, and CN115583942, each of which is incorporated herein by reference in its entirety, including the compound structures disclosed therein which are specifically incorporated herein by reference.

v. EIF4 Inhibitors

In some embodiments, compositions and methods described herein may include a compound of the present disclosure in combination with one or more eukaryotic initiation factor 4A (eIF4A) inhibitors. An eIF4A inhibitor may be administered or formulated in combination with a compound of the present disclosure and/or any additional therapeutic agent described herein. eIF4A is a critical component of the eukaryotic translation initiation complex, where it functions as an RNA helicase to unwind the secondary structure of mRNA and facilitate ribosome binding. eIF4A is required for the translation of many cancer-associated genes, making it an attractive therapeutic target for cancer treatment. In some embodiments, an eIF4A inhibitor is one or more zotatifin (eFT226), silvestrol, pateamine A, and rocaglates. In some embodiments, reference to the term eIF4A inhibitor includes any such eIF4A inhibitor disclosed in any one of the following patent applications: WO 2023034813, WO 2021195128, and WO 2017091585, each of which is incorporated herein by reference in its entirety, including the compound structures disclosed therein which are specifically incorporated herein by reference.

In some embodiments, compositions and methods described herein may include one or more eukaryotic initiation factor 4G (eIF4G) inhibitors. An eIF4G inhibitor may be administered or formulated in combination with a compound of the present disclosure and/or any additional therapeutic agent described herein. eIF4G family includes several proteins that are involved in the initiation of protein translation. eIF4G serves as a scaffold for other proteins, including eIF4E and eIF4A, to form the eIF4F complex, which is responsible for binding to the 5′ cap of mRNA and unwinding the secondary structure of the mRNA to allow ribosomal scanning and translation initiation. In some embodiments, an eIF4G inhibitor is one or more pateamine A, and hippuristanol.

### f) DNA Damage Response Inhibitors

Compositions and methods described herein may include a compound of the present disclosure in combination with one or more DNA damage response (DDR) inhibitors. The DDR pathway is a critical cellular pathway that is activated in response to DNA damage and is essential for maintaining genomic stability, thereby preventing the development of cancer. However, cancer cells often have defects in the DDR pathway, which makes them more sensitive to DDR inhibitors. DDR inhibitors have shown promise in preclinical studies as potential cancer therapeutics, particularly in combination with other agents.

i. Wee1 Inhibitors

In some embodiments, compositions and methods described herein may include one or more Wee1 inhibitors. A Wee1 inhibitor may be administered or formulated in combination with a compound of the disclosure and/or any additional therapeutic agent described herein. Wee1 is a kinase that plays a critical role in regulating the cell cycle by inhibiting the activity of cyclin-dependent kinases (CDKs) and preventing the progression of cells through the G2/M checkpoint. Wee1 is overexpressed in several cancer types and has been implicated in tumor growth and survival. In some embodiments, a Wee1 inhibitor is one or more of imp7068, adavosertib, or ZNL-02-096. In some embodiments, reference to the term Wee1 inhibitor includes any such Wee1 inhibitor disclosed in any one of the following patent applications: WO 2022011391, WO 2022247641, WO 2021043152, WO 2020221358, WO 2020083404, WO 2020192581, WO 2019085933, WO 2018133829, WO 2015115355, WO 2015183776, WO 2014085216, and CN 114831993, each of which is incorporated herein by reference in its entirety, including the compound structures disclosed therein which are specifically incorporated herein by reference.

ii. CHK Inhibitors

In some embodiments, compositions methods described herein may include one or more checkpoint kinase (CHK) inhibitors. A CHK inhibitor may be administered or formulated in combination with a compound of the disclosure and/or any additional therapeutic agent described herein. CHK1 kinase is a critical regulator of the cell cycle and the DNA damage response pathway. In some embodiments, the CHK inhibitor is a CHK1 inhibitor. In some embodiments, a CHK inhibitor is a CHK2 inhibitor. In some embodiments, a CHK1 inhibitor is one or more BBI-355, rabusertib, LY2606368, LY2880070, GDC-0575, MK-8776, BEBT-260, and PEP07. In some embodiments, reference to the term CHK1 inhibitor includes any such CHK1 inhibitor disclosed in any one of the following patent applications: WO 2024196923, WO 2024211271, WO 2024211270, WO 2024118564, WO 2023230477, WO 2022251502, WO 2021113661, WO 2021104461, WO 2019012030, WO 2010118390, WO 2008067027, WO 2002070494, CN119661557, and TW202126818, each of which is incorporated herein by reference in its entirety, including the compound structures disclosed therein which are specifically incorporated herein by reference.

iii. ATM Inhibitors

In some embodiments, compositions and methods described herein may include one or more ataxia telangiectasia mutated (ATM) inhibitors. An ATM inhibitor may be administered or formulated in combination with a compound of the disclosure and/or any additional therapeutic agent described herein. ATM plays a role in regulating the replication stress response and maintaining genomic stability. In some embodiments, an ATM inhibitor is one or more Iartesertib, AZD1390, AZD0156, KU-60019, M4076, M3541, WSD-0628, ZN-B-2262, SYH2051, and VE-821. In some embodiments, reference to the term ATM inhibitor includes any such ATM inhibitor disclosed in any one of the following patent applications: WO 2024189299, WO 2022058351, WO 2021197339, WO 2021098734, WO 2021260580, WO 2020193660, WO 2020063855, WO 2016155884, WO 2007026157, WO 2006085067, US 2016113935, CN 116440082, CN 117180432 and CN 115105596 each of which is incorporated herein by reference in its entirety, including the compound structures disclosed therein which are specifically incorporated herein by reference.

iv. ATR Inhibitors

In some embodiments, compositions and methods described herein may include one or more ataxia telangiectasia and Rad3-related (ATR) inhibitors. An ATR inhibitor may be administered or formulated in combination with a compound of the disclosure and/or any additional therapeutic agent described herein. In some embodiments, an ATR inhibitor is one or more berzosertib, gartisertib, camonsertib, ceralaertib, VE-821, RP-3500, AZ20, VX-970, abd110, VX-803, and elimusertib (BAY 1895344). In some embodiments, reference to the term ATR inhibitor includes any such ATR inhibitor disclosed in any one of the following patent applications: WO 2025019344, WO 2025019346, WO 2023138343, WO 2023126823, WO 2023109883, WO 2023016529, WO 2022237875, WO 2022268025, WO 2021012049, WO 2021023272, WO 2021260579, WO 2021228758, WO 2019050889, WO 2019154365, WO 2019036641, WO 2019133711, WO 2017059357, WO 2013049859, WO 2007046426, WO 2007015632, and CN113797341, each of which is incorporated herein by reference in its entirety, including the compound structures disclosed therein which are specifically incorporated herein by reference.

v. PARP Inhibitors

In some embodiments, compositions and methods described herein may include one or more Poly(ADP-ribose) polymerase (PARP) inhibitors. A PARP inhibitor may be administered or formulated in combination with a compound of the disclosure and/or any additional therapeutic agent described herein. There are 17 PARP (aka tankyrase) family members that have been identified. PARP enzymes play a critical role in DNA damage repair, particularly in the repair of single-strand DNA breaks. PARP inhibitors block the activity of PARP enzymes, leading to the accumulation of DNA damage and ultimately cell death. In some embodiments, a PARP inhibitor is one or more olaparib, rucaparib, niraparib, and veliparib (ABT-888). In some embodiments, reference to the term PARP inhibitor includes any such PARP inhibitor disclosed in any one of the following patent applications: WO 2025024581, WO 2025037273, WO 2025061057, WO 2024256377, WO 2024255782, WO 2023051812, WO 2023051807, WO 2023051716, WO 2023278592, WO 2022228387, WO 2022022664, WO 2022000946, WO 2022222921, WO 2021163530, WO 2020122034, WO 2020239097, WO 2020142583, WO 2020156577, WO 2020098774, WO 2020196712, WO 2019200382, WO 2018125961, WO 2018205938, WO 2018192576, WO 2018218025, WO 2017032289, WO 2017177838, WO 2017029601, WO 2017088723, WO 2016155655, WO 2015154630, WO 2013097225, WO 2012130166, WO 2011006794, WO 2009046205, WO 2009063244, WO 2008084261, WO 2007138351, WO 2006110816, WO 2005053662, WO 2005012524, CN113698356, CN 113603647, CN 115073544, CN 108938634, CN 104887680, CN 110343088, CN108976236, CN 117069731, CN 119185316, CN 119112794, and CN 107629071, each of which is incorporated herein by reference in its entirety, including the compound structures disclosed therein which are specifically incorporated herein by reference.

vi. DNA-PK Inhibitors

In some embodiments, compositions and methods described herein may include one or more DNA-dependent protein kinase (DNA-PK) inhibitors. A DNA-PK inhibitor may be administered or formulated in combination with a compound of the disclosure and/or any additional therapeutic agent described herein. DNA-PK is a serine/threonine protein kinase that plays a crucial role in DNA repair and maintenance of genome stability. In some embodiments, a DNA-PK inhibitor is one or more NU7441, AZD7648, VX-984, peposertib (M3814), and CC-115. In some embodiments, reference to the term DNA-PK inhibitor includes any such DNA-PK inhibitor disclosed in any one of the following patent applications: WO 2025023957, WO 2023220418, WO 2023215991, WO 2023165603, WO 2022187965, WO 2021197159, WO 2021260583, WO 2021204111, WO 2021104277, WO 2021098813, WO 2021022078, WO 2020259613, WO 2019143678, WO 2019143675, WO 2019201283, WO 2015058031, WO 2014159690, WO 2012028233, WO 2009010761, WO 2006032869, WO 2006109084, CN 112574179, CN 112300132, CN 115322209, and CN 112300126, each of which is incorporated herein by reference in its entirety, including the compound structures disclosed therein which are specifically incorporated herein by reference.

### g) Cell Cycle Inhibitors

Compositions and methods described herein may include a compound of the present disclosure in combination with one or more cell cycle inhibitors. Cell cycle inhibitors target specific proteins involved in regulating the cell cycle, which is the process by which a cell divides and replicates its DNA. Non-limiting examples cell cycle proteins include cyclin-dependent kinase (CDK), aurora kinase, and polo-like kinase (PLK). CDKs are a family of kinases that are involved in regulating the cell cycle. CDK inhibitors block the activity of these kinases, leading to cell cycle arrest and/or apoptosis. Aurora kinases are a family of serine/threonine kinases that play a critical role in regulating mitosis. Aurora kinase inhibitors block the activity of these kinases, leading to mitotic arrest and cell death. PLKs are a family of serine/threonine kinases that are involved in regulating multiple stages of the cell cycle. PLK inhibitors block the activity of these kinases, leading to cell cycle arrest and/or apoptosis.

i. CDK Inhibitors

In certain embodiments, a cell cycle inhibitor is a cyclin-dependent kinase (CDK) inhibitor. Cyclin-dependent kinases are a family of protein kinases that regulate cell division and proliferation. Cell cycle progression is controlled by cyclins and their associated cyclin-dependent kinases, such as CDK1, CDK2, CDK3, CDK4 and CDK6, while other CDKs such as CDK7, CDK8 and CDK9 are critical to transcription. CDK binding to cyclins forms heterodimeric complexes that phosphorylate their substrates on serine and threonine residues, which in turn initiates events required for cell-cycle transcription and progression. In some embodiments, a CDK inhibitor is a CDK2 inhibitor. In some embodiments, a CDK inhibitor is a CDK4/6 inhibitor. In some embodiments, a CDK inhibitor is a CDK7 inhibitor. In some embodiments, a CDK inhibitor is a CDK9 inhibitor. In some embodiments, a CDK inhibitor is one or more palbociclib, ribociclib, abemaciclib, and trilaciclib. In some embodiments, a CDK inhibitor is one or more of tagtociclib (PF-07104091), seliciclib, voruciclib (P1446A-05), BLU-222, dinaciclib, AT-7519, RGB286638, and AZD4573.

In some embodiments, reference to the term CDK inhibitor includes any such CDK inhibitor disclosed in any one of the following patent applications: WO 2025040170, WO 2025060620, WO 2024238574, WO 2024027825, WO 2024048541, WO 2022166793, WO 2022187611, WO 2022130304, WO 2021227906, WO 2021057867, WO 2020207260, WO 2020138370, WO 2020125513, WO 2020093011, WO 2020148635, WO 2020215156, WO 2020052627, WO 2017177837, WO 2017162215, WO 2017177836, WO 2017172826, WO 2016193939, WO 2016014904, WO 2016015598, WO 2016015605, WO 2015181737, WO 2012061156 A1, WO 2012038411, WO 2010020675, WO 2010125004, WO 2007139732, WO 2006024945, CN 114478529, CN 108794496, CN 105294737, CN107652284, KR 20180106188, and US 2017152269, each of which is incorporated herein by reference in its entirety, including the compound structures disclosed therein which are specifically incorporated herein by reference.

ii. Aurora Kinase Inhibitors

In some embodiments, compositions and methods described herein may include a compound of the present disclosure in combination with one or more aurora kinase inhibitors. An aurora kinase inhibitor may be administered or formulated in combination with a compound of the present disclosure and/or any additional therapeutic agent described herein. Aurora kinases are a family of serine/threonine kinases that play a critical role in regulating cell division and maintaining genomic stability. The Aurora kinase family consists of three members: Aurora A, Aurora B, and Aurora C. In some embodiments, an aurora kinase inhibitor is one or more palbociclib, ribociclib, and abemaciclib. In some embodiments, an aurora kinase inhibitor is one or more of alisertib, danusertib, barasertib, and MLN8237. In some embodiments, reference to the term aurora kinase inhibitor includes any such aurora kinase inhibitor disclosed in any one of the following patent applications: WO 2021110009, WO 2021008338, WO 2020112514, WO 2019129234, WO 2016077161, WO 2013143466, WO 2011103089, WO 2010081881, WO 2010133794, WO 2009134658, WO 2008001886, WO 2007095124, WO 2007003596, WO 2006129064, CN 114276227, CN 108078991, CN 106543155, CN 104211692, and CN 104098551, each of which is incorporated herein by reference in its entirety, including the compound structures disclosed therein which are specifically incorporated herein by reference.

iii. PLK Inhibitors

In some embodiments, compositions and methods described herein may include a compound of the present disclosure in combination with one or more polo-like kinase (PLK) inhibitors. A PLK inhibitor may be administered or formulated in combination with a compound of the present disclosure and/or any additional therapeutic agent described herein. PLKs are a family of serine/threonine kinases that play a crucial role in regulating cell division, DNA damage response, mitotic progression, and consists of four members: PLK1, PLK2, PLK3, and PLK4. In some embodiments, a PLK inhibitor is one or more of volasertib, onvansertib, BI 2536, and GSK461364. In some embodiments, reference to the term PLK inhibitor includes any such PLK inhibitor disclosed in any one of the following patent applications: WO 2011012534 A1, WO 2010065134, WO 2009130453, WO 2009042806, WO 2004043936, WO 2007030361, WO 2006021547, CN 115804777, and EP 2325185, each of which is incorporated herein by reference in its entirety, including the compound structures disclosed therein which are specifically incorporated herein by reference.

iv. Kinesin Superfamily of Microtubule Motor Protein Inhibitors

In some embodiments, compositions and methods described herein may include a compound of the present disclosure in combination with one or more Kinesin spindle protein (KSP) inhibitors. In some embodiments, compositions described herein may include one or more Kinesin family (KIF) inhibitors. In some embodiments, a KSP inhibitor may be administered or formulated in combination with a compound of the present disclosure and/or any additional therapeutic agent described herein. KSP and KIF are a subset of the kinesin superfamily of microtubule motor proteins. KSP, also known as Eg5, is a member of the kinesin superfamily of motor proteins that plays a critical role in mitotic spindle formation and cell division. KSP inhibitors selectively target rapidly dividing cancer cells by disrupting spindle formation and inducing mitotic arrest. In some embodiments, a KSP inhibitor is one or more of SB743921, monastrol, S-Trityl-L-cysteine (STLC), and filanesib (ARRY-520). In some embodiments, a KIF inhibitor is an inhibitor of a Kinesin-8 family microtubule motor protein. In some embodiments, the kinesin-8 family protein is KIF18A. In some embodiments, a KIF inhibitor is one or more of AMG650, BTB-1, K03861, and SJ000291942. In some embodiments, reference to the term kinesin superfamily of microtubule motor protein inhibitor includes any such kinesin superfamily of microtubule motor protein inhibitor disclosed in any one of the following patent applications: WO 2015114854, WO 2015114855, WO 2010084186, WO 2006101761, WO 2006110390, WO 2006044825, WO 2006078574, WO 2005060654, WO 2004092147, WO 2004037171, WO 2004058700, WO 2003050064, WO 2003105855, WO 2022037665, WO 2018114804, WO 2017162663, WO 2016207089, WO 2012073375, JP 2014162787, JP 2019189590, JP2013166713, and KR 20220145566, each of which is incorporated herein by reference in its entirety, including the compound structures disclosed therein which are specifically incorporated herein by reference.

v. DYRK1 Inhibitors

In some embodiments, compositions and methods described herein may include a compound of the present disclosure in combination with one or more Dual-specificity tyrosine phosphorylation-regulated kinase 1 (DYRK1) inhibitors. A DYRK1 inhibitor may be administered or formulated in combination with a compound of the present disclosure and/or any additional therapeutic agent described herein. DYRK1 is a member of the DYRK (dual-specificity tyrosine phosphorylation-regulated kinase) family of protein kinases. It plays essential roles in various cellular processes, including cell cycle regulation, neuronal development, and transcriptional control. In some embodiments, a DYRK1 inhibitor is one or more of harmine, INDY, D4476, and AZ191. In some embodiments, reference to the term DYRK1 inhibitor includes any such DYRK1 inhibitor disclosed in any one of the following patent applications: WO 2023277331 A1, WO 2023140846 A1, WO 2017181087 A1, each of which is incorporated herein by reference in its entirety, including the compound structures disclosed therein which are specifically incorporated herein by reference.

### h) Anti-Apoptotic Protein Inhibitors

Compositions and methods described herein may include a compound of the present disclosure in combination with one or more anti-apoptotic protein inhibitors. In some embodiments, an anti-apoptotic protein inhibitor may be administered or formulated in combination with a compound of the present disclosure and/or any additional therapeutic agent described herein. Anti-apoptotic inhibitors target proteins that play a role in preventing apoptosis, a form of programmed cell death. Apoptosis is a critical mechanism for eliminating damaged or unwanted cells. Anti-apoptotic proteins are a family of proteins that inhibit the apoptotic pathway, thereby preventing cell death. There are several known classes of anti-apoptotic inhibitors, including Bcl-2 inhibitors, XIAP inhibitors, survivin inhibitors, Mcl-1 inhibitors, and FLIP inhibitors. These inhibitors work by binding to specific anti-apoptotic proteins and preventing their activity, thereby promoting cell death in cancer cells. In some embodiments, compositions described herein may include one or more anti-apoptotic protein inhibitors. An anti-apoptotic protein inhibitor may be administered or formulated in combination with a compound of the disclosure and/or any additional therapeutic agent described herein. In some embodiments, the anti-apoptotic protein inhibitor includes a MCL-1 inhibitor. Non-limiting examples of MCL-1 inhibitors include, AMG-176, MIK665, and S63845. The myeloid cell leukemia-1 (MCL-1) protein is one of the key anti-apoptotic members of the B-cell lymphoma-2 (BCL-2) protein family. Over-expression of MCL-1 has been closely related to tumor progression as well as to resistance, not only to traditional chemotherapies but also to targeted therapeutics including BCL-2 inhibitors such as ABT-263. In some embodiments, the anti-apoptotic protein inhibitor includes a BCL protein inhibitor. Examples of BCL protein inhibitors include but are not limited to Venetoclax (Venclexta), Navitoclax (ABT-263), A-1331852, S63845, and AT-101.

### i) Autophagy Inhibitors

Compositions and methods described herein may include a compound of the present disclosure in combination with one or more autophagy inhibitors. In some embodiments, an autophagy inhibitor may be administered or formulated in combination with a compound of the present disclosure and/or any additional therapeutic agent described herein. Autophagy inhibitors include, but are not limited to chloroquine, 3-methyladenine, hydroxychloroquine (PLAQUENIL™), spautin-1, SAR405, bafilomycin A1, 5-amino-4-imidazole carboxamide riboside (AICAR), okadaic acid, autophagy-suppressive algal toxins which inhibit protein phosphatases of type 2A or type 1, analogues of cAMP, and drugs which elevate cAMP levels such as adenosine, LY204002, N6-mercaptopurine riboside, and vinblastine. In addition, antisense or siRNA that inhibits expression of proteins including but not limited to ATG5 (which are implicated in autophagy), may also be used. In some embodiments, the one or more additional therapies include an autophagy inhibitor.

### a) ULK Inhibitors

In some embodiments, compositions and methods described herein may include a compound of the present disclosure in combination with one or more Unc-51-like kinase (ULK) inhibitors. An ULK inhibitor may be administered or formulated in combination with a compound of the present disclosure and/or any additional therapeutic agent described herein. In some embodiments, a ULK inhibitor is a ULK1/2 inhibitor. In some embodiments, an ULK inhibitor is one or more of ULK-101, MRT68921, SBI-0206965, MRT67307, MRT68920, MRT68922, MRT199665, LY3009120, and Dorsomorphin.

### b) VPS Inhibitors

In some embodiments, compositions and methods described herein may include a compound of the present disclosure in combination with one or more Vacuolar protein sorting protein (VPS) inhibitors. A VPS inhibitor may be administered or formulated in combination with a compound of the present disclosure and/or any additional therapeutic agent described herein. VPS (proteins are a family of proteins that play a critical role in the process of autophagy by regulating the formation and function of autophagosomes, structures that engulf and transport cellular components to lysosomes for degradation. Dysregulation of VPS proteins has been implicated in various diseases, including cancer, neurodegenerative disorders, and infectious diseases. In some embodiments, a VPS inhibitor is a VPS34 inhibitor. In some embodiments, a VPS inhibitor is one or more of PIK-III, VPS34-IN1, SAR405, Spautin-1, and NSC185058.

### c) Macropinocytosis Inhibitors

In some embodiments, compositions and methods described herein may include a compound of the present disclosure in combination with one or more macropinocytosis inhibitors. A macropinocytosis inhibitor may be administered or formulated in combination with a compound of the disclosure and/or any additional therapeutic agent described herein. Macropinocytosis inhibitors are compounds that can block or reduce the process of macropinocytosis. In some embodiments, a macropinocytosis inhibitor is one or more of EIPA (ethylisopropylamiloride), Wortmannin, Amiloride, Apilimod, Dyngo-4a, and Latrunculin B.

### j) WNT/b-Catenin Pathway Inhibitors

Compositions and methods described herein may include a compound of the present disclosure in combination with one or more WNT/beta-catenin pathway inhibitors. In some embodiments, a WNT/beta-catenin pathway inhibitor may be administered or formulated in combination with a compound of the present disclosure and/or any additional therapeutic agent described herein. The WNT/beta-catenin pathway is an important signaling pathway that plays a crucial role in development, tissue homeostasis, and disease. Dysregulation of this pathway has been implicated in various cancers, making it an attractive target for cancer therapy. WNT/beta-catenin pathway inhibitors target various components of the pathway, including WNT ligands, receptors, and downstream effectors.

### i. β-Catenin Inhibitors

In some embodiments, compositions and methods described herein may include a compound of the present disclosure and one or more β-catenin inhibitors. A β-catenin inhibitor may be administered or formulated in combination with a compound of the present disclosure and/or any additional therapeutic agent described herein. Beta-catenin is a protein that plays an important role in the WNT signaling pathway, which regulates various cellular processes including cell proliferation, differentiation, and migration. In normal cells, β-catenin levels are tightly regulated by a destruction complex, which marks beta-catenin for degradation. However, in many cancer cells, the destruction complex is impaired, leading to the accumulation of beta-catenin in the nucleus and the activation of target genes involved in tumor growth and metastasis. In some embodiments, a WNT/b-catenin inhibitor is one or more of FOG-001, OMP-131R10, Foxy-5, LGK974, RXC004, ETC-159, OMP-54F28, Niclosamide, OMP-18R5, OTSA-101, BNC101, DKN-01, Sulindac, Pyrvinium, E7449, BC2059, PRI-724, SM08502, IWP1, IWP2, IWP3, IWP4, IWP12, IWP L6, C59, GNF-6231, GNF-1331, DK-520, DK-419, IgG-2919, Fz7-21, RHPD-P1, SR137892, 1094-0205, 2124-0331, 3235-0367, NSC36784, NSC654259, IgG-2919, Salinomycin, BMD4702, 3289-8625, J01-017a, FJ9, KY-02061, KY-02327, NSC668036, Peptide Pen-N3, SSTC3, CCT031374, TCS 183, XAV939, AZ1366, G007-LK, MSC2504877, G244-LM, IWR-1, JW74, JW55, K-756, NVP-TNKS656, MN-64, RK-287107, WIKI4, KY1220, KYA1797K, MSAB, PKF115-584, CGP049090, AV-65, PNU-74654, Windorphen, IQ-1 tegavivant, foscenvivant, PNPB-29, ZW4864, SAH-BCL9, Carnosic acid, xStAx-VHL, NRX-252114, Septuximab vedotin, PF-06647020, LGR5-mc-vc-PAB-MMAE, LGR5-NMS818, CWP232291, PRI-724 (also known as ICG-001), C-82, and BC2059. In some embodiments, reference to the term β-catenin inhibitor includes any such β-catenin inhibitor disclosed in any one of the following patent applications: CN 104388427 and CN 103830211, each of which is incorporated herein by reference in its entirety, including the compound structures disclosed therein which are specifically incorporated herein by reference.

ii. PORCN Inhibitors

In some embodiments, compositions and methods described herein may include a compound of the present disclosure in combination with one or more Porcupine (PORCN) inhibitors. A PORCN inhibitor may be administered or formulated in combination with a compound of the present disclosure and/or any additional therapeutic agent described herein. PORCN is a membrane-bound O-acyltransferase enzyme that plays a critical role in the WNT signaling pathway by mediating the palmitoylation of WNT ligands. This palmitoylation is essential for the secretion and signaling activity of WMT proteins. Inhibition of PORCN leads to reduced WNT signaling activity. In some embodiments, a PORCN inhibitor is one or more of LGK974 (WNT974), ETC-1922159, CGX1321, and CWP232291.

iii. GSK3 Inhibitors

In some embodiments, compositions and methods described herein may include a compound of the present disclosure in combination with one or more Glycogen synthase kinase (GSK3) inhibitors. A GSK3 inhibitor may be administered or formulated in combination with a compound of the present disclosure and/or any additional therapeutic agent described herein. The GSK3 family consists of two closely related serine/threonine kinases: GSK3α and GSK3β. These kinases are involved in numerous cellular processes, including glycogen metabolism, cell cycle regulation, and Wnt signaling. GSK inhibitors have been investigated as potential therapeutics for various diseases, including cancer, diabetes, Alzheimer's disease, and bipolar disorder. In some embodiments, a GSK3 inhibitor is one or more of Tideglusib, laduviglusib, LiCl (Lithium chloride), CHIR99021, SB216763, AZD1080, and LY2090314. In some embodiments, reference to the term GSK3 inhibitor includes any such GSK3 inhibitor disclosed in any one of the following patent applications: WO 2017153834, WO 2014059383, WO 2010012398, WO 2009017455, WO 2003037891, CN 107151235, and CN 102258783, each of which is incorporated herein by reference in its entirety, including the compound structures disclosed therein which are specifically incorporated herein by reference.

iv. CLK Inhibitors

In some embodiments, compositions and methods described herein may include a compound of the present disclosure in combination with one or more Cdc2-like kinase (CLK) inhibitors. A CLK inhibitor may be administered or formulated in combination with a compound of the present disclosure and/or any additional therapeutic agent described herein. LKs (Cdc2-like kinases) are a family of serine/threonine kinases that play a crucial role in pre-mRNA splicing, specifically in the regulation of alternative splicing. There are four members of the CLK family: CLK1, CLK2, CLK3, and CLK4. The CLK family of kinases have been shown to be involved in several diseases, including cancer, neurodegenerative disorders, and viral infections. In some embodiments, a CLK inhibitor is a CLK 2 inhibitor. In some embodiments, a CLK2 inhibitor is one or more of Lorecivivint, SM08502, SM04690, TG003, KH-CB19, Cmpd-1, T3.5, and CX-4945. In some embodiments, reference to the term CLK inhibitor includes any such CLK inhibitor disclosed in WO 2020006115, which is incorporated herein by reference in its entirety, including the compound structures disclosed therein which are specifically incorporated herein by reference.

### k) JAK/STAT Pathway Inhibitors

Compositions and methods described herein may include a compound of the present disclosure in combination with one or more JAK/STAT pathway inhibitors. In some embodiments, a JAK/STAT pathway inhibitor may be administered or formulated in combination with a compound of the present disclosure and/or any additional therapeutic agent described herein. The Janus kinase/signal transducer and activator of transcription (JAK/STAT) pathway is a signaling pathway involved in many cellular processes, including immune response, cell growth, and differentiation. Dysregulation of this pathway has been linked to various diseases, including inflammatory disorders, cancer, and autoimmune diseases. Inhibitors of the JAK/STAT pathway can be used for the treatment of these diseases. In some embodiments, a JAK/STAT pathway inhibitor is an inhibitor of JAK1, JAK2 and/or JAK3. In some embodiments, a JAK inhibitor is one or more of Ruxolitinib (JAKAFI®), Pacritinib, Fedratinib, Tofacitinib (XELJANZ®), Abrocitinib, Filgotinib, Oclacitinib, Peficitinib, Upadacitinib, Deucravacitinib, Delgocitinib, and Baricitinib (OLUMIANT®). In some embodiments, reference to the term JAK inhibitor includes any such JAK inhibitor disclosed in any one of the following patent applications: WO 2023011301, WO 2023201044, WO 2022143629, WO 2022251434, WO 2022067106, WO 2022033551, WO 2021244323, WO 2021238817, WO 2021238818, WO 2021178991, WO 2021136345, WO 2021190647, WO 2020219639, WO 2020182159, WO 2020155931, WO 2020038457, WO 2020219524, WO 2020173400, WO 2018204233, WO 2018204238, WO 2018169875, WO 2018117152, WO 2017215630, WO 2016070697, WO 2016027195, CN 117815195, CN117815367, and CN 115969796, each of which is incorporated herein by reference in its entirety, including the compound structures disclosed therein which are specifically incorporated herein by reference.

In some embodiments, the JAK/STAT pathway inhibitor is a STAT inhibitor. In some embodiments, the STAT inhibitor is an inhibitor of STAT3 and/or STAT5. In some embodiments, the STAT inhibitor is a STAT3 degrader. In some embodiments, the STAT3 degrader is KT-333. In some embodiments, the STAT inhibitor is one or more of TTI-101, C-188-9, WP1066, VVD-130850, LLL12B, STA-21, SD-36, Stattic, S31-201, OPB-31121, KT-333, and Napabucasin (BB1608). In some embodiments, reference to the term STAT inhibitor includes any such STAT inhibitor disclosed in any one of the following patent applications: WO 2024030628, WO 2023164680, WO 2023192960, WO 2023133336, WO2020206424, WO 2023107706, WO 2021150543, WO 2008151037, and CN 109288845, each of which is incorporated herein by reference in its entirety, including the compound structures disclosed therein which are specifically incorporated herein by reference.

### l) Epigenetic Modulators

Compositions and methods described herein may include a compound of the present disclosure in combination with one or more epigenetic modulators. Epigenetic modulators are a class of therapeutics that target enzymes responsible for modifying the structure and function of chromatin, the complex of DNA and proteins that make up chromosomes. These enzymes, including histone deacetylases (HDACs), histone methyltransferases (HMTs), and DNA methyltransferases (DNMTs), play critical roles in gene expression and regulation by modifying the packaging of DNA and affecting how it is read and transcribed. Epigenetic modulators work by altering the activity of these enzymes, either by inhibiting or enhancing their function, to regulate gene expression in specific ways. By targeting specific epigenetic modifications, such as acetylation, methylation, and DNA methylation, these therapies have the potential to treat a wide range of diseases, including cancer, inflammatory disorders, and neurological disorders.

i. HDAC Inhibitors

In some embodiments, compositions and methods described herein may include a compound of the present disclosure in combination with one or more histone deacetylase (HDAC) inhibitors. A HDAC inhibitor may be administered or formulated in combination with a compound of the present disclosure and/or any additional therapeutic agent described herein. There are several classes of HDACs, including class I, class IIa, class IIb, class III, and class IV. Class I HDACs are further divided into HDAC1, HDAC2, HDAC3, and HDAC8, while class IIa HDACs include HDAC4, HDAC5, HDAC7, and HDAC9. Class IIb HDACs consist of HDAC6 and HDAC10, and class III HDACs are known as sirtuins. HDAC inhibitors can target different classes of HDACs, and their specific effects on gene expression can vary depending on which HDACs they target. In some embodiments, a HDAC inhibitor is one or more of Vorinostat (ZOLINZA™), Romidepsin (ISTODAX™), Belinostat (BELEODAQ™), Panobinostat (FARYDAK™) Entinostat (MS-275), Valproic acid (DEPAKENE™), Trichostatin A (TSA), Sodium butyrate, and Mocetinostat (MGCD0103). Non-limiting examples of HDAC inhibitors include trichostatin, sodium butyrate, apicidan, suberoyl anilide hydroamic acid, vorinostat, LBH 589, romidepsin, ACY-1215, and Panobinostat. In some embodiments, reference to the term HDAC inhibitor includes any such HDAC inhibitor disclosed in any one of the following patent applications: WO 2022110958, WO 2021252628, WO 2019204550, WO 2018178060, WO 2016126724, WO 2014143666, WO 2013041480, and WO 2006120456, each of which is incorporated herein by reference in its entirety, including the compound structures disclosed therein which are specifically incorporated herein by reference.

ii. BET Inhibitors

In some embodiments, compositions and methods described herein may include a compound of the present disclosure in combination with one or more bromodomain and extra-terminal protein (BET) inhibitors. A BET inhibitor may be administered or formulated in combination with a compound of the present disclosure and/or any additional therapeutic agent described herein. BET (bromodomain and extra-terminal) proteins are a family of epigenetic reader proteins that recognize and bind to acetylated lysine residues on histones, leading to chromatin remodeling and gene expression regulation. There are four BET proteins in humans: BRD2, BRD3, BRD4, and BRDT. BET inhibitors specifically target the bromodomains of BET proteins, inhibiting their binding to acetylated lysine residues on histones and leading to alterations in gene expression. BET inhibitors are useful in the treatment of cancer and other diseases characterized by dysregulated gene expression. In some embodiments, a BET inhibitor is one or more of JQ1, I-BET762, OTX015, RVX-208, and CPI-0610. In some embodiments, reference to the term BET inhibitor includes any such BET inhibitor disclosed in any one of the following patent applications: WO 2022046682, WO 2022182857, WO 2021107657, WO 2021107656, WO 2020221006, WO 2020053660, WO 2018097977, WO 2017222977, WO 2017142881, WO 2015075665, WO 2015011084, and CN 113264930, each of which is incorporated herein by reference in its entirety, including the compound structures disclosed therein which are specifically incorporated herein by reference.

iii. EZH2 Inhibitors

In some embodiments, compositions and methods described herein may include a compound of the present disclosure in combination with one or more Enhancer of Zeste Homolog 2 (EZH2) inhibitors. An EZH2 inhibitor may be administered or formulated in combination with a compound of the present disclosure and/or any additional therapeutic agent described herein. EZH2 is a histone-lysine N-methyltransferase that is a member of the Polycomb repressive complex 2 (PRC2) family. EZH2 plays a crucial role in gene expression regulation, specifically by catalyzing the trimethylation of histone H3 at lysine 27 (H3K27me3), leading to transcriptional repression of target genes. EZH2 has been found to be overexpressed in several types of cancers and is associated with tumor progression and poor prognosis. In some embodiments, an EZH2 inhibitor is one or more of Tazemetostat, GSK2816126, and CPI-1205 (lirametostat). In some embodiments, reference to the term EZH2 inhibitor includes any such EZH2 inhibitor disclosed in any one of the following patent applications: WO 2023030299, WO 2022179584, WO 2020224607, WO 2021243060, WO 2021086069, WO 2019206155, WO 2018133795, WO 2018137639, WO 2017184999, WO 2017218953, WO 2016201328, WO 2015195848, WO 2013155317, WO 2013138361, and CN 114621191, each of which is incorporated herein by reference in its entirety, including the compound structures disclosed therein which are specifically incorporated herein by reference.

iv. Co-REST Inhibitors

In some embodiments, compositions and methods described herein may include a compound of the present disclosure in combination with one or more Co-REST inhibitors. A Co-REST inhibitor may be administered or formulated in combination with a compound of the present disclosure and/or any additional therapeutic agent described herein. Co-REST is a transcriptional co-repressor protein that interacts with a variety of transcription factors to regulate gene expression. Co-REST acts by recruiting histone deacetylases (HDACs) to chromatin, leading to the repression of gene expression. Inhibition of Co-REST has been proposed as a potential therapeutic strategy for the treatment of various diseases, including neurodegenerative disorders and cancer. In some embodiments, a co-REST inhibitor is one or more of Nocodazole, NSC 1892, and Anacardic acid.

v. EP300

In some embodiments, compositions and methods described herein may include a compound of the present disclosure in combination with one or more E1A-binding protein p300 (EP300) inhibitors. An EP300 inhibitor may be administered or formulated in combination with a compound of the present disclosure and/or any additional therapeutic agent described herein. EP300 is a transcriptional co-activator involved in the regulation of numerous cellular processes, including chromatin remodeling, DNA damage response, and cell cycle progression. EP300 acts as a histone acetyltransferase, catalyzing the transfer of acetyl groups to lysine residues on histone proteins, which leads to changes in chromatin structure and gene expression. EP300 activity has been implicated in diseases, such as cancer, cardiovascular and neurological disorders. In some embodiments, an EP300 inhibitor is one or more of C646, A-485, NU9056, and L002. In some embodiments, reference to the term EP300 inhibitor includes any such EP300 inhibitor disclosed in any one of the following patent applications: WO 2021213521 and WO 2016044694, each of which is incorporated herein by reference in its entirety, including the compound structures disclosed therein which are specifically incorporated herein by reference.

vi. LSD1

In some embodiments, compositions and methods described herein may include a compound of the present disclosure in combination with one or more Lysine-specific demethylase 1 (LSD1) inhibitors. A LSD1 inhibitor may be administered or formulated in combination with a compound of the present disclosure and/or any additional therapeutic agent described herein. LSD1 is an enzyme that plays a crucial role in regulating gene expression through histone modification. It specifically removes the methyl group from lysine 4 on histone 3, leading to gene repression. Dysregulation of LSD1 has been associated with various diseases including cancer and neurodegenerative disorders. In some embodiments, a LSD1 inhibitor is one or more of GSK2879552, IMG-7289, ORY-1001, IMG-8419, SP-2577, CC-90011, HCl-2509, and INCB059872. In some embodiments, reference to the term LSD1 inhibitor includes any such LSD1 inhibitor disclosed in any one of the following patent applications: WO 2021095840, WO 2021175079, WO 2021058024, WO 2020047198, WO 2020052649, WO 2020015745, WO 2020052647, WO 2018137644, WO 2017184934, WO 2017027678, WO 2017116558, WO 2017149463, WO 2016161282, WO 2015123465, WO 2015123424, WO 2013057322, WO 2013057320, WO 2012135113, CN 114805261, CN 111072610 CN107174584, CN 110478352, CN 106432248, and CN 106045881, each of which is incorporated herein by reference in its entirety, including the compound structures disclosed therein which are specifically incorporated herein by reference.

vii. PRMT5

In some embodiments, compositions and methods described herein may include a compound of the present disclosure in combination with one or more Protein arginine methyltransferase 5 (PRMT5) inhibitors. A PRMT5 inhibitor may be administered or formulated in combination with a compound of the present disclosure and/or any additional therapeutic agent described herein. PRMT5 is a member of the PRMT family, which catalyzes the transfer of a methyl group from S-adenosylmethionine (SAM) to the nitrogen atoms of arginine residues in target proteins. PRMT5 is involved in various biological processes, including gene expression regulation, signal transduction, and DNA repair. In some embodiments, a PRMT5 inhibitor is one or more of TNG908, TNG462, AMG193, GSK591, EPZ015666, TC-E 5003, and MS023. In some embodiments, reference to the term PRMT5 inhibitor includes any such PRMT5 inhibitor disclosed in any one of the following patent applications: WO 2023001133, WO 2022206964, WO 2022153161, WO 2021068953, WO 2021088992, WO 2020259478, WO 2020205660, WO 2020250123, WO 2020033288, WO 2019102494, WO 2019112719, WO 2019180631, WO 2018065365, WO 2017153186, WO 2017212385, WO 2017032840, WO 2016022605, WO2014100695, WO 2014145214, WO 2014100719, CN 111825656, CN 114558014, CN 11304554, and CN 112778275, each of which is incorporated herein by reference in its entirety, including the compound structures disclosed therein which are specifically incorporated herein by reference.

viii. MAT2A

In some embodiments, compositions and methods described herein may include a compound of the present disclosure in combination with one or more methionine adenosyltransferase 2A (MAT2A) inhibitors. A MAT2A inhibitor may be administered or formulated in combination with a compound of the present disclosure and/or any additional therapeutic agent described herein. MAT2A is an enzyme that catalyzes the production of S-adenosylmethionine (SAM), which is an important cofactor in many biological processes, including DNA methylation, protein methylation, and polyamine synthesis. Elevated MAT2A expression has been associated with various cancers. In some embodiments, a MAT2A inhibitor is one or more of cycloleucine and 2-hydroxy-4-methylthiobutanoic acid. In some embodiments, reference to the term MAT2A inhibitor includes any such MAT2A inhibitor disclosed in any one of the following patent applications: WO 2022256808, WO 2022256806, WO 2019191470, and CN 115716831, each of which is incorporated herein by reference in its entirety, including the compound structures disclosed therein which are specifically incorporated herein by reference.

ix. DOT1L

In some embodiments, compositions and methods described herein may include a compound of the present disclosure in combination with one or more Disruptor of Telomeric silencing 1-like (DOT1L) inhibitors. A DOT1L inhibitor may be administered or formulated in combination with a compound of the present disclosure and/or any additional therapeutic agent described herein. DOT1 L is a histone methyltransferase enzyme that catalyzes the methylation of lysine 79 on histone H3. This modification is associated with transcriptional elongation and is important for the maintenance of gene expression programs. The DOT1 L family includes enzymes that are involved in epigenetic regulation and transcriptional control, and their dysregulation has been linked to various diseases, including cancer. In some embodiments, a DOT1 L inhibitor is one or more of EPZ-5676 (pinometostat) and EPZ-004777. In some embodiments, reference to the term DOT1 L inhibitor includes any such DOT1 L inhibitor disclosed in any one of the following patent applications: WO 2016090271, WO 2014100662, and CN 108997480, each of which is incorporated herein by reference in its entirety, including the compound structures disclosed therein which are specifically incorporated herein by reference.

iix) UBA1

In some embodiments, compositions and methods described herein may include a compound of the present disclosure in combination with one or more ubiquitin-activating enzyme inhibitors (e.g., a UBA1 inhibitor). A UBA1 inhibitor may be administered or formulated in combination with a compound of the present disclosure and/or any additional therapeutic agent described herein. UBA1, also known as ubiquitin-activating enzyme 1, is a key enzyme involved in the ubiquitination process, a fundamental cellular mechanism for protein degradation and regulation. Ubiquitination involves the covalent attachment of ubiquitin molecules to target proteins, marking them for degradation by the proteasome or modulating their activity, localization, or interactions within the cell. Several inhibitors have been developed to modulate UBA1 activity, with the aim of disrupting ubiquitination-mediated processes in diseased cells. These inhibitors include but are not limited to adenosine-based inhibitors which typically compete with ATP for binding to the active site of UBA1, thereby preventing the activation of ubiquitin (e.g., PYR-41 and MLN7243); covalent inhibitors which form irreversible bonds with specific amino acid residues in the active site of UBA1, leading to inhibition of its activity (e.g., TAK-243 (formerly known as MLN4924)); allosteric inhibitors which bind to sites on UBA1 distinct from the active site, inducing conformational changes that inhibit its catalytic activity (e.g., compound 2i); and fragment-based inhibitors which are designed based on smaller molecular fragments that bind to UBA1. In some embodiments, a UBA1 inhibitor is one or more of PYR-41, MLN7243, and TAK-243. In some embodiments, reference to the term UBA1 inhibitor includes any such UBA1 inhibitor disclosed in any one of the following patent applications: WO 2016069393 A1, WO 2016069392 A1, and JP 2013237627 A2, each of which is incorporated herein by reference in its entirety, including the compound structures disclosed therein which are specifically incorporated herein by reference.

### m) Ribonucleotide Reductase Inhibitors

Compositions and methods described herein may include a compound of the present disclosure in combination with one or more ribonucleotide reductase inhibitors (RNRi). RNR inhibitors are a class of compounds that inhibit the enzyme ribonucleotide reductase, which is essential for DNA synthesis and repair. RNR catalyzes the conversion of ribonucleotides (RNA building blocks) into deoxyribonucleotides (DNA building blocks), providing the necessary precursors for DNA replication and repair in proliferating cells. By inhibiting RNR, these compounds effectively limit the production of deoxyribonucleotides, thereby preventing DNA synthesis and halting the proliferation of rapidly dividing cells, such as cancer cells.

RNR is composed of two subunits: the R1 large subunit (containing the catalytic site) and the R2 small subunit (containing a di-iron center critical for enzymatic activity). RRIs typically act by binding to either the active site on the R1 subunit or the iron-oxygen complex in the R2 subunit, leading to the inhibition of the enzyme's activity. In some embodiments, a RNR inhibitor is a nucleoside analog inhibitor, an iron chelator, or an allosteric inhibitor. In some embodiments, a RNR inhibitor useful according to the present disclosure include but are not limited to one or more of hydroxyurea, triapine, didox, GTI-2040, CPI-613 (devimistat), and clofarabine. In some embodiments, reference to the term RNR inhibitor includes any such RNR inhibitor disclosed in any one of the following patent applications: WO 2025049814, WO 2022059691, WO 2022059692, WO 2021034776, WO 2019106579, WO 2014205179, WO 2013105088, WO 199312782, U.S. Pat. Nos. 5,071,835, 5,405,850, 4,814,432, and WO 199518815 each of which is incorporated herein by reference in its entirety, including the compound structures disclosed therein which are specifically incorporated herein by reference.

### n) Additional Therapeutic Agents Useful for Combination Therapy

In some embodiments, compositions and methods described herein may include a compound of the present disclosure in combination with one or more Farnesyl transferase inhibitors. A farnesyl transferase inhibitor may be administered or formulated in combination with a compound of the present disclosure and/or any additional therapeutic agent described herein. Farnesyl transferase inhibitors (FTIs) are a class of drugs that target the farnesyl transferase enzyme, which plays a role in a process called protein prenylation. Protein prenylation is an important step in the process of activating certain proteins involved in signal transduction, cell growth, and differentiation. In some embodiments, a farnesyl transferase inhibitor is one or more of tipifarnib, lonafarnib, and rilapladib. In some embodiments, reference to the term farnesyl transferase inhibitor includes any such farnesyl transferase inhibitor disclosed in any one of the following patent applications: WO 2010057028, WO 2007042465, WO 200136395, WO 200064891, WO 200042849, WO 199938862, WO 199928315, WO 199829390, WO 199426723, CN 107312000, CN 107365310, KR 100375421, KR 100388790, each of which is incorporated herein by reference in its entirety, including the compound structures disclosed therein which are specifically incorporated herein by reference.

In some embodiments, compositions and methods described herein may include a compound of the present disclosure in combination with one or more casein kinase inhibitors. In some embodiments, a casein inhibitor is, SR-3029, a potent and ATP competitive CK16 and CK1E inhibitor.

In some embodiments, compositions and methods described herein may include one or more FLT3 inhibitors in combination with a compound of the present disclosure disclosed herein. FLT3 (Fms-like tyrosine kinase 3), also known as CD135, is a receptor tyrosine kinase (RTK) that plays a crucial role in regulating hematopoiesis, the process by which blood cells are formed. It is primarily expressed on hematopoietic stem cells (HSCs) and progenitor cells in the bone marrow, where it controls cell proliferation, survival, and differentiation. In some embodiments, a FLT3 inhibitor includes, but are not limited to, midostaurin, gilteritinib, sorafenib, quizartinib, crenolanib, ponatinib and quizartinib.

In some embodiments, compositions and methods described herein may include a compound of the present disclosure in combination with one or more one or more TGFb pathway inhibitors. In some embodiments, compositions and methods described herein may include one or more TGFb inhibitors. A TGFb inhibitor may be administered or formulated in combination with a compound of the present disclosure and/or any additional therapeutic agent described herein. TGFb (transforming growth factor beta) is a multifunctional cytokine involved in various cellular processes, including cell growth, differentiation, apoptosis, and immune response. Dysregulation of the TGFb signaling pathway has been implicated in various diseases, including cancer, fibrosis, and autoimmune disorders. In some embodiments, a TGFb inhibitor is one or more of galunisertib (LY2157299), and vactosertib (TEW-7197). In some embodiments, a TGFb inhibitor is one or more of Galunisertib, LY2157299, Fresolimumab, Lerdelimumab, Trabedersen, curcumin, resveratrol and small interfering RNA (siRNA) to silence TGFb receptor expression. In some embodiments, reference to the term TGFb inhibitor includes any such TGFb inhibitor disclosed in any one of the following patent applications: WO 2023043473, WO 2020104648, WO 2020128850, WO 2016140884, WO 2007018818, WO 2004024159, WO 200226935, WO 2002062753, WO 2002062776, and JP 2012087076, each of which is incorporated herein by reference in its entirety, including the compound structures disclosed therein which are specifically incorporated herein by reference.

In some embodiments, compositions and methods described herein may include a compound of the present disclosure in combination with one or more HSP90 inhibitors. A HSP90 inhibitor may be administered or formulated in combination with a compound of the present disclosure and/or any additional therapeutic agent described herein. HSP90, also known as heat shock protein 90, is a molecular chaperone that plays a critical role in regulating the folding, stability, and activity of a large number of client proteins involved in various cellular processes, including cell cycle progression, signal transduction, and apoptosis. In some embodiments, a HSP90 inhibitor is one or more of Geldanamycin and its derivatives (e.g., 17-AAG, 17-DMAG), KOS 953, Radicicol and its derivatives (e.g., PU-H71), SNX-2112, Ganetespib, AT13387, Onalespib, Luminespib, and KW-2478. In some embodiments, reference to the term HSP90 inhibitor includes any such HSP90 inhibitor disclosed in any one of the following patent applications: WO 2021137665, WO 2018200534, WO 2017151425, WO 2015200514, WO 2013053833, WO 2013009657, WO 2013119985, WO 2012138894, WO 2011044394, WO 2009097578, WO 2008115719, CN 105237533, and CN 104030904, each of which is incorporated herein by reference in its entirety, including the compound structures disclosed therein which are specifically incorporated herein by reference.

In some embodiments, compositions and methods described herein may include a compound of the present disclosure in combination with one or more Glutathione peroxidase 4 (GPX4) inhibitors. A GPX4 inhibitor may be administered or formulated in combination with a compound of the present disclosure and/or any additional therapeutic agent described herein. GPX4 is an antioxidant enzyme that plays a critical role in protecting cells against oxidative stress-induced cell death. GPX4 catalyzes the reduction of lipid hydroperoxides to their corresponding alcohols and acts as a regulator of ferroptosis, a form of regulated cell death driven by lipid peroxidation. In some embodiments, a GPX4 inhibitor is one or more of RSL3, ML162, DPI7, FINO2, MCB-613, CBS9106, ML210, ODSH, and TLN232. In some embodiments, reference to the term GPX4 inhibitor includes any such GPX4 inhibitor disclosed in any one of the following patent applications: WO 2021132592, US 2021244715, and KR 20220115536, each of which is incorporated herein by reference in its entirety, including the compound structures disclosed therein which are specifically incorporated herein by reference.

In some embodiments, compositions and methods described herein may include a compound of the present disclosure in combination with one or more NRF2 inhibitors. A NRF2 inhibitor may be administered or formulated in combination with a compound of the present disclosure and/or any additional therapeutic agent described herein. NRF2 is a transcription factor that regulates the expression of genes involved in the cellular antioxidant response, detoxification, and other cytoprotective pathways. It plays a critical role in cellular defense mechanisms against oxidative stress and other forms of cellular damage. In some embodiments, a NRF2 inhibitor is one or more of ML385, Brusatol, CDDO-Im, RTA-408, and trigonelline. In some embodiments, reference to the term NRF2 inhibitor includes any such NRF2 inhibitor disclosed in any one of the following patent applications: WO 2023051088, WO 2021202720, KR 2022013610, and CN 107519168, each of which is incorporated herein by reference in its entirety, including the compound structures disclosed therein which are specifically incorporated herein by reference.

In some embodiments, compositions and methods described herein may include a compound of the present disclosure in combination with one or more TEA domain (TEAD) inhibitors. A TEAD inhibitor may be administered or formulated in combination with a compound of the present disclosure and/or any additional therapeutic agent described herein. TEAD is a family of transcription factors that play a key role in regulating gene expression during embryonic development and tissue homeostasis. The four members of the TEAD family (TEAD1-4) are transcriptional co-activators that bind to DNA through their conserved TEA domain and interact with other transcription factors to activate the expression of target genes. In some embodiments, a TEAD inhibitor is one or more of VT3989, VT-107, a pan-TEAD, VT-104, Verteporfin, CA3, IAG933, K-975, IK-595, and Statins (see, e.g., Chapeau, Emilie and Schmelzle, Tobias (2023) IAG933, an oral selective YAP1-TAZ/pan-TEAD protein-protein interaction inhibitor (PPli) with pre-clinical activity in monotherapy and combinations with MAPK inhibitors. Nature cancer). In some embodiments, reference to the term TEAD inhibitor includes any such TEAD inhibitor disclosed in any one of the following patent applications: WO 2023280254, WO 2023031781, WO 2022258040, WO 2020070181 WO 2018185266, and WO 2017064277, each of which is incorporated herein by reference in its entirety, including the compound structures disclosed therein which are specifically incorporated herein by reference.

In some embodiments, compositions and methods described herein may include a compound of the present disclosure in combination with one or more NOTCH/Gamma secretase inhibitors. A NOTCH/Gamma secretase inhibitor may be administered or formulated in combination with a compound of the present disclosure and/or any additional therapeutic agent described herein. In some embodiments, a NOTCH/Gamma secretase inhibitor is nirogacestat. In some embodiments, reference to the term NOTCH/Gamma secretase inhibitor includes any such NOTCH/Gamma secretase inhibitor disclosed in any one of the following patent applications: WO 2020208572, WO 2017200969, WO 2014047390, WO 2014047372, WO 2011041336, WO 2010090954, WO 2009008980, WO 2009087130, WO 2007110335, CN 103664904, CN 105560244, and KR 20200077480, each of which is incorporated herein by reference in its entirety, including the compound structures disclosed therein which are specifically incorporated herein by reference.

In some embodiments, compositions and methods described herein may include a compound of the present disclosure in combination with one or more Hedgehog inhibitors. A hedgehog inhibitor may be administered or formulated in combination with A compound of the present disclosure and/or any additional therapeutic agent described herein. The hedgehog (Hh) family of proteins are secreted signaling molecules that play a crucial role in embryonic development and tissue homeostasis in adults. The Hh signaling pathway is involved in regulating cell growth, differentiation, and survival. In some embodiments, a hedgehog inhibitor is one or more of Vismodegib (ERIVEDGE®), Sonidegib (ODOMZO®), and Glasdegib (DAURISMO™). In some embodiments, reference to the term hedgehog inhibitor includes any such hedgehog inhibitor disclosed in any one of the following patent applications: WO 2011063309, and CN 107163028, each of which is incorporated herein by reference in its entirety, including the compound structures disclosed therein which are specifically incorporated herein by reference.

Compositions and methods described herein may include a compound of the present disclosure in combination with one or more NFkB pathway inhibitors. An NFkB inhibitor may be administered or formulated in combination with a compound of the present disclosure and/or any additional therapeutic agent described herein. NF-kappa B (NFκB) is a family of transcription factors involved in regulating various cellular processes, including inflammation, immunity, cell survival, and proliferation. Non-limiting examples of NFkB inhibitors include Bortezomib (Velcade), Curcumin, Parthenolide, IKK inhibitors (e.g., IKK-16, BAY 11-7082), Resveratrol, Andrographolide and Proteasome inhibitors (e.g., MG132, lactacystin).

In some embodiments, the additional therapy is the administration of side-effect limiting agents (e.g., agents intended to lessen the occurrence or severity of side effects of treatment. For example, in some embodiments, a compound of the present disclosure can also be used in combination with a therapeutic agent that treats nausea. Examples of agents that can be used to treat nausea include: dronabinol, granisetron, metoclopramide, ondansetron, and prochlorperazine, or pharmaceutically acceptable salts thereof.

In some embodiments, the one or more additional therapies includes a non-drug treatment (e.g., surgery or radiation therapy). In some embodiments, the one or more additional therapies includes a therapeutic agent (e.g., a compound or biologic that is an anti-angiogenic agent, signal transduction inhibitor, antiproliferative agent, glycolysis inhibitor, or autophagy inhibitor). In some embodiments, the one or more additional therapies includes a non-drug treatment (e.g., surgery or radiation therapy) and a therapeutic agent (e.g., a compound or biologic that is an anti-angiogenic agent, signal transduction inhibitor, antiproliferative agent, glycolysis inhibitor, or autophagy inhibitor).

Examples of non-drug treatments include, but are not limited to, radiation therapy, cryotherapy, hyperthermia, surgery (e.g., surgical excision of tumor tissue), and T cell adoptive transfer (ACT) therapy.

In some embodiments, a compound of the present disclosure may be used as an adjuvant therapy after surgery. In some embodiments, a compound of the present disclosure may be used as a neo-adjuvant therapy prior to surgery.

Radiation therapy may be used for inhibiting abnormal cell growth or treating a hyperproliferative disorder, such as cancer, in a subject (e.g., mammal (e.g., human)). Techniques for administering radiation therapy are known in the art. Radiation therapy can be administered through one of several methods, or a combination of methods, including, without limitation, external-beam therapy, internal radiation therapy, implant radiation, stereotactic radiosurgery, systemic radiation therapy, radiotherapy, and permanent or temporary interstitial brachy therapy. The term “brachy therapy,” as used herein, refers to radiation therapy delivered by a spatially confined radioactive material inserted into the body at or near a tumor or other proliferative tissue disease site. The term is intended, without limitation, to include exposure to radioactive isotopes (e.g., At-211, I-131, I-125, Y-90, Re-186, Re-188, Sm-153, Bi-212, P-32, and radioactive isotopes of Lu). Suitable radiation sources for use as a cell conditioner of the present disclosure include both solids and liquids. By way of non-limiting example, the radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, or Y-90. Moreover, the radionuclide(s) can be embodied in a gel or radioactive micro spheres.

In some embodiments, a compound of the present disclosure can render abnormal cells more sensitive to treatment with radiation for purposes of killing or inhibiting the growth of such cells. Accordingly, this disclosure further relates to a method for sensitizing abnormal cells in a mammal to treatment with radiation which comprises administering to the mammal an amount of a compound of the present disclosure, which amount is effective to sensitize abnormal cells to treatment with radiation. The amount of the compound in this method can be determined according to the means for ascertaining effective amounts of such compounds described herein. In some embodiments, a compound of the present disclosure may be used as an adjuvant therapy after radiation therapy or as a neo-adjuvant therapy prior to radiation therapy.

In some embodiments, the non-drug treatment is a T cell adoptive transfer (ACT) therapy. In some embodiments, the T cell is an activated T cell. The T cell may be modified to express a chimeric antigen receptor (CAR). CAR modified T (CAR-T) cells can be generated by any method known in the art. For example, the CAR-T cells can be generated by introducing a suitable expression vector encoding the CAR to a T cell. Prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present disclosure, any number of T cell lines available in the art may be used. In some embodiments, the T cell is an autologous T cell. Whether prior to or after genetic modification of the T cells to express a desirable protein (e.g., a CAR), the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 7,572,631; 5,883,223; 6,905,874; 6,797,514; and 6,867,041.

In some embodiments, compositions and methods described herein may include a compound of the present disclosure in combination with one or more Claudin-18 targeting agents. A Claudin-18 targeting agents may be administered or formulated in combination with a compound of the present disclosure and/or any additional therapeutic agent described herein. Claudin-18 (e.g., claudin 18.2; CLDN18.2) has become a promising target for the treatment of patients with digestive malignancies, such as gastric cancer (GC), gastroesophageal junction (GEJ) cancer, esophageal cancer, and pancreatic cancer, because of its limited expression in healthy tissues and abnormal overexpression in a range of malignancies. Multiple clinical trials of CLDN18.2-targeted therapies, including monoclonal antibodies, bispecific antibodies, antibody-drug conjugates (ADCs), and chimeric antigen receptor (CAR) T-cell therapies, are ongoing, with some showing promising early results. Malignant transformation of gastric epithelial tissue leads to disruption of cell polarity and then to exposure of CLDN18.2 epitopes on the cell surface. Although targeted monoclonal antibodies are largely unable to access CLDN18.2 located in tight-junction supramolecular complexes in normal tissue, the perturbations in cell polarity that expose CLDN18.2 epitopes may theoretically enable CLDN18.2 targeted agents to bind to CLDN18.2 in malignant tissues with minimal off-target effects, making CLDN18.2 an attractive target for therapy. In some embodiments, a Claudin-18 targeting agent is one or more of Zolbetuximab, ASKB589, Osemitamab (TST001), PT886 (a bispecific antibody that targets CLDN18.2 and CD47), TJ-CD4B, CMG901 (an ADC that is composed of an antiCLDN18.2 monoclonal antibody joined to a cytotoxic payload, monomethyl auristatin E), and CT041 (autologous T cells genetically engineered to express a CLDN18.2-targeted CAR). In some embodiments, reference to the term Claudin-18 targeting agent includes any such Claudin-18 targeting agent disclosed in any one of the following patent applications: WO 2024081544, WO 2024131683, WO 2024137619, WO 2024140670, WO 2024136594, WO 2023034922, WO 2023046202, WO 2022203090, WO 2022133169, WO 2022100613, WO 2022256449, WO 2022136642, WO 2021155380, WO 2021129765, WO 2021011885, WO 2021058000, WO 2021218874, WO 2021027850, WO 2020156554, WO 2020025792, WO 2020114480, WO 2020211792, WO 2020239005, WO 2019219089, WO 2018157147, WO 2018108106, WO 2016166122, WO 2014146778, CN 118290582, CN118203658, and CN 118286201, each of which is incorporated herein by reference in its entirety, including the compound structures disclosed therein which are specifically incorporated herein by reference.

In some embodiments, a therapeutic agent for combination therapy may be a steroid. Accordingly, in some embodiments, the one or more additional therapies includes a steroid. Suitable steroids may include, but are not limited to, 21-acetoxypregnenolone, alclometasone, algestone, amcinonide, beclomethasone, betamethasone, budesonide, chloroprednisone, clobetasol, clocortolone, cloprednol, corticosterone, cortisone, cortivazol, deflazacort, desonide, desoximetasone, dexamethasone, diflorasone, diflucortolone, difuprednate, enoxolone, fluazacort, fiucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, mazipredone, medrysone, meprednisone, methylprednisolone, mometasone furoate, paramethasone, prednicarbate, prednisolone, prednisolone 25-diethylaminoacetate, prednisolone sodium phosphate, prednisone, prednival, prednylidene, rimexolone, tixocortol, triamcinolone, triamcinolone acetonide, triamcinolone benetonide, triamcinolone hexacetonide, and salts or derivatives thereof.

Further examples of therapeutic agents that may be used in combination therapy with a compound of the present disclosure include compounds described in the following patents: U.S. Pat. Nos. 6,258,812, 6,630,500, 6,515,004, 6,713,485, 5,521,184, 5,770,599, 5,747,498, 5,990,141, 6,235,764, and 8,623,885, and International Patent Applications WO 200137820, WO 200132651, WO 200268406, WO 200266470, WO 200255501, WO 200405279, WO 200407481, WO 200407458, WO 200409784, WO 200259110, WO 199945009, WO 2000/59509, WO 199961422, WO 200012089, and WO 200002871.

An additional therapeutic agent may be a biologic (e.g., cytokine (e.g., interferon or an interleukin such as IL-2)) used in treatment of cancer or symptoms associated therewith. In some embodiments, the biologic is an immunoglobulin-based biologic, e.g., a monoclonal antibody (e.g., a humanized antibody, a fully human antibody, an Fc fusion protein, or a functional fragment thereof) that agonizes a target to stimulate an anti-cancer response or antagonizes an antigen important for cancer. Also included are antibody-drug conjugates.

An additional therapeutic agent may be an immune modulatory agent. For example, an additional therapeutic agent may be a T-cell checkpoint inhibitor. In one embodiment, the checkpoint inhibitor is an inhibitory antibody (e.g., a monospecific antibody such as a monoclonal antibody). The antibody may be, e.g., humanized or fully human. In some embodiments, the checkpoint inhibitor is a fusion protein, e.g., an Fc-receptor fusion protein. In some embodiments, the checkpoint inhibitor is an agent, such as an antibody, which interacts with a checkpoint protein. In some embodiments, the checkpoint inhibitor is an agent, such as an antibody, which interacts with the ligand of a checkpoint protein. In some embodiments, the checkpoint inhibitor is an inhibitor (e.g., an inhibitory antibody or small molecule inhibitor) of CTLA-4 (e.g., an anti-CTLA-4 antibody or fusion a protein). In some embodiments, the checkpoint inhibitor is an inhibitor or antagonist (e.g., an inhibitory antibody or small molecule inhibitor) of PD-1. In some embodiments, the checkpoint inhibitor is an inhibitor or antagonist (e.g., an inhibitory antibody or small molecule inhibitor) of PD-L1. In some embodiments, the checkpoint inhibitor is an inhibitor or antagonist (e.g., an inhibitory antibody or Fc fusion or small molecule inhibitor) of PD-L2 (e.g., a PD-L2/Ig fusion protein). In some embodiments, the checkpoint inhibitor is an inhibitor or antagonist (e.g., an inhibitory antibody or small molecule inhibitor) of B7-H3, B7-H4, BTLA, HVEM, TIM3, GAL9, LAG3, VISTA, KIR, 2B4, CD160, CGEN-15049, CHK 1, CHK2, A2aR, A2bR, A2aR/A2bR, B-7 family ligands, or a combination thereof. In some embodiments, the checkpoint inhibitor is pembrolizumab, nivolumab, PDR001 (NVS), REGN2810 (Sanofi/Regeneron), a PD-L1 antibody such as, e.g., avelumab, durvalumab, atezolizumab, pidilizumab, JNJ-63723283 (JNJ), BGB-A317 (BeiGene & Celgene) or a checkpoint inhibitor disclosed in Preusser, M. et al. (2015) Nat. Rev. Neurol., including, without limitation, ipilimumab, tremelimumab, nivolumab, pembrolizumab, AMP224, AMP514/MEDI0680, BMS936559, MEDI4736, MPDL3280A, MSB0010718C, BMS986016, IMP321, lirilumab, IPH2101, 1-7F9, and KW-6002. Non-limiting examples of immune modulatory agent includes targets identified in Table 2.

TABLE 2
Exemplary Immune Modulatory Targets

| | | | |
|---|---|---|---|
| CTLA-4 | Inhibitory Receptor | A2aR, A2bR or both | Inhibitory Receptor |
| | | A2aR/A2bR | |
| PD-1 | Inhibitory Receptor | CD73 | Inhibitory Receptor |
| PD-L1 | Ligand for PD-1 | CD39 | Inhibitory Receptor |
| LAG-3 | Inhibitory Receptor | PVRIG | Inhibitory Receptor |
| B7.1 | Costimulatory Molecule | IDO | Inhibitory enzyme |
| B7-H3 | Inhibitory Ligand | CSF1R | Inhibitory Receptor |
| B7-H4 | Inhibitory Ligand | LIF | Inhibitory Cytokine |
| TIM3 | Inhibitory Receptor | CD47 | Inhibitory Receptor |
| VISTA | Inhibitory Receptor | SIRPa | Inhibitory Receptor |
| CD137 | Costimulatory Molecule | IL-2 | Effector Cytokines |
| OX-40 | Costimulatory Receptor | IL-15 | Effector Cytokines |
| CD40 agonist | Costimulatory Molecule | IL-12 | Effector Cytokines |
| CD40 agonist + | Costimulatory Molecule | TREM2 | Receptor |
| FLT3 ligand | | | |
| CD27 | Costimulatory Receptor | TGFb | Multifunctional Cytokine |
| CCR4 | Costimulatory Receptor | CD73/TGFb trap | Multifunctional Cytokine |
| GITR | Costimulatory Receptor | TCR-T cells directed to | Cell therapy |
| | | KRAS^{MUT}, mesothelin, or | |
| | | PRAME | |
| NKG2D | Activating Receptor | mRNA cancer vaccines | vaccines |
| KIR | Costimulatory Receptor | BiTEs | Bi-specific T-cell engager |
| NKG2A | Inhibitory Receptor | Dual EP2/EP4 inhibitor | E-prostanoid receptor |
| ENPP1 | Inhibitory Receptor | Gamma delta T Cells | Cell therapy |
| TIGIT | Inhibitory Receptor | NK cells | Cell therapy |

CTLA4, cytotoxic T-lymphocyte-associated antigen 4; LAG3, lymphocyte activation gene 3; PD-1, programmed cell death protein 1; PD-L1, PD-1 ligand; TIM3, T cell membrane protein 3; VISTA, V-domain immunoglobulin (Ig)-containing suppressor of T-cell activation; KIR, killer IgG-like receptor, APC (Antigen Presenting Cells); TREM2 (Triggering receptor expressed on myeloid cells 2); TGF-b (Transforming growth factor beta)

In some embodiments, compositions and methods described herein may include a compound of the disclosure in combination with one or more immune checkpoint inhibitor (ICI). An immune checkpoint inhibitor may be administered or formulated in combination with a compound as described herein.

Immune checkpoints refer to a plethora of inhibitory pathways hardwired into the immune system, which, under normal physiological conditions are crucial for maintaining self-tolerance and modulating the duration and amplitude of physiological immune responses in peripheral tissues to minimize collateral tissue damage in response to pathogenic infection. However, the expression of immune checkpoint proteins is often dysregulated by tumors as an important immune resistance and escape mechanism.

Because many of the immune checkpoints are initiated by ligand-receptor interactions, they can be readily blocked by antibodies or modulated by recombinant forms of ligands or receptors. Thus, inhibition of these pathways has been used to activate therapeutic anti-tumor immunity. For example, cytotoxic T-lymphocyte-associated antigen 4 (CTLA-4) antibodies were the first of this class of immunotherapeutics to achieve US Food and Drug Administration (FDA) approval. Preliminary clinical findings with inhibitors of additional immune-checkpoint proteins, such as programmed cell death protein 1 (PD-1), indicate broad and diverse opportunities to enhance anti-tumor immunity with the potential to produce durable clinical responses.

T cell activation through blockade of immune checkpoints has been a major focus of efforts to therapeutically manipulate endogenous anti-tumor immunity, owing to the capacity of T cells for the selective recognition of peptides derived from proteins in all cellular compartments; their capacity to directly recognize and kill antigen-expressing cells (by CD8+ effector T cells; also known as cytotoxic T lymphocytes (CTLs)); and their ability to orchestrate diverse immune responses (by CD4+ helper T cells), which integrate adaptive and innate effector mechanisms. Thus, agonists of co-stimulatory receptors or antagonists of inhibitory signals, both of which result in the amplification of antigen-specific T cell responses, are currently agents of interest in clinical testing.

ICIs approved or in development include, but are not limited to, YERVOY® (ipilimumab), OPDIVO® (nivolumab), KEYTRUDA® (pembrolizumab), tremelimumab, galiximab, MDX-1106, BMS-936558, MED14736, MPDL3280A, MED16469, BMS-986016, BMS-663513, PF-05082566, IPH2101, KW-0761, CDX-1127, CP-870, CP-893, GSK2831781, MSB0010718C, MK3475, CT-011, AMP-224, MDX-1105, IMP321, and MGA271, as well as numerous other antibodies or fusion proteins directed to the immune checkpoint proteins noted in Table 1. Common immune checkpoint proteins that may be targeted by ICIs include, but are not limited to B7.1, B7-H3, LAG3, CD137, KIR, CCR4, CD27, OX40, GITR, CD40, CTLA4, PD-1, and PD-L1. In some embodiments, the immune checkpoint inhibitor is an inhibitor of a target selected from the group comprising or consisting of programmed cell death protein-1, ligand of PD-1, cytotoxic T-lymphocyte-associated protein 4 (CTLA-4), T cell immunoglobulin and mucin-domain containing-3 (TIM-3), V-domain Ig suppressor of T cell activation (VISTA), lymphocyte-activation gene 3 (LAG-3), T cell immunoglobulin and ITIM domain (TIGIT), B7 homolog 3 protein (B7-H3), B- and T-lymphocyte attenuator (BTLA), Sialic acid binding Ig-like lectin 15 (Siglec-15), cytokine-inducible SH2-containing protein (CISH), and combination thereof.

In some embodiments, the ICI therapy is selected from one or more of anti-PD-1, anti-PD-L1, anti-CTLA-4, anti-LAG3, anti-B7.1, anti-B7H3, anti-B7H4, anti-TIM3, anti-VISTA, anti-CD137, anti-OX40, anti-CD40, anti-CD27, anti-CCR4, anti-GITR, anti-NKG2D, and anti-KIR. In some embodiments, the ICI therapy is an antibody (e.g., a monoclonal antibody selective for any of the targets in Table 1). In some embodiments the ICI is an anti-PD-1 antibody. The antibody may be, e.g., humanized or fully human.

In some embodiments, the checkpoint inhibitor is a fusion protein, e.g., an Fc-receptor fusion protein. In some embodiments, the checkpoint inhibitor is an agent, such as an antibody, which interacts with a checkpoint protein. In some embodiments, the checkpoint inhibitor is an agent, such as an antibody, which interacts with the ligand of a checkpoint protein. In some embodiments, the checkpoint inhibitor is an inhibitor (e.g., an inhibitory antibody or small molecule inhibitor) of CTLA-4 (e.g., an anti-CTLA-4 antibody or fusion a protein). In some embodiments, the checkpoint inhibitor is an inhibitor or antagonist (e.g., an inhibitory antibody or small molecule inhibitor) of PD-1. In some embodiments, the checkpoint inhibitor is an inhibitor or antagonist (e.g., an inhibitory antibody or small molecule inhibitor) of PD-L1. In some embodiments, the checkpoint inhibitor is an inhibitor or antagonist (e.g., an inhibitory antibody or Fc fusion or small molecule inhibitor) of PD-L2 (e.g., a PD-L2/Ig fusion protein). In some embodiments, the checkpoint inhibitor is an inhibitor or antagonist (e.g., an inhibitory antibody or small molecule inhibitor) of B7-H3, B7-H4, BTLA, HVEM, TIM3, GAL9, LAG3, VISTA, KIR, 2B4, CD160, CGEN-15049, CHK 1, CHK2, A2aR, B-7 family ligands, or a combination thereof. In some embodiments, the checkpoint inhibitor is pembrolizumab, nivolumab, PDR001 (NVS), REGN2810 (Sanofi/Regeneron), a PD-L1 antibody such as, e.g., avelumab, durvalumab, atezolizumab, pidilizumab, JNJ-63723283 (JNJ), BGB-A317 (also known as tislelizumab; BeiGene & Celgene) or a checkpoint inhibitor disclosed in Preusser, M. et al. (2015) Nat. Rev. Neurol., including, without limitation, ipilimumab, tremelimumab, nivolumab, pembrolizumab, AMP224, AMP514/MEDI0680, BMS936559, MEDI4736, MPDL3280A, MSB0010718C, BMS986016, IMP321, lirilumab, IPH2101, 1-7F9, and KW-6002.

In some embodiments, the immune checkpoint inhibitor is an inhibitor of programmed cell death protein-1 (PD-1) or an inhibitor of the ligand of PD-1 (PDL-1).

Programmed cell death protein-1 is herein interchangeably referred to as PD-1, PD1, PDCD1, PDCD-1, SLEB2, SLE1 and CD279.

In humans, PD-1 typically has the sequence as disclosed in UniProtKB Ref. Q15116, incorporated herein by reference.

Programmed death-ligand 1 is herein interchangeably referred to as PDL-1, PD-L1, PDL1, PDCD1 L1, PDCD1LG1, CD274, B7-H1, B7-H, B7H1.

In humans, PD-L1 typically has the sequence as disclosed in UniProtKB Ref. Q9NZQ7, incorporated herein by reference.

In some embodiments, the anti-PD1 antibody is cemiplimab, nivolumab, pembrolizumab, pidilizumab, spartalizumab, camrelizumab, sintilimab, tislelizumab, toripalimab, dostarlimab, sasanlimab, retifanlimab, tebotelimab, ABBV-181, AK 04, AK105, BCD-100, BI-754091, CBT-501, CC-90006, GLS-010, HLX10, IBI-308, JNJ-3283, JS001, LZM009, MEDI0680 (AMP-514), REGN-2810, SHR-1210, Sym021, TSR-042, or XmAb20717.

In some embodiments, the PD-1 inhibitor is a bispecific antibody specific for PD-1 and VEGF. In some embodiments, the bispecific antibody is ivonescimab (SMT112). In some embodiments, the bispecific antibody is BNT327.

In some embodiments, the anti-PDL1 antibody is atezolizumab, avelumab, durvalumab, envafolimab, FS118, BCD-135, BGB-A333, BGBA-317, CBT-502, CK-301, CS1001, FAZ053, MDX-1105, MSB2311, SHR-1316, M7824, LY3415244, CA-170, or CX-07Z.

An additional therapeutic agent may be an anti-TIGIT antibody, such as MBSA43, BMS-986207, MK-7684, COM902, AB154, MTIG7192A or OMP-313M32 (etigilimab).

In some embodiments, the combination therapy includes a compound of the disclosure and an anti-CCR8 antibody. In an embodiment, the anti-CCR8 antibody is an afucosylated antibody. In an embodiment, the anti-CCR8 antibody is a depleting antibody. In an embodiment, the anti-CCR8 antibody has ADCC activity. In an embodiment, the anti-CCR8 antibody is a neutralizing antibody. In an embodiment, the anti-CCR8 antibody is not a neutralizing antibody. In an embodiment, the anti-CCR8 antibody is BMS-986340. In an embodiment, the anti-CCR8 antibody is GS-1811. In an embodiment, the anti-CCR8 antibody is ABBV-514. In an embodiment, the anti-CCR8 antibody is LM-108. In an embodiment, the anti-CCR8 antibody is S-531011. In an embodiment, the anti-CCR8 antibody is BAY3375968. In an embodiment, the anti-CCR8 antibody is SRFI 14. In an embodiment, the anti-CCR8 antibody is CM369. In an embodiment, the anti-CCR8 antibody is ZL-1218. In an embodiment, the anti-CCR8 antibody is IPG0521. In an embodiment, the anti-CCR8 antibody is an anti-CCR8 antibody disclosed in WO 2025076288, WO 2022256563, WO2022004760, WO2022136649, WO 2021142002, WO 2021194942, WO 2021260206, WO 2021260208, WO 2021260210, WO 2021260209, WO 2021152186, WO 2020138489, and WO 2018181425 which are incorporated herein by reference including the structures disclosed therein.

In some embodiments, the combination therapy includes a compound of the disclosure and a cancer vaccine composition. In some embodiments, the cancer vaccine composition is ELI-002 2P, ELI-002 7P, HB-700, mRNA-4157, mRNA-5671, BNT111, GVAX Pancreas, IMA901, DCVax, SOT101, Sipuleucel-T, PROSTVAC-VF or TG01.

In some embodiments, the combination therapy includes a compound of the disclosure and an additional therapy or therapeutic agent selected from group consisting of RAS pathway targeted therapeutic agents, kinase-targeted therapeutics, mTORC1 inhibitors or degraders, YAP inhibitors or degraders, proteasome inhibitors or degraders, HSP90 inhibitors or degraders, farnesyl transferase inhibitors or degraders, PTEN inhibitors or degraders, signal transduction pathway inhibitors or degraders, checkpoint inhibitors, modulators of the apoptosis pathway, chemotherapeutics, angiogenesis-targeted therapies, immune-targeted agents, radiotherapy, and combinations thereof.

An additional therapeutic agent may be an agent that treats cancer or symptoms associated therewith (e.g., a cytotoxic agent, non-peptide small molecules, or other compound useful in the treatment of cancer or symptoms associated therewith, collectively, an “anti-cancer agent”). Anti-cancer agents can be, e.g., chemotherapeutics or targeted therapy agents.

Anti-cancer agents include mitotic inhibitors, intercalating antibiotics, growth factor inhibitors, cell cycle inhibitors, enzymes, topoisomerase inhibitors, biological response modifiers, alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Further anti-cancer agents include leucovorin (LV), irinotecan, oxaliplatin, capecitabine, paclitaxel, and doxetaxel. In some embodiments, the one or more additional therapies includes two or more anti-cancer agents. The two or more anti-cancer agents can be used in a cocktail to be administered in combination or administered separately. Suitable dosing regimens of combination anti-cancer agents are known in the art and described in, for example, Saltz et al., Proc. Am. Soc. Clin. Oncol. 18:233a (1999), and Douillard et al., Lancet 355(9209):1041-1047 (2000).

Other non-limiting examples of anti-cancer agents include Gleevec® (Imatinib Mesylate); KYPROLIS® (carfilzomib); VELCADE® (bortezomib); Casodex (bicalutamide); IRESSA® (gefitinib); alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; sarcodictyin A; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics (e.g., calicheamicin, such as calicheamicin gammall and calicheamicin omegall (see, e.g., Agnew, Chem. Intl. Ed Engl. 33:183-186 (1994)); dynemicin such as dynemicin A; bisphosphonates such as clodronate; an esperamicin; neocarzinostatin chromophore and related chromoprotein enediyne antiobiotic chromophores, aclacinomysins, actinomycin, authramycin, azaserine, bleomycins, cactinomycin, calicheamicin, carabicin, caminomycin, carminomycin, carzinophilin, chromomycins, dactinomycin, daunorubicin, detorubicin, 6-diazo-5-oxo-L-norleucine, adriamycin (doxorubicin), morpholino-doxorubicin, cyanomorpholino-doxorubicin, 2-pyrrolino-doxorubicin, deoxydoxorubicin, epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenishers such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone such as epothilone B; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes such as T-2 toxin, verracurin A, roridin A and anguidine; urethane; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel), ABRAXANE® (cremophor-free, albumin-engineered nanoparticle formulation of paclitaxel), and TAXOTERE® (doxetaxel); chloranbucil; tamoxifen (NOLVADEX™); raloxifene; aromatase inhibiting 4(5)-imidazoles; 4-hydroxytamoxifen; trioxifene; keoxifene; LY 117018; onapristone; toremifene (FARESTON®); flutamide, nilutamide, bicalutamide, leuprolide, goserelin; chlorambucil; GEMZAR® gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; NAVELBINE® (vinorelbine); novantrone; teniposide; edatrexate; daunomycin; aminopterin; ibandronate; irinotecan (e.g., CPT-11); topoisomerase inhibitor RFS 2000; difluoromethylornithine (DMFO); retinoids such as retinoic acid; esperamicins; capecitabine (e.g., XELODA®); and pharmaceutically acceptable salts of any of the above.

Additional non-limiting examples of anti-cancer agents include trastuzumab (HERCEPTIN®), bevacizumab (AVASTIN®), cetuximab (ERBITUX®), rituximab (RITUXAN®), TAXOL®, ARIMIDEX®, ABVD, avicine, abagovomab, acridine carboxamide, adecatumumab, 17-N-allylamino-17-demethoxygeldanamycin, alpharadin, alvocidib, 3-aminopyridine-2-carboxaldehyde thiosemicarbazone, amonafide, anthracenedione, anti-CD22 immunotoxins, antineoplastics (e.g., cell-cycle nonspecific antineoplastic agents, and other antineoplastics described herein), antitumorigenic herbs, apaziquone, atiprimod, azathioprine, belotecan, bendamustine, BIBW 2992, biricodar, brostallicin, bryostatin, buthionine sulfoximine, CBV (chemotherapy), calyculin, dichloroacetic acid, discodermolide, elsamitrucin, enocitabine, eribulin, exatecan, exisulind, ferruginol, forodesine, fosfestrol, ICE chemotherapy regimen, IT-101, imexon, imiquimod, indolocarbazole, irofulven, Ianiquidar, larotaxel, lenalidomide, lucanthone, lurtotecan, mafosfamide, mitozolomide, nafoxidine, nedaplatin, olaparib, ortataxel, PAC-1, pawpaw, pixantrone, proteasome inhibitors, rebeccamycin, resiquimod, rubitecan, SN-38, salinosporamide A, sapacitabine, Stanford V, swainsonine, talaporfin, tariquidar, tegafur-uracil, temodar, tesetaxel, triplatin tetranitrate, tris(2-chloroethyl)amine, troxacitabine, uramustine, vadimezan, vinflunine, ZD6126, and zosuquidar.

Further non-limiting examples of anti-cancer agents include natural products such as vinca alkaloids (e.g., vinblastine, vincristine, and vinorelbine), epidipodophyllotoxins (e.g., etoposide and teniposide), antibiotics (e.g., dactinomycin (actinomycin D), daunorubicin, and idarubicin), anthracyclines, mitoxantrone, bleomycins, plicamycin (mithramycin), mitomycin, enzymes (e.g., L-asparaginase which systemically metabolizes L-asparagine and deprives cells which do not have the capacity to synthesize their own asparagine), antiplatelet agents, antiproliferative/antimitotic alkylating agents such as nitrogen mustards (e.g., mechlorethamine, cyclophosphamide and analogs, melphalan, and chlorambucil), ethylenimines and methylmelamines (e.g., hexamethylmelaamine and thiotepa), alkyl sulfonates (e.g., busulfan), nitrosoureas (e.g., carmustine (BCNU) and analogs, and streptozocin), trazenes-dacarbazinine (DTIC), antiproliferative/antimitotic antimetabolites such as folic acid analogs, pyrimidine analogs (e.g., fluorouracil, floxuridine, and cytarabine), purine analogs and related inhibitors (e.g., mercaptopurine, thioguanine, pentostatin, and 2-chlorodeoxyadenosine), aromatase inhibitors (e.g., anastrozole, exemestane, and letrozole), and platinum coordination complexes (e.g., cisplatin and carboplatin), procarbazine, hydroxyurea, mitotane, aminoglutethimide, DNA binding agents (e.g., ZALYPSIS®), PI3K inhibitors such as PI3K delta inhibitor (e.g., GS-1101 and TGR-1202), PI3K delta and gamma inhibitor (e.g., CAL-130), copanlisib, alpelisib and idelalisib; multi-kinase inhibitor (e.g., TGO2 and sorafenib), hormones (e.g., estrogen) and hormone agonists such as leutinizing hormone releasing hormone (LHRH) agonists (e.g., goserelin, leuprolide and triptorelin), BAFF-neutralizing antibody (e.g., LY2127399), IKK inhibitors, p38MAPK inhibitors, anti-IL-6 (e.g., CNT0328), telomerase inhibitors (e.g., GRN 163L), cell surface monoclonal antibodies (e.g., anti-CD38 (HUMAX-CD38), anti-CSI (e.g., elotuzumab, PI13K/Akt inhibitors (e.g., perifosine), PKC inhibitors (e.g., enzastaurin), FTIs (e.g., ZARNESTRA™), anti-CD138 (e.g., BT062), Torcl/2 specific kinase inhibitors (e.g., INK128), ER/UPR targeting agents (e.g., MKC-3946), and cFMS inhibitors (e.g., ARRY-382).

In some embodiments, an anti-cancer agent is selected from mechlorethamine, camptothecin, ifosfamide, tamoxifen, raloxifene, gemcitabine, NAVELBINE@, sorafenib, or any analog or derivative variant of the foregoing. In some embodiments, the anti-cancer agent is JAB-3312.

In some embodiments, an anti-cancer agent is a PD-1 or PD-L1 antagonist.

In some embodiments, additional therapeutic agents include ALK inhibitors, HER2 inhibitors, EGFR inhibitors, IGF-1R inhibitors, MEK inhibitors, PI3K inhibitors, AKT inhibitors, TOR inhibitors, MCL-1 inhibitors, BCL-2 inhibitors, SHP2 inhibitors, proteasome inhibitors, and immune modulatory therapies, such as an immune checkpoint inhibitor. In some embodiments, a therapeutic agent may be a pan-RTK inhibitor, such as afatinib.

In some embodiments, the additional therapeutic agent is selected from the group consisting of a MEK inhibitor, a HER2 inhibitor, a SHP2 inhibitor, a CDK4/6 inhibitor, an mTOR inhibitor, a SOS1 inhibitor, and a PD-L1 inhibitor. In some embodiments, the additional therapeutic agent is selected from the group consisting of a MEK inhibitor, a SHP2 inhibitor, and a PD-L1 inhibitor. See, e.g., Hallin et al., Cancer Discovery, DOI: 10.1158/2159-8290 (Oct. 28, 2019) and Canon et al., Nature, 575:217 (2019). In some embodiments, a RAS(ON) inhibitor of the present disclosure is used in combination with a MEK inhibitor and a SOS1 inhibitor. In some embodiments, a RAS(ON) inhibitor of the present disclosure is used in combination with a PD-L1 inhibitor and a SOS1 inhibitor. In some embodiments, a RAS(ON) inhibitor of the present disclosure is used in combination with a PD-L1 inhibitor and a SHP2 inhibitor. In some embodiments, a RAS(ON) inhibitor of the present disclosure is used in combination with a MEK inhibitor and a SHP2 inhibitor. In some embodiments, the cancer is colorectal cancer, and the treatment comprises administration of a Ras inhibitor of the present disclosure in combination with a second or third therapeutic agent.

Proteasome inhibitors include, but are not limited to, carfilzomib (KYPROLIS®), bortezomib (VELCADE®), and oprozomib.

Immune therapies include, but are not limited to, monoclonal antibodies, immunomodulatory imides (IMiDs), GITR agonists, genetically engineered T-cells (e.g., CAR-T cells), bispecific antibodies (e.g., BiTEs), and anti-PD-1, anti-PD-L1, anti-CTLA4, anti-LAGI, and anti-OX40 agents).

Immunomodulatory agents (IMiDs) are a class of immunomodulatory drugs (drugs that adjust immune responses) containing an imide group. The IMiD class includes thalidomide and its analogues (lenalidomide, pomalidomide, and apremilast).

Exemplary anti-PD-1 antibodies and methods for their use are described by Goldberg et al., Blood 2007, 110(1):186-192; Thompson et al., Clin. Cancer Res. 2007, 13(6):1757-1761; and WO 2006121168 A1), as well as described elsewhere herein.

GITR agonists include, but are not limited to, GITR fusion proteins and anti-GITR antibodies (e.g., bivalent anti-GITR antibodies), such as, a GITR fusion protein described in U.S. Pat. Nos. 6,111,090, 8,586,023, WO 2010003118 and WO 2011090754; or an anti-GITR antibody described, e.g., in U.S. Pat. No. 7,025,962, EP 1947183, U.S. Pat. Nos. 7,812,135, 8,388,967, 8,591,886, 7,618,632, EP 1866339, and WO 2011028683, WO 2013039954, WO 2005007190, WO 2007133822, WO 2005055808, WO 199940196, WO 200103720, WO 199920758, WO 2006083289, WO 2005115451, and WO 2011051726.

Another example of a therapeutic agent that may be used in combination with a compound of the present disclosure is an anti-angiogenic agent. Anti-angiogenic agents are inclusive of, but not limited to, in vitro synthetically prepared chemical compositions, antibodies, antigen binding regions, radionuclides, and combinations and conjugates thereof. An anti-angiogenic agent can be an agonist, antagonist, allosteric modulator, toxin or, more generally, may act to inhibit or stimulate its target (e.g., receptor or enzyme activation or inhibition), and thereby promote cell death or arrest cell growth. In some embodiments, the one or more additional therapies include an anti-angiogenic agent.

Anti-angiogenic agents can be MMP-2 (matrix-metalloproteinase 2) inhibitors, MMP-9 (matrix-metalloprotienase 9) inhibitors, and COX-II (cyclooxygenase 11) inhibitors. Non-limiting examples of anti-angiogenic agents include rapamycin, temsirolimus (CCI-779), everolimus (RAD001), sorafenib, sunitinib, and bevacizumab. Examples of useful COX-II inhibitors include alecoxib, valdecoxib, and rofecoxib. Examples of useful matrix metalloproteinase inhibitors are described in WO 199633172, WO 199627583, WO 199807697, WO 199803516, WO 199834918, WO 199834915, WO 199833768, WO 199830566, WO 199005719, WO 199952910, WO 199952889, WO 199929667, WO 1999007675, EP 0606046, EP 0780386, EP 1786785, EP 1181017, EP 0818442, EP 1004578, and US 20090012085, and U.S. Pat. Nos. 5,863,949 and 5,861,510. Preferred MMP-2 and MMP-9 inhibitors are those that have little or no activity inhibiting MMP-1. More preferred, are those that selectively inhibit MMP-2 or AMP-9 relative to the other matrix-metalloproteinases (i.e., MAP-1, MMP-3, MMP-4, MMP-5, MMP-6, MMP-7, MMP-8, MMP-10, MMP-11, MMP-12, and MMP-13). Some specific examples of MMP inhibitors are AG-3340, RO 32-3555, and RS 13-0830.

Further exemplary anti-angiogenic agents include KDR (kinase domain receptor) inhibitory agents (e.g., antibodies and antigen binding regions that specifically bind to the kinase domain receptor), EGFR inhibitory agents (e.g., antibodies or antigen binding regions that specifically bind thereto) such as VECTIBIX® (panitumumab), erlotinib (TARCEVA®), anti-Angl and anti-Ang2 agents (e.g., antibodies or antigen binding regions specifically binding thereto or to their receptors, e.g., Tie2/Tek), and anti-Tie2 kinase inhibitory agents (e.g., antibodies or antigen binding regions that specifically bind thereto). Other anti-angiogenic agents include Campath, IL-8, B-FGF, Tek antagonists (US 20030162712; U.S. Pat. No. 6,413,932), anti-TWEAK agents (e.g., specifically binding antibodies or antigen binding regions, or soluble TWEAK receptor antagonists; see U.S. Pat. No. 6,727,225), ADAM distintegrin domain to antagonize the binding of integrin to its ligands (US 20020042368), specifically binding anti-eph receptor or anti-ephrin antibodies or antigen binding regions (U.S. Pat. Nos. 5,981,245; 5,728,813; 5,969,110; 6,596,852; 6,232,447; 6,057,124 and patent family members thereof), and anti-PDGF-BB antagonists (e.g., specifically binding antibodies or antigen binding regions) as well as antibodies or antigen binding regions specifically binding to PDGF-BB ligands, and PDGFR kinase inhibitory agents (e.g., antibodies or antigen binding regions that specifically bind thereto). Additional anti-angiogenic agents include: SD-7784 (Pfizer, USA); cilengitide (Merck KGaA, Germany, EPO 0770622); pegaptanib octasodium, (Gilead Sciences, USA); Alphastatin, (BioActa, UK); M-PGA, (Celgene, USA, U.S. Pat. No. 5,712,291); ilomastat, (Arriva, USA, U.S. Pat. No. 5,892,112); emaxanib, (Pfizer, USA, U.S. Pat. No. 5,792,783); vatalanib, (Novartis, Switzerland); 2-methoxyestradiol (EntreMed, USA); TLC ELL-12 (Elan, Ireland); anecortave acetate (Alcon, USA); alpha-D148 Mab (Amgen, USA); CEP-7055 (Cephalon, USA); anti-Vn Mab (Crucell, Netherlands), DACantiangiogenic (ConjuChem, Canada); Angiocidin (InKine Pharmaceutical, USA); KM-2550 (Kyowa Hakko, Japan); SU-0879 (Pfizer, USA); CGP-79787 (Novartis, Switzerland, EP 0970070); ARGENT technology (Ariad, USA); YIGSR-Stealth (Johnson & Johnson, USA); fibrinogen-E fragment (BioActa, UK); angiogenic inhibitor (Trigen, UK); TBC-1635 (Encysive Pharmaceuticals, USA); SC-236 (Pfizer, USA); ABT-567 (Abbott, USA); Metastatin (EntreMed, USA); maspin (Sosei, Japan); 2-methoxyestradiol (Oncology Sciences Corporation, USA); ER-68203-00 (IV AX, USA); BeneFin (Lane Labs, USA); Tz-93 (Tsumura, Japan); TAN-1120 (Takeda, Japan); FR-111142 (Fujisawa, Japan, JP 02233610); platelet factor 4 (RepliGen, USA, EP 407122); vascular endothelial growth factor antagonist (Borean, Denmark); bevacizumab (pINN) (Genentech, USA); angiogenic inhibitors (SUGEN, USA); XL 784 (Exelixis, USA); XL 647 (Exelixis, USA); MAb, alpha5beta3 integrin, second generation (Applied Molecular Evolution, USA and MedImmune, USA); enzastaurin hydrochloride (Lilly, USA); CEP 7055 (Cephalon, USA and Sanofi-Synthelabo, France); BC 1 (Genoa Institute of Cancer Research, Italy); rBPI 21 and BPI-derived antiangiogenic (XOMA, USA); PI 88 (Progen, Australia); cilengitide (Merck KGaA, German; Munich Technical University, Germany, Scripps Clinic and Research Foundation, USA); AVE 8062 (Ajinomoto, Japan); AS 1404 (Cancer Research Laboratory, New Zealand); SG 292, (Telios, USA); Endostatin (Boston Childrens Hospital, USA); ATN 161 (Attenuon, USA); 2-methoxyestradiol (Boston Childrens Hospital, USA); ZD 6474, (AstraZeneca, UK); ZD 6126, (Angiogene Pharmaceuticals, UK); PPI 2458, (Praecis, USA); AZD 9935, (AstraZeneca, UK); AZD 2171, (AstraZeneca, UK); vatalanib (pINN), (Novartis, Switzerland and Schering AG, Germany); tissue factor pathway inhibitors, (EntreMed, USA); pegaptanib (Pinn), (Gilead Sciences, USA); xanthorrhizol, (Yonsei University, South Korea); vaccine, gene-based, VEGF-2, (Scripps Clinic and Research Foundation, USA); SPV5.2, (Supratek, Canada); SDX 103, (University of California at San Diego, USA); PX 478, (ProIX, USA); METASTATIN, (EntreMed, USA); troponin I, (Harvard University, USA); SU 6668, (SUGEN, USA); OXI 4503, (OXiGENE, USA); o-guanidines, (Dimensional Pharmaceuticals, USA); motuporamine C, (British Columbia University, Canada); CDP 791, (Celltech Group, UK); atiprimod (pINN), (GlaxoSmithKline, UK); E 7820, (Eisai, Japan); CYC 381, (Harvard University, USA); AE 941, (Aeterna, Canada); vaccine, angiogenic, (EntreMed, USA); urokinase plasminogen activator inhibitor, (Dendreon, USA); oglufanide (pINN), (Melmotte, USA); HIF-lalfa inhibitors, (Xenova, UK); CEP 5214, (Cephalon, USA); BAY RES 2622, (Bayer, Germany); Angiocidin, (InKine, USA); A6, (Angstrom, USA); KR 31372, (Korea Research Institute of Chemical Technology, South Korea); GW 2286, (GlaxoSmithKline, UK); EHT 0101, (ExonHit, France); CP 868596, (Pfizer, USA); CP 564959, (OSI, USA); CP 547632, (Pfizer, USA); 786034, (GlaxoSmithKline, UK); KRN 633, (Kirin Brewery, Japan); drug delivery system, intraocular, 2-methoxyestradiol; anginex (Maastricht University, Netherlands, and Minnesota University, USA); ABT 510 (Abbott, USA); AAL 993 (Novartis, Switzerland); VEGI (ProteomTech, USA); tumor necrosis factor-alpha inhibitors; SU 11248 (Pfizer, USA and SUGEN USA); ABT 518, (Abbott, USA); YH16 (Yantai Rongchang, China); S-3APG (Boston Childrens Hospital, USA and EntreMed, USA); MAb, KDR (ImClone Systems, USA); MAb, alpha5 beta (Protein Design, USA); KDR kinase inhibitor (Celltech Group, UK, and Johnson & Johnson, USA); GFB 116 (South Florida University, USA and Yale University, USA); CS 706 (Sankyo, Japan); combretastatin A4 prodrug (Arizona State University, USA); chondroitinase AC (IBEX, Canada); BAY RES 2690 (Bayer, Germany); AGM 1470 (Harvard University, USA, Takeda, Japan, and TAP, USA); AG 13925 (Agouron, USA); Tetrathiomolybdate (University of Michigan, USA); GCS 100 (Wayne State University, USA) CV 247 (Ivy Medical, UK); CKD 732 (Chong Kun Dang, South Korea); irsogladine, (Nippon Shinyaku, Japan); RG 13577 (Aventis, France); WX 360 (Wilex, Germany); squalamine, (Genaera, USA); RPI 4610 (Sirna, USA); heparanase inhibitors (InSight, Israel); KL 3106 (Kolon, South Korea); Honokiol (Emory University, USA); ZK CDK (Schering AG, Germany); ZK Angio (Schering AG, Germany); ZK 229561 (Novartis, Switzerland, and Schering AG, Germany); XMP 300 (XOMA, USA); VGA 1102 (Taisho, Japan); VE-cadherin-2 antagonists(ImClone Systems, USA); Vasostatin (National Institutes of Health, USA); Flk-1 (ImClone Systems, USA); TZ 93 (Tsumura, Japan); TumStatin (Beth Israel Hospital, USA); truncated soluble FLT 1 (vascular endothelial growth factor receptor 1) (Merck & Co, USA); Tie-2 ligands (Regeneron, USA); and thrombospondin 1 inhibitor (Allegheny Health, Education and Research Foundation, USA).

Further examples of therapeutic agents that may be used in combination with a compound of the present disclosure include agents (e.g., antibodies, antigen binding regions, or soluble receptors) that specifically bind and inhibit the activity of growth factors, such as antagonists of hepatocyte growth factor (HGF, also known as Scatter Factor), and antibodies or antigen binding regions that specifically bind its receptor, c-Met.

Another example of a therapeutic agent that may be used in combination with a compound of the present disclosure is an anti-neoplastic agent. In some embodiments, the one or more additional therapies include an anti-neoplastic agent. Non-limiting examples of anti-neoplastic agents include acemannan, aclarubicin, aldesleukin, alemtuzumab, alitretinoin, altretamine, amifostine, aminolevulinic acid, amrubicin, amsacrine, anagrelide, anastrozole, ancer, ancestim, arglabin, arsenic trioxide, BAM-002 (Novelos), bexarotene, bicalutamide, broxuridine, capecitabine, celmoleukin, cetrorelix, cladribine, clotrimazole, cytarabine ocfosfate, DA 3030 (Dong-A), daclizumab, denileukin diftitox, deslorelin, dexrazoxane, dilazep, docetaxel, docosanol, doxercalciferol, doxifluridine, doxorubicin, bromocriptine, carmustine, cytarabine, fluorouracil, HIT diclofenac, interferon alfa, daunorubicin, doxorubicin, tretinoin, edelfosine, edrecolomab, eflornithine, emitefur, epirubicin, epoetin beta, etoposide phosphate, exemestane, exisulind, fadrozole, filgrastim, finasteride, fludarabine phosphate, formestane, fotemustine, gallium nitrate, gemcitabine, gemtuzumab zogamicin, gimeracil/oteracil/tegafur combination, glycopine, goserelin, heptaplatin, human chorionic gonadotropin, human fetal alpha fetoprotein, ibandronic acid, idarubicin, (imiquimod, interferon alfa, interferon alfa, natural, interferon alfa-2, interferon alfa-2a, interferon alfa-2b, interferon alfa-NI, interferon alfa-n3, interferon alfacon-1, interferon alpha, natural, interferon beta, interferon beta-Ia, interferon beta-Ib, interferon gamma, natural interferon gamma-la, interferon gamma-Ib, interleukin-1 beta, iobenguane, irinotecan, irsogladine, Ianreotide, LC 9018 (Yakult), leflunomide, lenograstim, lentinan sulfate, letrozole, leukocyte alpha interferon, leuprorelin, levamisole+fluorouracil, liarozole, lobaplatin, lonidamine, lovastatin, masoprocol, melarsoprol, metoclopramide, mifepristone, miltefosine, mirimostim, mismatched double stranded RNA, mitoguazone, mitolactol, mitoxantrone, molgramostim, nafarelin, naloxone+pentazocine, nartograstim, nedaplatin, nilutamide, noscapine, novel erythropoiesis stimulating protein, NSC 631570 octreotide, oprelvekin, osaterone, oxaliplatin, paclitaxel, pamidronic acid, pegaspargase, peginterferon alfa-2b, pentosan polysulfate sodium, pentostatin, picibanil, pirarubicin, rabbit antithymocyte polyclonal antibody, polyethylene glycol interferon alfa-2a, porfimer sodium, raloxifene, raltitrexed, rasburiembodiment, rhenium Re 186 etidronate, RII retinamide, rituximab, romurtide, samarium (153 Sm) lexidronam, sargramostim, sizofiran, sobuzoxane, sonermin, strontium-89 chloride, suramin, tasonermin, tazarotene, tegafur, temoporfin, temozolomide, teniposide, tetrachlorodecaoxide, thalidomide, thymalfasin, thyrotropin alfa, topotecan, toremifene, tositumomab-iodine 131, trastuzumab, treosulfan, tretinoin, trilostane, trimetrexate, triptorelin, tumor necrosis factor alpha, natural, ubenimex, bladder cancer vaccine, Maruyama vaccine, melanoma lysate vaccine, valrubicin, verteporfin, vinorelbine, virulizin, zinostatin stimalamer, or zoledronic acid; abarelix; AE 941 (Aeterna), ambamustine, antisense oligonucleotide, bcl-2 (Genta), APC 8015 (Dendreon), decitabine, dexaminoglutethimide, diaziquone, EL 532 (Elan), EM 800 (Endorecherche), eniluracil, etanidazole, fenretinide, filgrastim SD01 (Amgen), fulvestrant, galocitabine, gastrin 17 immunogen, HLA-B7 gene therapy (Vical), granulocyte macrophage colony stimulating factor, histamine dihydrochloride, ibritumomab tiuxetan, ilomastat, IM 862 (Cytran), interleukin-2, iproxifene, LDI 200 (Milkhaus), leridistim, lintuzumab, CA 125 MAb (Biomira), cancer MAb (Japan Pharmaceutical Development), HER-2 and Fc MAb (Medarex), idiotypic 105AD7 MAb (CRC Technology), idiotypic CEA MAb (Trilex), LYM-1-iodine 131 MAb (Techni clone), polymorphic epithelial mucin-yttrium 90 MAb (Antisoma), marimastat, menogaril, mitumomab, motexafin gadolinium, MX 6 (Galderma), nelarabine, nolatrexed, P 30 protein, pegvisomant, pemetrexed, porfiromycin, prinomastat, RL 0903 (Shire), rubitecan, satraplatin, sodium phenylacetate, sparfosic acid, SRL 172 (SR Pharma), SU 5416 (SUGEN), TA 077 (Tanabe), tetrathiomolybdate, thaliblastine, thrombopoietin, tin ethyl etiopurpurin, tirapazamine, cancer vaccine (Biomira), melanoma vaccine (New York University), melanoma vaccine (Sloan Kettering Institute), melanoma oncolysate vaccine (New York Medical College), viral melanoma cell lysates vaccine (Royal Newcastle Hospital), or valspodar.

Additional examples of therapeutic agents that may be used in combination with a compound of the present disclosure include ivonescimab, ipilimumab (YERVOY®); tremelimumab; galiximab; nivolumab, also known as BMS-936558 (OPDIVO®); pembrolizumab (KEYTRUDA®); avelumab (BAVENCIO®); AMP224; BMS-936559; MPDL3280A, also known as RG7446; MEDI-570; AMG557; MGA271; IMP321; BMS-663513; PF-05082566; CDX-1127; anti-OX40 (Providence Health Services); huMAbOX40L; atacicept; CP-870893; lucatumumab; dacetuzumab; muromonab-CD3; ipilumumab; MEDI4736 (IMFINZI®); MSB0010718C; AMP 224; adalimumab (HUMIRA®); ado-trastuzumab emtansine (KADCYLA®); aflibercept (EYLEA®); alemtuzumab (CAMPATH®); basiliximab (SIMULECT®); belimumab (BENLYSTA®); basiliximab (SIMULECT®); belimumab (BENLYSTA®); brentuximab vedotin (ADCETRIS®); canakinumab (ILARIS®); certolizumab pegol (CIMZIA®); daclizumab (ZENAPAX®); daratumumab (DARZALEX®); denosumab (PROLIA®); eculizumab (SOLIRIS®); efalizumab (RAPTIVA®); gemtuzumab ozogamicin (MYLOTARG®); golimumab (SIMPONI®); ibritumomab tiuxetan (ZEVALIN®); infliximab (REMICADE®); motavizumab (NUMAX®); natalizumab (TYSABRI®); obinutuzumab (GAZYVA®); ofatumumab (ARZERRA®); omalizumab (XOLAIR®); palivizumab (SYNAGIS®); pertuzumab (PERJETA®); ranibizumab (LUCENTIS®); raxibacumab (ABTHRAX®); tocilizumab (ACTEMRA®); tositumomab; tositumomab-i-131; tositumomab and tositumomab-i-131 (BEXXAR®); ustekinumab (STELARA®); AMG 102; AMG 386; AMG 479; AMG 655; AMG 706; AMG 745; and AMG 951.

The compounds described herein can be used in combination with the agents disclosed herein or other suitable agents, depending on the condition being treated. Hence, in some embodiments the one or more compounds of the disclosure will be co-administered with other therapies as described herein. When used in combination therapy, the compounds described herein may be administered with the second agent simultaneously or separately. This administration in combination can include simultaneous administration of the two agents in the same dosage form, simultaneous administration in separate dosage forms, and separate administration. That is, a compound described herein and any of the agents described herein can be formulated together in the same dosage form and administered simultaneously. Alternatively, a compound of the invention and any of the therapies described herein can be simultaneously administered, wherein both the agents are present in separate formulations. In another alternative, a compound of the present disclosure can be administered and followed by any of the therapies described herein, or vice versa. In some embodiments of the separate administration protocol, a compound of the invention and any of the therapies described herein are administered a few minutes apart, or a few hours apart, or a few days apart.

In some embodiments of any of the methods described herein, the first therapy (e.g., a compound of the disclosure) and one or more additional therapies are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours, up to 24 hours, or up to 1-7, 1-14, 1-21 or 1-30 days before or after the one or more additional therapies.

The disclosure also features kits including (a) a pharmaceutical composition including an agent (e.g., a compound of the invention) described herein, and (b) a package insert with instructions to perform any of the methods described herein. In some embodiments, the kit includes (a) a pharmaceutical composition including an agent (e.g., a compound of the invention) described herein, (b) one or more additional therapies (e.g., non-drug treatment or therapeutic agent), and (c) a package insert with instructions to perform any of the methods described herein.

As one aspect of the present disclosure contemplates the treatment of the disease or symptoms associated therewith with a combination of pharmaceutically active compounds that may be administered separately, the invention further relates to combining separate pharmaceutical compositions in kit form. The kit may comprise two separate pharmaceutical compositions: a compound of the present invention, and one or more additional therapies. The kit may comprise a container for containing the separate compositions such as a divided bottle or a divided foil packet. Additional examples of containers include syringes, boxes, and bags. In some embodiments, the kit may comprise directions for the use of the separate components. The kit form is particularly advantageous when the separate components are preferably administered in different dosage forms (e.g., oral and parenteral), are administered at different dosage intervals, or when titration of the individual components of the combination is desired by the prescribing health care professional.

### EMBODIMENTS

Embodiment 1: A method of treating a RAS protein-related disorder in a human subject in need thereof, the method comprising orally administering a total daily dose of 150 mg to 1200 mg of Compound A

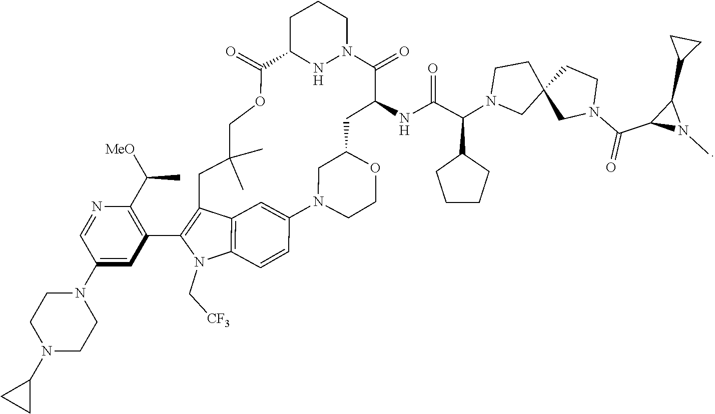

Embodiment 2: The method of embodiment 1, wherein the method comprises administering a total daily dose of 175 mg to 1200 mg of Compound A to the subject.

Embodiment 3: The method of embodiment 1, wherein the method comprises administering a total daily dose of 200 mg to 1200 mg of Compound A to the subject.

Embodiment 4: The method of embodiment 1, wherein the method comprises administering a total daily dose of 225 mg to 1200 mg of Compound A to the subject.

Embodiment 5: The method of embodiment 1, wherein the method comprises administering a total daily dose of 250 mg to 1200 mg of Compound A to the subject.

Embodiment 6: The method of embodiment 1, wherein the method comprises administering a total daily dose of 275 mg to 1200 mg of Compound A to the subject.

Embodiment 7: The method of embodiment 1, wherein the method comprises administering a total daily dose of 300 mg to 1200 mg of Compound A to the subject.

Embodiment 8: The method of embodiment 1, wherein the method comprises administering a total daily dose of 325 mg to 1200 mg of Compound A to the subject.

Embodiment 9: The method of embodiment 1, wherein the method comprises administering a total daily dose of 350 mg to 1200 mg of Compound A to the subject.

Embodiment 10: The method of embodiment 1, wherein the method comprises administering a total daily dose of 375 mg to 1200 mg of Compound A to the subject.

Embodiment 11: The method of embodiment 1, wherein the method comprises administering a total daily dose of 400 mg to 1200 mg of Compound A to the subject.

Embodiment 12: The method of embodiment 1, wherein the method comprises administering a total daily dose of 425 mg to 1200 mg of Compound A to the subject.

Embodiment 13: The method of embodiment 1, wherein the method comprises administering a total daily dose of 450 mg to 1200 mg of Compound A to the subject.

Embodiment 14: The method of embodiment 1, wherein the method comprises administering a total daily dose of 475 mg to 1200 mg of Compound A to the subject.

Embodiment 15: The method of embodiment 1, wherein the method comprises administering a total daily dose of 500 mg to 1200 mg of Compound A to the subject.

Embodiment 16: The method of embodiment 1, wherein the method comprises administering a total daily dose of 525 mg to 1200 mg of Compound A to the subject.

Embodiment 17: The method of embodiment 1, wherein the method comprises administering a total daily dose of 550 mg to 1200 mg of Compound A to the subject.

Embodiment 18: The method of embodiment 1, wherein the method comprises administering a total daily dose of 575 mg to 1200 mg of Compound A to the subject.

Embodiment 19: The method of embodiment 1, wherein the method comprises administering a total daily dose of 600 mg to 1200 mg of Compound A to the subject.

Embodiment 20: The method of embodiment 1, wherein the method comprises administering a total daily dose of 625 mg to 1200 mg of Compound A to the subject.

Embodiment 21: The method of embodiment 1, wherein the method comprises administering a total daily dose of 650 mg to 1200 mg of Compound A to the subject.

Embodiment 22: The method of embodiment 1, wherein the method comprises administering a total daily dose of 675 mg to 1200 mg of Compound A to the subject.

Embodiment 23: The method of embodiment 1, wherein the method comprises administering a total daily dose of 700 mg to 1200 mg of Compound A to the subject.

Embodiment 24: The method of embodiment 1, wherein the method comprises administering a total daily dose of 725 mg to 1200 mg of Compound A to the subject.

Embodiment 25: The method of embodiment 1, wherein the method comprises administering a total daily dose of 750 mg to 1200 mg of Compound A to the subject.

Embodiment 26: The method of embodiment 1, wherein the method comprises administering a total daily dose of 775 mg to 1200 mg of Compound A to the subject.

Embodiment 27: The method of embodiment 1, wherein the method comprises administering a total daily dose of 800 mg to 1200 mg of Compound A to the subject.

Embodiment 28: The method of embodiment 1, wherein the method comprises administering a total daily dose of 825 mg to 1200 mg of Compound A to the subject.

Embodiment 29: The method of embodiment 1, wherein the method comprises administering a total daily dose of 850 mg to 1200 mg of Compound A to the subject.

Embodiment 30: The method of embodiment 1, wherein the method comprises administering a total daily dose of 875 mg to 1200 mg of Compound A to the subject.

Embodiment 31: The method of embodiment 1, wherein the method comprises administering a total daily dose of 900 mg to 1200 mg of Compound A to the subject.

Embodiment 32: The method of embodiment 1, wherein the method comprises administering a total daily dose of 925 mg to 1200 mg of Compound A to the subject.

Embodiment 33: The method of embodiment 1, wherein the method comprises administering a total daily dose of 975 mg to 1200 mg of Compound A to the subject.

Embodiment 34: The method of embodiment 1, wherein the method comprises administering a total daily dose of 1000 mg to 1200 mg of Compound A to the subject.

Embodiment 35: The method of embodiment 1, wherein the method comprises administering a total daily dose of 1025 mg to 1200 mg of Compound A to the subject.

Embodiment 36: The method of embodiment 1, wherein the method comprises administering a total daily dose of 1050 mg to 1200 mg of Compound A to the subject.

Embodiment 37: The method of embodiment 1, wherein the method comprises administering a total daily dose of 1075 mg to 1200 mg of Compound A to the subject.

Embodiment 38: The method of embodiment 1, wherein the method comprises administering a total daily dose of 1100 mg to 1200 mg of Compound A to the subject.

Embodiment 39: The method of embodiment 1, wherein the method comprises administering a total daily dose of 1125 mg to 1200 mg of Compound A to the subject.

Embodiment 40: The method of embodiment 1, wherein the method comprises administering a total daily dose of 1150 mg to 1200 mg of Compound A to the subject.

Embodiment 41: The method of embodiment 1, wherein the method comprises administering a total daily dose of 1175 mg to 1200 mg of Compound A to the subject.

Embodiment 42: The method of embodiment 1, wherein the method comprises administering a total daily dose of 150 mg of Compound A to the subject.

Embodiment 43: The method of embodiment 1 to 7, wherein the method comprises administering a total daily dose of 300 mg of Compound A to the subject.

Embodiment 44: The method of any one of embodiments 1 to 13, wherein the method comprises administering a total daily dose of 450 mg of Compound A to the subject.

Embodiment 45: The method of any one of embodiments 1 to 19, wherein the method comprises administering a total daily dose of 600 mg of Compound A to the subject.

Embodiment 46: The method of any one of embodiments 1 to 31, wherein the method comprises administering a total daily dose of 900 mg of Compound A to the subject.

Embodiment 47: The method of any one of embodiments 1 to 41, wherein the method comprises administering a total daily dose of 1200 mg of Compound A to the subject.

Embodiment 48: The method of any one of embodiments 1 to 47, wherein Compound A is administered to the subject daily.

Embodiment 49: The method of embodiment 48, wherein Compound A is administered to the subject once or twice per day.

Embodiment 50: The method of embodiment 49, wherein Compound A is administered to the subject twice per day.

Embodiment 51: The method of embodiment 1, wherein the method comprises administering 150 mg of Compound A to the subject twice per day.

Embodiment 52: The method of embodiment 1, wherein the method comprises administering 300 mg of Compound A to the subject twice per day.

Embodiment 53: The method of embodiment 1, wherein the method comprises administering 450 mg of Compound A to the subject twice per day.

Embodiment 54: The method of embodiment 1, wherein the method comprises administering 600 mg of Compound A to the subject twice per day.

Embodiment 55: The method of any one of embodiments 1 to 47, wherein Compound A is administered to the subject more than twice per day.

Embodiment 56: The method of any one of embodiments 1 to 55, wherein the RAS protein-related disorder is a cancer.

Embodiment 57: The method of embodiment 56, wherein the cancer comprises a RAS mutation.

Embodiment 58: The method of embodiment 57, wherein the RAS mutation is at position 12.

Embodiment 59: The method of embodiment 58, wherein the RAS mutation is G12D.

Embodiment 60: The method of any one of embodiments 56 to 59, wherein the cancer is pancreatic cancer (e.g., pancreatic adenocarcinoma or pancreatic ductal adenocarcinoma).

Embodiment 61: The method of any one of embodiments 56 to 59, wherein the cancer is lung cancer.

Embodiment 62: The method of any one of embodiments 56 to 59, wherein the cancer is colorectal cancer.

Embodiment 63: The method of embodiment 61, wherein the lung cancer is non-small cell lung cancer.

Embodiment 64: The method of any one of embodiments 1 to 63, wherein the RAS protein is KRAS.

Embodiment 65: The method or use of any one of embodiments 56 to 64, wherein the method further comprises administering an additional anticancer therapy.

Embodiment 66: The method of embodiment 65, wherein the additional anticancer therapy is an EGFR inhibitor, a second RAS inhibitor, a SHP2 inhibitor, a SOS1 inhibitor, a Raf inhibitor, a MEK inhibitor, an ERK inhibitor, a PI13K inhibitor, a PTEN inhibitor, an AKT inhibitor, an mTORC1 inhibitor, a BRAF inhibitor, an immunotherapy, an immune checkpoint inhibitor, a CDK4/6 inhibitor, a HER2 inhibitor, or a combination thereof.

Embodiment 67: The method of embodiment 65 or 66, wherein the additional anticancer therapy is a RAS(ON) multi-selective inhibitor, a pan-KRAS inhibitor, and/or a KRAS^{G12D}(OFF) inhibitor.

Embodiment 68: The method of embodiment 66, wherein the second RAS inhibitor is a RAS(ON) G12C-selective inhibitor.

Embodiment 69: The method of embodiment 66, wherein the second RAS inhibitor is a RAS(ON) G12V-selective inhibitor.

Embodiment 70: The method of embodiment 65 or 66, wherein the additional anticancer therapy is pembrolizumab or a biosimilar thereof.

Embodiment 71: The method of embodiment 65 or 66, wherein the additional anticancer therapy is ivonescimab or a biosimilar thereof.

Embodiment 72: The method of embodiment 65 or 66, wherein the additional anticancer therapy is cetuximab or a biosimilar thereof.

Embodiment 73: The method of embodiment 65 or 66, wherein the additional anticancer therapy is a chemotherapeutic agent.

Embodiment 74: The method of any one of embodiments 1 to 73, wherein Compound A is administered without food.

Embodiment 75: The method of any one of embodiments 1 to 73, wherein the subject does not consume food for at least 4 hours after administration of Compound A.

Embodiment 76: The method of any one of embodiments 1 to 73, wherein the subject does not consume food for at least 8 hours prior to administration of Compound A.

Embodiment 77: The method of any one of embodiments 1 to 73, wherein the subject does not consume food for at least 2 hours prior to administration of Compound A and the subject does not consume food for at least 4 hours after administration of Compound A.

Embodiment 78: The method of any one of embodiments 1 to 73, wherein the subject is in a fasted state upon administration of Compound A.

Embodiment 79: The method of embodiment 78, wherein the subject does not consume water 1 hour prior to administration of Compound A and/or 1 hour after administration of Compound A.

Embodiment 80: The method of any one of embodiments 1 to 79, wherein the subject has locally advanced cancer.

Embodiment 81: The method of any one of embodiments 1 to 79, wherein the subject has metastatic cancer.

Embodiment 82: The method of any one of embodiments 1 to 81, wherein the subject does not have brain metastases prior to administration of Compound A.

Embodiment 83: The method of any one of embodiments 1 to 82, wherein the subject has not previously been treated with a cancer therapy, such that Compound A is a first-line therapy.

Embodiment 84: The method of any one of embodiments 1 to 82, wherein the subject has been treated with one prior cancer therapy, such that Compound A is a second-line therapy.

Embodiment 85: The method of any one of embodiments 1 to 82, wherein the subject has been treated with two prior cancer therapy, such that Compound A is a third-line therapy.

Embodiment 86: The method of any one of embodiments 1 to 85, wherein Compound A is administered in a treatment cycle of 28 days.

Embodiment 87: The method of embodiment 86, wherein the subject undergoes 1, 2, 3, 4, 5, 6, or more treatment cycles.

Embodiment 88: The method of any one of embodiments 1 to 87, wherein the subject exhibits an Eastern Cooperative Oncology Group (ECOG) performance status of 0, 1, or 2.

Embodiment 89: The method of any one of embodiments 1 to 88, wherein the subject is administered Compound A for at least 1 month.

Embodiment 90: The method of any one of embodiments 1 to 88, wherein the subject is administered Compound A for at least 3 months.

Embodiment 91: The method of any one of embodiments 1 to 88, wherein the subject is administered Compound A for at least 6 months.

Embodiment 92: The method of any one of embodiments 1 to 88, wherein the patient exhibits a progression free survival (PFS) of at least 3 months.

Embodiment 93: The method of any one of embodiments 1 to 88, wherein the patient exhibits a progression free survival (PFS) of at least 6 months.

Embodiment 94: The method of any one of embodiments 1 to 88, wherein the patient exhibits a progression free survival (PFS) of at least 8 months.

Embodiment 95: The method of any one of embodiments 1 to 88, wherein the patient exhibits a progression free survival (PFS) of at least 10 months.

Embodiment 96: The use of Compound A in a method of any one of embodiments 1 to 95.

Embodiment 97: The use of Compound A in the manufacture of a medicament for use in a method of any one of embodiments 1 to 95.

Embodiment 98: A method of avoiding an adverse drug interaction with Compound A in a human subject in need of Compound A, the method comprising administering to the subject a therapeutically effective amount of Compound A, and avoiding co-administration of a cytochrome p450 (CYP) 3A4 inhibitor, wherein said subject is also in need of the CYP3A4 inhibitor.

Embodiment 99: The method of embodiment 98, wherein the therapeutically effective amount of Compound A is a total daily dose between 100 mg to 1400 mg of Compound A.

Embodiment 100: A method of avoiding an adverse drug interaction with Compound A in a human subject in need of Compound A, comprising discontinuing administration of a CYP3A4 inhibitor to avoid an adverse drug interaction with Compound A, and administering to the subject a therapeutically effective amount of Compound A.

Embodiment 101: The method of embodiment 100, wherein the therapeutically effective amount of Compound A is a total daily dose between 100 mg to 1400 mg of Compound A.

Embodiment 102: The method of embodiments 100 or 101, wherein the CYP3A4 inhibitor is discontinued within 1 month prior to being administered Compound A.

Embodiment 103: The method of embodiments 100 or 101, wherein the CYP3A4 inhibitor is discontinued within 2 weeks prior to being administered Compound A.

Embodiment 104: The method of any one of embodiments 98 to 103, wherein the subject is advised that co-administration of Compound A and the CYP3A4 inhibitor can alter the therapeutic effect or adverse reaction profile of Compound A.

Embodiment 105: A method of administering Compound A to a human subject with cancer, wherein said subject is also in need of a CYP3A4 inhibitor, comprising administering to the subject a total daily dose between 100 mg to 1400 mg of Compound Awhile avoiding CYP3A4 inhibitor co-administration, and any one or more of the following: (a) advising the subject that the CYP3A4 inhibitor should be avoided or discontinued, (b) advising the subject that co-administration of Compound A with the CYP3A4 inhibitor can alter the therapeutic effect or adverse reaction profile of Compound A, (c) advising the subject that use of Compound A in subjects being treated with the CYP3A4 inhibitor is contraindicated, or (d) advising the subject that CYP3A4 inhibitors should be used with caution in subjects receiving Compound A due to the potential for an adverse reaction profile.

Embodiment 106: The method of embodiment 105, further comprising avoiding administering the CYP3A4 inhibitor.

Embodiment 107: The method of embodiment 105, further comprising discontinuing administration of the CYP3A4 inhibitor.

Embodiment 108: A method of avoiding an adverse drug interaction with Compound A in a human subject in need of Compound A, the method comprising administering to the subject a therapeutically effective amount of Compound A, and avoiding co-administration of a cytochrome p450 (CYP) 3A4 substrate, wherein said subject is also in need of the CYP3A4 substrate.

Embodiment 109: The method of embodiment 108, wherein the therapeutically effective amount of Compound A is a total daily dose between 100 mg to 1400 mg of Compound A.

Embodiment 110: A method of avoiding an adverse drug interaction with Compound A in a human subject in need of Compound A, comprising discontinuing administration of a CYP3A4 substrate to avoid an adverse drug interaction with Compound A, and administering to the subject a therapeutically effective amount of Compound A.

Embodiment 111: The method of embodiment 110, wherein the therapeutically effective amount of Compound A is a total daily dose between 100 mg to 1400 mg of Compound A.

Embodiment 112: The method of embodiments 110 or 111, wherein the CYP3A4 substrate is discontinued within 1 month prior to being administered Compound A.

Embodiment 113: The method of embodiments 110 or 111, wherein the CYP3A4 substrate is discontinued within 2 weeks prior to being administered Compound A.

Embodiment 114: The method of any one of embodiments 108 to 113, wherein the subject is advised that co-administration of Compound A and the CYP3A4 substrate can alter the therapeutic effect or adverse reaction profile of Compound A.

Embodiment 115: A method of administering Compound A to a human subject with cancer, wherein said subject is also in need of a CYP3A4 substrate, comprising administering to the subject a total daily dose between 100 mg to 1400 mg of Compound Awhile avoiding CYP3A4 substrate co-administration, and any one or more of the following: (a) advising the subject that the CYP3A4 substrate should be avoided or discontinued, (b) advising the subject that co-administration of Compound A with the CYP3A4 substrate can alter the therapeutic effect or adverse reaction profile of Compound A, (c) advising the subject that use of Compound A in subjects being treated with the CYP3A4 substrate is contraindicated, or (d) advising the subject that CYP3A4 substrates should be used with caution in subjects receiving Compound A due to the potential for an adverse reaction profile.

Embodiment 116: The method of embodiment 115, further comprising avoiding administering the CYP3A4 substrate.

Embodiment 117: The method of embodiment 115, further comprising discontinuing administration of the CYP3A4 substrate.

Embodiment 118: A method of avoiding an adverse drug interaction with Compound A in a human subject in need of Compound A, the method comprising administering to the subject a therapeutically effective amount of Compound A, and avoiding co-administration of a cytochrome p450 (CYP) 3A4 inducer, wherein said subject is also in need of the CYP3A4 inducer.

Embodiment 119: The method of embodiment 118, wherein the therapeutically effective amount of Compound A is a total daily dose between 100 mg to 1400 mg of Compound A.

Embodiment 120: A method of avoiding an adverse drug interaction with Compound A in a human subject in need of Compound A, comprising discontinuing administration of a CYP3A4 inducer to avoid an adverse drug interaction with Compound A, and administering to the subject a therapeutically effective amount of Compound A.

Embodiment 121: The method of embodiment 120, wherein the therapeutically effective amount of Compound A is a total daily dose between 100 mg to 1400 mg of Compound A.

Embodiment 122: The method of embodiments 120 or 121, wherein the CYP3A4 inducer is discontinued within 1 month prior to being administered Compound A.

Embodiment 123: The method of embodiments 120 or 121, wherein the CYP3A4 inducer is discontinued within 2 weeks prior to being administered Compound A.

Embodiment 124: The method of any one of embodiments 118 to 123, wherein the subject is advised that co-administration of Compound A and the CYP3A4 inducer can alter the therapeutic effect or adverse reaction profile of Compound A.

Embodiment 125: A method of administering Compound A to a human subject with cancer, wherein said subject is also in need of a CYP3A4 inducer, comprising administering to the subject a total daily dose between 100 mg to 1400 mg of Compound Awhile avoiding CYP3A4 inducer co-administration, and any one or more of the following: (a) advising the subject that the CYP3A4 inducer should be avoided or discontinued, (b) advising the subject that co-administration of Compound A with the CYP3A4 inducer can alter the therapeutic effect or adverse reaction profile of Compound A, (c) advising the subject that use of Compound A in subjects being treated with the CYP3A4 inducer is contraindicated, or (d) advising the subject that CYP3A4 inducers should be used with caution in subjects receiving Compound A due to the potential for an adverse reaction profile.

Embodiment 126: The method of embodiment 125, further comprising avoiding administering the CYP3A4 inducer.

Embodiment 127: The method of embodiment 125, further comprising discontinuing administration of the CYP3A4 inducer.

Embodiment 128: A method of avoiding an adverse drug interaction with Compound A in a human subject in need of Compound A, the method comprising administering to the subject a therapeutically effective amount of Compound A, and avoiding co-administration of a proton pump inhibitor (PPI), wherein said subject is also in need of the PPI.

Embodiment 129: The method of embodiment 128, wherein the therapeutically effective amount of Compound A is a total daily dose between 100 mg to 1400 mg of Compound A.

Embodiment 130: A method of avoiding an adverse drug interaction with Compound A in a human subject in need of Compound A, comprising discontinuing administration of a PPI to avoid an adverse drug interaction with Compound A, and administering to the subject a therapeutically effective amount of Compound A.

Embodiment 131: The method of embodiment 130, wherein the therapeutically effective amount of Compound A is a total daily dose between 100 mg to 1400 mg of Compound A.

Embodiment 132: The method of embodiments 130 or 131, wherein the PPI is discontinued within 1 month prior to being administered Compound A.

Embodiment 133: The method of embodiments 130 or 131, wherein the PPI is discontinued within 2 weeks prior to being administered Compound A.

Embodiment 134: The method of any one of embodiments 128 to 133, wherein the subject is advised that co-administration of Compound A and the PPI can alter the therapeutic effect or adverse reaction profile of Compound A.

Embodiment 135: A method of administering Compound A to a human subject with cancer, wherein said subject is also in need of a PPI, comprising administering to the subject a total daily dose between 100 mg to 1400 mg of Compound A while avoiding PPI co-administration, and any one or more of the following: (a) advising the subject that the PPI should be avoided or discontinued, (b) advising the subject that co-administration of Compound A with the PPI can alter the therapeutic effect or adverse reaction profile of Compound A, (c) advising the subject that use of Compound A in subjects being treated with the PPI is contraindicated, or (d) advising the subject that PPIs should be used with caution in subjects receiving Compound A due to the potential for an adverse reaction profile.

Embodiment 136: The method of embodiment 135, further comprising avoiding administering the PPI.

Embodiment 137: The method of embodiment 135, further comprising discontinuing administration of the PPI.

Embodiment 138: A method of avoiding an adverse drug interaction with Compound A in a human subject in need of Compound A, the method comprising administering to the subject a therapeutically effective amount of Compound A, and avoiding co-administration of a H-2 receptor antagonist (H2 blocker), wherein said subject is also in need of the H2 blocker.

Embodiment 139: The method of embodiment 138, wherein the therapeutically effective amount of Compound A is a total daily dose between 100 mg to 1400 mg of Compound A.

Embodiment 140: A method of avoiding an adverse drug interaction with Compound A in a human subject in need of Compound A, comprising discontinuing administration of a H2 blocker to avoid an adverse drug interaction with Compound A, and administering to the subject a therapeutically effective amount of Compound A.

Embodiment 141: The method of embodiment 140, wherein the therapeutically effective amount of Compound A is a total daily dose between 100 mg to 1400 mg of Compound A.

Embodiment 142: The method of embodiments 140 or 141, wherein the H2 blocker is discontinued within 1 month prior to being administered Compound A.

Embodiment 143: The method of embodiments 140 or 141, wherein the H2 blocker is discontinued within 2 weeks prior to being administered Compound A.

Embodiment 144: The method of any one of embodiments 138 to 143, wherein the subject is advised that co-administration of Compound A and the H2 blocker can alter the therapeutic effect or adverse reaction profile of Compound A.

Embodiment 145: A method of administering Compound A to a human subject with cancer, wherein said subject is also in need of a H2 blocker, comprising administering to the subject a total daily dose between 100 mg to 1400 mg of Compound A while avoiding H2 blocker co-administration, and any one or more of the following: (a) advising the subject that the H2 blocker should be avoided or discontinued, (b) advising the subject that co-administration of Compound A with the H2 blocker can alter the therapeutic effect or adverse reaction profile of Compound A, (c) advising the subject that use of Compound A in subjects being treated with the H2 blocker is contraindicated, or (d) advising the subject that H2 blockers should be used with caution in subjects receiving Compound A due to the potential for an adverse reaction profile.

Embodiment 146: The method of embodiment 145, further comprising avoiding administering the H2 blocker.

Embodiment 147: The method of embodiment 145, further comprising discontinuing administration of the H2 blocker.

Embodiment 148: A method of avoiding an adverse drug interaction with Compound A in a human subject in need of Compound A, the method comprising administering to the subject a therapeutically effective amount of Compound A, and avoiding co-administration of a disease-modifying antirheumatic drug (DMARD), wherein said subject is also in need of the DMARD.

Embodiment 149: The method of embodiment 148, wherein the therapeutically effective amount of Compound A is a total daily dose between 100 mg to 1400 mg of Compound A.

Embodiment 150: A method of avoiding an adverse drug interaction with Compound A in a human subject in need of Compound A, comprising discontinuing administration of a DMARD to avoid an adverse drug interaction with Compound A, and administering to the subject a therapeutically effective amount of Compound A.

Embodiment 151: The method of embodiment 150, wherein the therapeutically effective amount of Compound A is a total daily dose between 100 mg to 1400 mg of Compound A.

Embodiment 152: The method of embodiments 150 or 151, wherein the DMARD is discontinued within 1 month prior to being administered Compound A.

Embodiment 153: The method of embodiments 150 or 151, wherein the DMARD is discontinued within 2 weeks prior to being administered Compound A.

Embodiment 154: The method of any one of embodiments 148 to 153, wherein the subject is advised that co-administration of Compound A and the DMARD can alter the therapeutic effect or adverse reaction profile of Compound A.

Embodiment 155: A method of administering Compound A to a human subject with cancer, wherein said subject is also in need of a DMARD, comprising administering to the subject a total daily dose between 100 mg to 1400 mg of Compound A while avoiding DMARD co-administration, and any one or more of the following: (a) advising the subject that the DMARD should be avoided or discontinued, (b) advising the subject that co-administration of Compound A with the DMARD can alter the therapeutic effect or adverse reaction profile of Compound A, (c) advising the subject that use of Compound A in subjects being treated with the DMARD is contraindicated, or (d) advising the subject that DMARD should be used with caution in subjects receiving Compound A due to the potential for an adverse reaction profile.

Embodiment 156: The method of embodiment 155, further comprising avoiding administering the DMARD.

Embodiment 157: The method of embodiment 155, further comprising discontinuing administration of the DMARD.

Embodiment 158: A method of avoiding an adverse drug interaction with Compound A in a human subject in need of Compound A, the method comprising administering to the subject a therapeutically effective amount of Compound A, and avoiding co-administration of an immune suppressive agent, wherein said subject is also in need of the immune suppressive agent.

Embodiment 159: The method of embodiment 158, wherein the therapeutically effective amount of Compound A is a total daily dose between 100 mg to 1400 mg of Compound A.

Embodiment 160: A method of avoiding an adverse drug interaction with Compound A in a human subject in need of Compound A, comprising discontinuing administration of an immune suppressive agent to avoid an adverse drug interaction with Compound A, and administering to the subject a therapeutically effective amount of Compound A.

Embodiment 161: The method of embodiment 160, wherein the therapeutically effective amount of Compound A is a total daily dose between 100 mg to 1400 mg of Compound A.

Embodiment 162: The method of embodiments 160 or 161, wherein the immune suppressive agent is discontinued within 1 month prior to being administered Compound A.

Embodiment 163: The method of embodiments 160 or 161, wherein the immune suppressive agent is discontinued within 2 weeks prior to being administered Compound A.

Embodiment 164: The method of any one of embodiments 158 to 163, wherein the subject is advised that co-administration of Compound A and the immune suppressive agent can alter the therapeutic effect or adverse reaction profile of Compound A.

Embodiment 165: A method of administering Compound A to a human subject with cancer, wherein said subject is also in need of an immune suppressive agent, comprising administering to the subject a total daily dose between 100 mg to 1400 mg of Compound A while avoiding immune suppressive agent co-administration, and any one or more of the following: (a) advising the subject that the immune suppressive agent should be avoided or discontinued, (b) advising the subject that co-administration of Compound A with the immune suppressive agent can alter the therapeutic effect or adverse reaction profile of Compound A, (c) advising the subject that use of Compound A in subjects being treated with the immune suppressive agent is contraindicated, or (d) advising the subject that immune suppressive agents should be used with caution in subjects receiving Compound A due to the potential for an adverse reaction profile.

Embodiment 166: The method of embodiment 165, further comprising avoiding administering the immune suppressive agent.

Embodiment 167: The method of embodiment 165, further comprising discontinuing administration of the immune suppressive agent.

Embodiment 168: A method of avoiding an adverse drug interaction with Compound A in a human subject in need of Compound A, the method comprising administering to the subject a therapeutically effective amount of Compound A, and avoiding co-administration of a P-glycoprotein (P-gp) substrate, wherein said subject is also in need of the P-gp substrate.

Embodiment 169: The method of embodiment 168, wherein the therapeutically effective amount of Compound A is a total daily dose between 100 mg to 1400 mg of Compound A.

Embodiment 170: A method of avoiding an adverse drug interaction with Compound A in a human subject in need of Compound A, comprising discontinuing administration of a P-gp substrate to avoid an adverse drug interaction with Compound A, and administering to the subject a therapeutically effective amount of Compound A.

Embodiment 171: The method of embodiment 170, wherein the therapeutically effective amount of Compound A is a total daily dose between 100 mg to 1400 mg of Compound A.

Embodiment 172: The method of embodiments 170 or 171, wherein the P-gp substrate is discontinued within 1 month prior to being administered Compound A.

Embodiment 173: The method of embodiments 170 or 171, wherein the P-gp substrate is discontinued within 2 weeks prior to being administered Compound A.

Embodiment 174: The method of any one of embodiments 168 to 173, wherein the subject is advised that co-administration of Compound A and the P-gp substrate can alter the therapeutic effect or adverse reaction profile of Compound A.

Embodiment 175: A method of administering Compound A to a human subject with cancer, wherein said subject is also in need of a P-gp substrate, comprising administering to the subject a total daily dose between 100 mg to 1400 mg of Compound A while avoiding P-gp substrate co-administration, and any one or more of the following: (a) advising the subject that the P-gp substrate should be avoided or discontinued, (b) advising the subject that co-administration of Compound A with the P-gp substrate can alter the therapeutic effect or adverse reaction profile of Compound A, (c) advising the subject that use of Compound A in subjects being treated with the P-gp substrate is contraindicated, or (d) advising the subject that P-gp substrates should be used with caution in subjects receiving Compound A due to the potential for an adverse reaction profile.

Embodiment 176: The method of embodiment 175, further comprising avoiding administering the P-gp substrate.

Embodiment 177: The method of embodiment 175, further comprising discontinuing administration of the P-gp substrate.

Embodiment 178: A method of avoiding an adverse drug interaction with Compound A in a human subject in need of Compound A, the method comprising administering to the subject a therapeutically effective amount of Compound A, and avoiding co-administration of a P-glycoprotein (P-gp) inhibitor, wherein said subject is also in need of the P-gp inhibitor.

Embodiment 179: The method of embodiment 178, wherein the therapeutically effective amount of Compound A is a total daily dose between 100 mg to 1400 mg of Compound A.

Embodiment 180: A method of avoiding an adverse drug interaction with Compound A in a human subject in need of Compound A, comprising discontinuing administration of a P-gp inhibitor to avoid an adverse drug interaction with Compound A, and administering to the subject a therapeutically effective amount of Compound A.

Embodiment 181: The method of embodiment 180, wherein the therapeutically effective amount of Compound A is a total daily dose between 100 mg to 1400 mg of Compound A.

Embodiment 182: The method of embodiments 180 or 181, wherein the P-gp inhibitor is discontinued within 1 month prior to being administered Compound A.

Embodiment 183: The method of embodiments 180 or 181, wherein the P-gp inhibitor is discontinued within 2 weeks prior to being administered Compound A.

Embodiment 184: The method of any one of embodiments 178 to 183, wherein the subject is advised that co-administration of Compound A and the P-gp inhibitor can alter the therapeutic effect or adverse reaction profile of Compound A.

Embodiment 185: A method of administering Compound A to a human subject with cancer, wherein said subject is also in need of a P-gp inhibitor, comprising administering to the subject a total daily dose between 100 mg to 1400 mg of Compound A while avoiding P-gp inhibitor co-administration, and any one or more of the following: (a) advising the subject that the P-gp inhibitor should be avoided or discontinued, (b) advising the subject that co-administration of Compound A with the P-gp inhibitor can alter the therapeutic effect or adverse reaction profile of Compound A, (c) advising the subject that use of Compound A in subjects being treated with the P-gp inhibitor is contraindicated, or (d) advising the subject that P-gp inhibitors should be used with caution in subjects receiving Compound A due to the potential for an adverse reaction profile.

Embodiment 186: The method of embodiment 185, further comprising avoiding administering the P-gp inhibitor.

Embodiment 187: The method of embodiment 185, further comprising discontinuing administration of the P-gp inhibitor.

Embodiment 188: A method of avoiding an adverse drug interaction with Compound A in a human subject in need of Compound A, the method comprising administering to the subject a therapeutically effective amount of Compound A, and avoiding co-administration of an organic anion-transporting polypeptide 1B1 or 1B3 (OATP1B1 or OATP1B3) substrate, wherein said subject is also in need of the OATP1B1 or OATP1B3 substrate.

Embodiment 189: The method of embodiment 188, wherein the therapeutically effective amount of Compound A is a total daily dose between 100 mg to 1400 mg of Compound A.

Embodiment 190: A method of avoiding an adverse drug interaction with Compound A in a human subject in need of Compound A, comprising discontinuing administration of an OATP1B1 or OATP1B3 substrate to avoid an adverse drug interaction with Compound A, and administering to the subject a therapeutically effective amount of Compound A.

Embodiment 191: The method of embodiment 190, wherein the therapeutically effective amount of Compound A is a total daily dose between 100 mg to 1400 mg of Compound A.

Embodiment 192: The method of embodiments 190 or 191, wherein the OATP1B1 or OATP1B3 substrate is discontinued within 1 month prior to being administered Compound A.

Embodiment 193: The method of embodiments 190 or 191, wherein the OATP1B1 or OATP1B3 substrate is discontinued within 2 weeks prior to being administered Compound A.

Embodiment 194: The method of any one of embodiments 188 to 193, wherein the subject is advised that co-administration of Compound A and the OATP1B1 or OATP1B3 substrate can alter the therapeutic effect or adverse reaction profile of Compound A.

Embodiment 195: A method of administering Compound A to a human subject with cancer, wherein said subject is also in need of an OATP1B1 or OATP1B3 substrate, comprising administering to the subject a total daily dose between 100 mg to 1400 mg of Compound A while avoiding OATP1B1 or OATP1B3 co-administration, and any one or more of the following: (a) advising the subject that the OATP1B1 or OATP1B3 substrate should be avoided or discontinued, (b) advising the subject that co-administration of Compound A with the OATP1B1 or OATP1B3 substrate can alter the therapeutic effect or adverse reaction profile of Compound A, (c) advising the subject that use of Compound A in subjects being treated with the OATP1B1 or OATP1B3 substrate is contraindicated, or (d) advising the subject that OATP1B1 or OATP1B3 substrates should be used with caution in subjects receiving Compound A due to the potential for an adverse reaction profile.

Embodiment 196: The method of embodiment 195, further comprising avoiding administering the OATP1B1 or OATP1B3 substrate.

Embodiment 197: The method of embodiment 195, further comprising discontinuing administration of the OATP1B1 or OATP1B3 substrate.

Embodiment 198: A method of avoiding an adverse drug interaction with Compound A in a human subject in need of Compound A, the method comprising administering to the subject a therapeutically effective amount of Compound A, and avoiding co-administration of a breast cancer resistance protein B3 (BCRP) substrate, wherein said subject is also in need of the BCRP substrate.

Embodiment 199: The method of embodiment 198, wherein the therapeutically effective amount of Compound A is a total daily dose between 100 mg to 1400 mg of Compound A.

Embodiment 200: A method of avoiding an adverse drug interaction with Compound A in a human subject in need of Compound A, comprising discontinuing administration of a BCRP substrate to avoid an adverse drug interaction with Compound A, and administering to the subject a therapeutically effective amount of Compound A.

Embodiment 201: The method of embodiment 200, wherein the therapeutically effective amount of Compound A is a total daily dose between 100 mg to 1400 mg of Compound A.

Embodiment 202: The method of embodiments 200 or 201, wherein the BCRP substrate is discontinued within 1 month prior to being administered Compound A.

Embodiment 203: The method of embodiments 200 or 201, wherein the BCRP substrate is discontinued within 2 weeks prior to being administered Compound A.

Embodiment 204: The method of any one of embodiments 198 to 203, wherein the subject is advised that co-administration of Compound A and the BCRP substrate can alter the therapeutic effect or adverse reaction profile of Compound A.

Embodiment 205: A method of administering Compound A to a human subject with cancer, wherein said subject is also in need of a BCRP substrate, comprising administering to the subject a total daily dose between 100 mg to 1400 mg of Compound Awhile avoiding BCRP substrate co-administration, and any one or more of the following: (a) advising the subject that the BCRP substrate should be avoided or discontinued, (b) advising the subject that co-administration of Compound A with the BCRP substrate can alter the therapeutic effect or adverse reaction profile of Compound A, (c) advising the subject that use of Compound A in subjects being treated with the BCRP substrate is contraindicated, or (d) advising the subject that BCRP substrates should be used with caution in subjects receiving Compound A due to the potential for an adverse reaction profile.

Embodiment 206: The method of embodiment 205, further comprising avoiding administering the BCRP substrate.

Embodiment 207: The method of embodiment 205, further comprising discontinuing administration of the BCRP substrate.

Embodiment 208: A method of avoiding an adverse drug interaction with Compound A in a human subject in need of Compound A, the method comprising administering to the subject a therapeutically effective amount of Compound A, and avoiding co-administration of a drug known to prolong QTc interval, wherein said subject is also in need of the drug known to prolong QTc interval.

Embodiment 209: The method of embodiment 208, wherein the therapeutically effective amount of Compound A is a total daily dose between 100 mg to 1400 mg of Compound A.

Embodiment 210: A method of avoiding an adverse drug interaction with Compound A in a human subject in need of Compound A, comprising discontinuing administration of a drug known to prolong QTc interval to avoid an adverse drug interaction with Compound A, and administering to the subject a therapeutically effective amount of Compound A.

Embodiment 211: The method of embodiment 210, wherein the therapeutically effective amount of Compound A is a total daily dose between 100 mg to 1400 mg of Compound A.

Embodiment 212: The method of embodiments 210 or 211, wherein the drug known to prolong QTc interval is discontinued within 1 month prior to being administered Compound A.

Embodiment 213: The method of embodiments 210 or 211, wherein the drug known to prolong QTc interval is discontinued within 2 weeks prior to being administered Compound A.

Embodiment 214: The method of any one of embodiments 208 to 213, wherein the subject is advised that co-administration of Compound A and the drug known to prolong QTc interval can alter the therapeutic effect or adverse reaction profile of Compound A.

Embodiment 215: A method of administering Compound A to a human subject with cancer, wherein said subject is also in need of a drug known to prolong QTc interval, comprising administering to the subject a total daily dose between 100 mg to 1400 mg of Compound Awhile avoiding co-administration of a drug known to prolong QTc interval, and any one or more of the following: (a) advising the subject that the drug known to prolong QTc interval should be avoided or discontinued, (b) advising the subject that co-administration of Compound A with the drug known to prolong QTc interval can alter the therapeutic effect or adverse reaction profile of Compound A, (c) advising the subject that use of Compound A in subjects being treated with the drug known to prolong QTc interval is contraindicated, or (d) advising the subject that the drug known to prolong QTc interval should be used with caution in subjects receiving Compound A due to the potential for an adverse reaction profile.

Embodiment 216: The method of embodiment 215, further comprising avoiding administering the drug known to prolong QTc interval.

Embodiment 217: The method of embodiment 215, further comprising discontinuing administration of the drug known to prolong QTc interval.

Embodiment 218: A method of reducing a treatment related adverse event in a human subject in need of Compound A, the method comprising administering to the subject a total daily dose between 100 mg to 1400 mg of Compound A.

Embodiment 219: A method of reducing nausea in a human subject in need of Compound A, the method comprising administering to the subject a total daily dose between 100 mg to 1400 mg of Compound A.

Embodiment 220: A method of reducing diarrhea in a human subject in need of Compound A, the method comprising administering to the subject a total daily dose between 100 mg to 1400 mg of Compound A.

Embodiment 221: A method of reducing vomiting in a human subject in need of Compound A, the method comprising administering to the subject a total daily dose between 100 mg to 1400 mg of Compound A.

Embodiment 222: A method of reducing fatigue in a human subject in need of Compound A, the method comprising administering to the subject a total daily dose between 100 mg to 1400 mg of Compound A.

Embodiment 223: A method of reducing rash in a human subject in need of Compound A, the method comprising administering to the subject a total daily dose between 100 mg to 1400 mg of Compound A.

Embodiment 224: A method of reducing an increase in alanine transaminase in a human subject in need of Compound A, the method comprising administering to the subject a total daily dose between 100 mg to 1400 mg of Compound A.

Embodiment 225: A method of reducing an increase in aspartate transferase in a human subject in need of Compound A, the method comprising administering to the subject a total daily dose between 100 mg to 1400 mg of Compound A.

Embodiment 226: The method of any one of embodiments 218 to 225, wherein the method further comprises reducing the dose of Compound A from a total daily dose of 1400 mg to a total daily dose of 1200 mg.

Embodiment 227: The method of any one of embodiments 218 to 225, wherein the method further comprises reducing the dose of Compound A from a total daily dose of 1200 mg to a total daily dose of 900 mg.

Embodiment 228: The method of any one of embodiments 218 to 225, wherein the method further comprises reducing the dose of Compound A from a total daily dose of 900 mg to a total daily dose of 600 mg.

Embodiment 229: The method of any one of embodiments 218 to 225, wherein the method further comprises reducing the dose of Compound A from a total daily dose of 600 mg to a total daily dose of 300 mg.

Embodiment 230: The method of any one of embodiments 98 to 229, wherein the subject has cancer.

Embodiment 231: The method of embodiment 230, wherein the cancer comprises a RAS mutation.

Embodiment 232: The method of embodiment 231, wherein the RAS mutation is G12D.

Embodiment 233: The method of embodiments 231 or 232, wherein the RAS protein is KRAS.

Embodiment 234: The method of any one of embodiments 230 to 233, wherein the cancer is locally advanced or metastatic.

Embodiment 235: The method of any one of embodiments 230 to 234, wherein the cancer is pancreatic cancer (e.g., pancreatic adenocarcinoma or pancreatic ductal adenocarcinoma).

Embodiment 236: The method of any one of embodiments 230 to 234, wherein the cancer is lung cancer.

Embodiment 237: The method of any one of embodiments 230 to 234, wherein the cancer is colorectal cancer.

Embodiment 238. A method of treating a subject in need of a therapy comprising Compound A, the method comprising, administering Compound A at an initial dose and, upon the occurrence of an adverse event, administering a reduced dose of Compound A.

Embodiment 239. The method of embodiment 238, wherein the adverse event comprises a treatment-related adverse event, intolerance, or clinical finding warranting dose modification.

Embodiment 240. The method of embodiment 238 or 239, wherein the adverse event prompting dose reduction is fatigue, gastrointestinal toxicity, rash, or elevated liver enzymes.

Embodiment 241. The method of any one of embodiments 238 to 240, wherein the dose reduction maintains therapeutic exposure while minimizing further toxicity.

Embodiment 242. The method of any one of embodiments 238 to 241, wherein the dose reduction occurs after temporary interruption of treatment with Compound A or without interruption, depending on the severity of the adverse event.

Embodiment 243. The method of any one of embodiments 238 to 242, wherein the dose reductions are implemented sequentially or as a single step.

Embodiment 244. The method of any one of embodiments 238 to 243, wherein the administering of Compound A occurs on a once daily (QD) or twice daily (BID) dosing regimen.

Embodiment 245. The method of any one of embodiments 238 to 244, wherein the reduced dose is maintained until the adverse event resolves or stabilizes.

Embodiment 246. The method of any one of embodiments 238 to 245, further comprising re-escalating to the initial dose or further reducing the dose as clinically indicated.

Embodiment 247. The method of any one of embodiments 238 to 246, comprising administering Compound A to the subject at a dose of about 1200 mg (QD or BID) and, upon observation of an adverse event, administering to the subject Compound A at a subsequent dose of about 900 mg (QD or BID).

Embodiment 248. The method of any one of embodiments 238 to 247, comprising administering Compound A to the subject at a dose of about 900 mg (QD or BID) and, upon occurrence of an adverse event, administering to the subject Compound A at a subsequent dose of about 600 mg (QD or BID).

Embodiment 249. The method of any one of embodiments 238 to 248, comprising administering Compound A to the subject at a dose of about 600 mg (QD or BID) and, upon occurrence of an adverse event, administering to the subject Compound A at a subsequent dose of about 300 mg (QD or BID).

Embodiment 250. The method of any one of embodiments 238 to 249, wherein the subject is monitored for the occurrence, severity, and resolution of adverse events throughout the course of treatment.

Embodiment 251. The method of embodiment 250, wherein monitoring comprises clinical assessment, physical examination, laboratory testing, or patient-reported outcomes.

Embodiment 252. The method of any one of embodiments 238 to 251, further comprising re-initiating treatment at the next lower dose following temporary discontinuation.

Embodiment 253. The method of any one of embodiments 238 to 252, wherein the reduced dose is maintained for the remainder of therapy.

Embodiment 254. The method of any one of embodiments 238 to 253, wherein the dose-reduction sequence proceeds stepwise through one or more levels, for example from about 1200 mg total daily dose, to about 900 mg total daily dose, to about 600 mg total daily dose, to about 300 mg total daily dose, as necessary to maintain tolerability while preserving clinical efficacy.

Embodiment 255: Use of Compound A in any one of the methods of embodiments 98 to 254.

Embodiment 256: Use of Compound A in the manufacture of a medicament for use in any one of the methods of embodiments 98 to 254.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure or scope of the appended claims.

### Example 1: Phase 1 Study Design for Compound A Monotherapy in Subjects Having Advanced KRAS^{G12D}-Mutant Solid Tumors

As described herein, Compound A is a potent, mutant-selective covalent, and orally bioavailable tri-complex inhibitor of the active guanosine-5′-triphosphate (GTP)-bound state of the mutant KRAS^{G12D} protein (KRAS^{G12D}(ON)). Compound A binds to an intracellular protein, cyclophilin A (CypA), that is ubiquitously and abundantly expressed in normal tissues and tumors. This inhibitory binary complex binds to RAS(ON), forming a noncovalent tri-complex. In the case of KRAS^{GI2D}, this results in irreversible covalent modification of the unique Asp-12 residue by Compound A and prevention of downstream signaling.

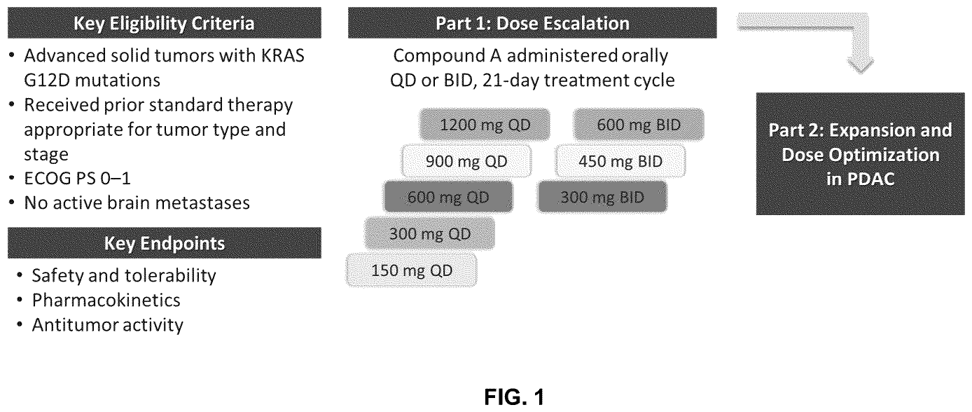

This Example describes the study design of a Phase 1/1b study of Compound A in patients with advanced KRAS^{G12C} solid tumors with the first patient dosed in September 2023. There are two components in this study: Part 1—Dose Escalation and Part 2—Dose Expansion (FIG. 1). In Part 1—Dose Escalation, an assessment of safety and tolerability are conducted, and the candidate recommended Phase 2 dose and schedule (RP2DS) are determined for further testing in the Dose Expansion Part. Part 2 of the study evaluates the safety and antitumor effects of the candidate RP2DS in KRAS^{G12D}-mutant solid tumors. Approximately 274 participants are enrolled across both Part 1 and Part 2 of the study.

Dose expansion cohorts include but are not limited to participants with KRAS^{G12D}_mutant advanced solid tumors with NSCLC, PDAC, and colorectal cancer (CRC). A final estimate of the RP2DS is evaluated and confirmed including all subjects from the dose escalation and dose expansion cohorts. All toxicities including those during Cycle 1 and late toxicities outside Cycle 1 are considered in the determination of RP2DS.

### Part 1: Dose Escalation Design

Dose escalation is guided by the Bayesian optimal interval (BOIN) design, with a target dose limiting toxicity (DLT) rate of 0.3 and an acceptable toxicity interval of (0.236, 0.333). Hence, if the observed toxicity rate at the current dose level is <0.236, then the dose is escalated for the next cohort. If the observed toxicity rate is >0.333, then the dose is de-escalated for the next cohort. DLT assessment is performed on the DLT-evaluable population. Enrollment began with an initial cohort size of 2 to 4 participants in the first 2 lower dose levels (s 300 mg), and 3 to 4 participants in higher dose levels. In case 3 or 4 participants are enrolled in the lower dose levels but the 3rd and 4t^{h} participants do not complete DLT period (Cycle 1) due to non-AE reasons or due to AEs that are not related (possibly, probably, or definitely) to study treatment, these participants will not be replaced, and dose escalation may proceed based on zero DLT. A minimum of 6 DLT-evaluable participants will be evaluated at the maximum tolerated dose (MTD), if reached.

The starting dose of Compound A was 150 mg QD administered PO. The dose levels for dose escalation are summarized in Table 2. The initial schedule for dose exploration was QD dosing; however, the alternative schedule (i.e., BID) is explored in parallel with QD dosing. The first cycle of treatment (i.e., first 21 days after treatment initiation) constitutes the DLT evaluation period.

TABLE 2
Dose levels during dose escalation of Compound A.

| | Total Daily | | QD | BID |
|---|---|---|---|---|
| Dose Group | Dose | | Schedule | Schedule |
| 1 (starting dose) | 150 | mg | 150 mg | |
| 2 | 300 | mg | 300 mg QD | 150 mg BID |
| 3 | 600 | mg | 600 mg QD | 300 mg BID |
| 4 | 900 | mg | 900 mg QD | 450 mg BID |
| 5 | 1200 | mg | 1200 mg QD | 600 mg BID |

Abbreviations:
BID = twice daily;
QD = once daily

In addition to the planned dose levels, further dose exploration (e.g., intermediate dose levels or doses beyond 1200 mg total daily dose if MTD is not reached) may also be implemented based on the Data Monitoring Committee (DMC) recommendation and emerging safety and/or PK data. Safety signals are closely monitored, and smaller escalation increments may be implemented.

Upon selection of the RP2DS, all participants who continue on study treatment may have the option of receiving the RP2DS.

### Backfill Cohorts

To better characterize the safety, PK, and preliminary activity of Compound A within specific histotypes, additional participants are enrolled in dose exploration backfill groups, which open once a dose level clears DLT evaluation and it is considered safe and tolerable. The backfill slots are reserved for participants who have KRAS^{G12D}-mutant solid tumors within specific histotype. Backfill enrollment may be concurrent with dose exploration, but enrollment priority will be given to dose exploration if there is a participant who qualifies for both backfill and dose exploration. Randomization may be employed to assign participants if multiple doses/schedules are open simultaneously for backfilling to inform dose optimization.

### Part 2: Dose Expansion

Part 2 enrolls one or more cohorts for evaluation of the candidate RP2DS(s). Part 2 expansion cohorts are initially planned to include participants with KRAS^{G12D} mutant solid tumors (e.g., NSCLC, PDAC, and CRC). If additional RP2DS candidates and/or KRAS^{G12}D-mutant cancer populations are to be explored, additional expansion cohorts may be added.

Two or more RP2DS candidates may be selected for further dose optimization and if they are opened simultaneously, randomization will be employed to assign participants to 1 of the candidate RP2DS. Randomization may be implemented via dynamically balancing key baseline characteristics between doses/schedules considering participants that were already dosed at the applicable doses/schedules.

Enrollment in Part 2 Dose Expansion may start when an initial candidate RP2DS is selected during Part 1 and may occur in parallel with Part 1.

### Food Effect

Food effect is assessed in Part 1 of this study and may be assessed in Part 2. Up to 36 evaluable participants (12 in Part 1 and 24 in Part 2) may be enrolled to evaluate food effect.

In Part 1 (Dose Exploration), the dose level for food effect evaluation are based on preceding PK data. Only dose levels that: a) have cleared DLT evaluation, b) are considered tolerable, and c) have measurable blood Compound A concentrations at multiple time points are open to food effect evaluation. The food effect dose groups are excluded from backfill enrollment and are not be included in the DLT analysis.

In Part 1 Food Effect cohort, Compound A PK is evaluated in subjects administered study treatment as follows:
One single test dose in the fed state 3 days prior to the start of Cycle 1 Day 1 (C1D1), which will be referred to as C1D-3;
Regular dosing schedule in the fasted state on C1D1 and thereafter.

In Part 2 Food Effect cohort, Compound A PK is evaluated in subjects administered study treatment preferably at the RP2DS dose, with the sequences as in Part 1.

All screening and baseline assessments are completed prior to administration of study treatment on C1D-3, and treatment-emergent adverse events (TEAEs) and other safety data collected starting on C1D-3 after administration of study treatment.

Fast stands for no food or drink (except for water) consumption. The participant should take Compound A orally once (or twice) daily at approximately the same time. For BID dosing, 2nd dose of the day should be taken 10-12 hours after the 1st dose. Compound A should be taken with 240 mL (8 fluid ounces) of water. Participant should fast at least 2 hours prior to and 1 hour after taking Compound A and water may be taken ad libitum during the fasting. This applies for QD and BID dosing.

For evaluation of food effect, participants are given a dose of Compound A under fed and fasted conditions as follows:

For the fed condition, following an overnight fast of at least 10 hours, participants start to consume a standard meal with moderate fat content at 30 minutes before administration of Compound A.

Participants should eat the meal in 30 minutes or less. The study Compound A should be administered with water. Additional water is allowed ad libitum except for 1 hour before and 1 hour after administration of Compound A. No food is allowed for at least 4 hours after administration of Compound A.

For the fasted condition, following an overnight fast of at least 10 hours, Compound A is administered to participants with water. Additional water is allowed ad libitum except for 1 hour before and 1 hour after administration of Compound A. No food is allowed for at least 4 hours after the Compound A is administered.

### Inclusion Criteria

Participants are eligible to be included in the study only if all the following criteria apply and none of the exclusion criteria are met.
1. Participants must be ≥18 years of age.
2. Pathologically documented, locally advanced or metastatic solid tumor malignancy with KRAS^{G12D}-mutations previously identified (independent of this trial) based on assays of circulating tumor DNA (ctDNA) or tumor tissue (obtained within 5 years of C1D1) performed in a Clinical Laboratory Improvement Amendments (CLIA)-certified laboratory (US) or equivalent per local regulation.
3. Participants must have received and progressed or been intolerant to prior standard therapy (including targeted therapy) appropriate for tumor type and stage (disease specific criteria specified below).
   Participants with no more than 3 prior lines of systemic therapy may be enrolled into Part 2 (Dose Expansion). A participant with more than 3 prior lines of systemic therapy may be enrolled into Part 2 (Dose Expansion) only upon the approval of the Medical Monitor. Participants with no prior standard therapy may be enrolled into a dedicated expansion cohort initiated with approval of the DMC/IDMC.
   Participants eligible for, but choosing to forgo, approved standard-of-care prior therapy (including targeted therapy) will sign informed consent documenting their decision.
   Participants with histologically documented NSCLC-specific inclusion:
      For both Part 1 (Dose Exploration) and Part 2 (Dose Expansion), participants must have been treated with BOTH immunotherapy and chemotherapy either given concurrently or sequentially.
   Participants with histologically documented CRC-specific inclusion:
      For both Part 1 (Dose Exploration) and Part 2 (Dose Expansion), participants must have been treated with at least 1 line of standard chemotherapy (e.g., fluoropyrimidine, oxaliplatin, and irinotecan) Participants with microsatellite-unstable tumors must have been treated with an immune checkpoint inhibitor unless contraindicated.
   Participants with other histologically documented solid tumor-specific inclusion
(including pancreatic ductal adenocarcinoma [PDAC]). For both Part 1 (Dose Exploration) and Part 2 (Dose Expansion), participants must have been treated with at
least 1 prior systemic therapy.
4. Participants should provide the most recent archival tumor samples collected within 5 years of C1D1. Participants who do not have archival tumor tissue samples at the time of study entry and who are unable or unwilling to undergo pretreatment tumor biopsy may still be eligible for the study following discussion with the Sponsor Medical Monitor. Fresh pretreatment tumor biopsies are not required for enrollment, but participants are encouraged to undergo pretreatment tumor biopsies when possible.
5. Participant must have measurable disease per Response Evaluation Criteria in Solid Tumors (version 1.1) (RECIST [v1.1]) criteria using either a computed tomography (CT) or magnetic resonance imaging (MRI) scan.
6. Participant must have a life expectancy of at least 3 months from start of treatment.
7. The participant's Eastern Cooperative Oncology Group (ECOG) performance status (PS) is 0 to 1 with no deterioration in PS (where it is >1) 2 weeks prior to C1D1. Rescreening is required if PS is >1 for any reason prior to C1D1 (C1D-3 for food effect dose groups).
8. Participant must have the ability to ingest and retain medications PO.
9. Participant must have adequate hematological and biological function, as follows:
   Absolute neutrophil count (ANC)≥1.5×10⁹/L without use of hematopoietic growth factors.
   Hemoglobin ≥9 g/dL; participant must not have received an RBC transfusion within 14 days of screening.
   Platelets ≥100×10⁹/L; participant must not have received a platelet transfusion within 7 days of screening.
   Hematologic criteria must be met without the use of hematopoietic growth factors within 7 days of the screening lab measurement for short acting growth factors and/or within 14 days of screening for long-acting growth factors (i.e., terminal elimination t½>48 hours [e.g., pegfilgrastim]).
10. Participant must have hepatic function as follows:
   AST and ALT s 3× upper limit of normal (ULN) (if liver metastases are present, ≤5×ULN).
   Total Bilirubin s 1.5×ULN (<2.0×ULN for participants with documented Gilbert's syndrome; <3.0×ULN for participants for whom the indirect bilirubin level suggests an extrahepatic source of elevation).
   Serum albumin ≥2.5 g/dL.
11. Participant must have renal function as follows: renal clearance as estimated glomerular filtration rate (eGFR) by Modification of Diet in Renal Disease (MDRD) Study Equation, or measured by 24-hour urine collection >60 mL/min).
12. Participant must have coagulation function as follows: prothrombin time (PT)/international normalized ratio (INR) and activated partial thromboplastin time (aPTT)/partial thromboplastin time (PTT)<1.5×ULN, or within a stable target range if taking prophylactic anticoagulant(s).
13. Participant must have recovered from all toxicities/AEs from prior anticancer therapy to Grade 1, normal or baseline except for alopecia. (Participants with other clinically non-significant Grade 1 to 2 toxicities may be allowed on study after discussion with the Sponsor Medical Monitor).
14. Participants with hypothyroidism must be on a stable dose of thyroid replacement.

### Additional Specific Inclusion Criteria for Participants in a Food Effect Cohort

Participants must be able to consume a standardized meal within 30 minutes.

Participants must be able to fast for >12 hours.

### Exclusion Criteria

Participants are excluded from the study if any of the following criteria apply.
1. Participant has primary CNS tumors.
2. Participant has known or suspected leptomeningeal metastases or spinal cord compression. Participants with symptomatic, active, or untreated brain metastases will also be excluded. However, a participant with previously treated (treatment may include clinical observation) or resected brain metastases will be allowed if ALL the following conditions are met:
   a. Radiation therapy ending at least 2 weeks prior to C1D1
   b. No evidence of clinical or radiographic disease progression as determined by a brain MRI performed within 28 days of C1D1
   c. Asymptomatic on a stable dose of anticonvulsant therapy and/or on a stable dose of steroids (if applicable) at least 28 days prior to C1D1.
3. Participant with uncontrolled seizure disorder refractory to antiepileptics.
4. Participants with a prior history of (<5 years) or concurrent malignancy are excluded. Exceptions may include prior malignancies considered to be clinically insignificant and for which no systemic anticancer treatment is required (e.g., basal cell or squamous cell carcinoma of the skin post curative surgical resection; carcinoma in situ of the cervix post-curative surgical resection).
5. Participant who has any of the following cardiac abnormalities:
   a. Medically uncontrolled hypertension (≥160 mmHg systolic blood pressure or ≥100 mmHg diastolic blood pressure) based on an average of 3 readings
   b. Congestive heart failure Class ≥2, as defined by the New York Heart Association
   c. Acute coronary syndrome (including unstable angina, coronary artery stenting, or angioplasty, bypass grafting within 6 months of screening); myocardial infarction within 6 months of anticipated C1D1
   d. History or evidence of current, uncontrolled, clinically significant, unstable arrhythmias
      Participant with medically controlled atrial fibrillation >1 month prior to Study Day 1 is eligible
      Participant who has a pacemaker in place to control atrial arrhythmias is a candidate for the study
   e. History of congenital long QT syndrome or prolonged QTc >470 msec for females and males using Fridericia's formula (unless a pacemaker is in place or additional clinically non-significant condition such as bundle-branch block necessitating use of an alternate formula per cardiologist calculation) or uncorrectable abnormalities in serum electrolytes (i.e., sodium, potassium, calcium, magnesium, phosphorus).
   An average of triplicate readings for assessing QTc interval may be used.
   f. Participant with an implantable defibrillator is not eligible to participate in the study
   g. Current cardiomyopathy or history within the past 12 months prior to anticipated C1D1
   h. Baseline left ventricular ejection fraction (LVEF)<50%.
6. Participant has a prior history of interstitial lung disease (ILD) or non-infectious pneumonitis which required treatment with corticosteroids or any active ILD or pneumonitis. Participants with a history of resolved radiation pneumonitis without interstitial lung disease may be considered with Sponsor Medical Monitor discussion.
7. Participant with prior thoracic radiotherapy within 2 months of start of treatment.
8. Participant has a history of cerebrovascular accident or transient ischemic attack within the previous 6 months of signing the ICF.
9. Participant was previously treated with an investigational KRAS^{G12D} inhibitor, pan- or multi-RAS inhibitor, or had prior therapy with any direct RAS-targeted therapy (e.g., degraders and inhibitors). Participants previously/currently enrolled in any other Revolution Medicines RAS-directed investigational trial are also ineligible. Additionally prior therapy with other MAPK pathway targeted therapy is prohibited (for example EGFR or BRAF targeted therapy).
10. Participant has a known impairment of gastrointestinal function that may significantly alter the absorption of Compound A (e.g., uncontrolled nausea and vomiting, diarrhea, malabsorption syndrome, inflammatory bowel disease, gastrectomy, or small bowel resection).
11. Participant has a history of severe allergic reactions to any of the study treatment components/excipients.
12. Participant has had a major surgical procedure(s) s 28 days or non-study-related minor procedure(s) s 7 days prior to C1D1; in all cases, the participant must be sufficiently recovered and stable before treatment administration.
13. Any participant with symptomatic pulmonary embolism or pulmonary embolism not being treated with a stable dose of anticoagulants.
14. Participant was previously treated with an investigational KRASG12D inhibitor, pan- or multi-RAS inhibitor, or had prior therapy with any direct RAS-targeted therapy (e.g., degraders and inhibitors). Participants previously/currently enrolled in any other Revolution Medicines RAS-directed investigational trial are also ineligible. Additionally prior therapy with other MAPK pathway targeted therapy is prohibited (for example EGFR or BRAF targeted therapy).
15. Participant had treatment with tyrosine kinase inhibitor (TKI), chemotherapy, biologics/monoclonal antibodies <21 days or 5 half-lives (whichever is shorter, before C1D1).
16. Participant had treatment with non-thoracic radiation therapy <14 days before C1D1.
17. Participant had treatment with immunotherapy (e.g., checkpoint inhibitors)<28 days before C1D1, depending on cycle length of the immunotherapy.
18. Participant had treatment with any other anticancer treatments including investigational agents that do not fit in the above categories <28 days before C1D1 (excluding COVID-19 vaccines).
19. Participant requires treatment with proton pump inhibitors (PPIs) or H2 receptor antagonists (H2-blockers). Participants using an antacid or other acid reducing agent must have a 24-hour wash out period prior to initiation of study treatment.
20. Participant received medications that are known to prolong QTc interval within 7 days prior to C1D1 or requires treatment with medications that are known to prolong QTc interval.
21. Participant received medication that is a strong CYP3A inhibitor within 7 days or a strong CYP3A inducer within 14 days prior to C1D1.
22. Participant requires medication that is a P-gp inhibitor and/or a sensitive P-gp or BCRP substrate during study treatment.
23. Participant requires treatment with cyclosporin A or derivative.

### Dose Reduction Steps for Compound A

Table 3 summarizes the dose reduction steps for Compound A.

TABLE 3
Dose Reduction Steps for Compound A

| Dose Reduction Level | Compound A |
|---|---|
| 4 | TBD (if applicable)^{a} |
| 3 | 1200 mg (QD or BID) |
| 2 | 900 mg (QD or BID) |
| 1 | 600 mg (QD or BID) |
| −1 | 300 mg (QD or BID) |

^{a}If a dose is escalated beyond 1200 mg.
Abbreviations:
BID = twice daily;
QD = once daily

### Prohibited Concomitant Therapy

The following medications are prohibited while on study treatment:
1. PPIs and H2-blockers.
2. Medications which are known to prolong QTc.
3. Disease modifying agents
4. Non-physiologic doses of corticosteroids
5. Immunosuppressive drugs
6. Anti-cancer therapy (except study treatment)
7. Strong CYP3A inhibitors and inducers
8. P-gp inhibitors or a sensitive P-gp or BCRP substrates that were not reviewed and approved by the Sponsor Medical Monitor
9. Radiation therapy: In cases of symptomatic non-target lesions that are amenable to radiation therapy, participants may be allowed to receive palliative radiation (holding RMC-9805 one day prior to, during, and one day after radiation treatment) and continue on study treatment with agreement from the Sponsor Medical Monitor.

Avoid concomitant administration of CYP3A4 and/or P-gp substrates (digoxin) with a narrow therapeutic index.

### Efficacy Assessment

Participants will be assessed for response using RECIST (v1.1). All measurable and non-measurable disease must be documented at screening and reassessed at each subsequent tumor evaluation. Response assessments will be assessed by the Investigator based on the most appropriate imaging method: CT scans (with contrast) or MRI. Imaging should include chest and abdomen (neck and pelvis are to be included depending on primary tumor type at the site of metastasis and Investigator assessment). At the Investigator's discretion, the imaging studies may be repeated at any time if disease progression is suspected. Additional studies, such as positron emission tomography (PET) scans or bone scans to confirm presence of new lesions, should be performed if clinically indicated but should not replace the primary method of tumor imaging employed at screening. Care should be taken to repeat the same modality used at screening throughout the study and to ensure all anatomy imaged at screening is again imaged at follow-up scans for any given participant.

### Pharmacokinetic and Biomarker Assessments

Whole blood samples, collected according to the PK sampling schedules, will be measured for concentrations of Compound A using validated LC-MS/MS methods. For intensive PK sampling days (C1D1/C1D2 and C1D15/C1D16), certain time points may be skipped for later cohorts if emerging clinical PK data suggest so. Revised PK sampling schedule will be provided to sites at that time.

Whole blood samples may be used for Compound A, and its metabolites quantification and metabolic profiling, if deemed necessary. Unused blood from samples collected for drug concentration analyses may also be used for biomarker assessments.

All participants are required to submit the most recent archival tissues within 5 years before start of treatment. Participants who do not have archival tissue are encouraged to undergo a tumor biopsy, when possible, but it is not required for enrollment. Paired pretreatment and/or on-study treatment and/or post study treatment fresh tumor samples may optionally be collected from all participants.

Blood samples along with archival, optional pretreatment, on study treatment and/or post study treatment biopsies will be analyzed for pharmacodynamic/predictive/resistant markers as well as confirmatory mutation assessment. Tests may be designed to identify participants most likely to respond to Compound A, to investigate and further understand the pharmacodynamic evidence and biological impact of Compound A by characterization of changes in levels of gene (DNA or RNA) or protein expression of specific markers.

### Example 2: Safety, Pharmacokinetics, and Antitumor Activity of Compound A, an Oral, RAS(ON) G12D-Selective, Tri-Complex Inhibitor in Patients with KRAS G12D Pancreatic Ductal Adenocarcinoma from a Phase 1 Study in Advanced Solid Tumors

Currently, most individuals with G12D mutations have no approved (or effective) RAS-targeted therapeutic options. This represents a large patient population that could potentially benefit from Compound A treatment. Treatment options for KRAS^{G12D}_mutant patient populations in NSCLC, PDAC, and CRC primarily include chemotherapy regimens and surgery, which often provide modest benefit.

RMC-9805-001 is a first-in-human study of Compound A in adults with advanced KRAS G12D-mutant solid tumors. As described in Example 1, the study is enrolling patients who have advanced solid tumors with KRAS G12D mutations, have received prior standard therapy, and have an ECOG PS 0-1 with no active brain metastases. The primary endpoint for the study is safety and tolerability of Compound A. Additional endpoints include pharmacokinetics (PK) and antitumor activity. The study consists of 2 parts: dose escalation of Compound A monotherapy followed by dose expansion and optimization (FIG. 1). In Part 1 patients received escalating doses of Compound A across 5 dose levels ranging from 150 mg to 1200 mg on a once daily schedule (QD) and 300 mg to 600 mg on a twice daily schedule (BID). No DLTs were observed at any dose or schedule, and the Maximum Tolerated Dose was not reached. Part 2 is further evaluating the safety/tolerability and PK of Compound A, as well as the preliminary anti-tumor activity for specific tumor types, with initial focus on PDAC.

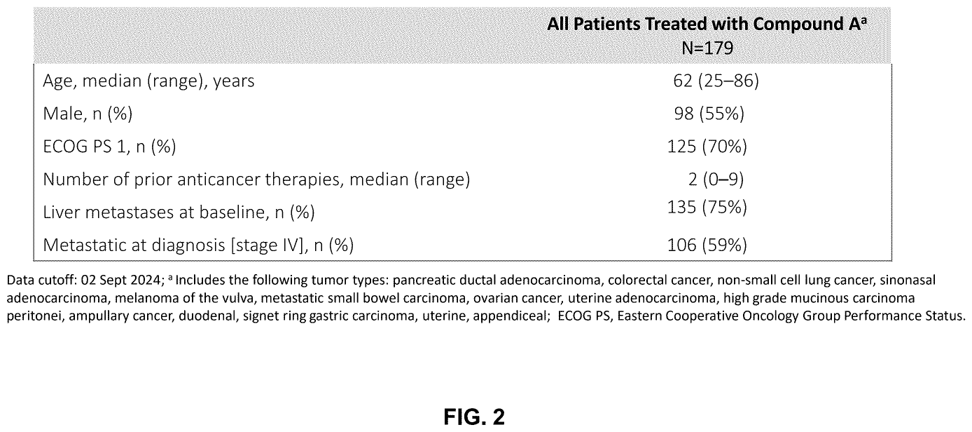

A total of 179 patients with KRAS G12D mutant solid tumors received Compound A as of the data cutoff date of Sep. 2, 2024. The baseline demographics are consistent with a Phase 1 study population with advanced or metastatic solid tumors (FIG. 2). Patients had a median age of 62 years, 70% had an ECOG PS of 1, the median prior lines of anticancer therapies was 2, and 75% of patients had liver metastasis at baseline.

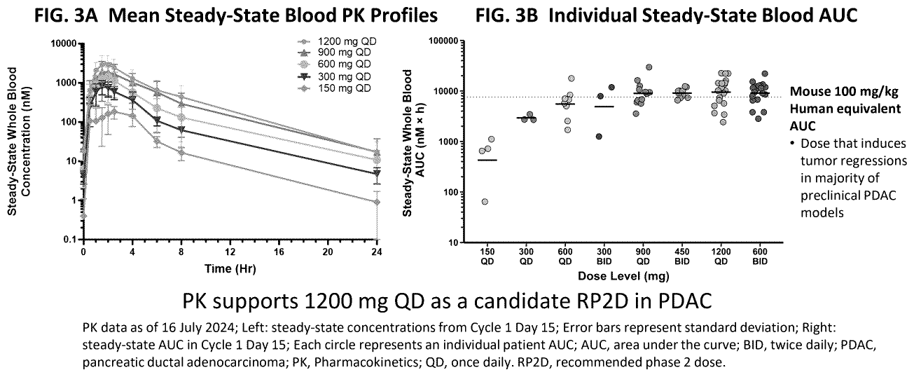

Compound A exhibits a dose-dependent increase in exposure from 150 mg to 1200 mg once daily dose. Comparable exposure is observed between QD and BID schedules at equivalent daily dose levels. The PK data supports a 1200 mg once daily dose as a candidate recommended phase 2 dose and schedule in patients with PDAC (FIG. 3A and FIG. 3B).

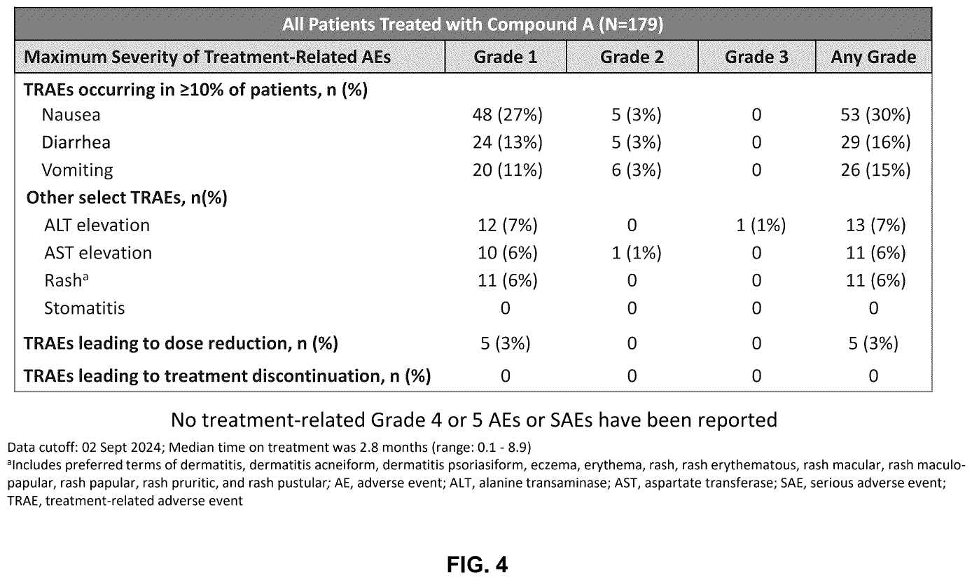

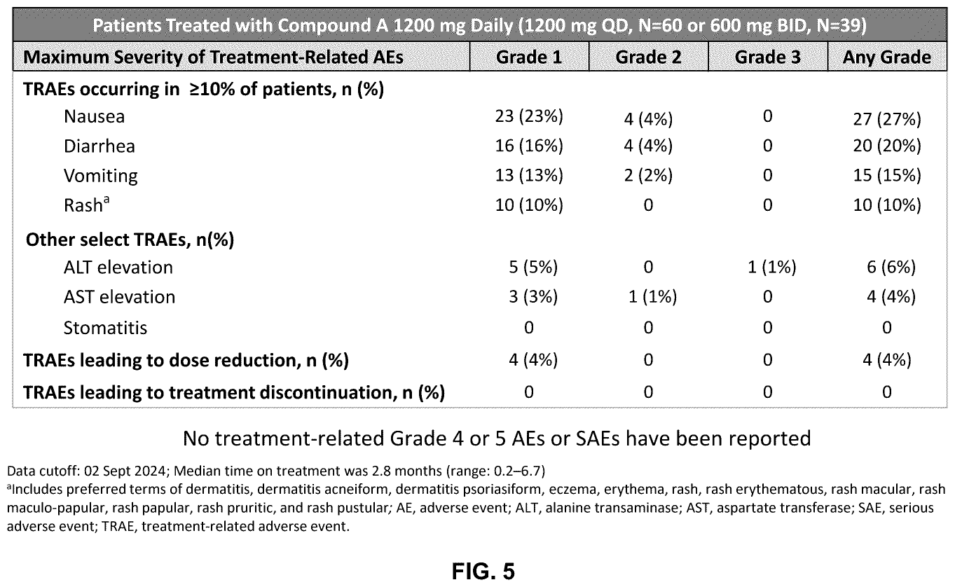

Compound A is generally well-tolerated across all dose levels. Nausea, diarrhea, and vomiting were the most commonly reported TRAEs, all of which were grade 1 or 2 in severity and clinically manageable. In fact, over 80% of nausea had been Grade 1, and around half of these events occurred and resolved in the first 4 days of starting treatment. TRAEs of interest were ALT or AST elevation. Only 6-7% of patients experienced primarily grade 1 events and only 1 patient experienced a Grade 3 event. Rash was infrequent and limited to grade 1. Stomatitis was not observed. Only 3% of patients had a TRAE that led to a dose reduction, and no patients had a TRAE that led to dose discontinuation. No treatment-related Grade 4 or 5 AEs or SAEs were reported (FIG. 4). The Compound A candidate RP2D daily dose of 1200 mg, which includes 60 patients at 1200 mg QD and 39 patients at 600 mg BID, was also generally well-tolerated. Nausea, diarrhea, vomiting, and rash were TRAEs that occurred in 10% or more of patients and were primarily grade 1 in severity. ALT or AST elevation was relatively infrequent with observed events mostly Grade 1 in severity. Only 4% of patients had a TRAE that led to a dose reduction, and no patients had a TRAE that led to dose discontinuation (FIG. 5).

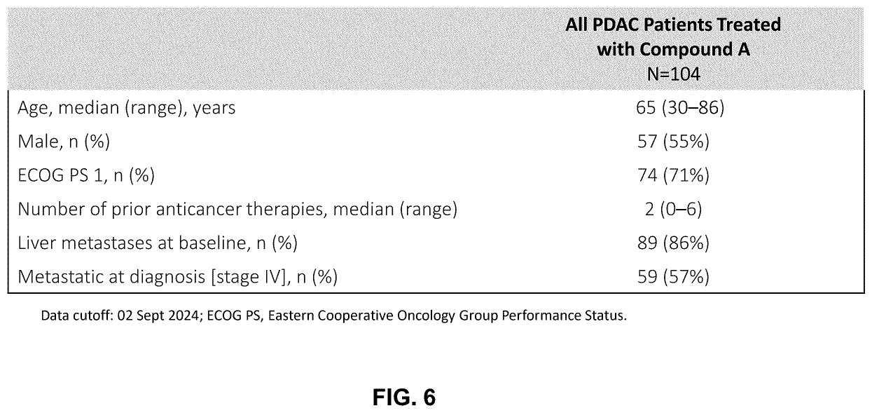

In total 104 PDAC patients received RMC-9805 across all dose levels. The baseline demographics are consistent with a Phase 1 study PDAC population. The median age was 65 years old, 71% had an ECOG PS of 1, the median lines of prior anti-cancer therapies was 2, and 86% of patients had liver metastases at baseline (FIG. 6).

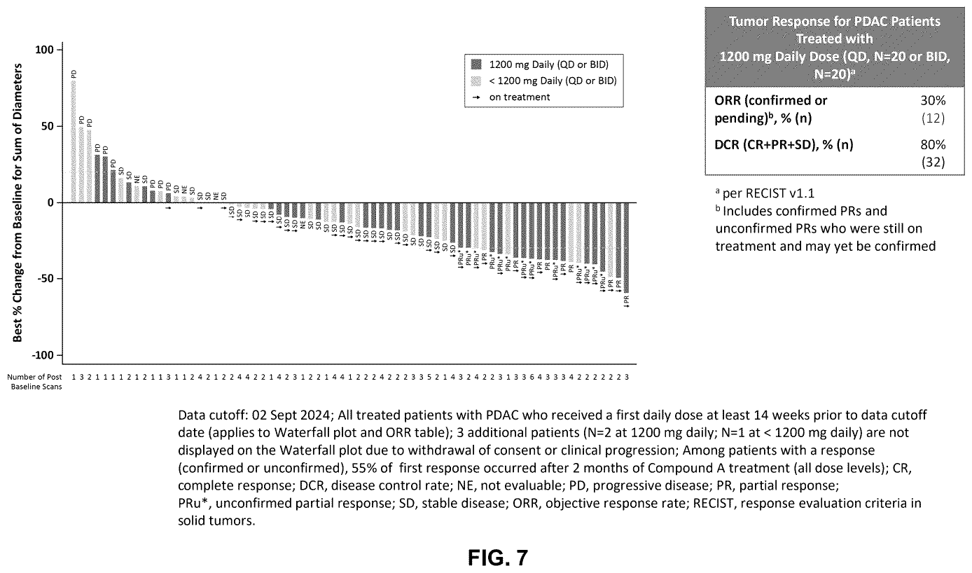

FIG. 7 shows a waterfall plot of best percentage change in tumor burden using RECIST v1.1 for all patients with PDAC who received a first daily dose of Compound A at least 14 weeks prior to the data cutoff date. The dark grey bars represent patients treated with Compound A 1200 mg daily and the light grey bars represent patients treated with less than 1200 mg daily. Among patients with KRAS G12D PDAC treated with the 1200 mg daily dose, the ORR was 30%, which includes patients with a confirmed response or response that is pending confirmation. The disease control rate was 80%. Notably, among patients with a response, 55% of first response occurred after 2 months of Compound A treatment.

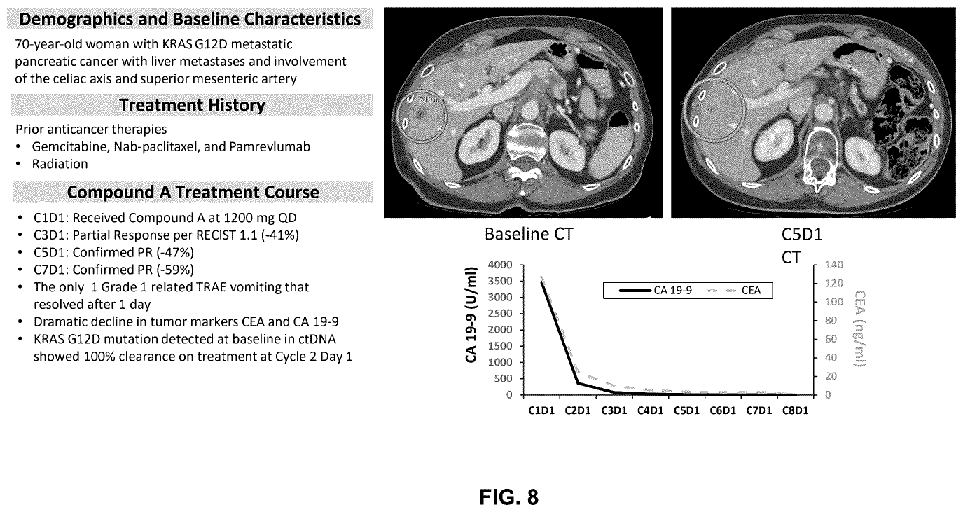

FIG. 8 shows a case report involving a patient treated with Compound A 1200 mg once daily. This is a 70-year-old woman with KRAS G12D metastatic pancreatic cancer with liver metastases and involvement of the celiac axis and superior mesenteric artery. Her prior anticancer therapies included gemcitabine, nab-paclitaxel, and pamrevlumab, as well as radiation therapy. At cycle 3 day 1, she achieved a partial response per RECIST v1.1 with a 47% decrease in tumor size. At cycle 5 day 1, the PR was confirmed and the lesion continued to decrease in size with her best overall response thus far occurring at cycle 7 day 1 with a 59% reduction. The only TRAE experienced was Grade 1 vomiting that resolved after 1 day. There was a rapid and sustained decline in the tumor markers CEA and CA 19-9. Similarly, KRAS G12D mutation detected at baseline in ctDNA showed 100% clearance on treatment at Cycle 2 Day 1.

### Example 3: Preliminary Safety and Antitumor Activity of Compound A, an Oral, RAS(ON) G12D-Selective, Tri-Complex Inhibitor in Patients with KRAS G12D Non-Small Cell Lung Cancer (NSCLC) from a Phase 1 Study in Advanced Solid Tumors

This Example reflects updated clinical data as of Dec. 2, 2024. Earlier data is provided in Example 2 (data cutoff date: Sep. 2, 2024). Ongoing treatment demonstrates encouraging antitumor activity, durability of response, and favorable safety and tolerability data for the Compound A in patients with KRAS G12D NSCLC.

A Phase 1, first-in-human clinical study (RMC-9805-001) is ongoing in patients with advanced solid tumors harboring the KRAS G12D mutation. The study consisted of a dose escalation phase followed by a dose expansion and optimization phase. Eligible patients had advanced solid tumors with KRAS G12D mutations, prior systemic therapy, ECOG performance status (PS) of 0 or 1, and no active brain metastases. In the dose escalation portion, patients received Compound A orally either once daily (QD) at doses ranging from 150 mg to 1200 mg, or twice daily (BID) at doses ranging from 300 mg to 600 mg (see Example 1). No dose-limiting toxicities (DLTs) were observed at any dose level or schedule, and the maximum tolerated dose (MTD) was not reached. A once-daily dose of 1200 mg was selected as the candidate recommended Phase 2 dose (RP2D).

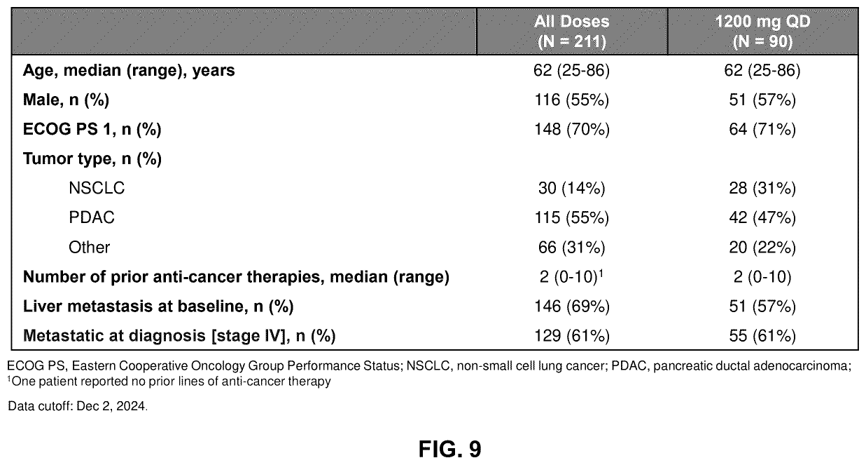

As of the data cutoff on Dec. 2, 2024, a total of 211 patients with KRAS G12D-mutant solid tumors had been treated with Compound A. Of these, 90 patients received the 1200 mg QD dose. The baseline characteristics for the full population and for the 1200 mg QD subgroup are shown in FIG. 9. The median age was 62 years, with 55% male. ECOG PS was 1 in 70% of patients. Tumor types included pancreatic ductal adenocarcinoma (PDAC) (55%) and NSCLC (14%). Patients had received a median of two prior lines of systemic therapy, and 69% presented with liver metastases at baseline.

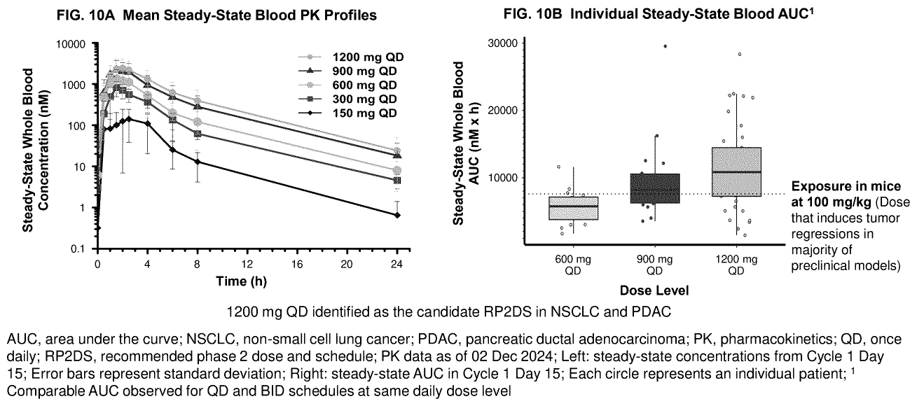

Pharmacokinetic analysis demonstrated dose-dependent increases in systemic exposure between 300 mg and 1200 mg QD. Mean steady-state concentrations and individual AUC values measured on Cycle 1 Day 15 are shown in FIG. 10A and FIG. 10B. Drug exposures exceeded the threshold associated with tumor regression in murine models, thereby supporting the biological relevance of the clinical dose.

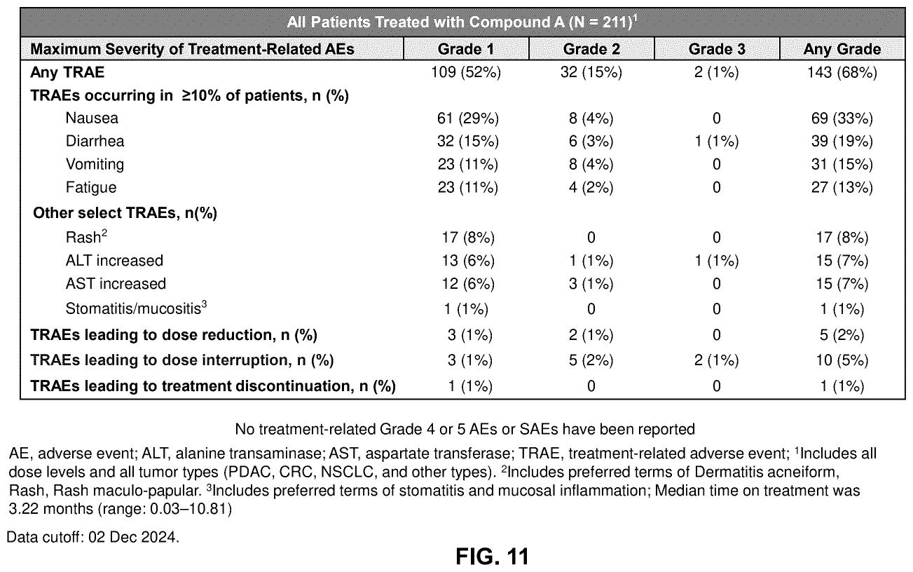

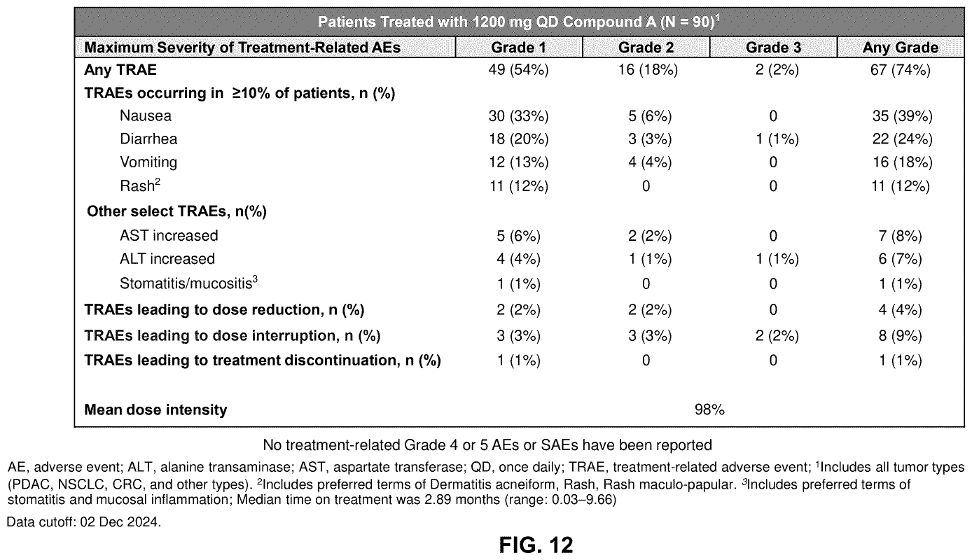

Compound A is generally well tolerated at all dose levels. The incidence of treatment-related adverse events (TRAEs) occurring in >10% of patients is summarized in FIG. 11. The most commonly observed TRAEs included nausea, diarrhea, vomiting, and fatigue, which were primarily Grade 1 or 2 in severity. Nausea was mostly Grade 1 (85%) and typically occurred and resolved within the first four days of treatment initiation. Only two Grade 3 TRAEs were observed. No treatment-related Grade 4 or 5 events occurred. Events of interest for this class of compounds, including rash, ALT/AST elevation, and mucositis, were infrequent and low-grade. The safety profile in patients receiving the candidate RP2D of 1200 mg QD (n=90) remained consistent with the overall patient population. TRAEs, including nausea, diarrhea, vomiting, and rash, were again primarily Grade 1. Transaminase elevations were infrequent and mostly low-grade. Only 4% of patients required a dose reduction, 9% had a dose interruption, and one patient discontinued due to a TRAE (FIG. 12).

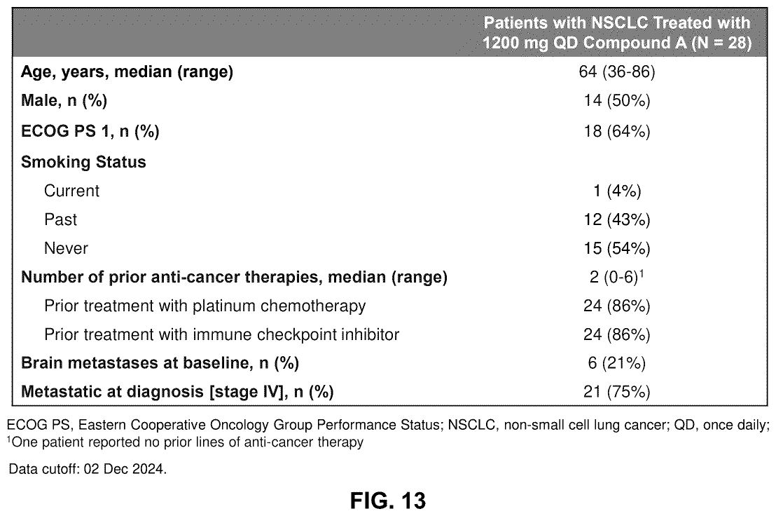

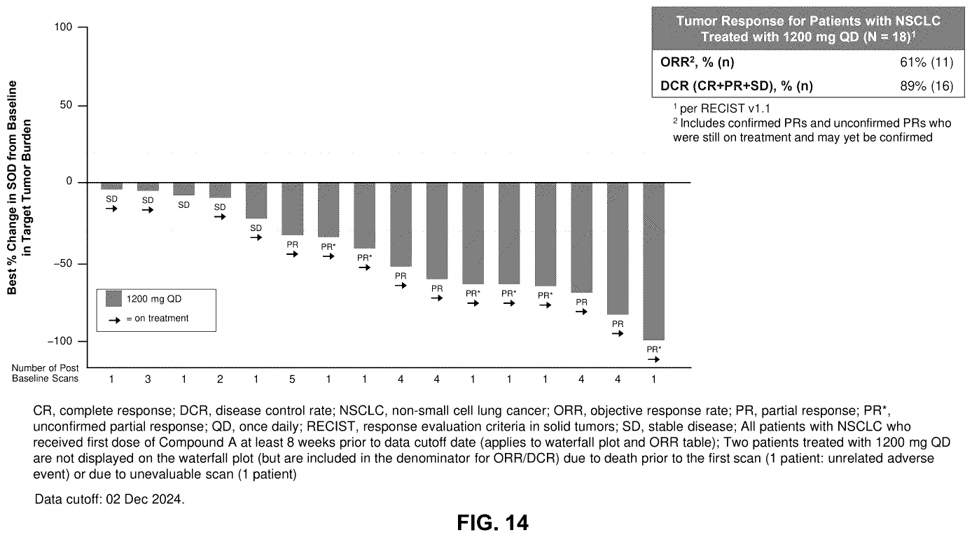

Twenty-eight patients with KRAS G12D-mutant NSCLC were treated with Compound A at the 1200 mg QD dose. Baseline demographics for this NSCLC subgroup are presented in FIG. 13. The median age was 64 years, with 50% male and 64% having an ECOG PS of 1. Notably, 54% of patients were never-smokers. The majority had received prior platinum-based chemotherapy and/or immune checkpoint inhibitors. Approximately 75% had metastatic disease at the time of diagnosis, and 21% had brain metastases at baseline. Clinical responses in the NSCLC subgroup were encouraging. Among the 18 patients who had received at least 8 weeks of treatment, the ORR was 61% and the DCR was 89% (FIG. 14). Sixteen patients were evaluable for radiographic response, with two additional patients included in the overall ORR and DCR calculations despite being unevaluable radiographically.

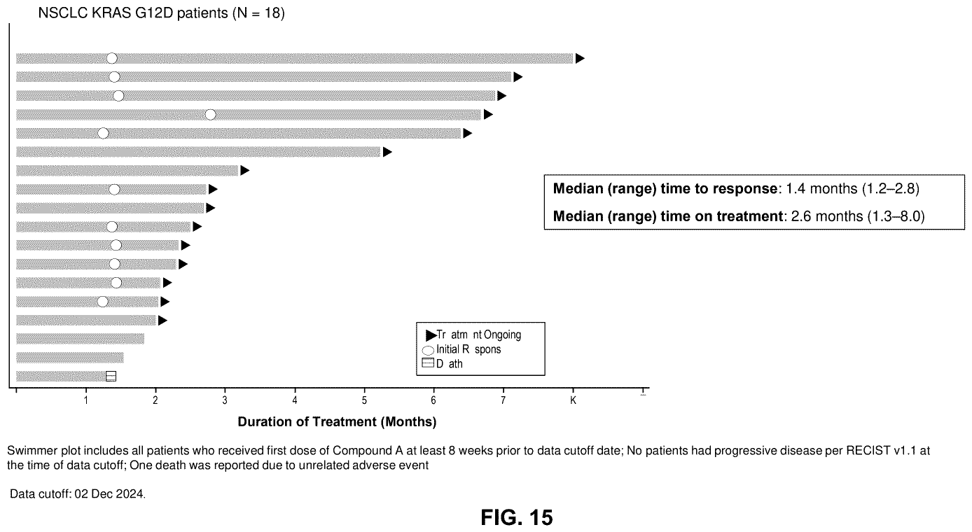

Most patients achieved a response at the first imaging assessment, with a median time to response of 1.4 months. The median treatment duration was 2.6 months, and several patients continued on treatment beyond five months at the time of analysis (FIG. 15).

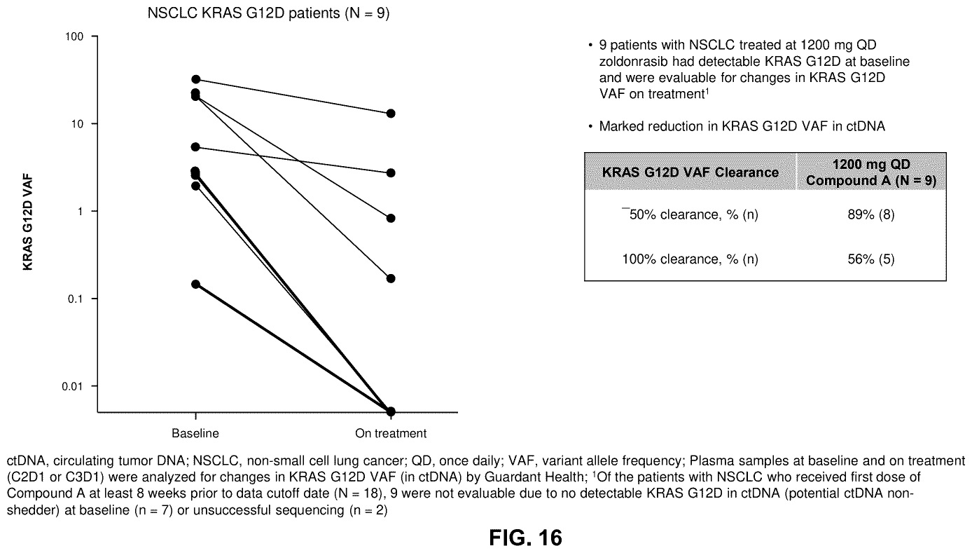

Pharmacodynamic assessment of KRAS G12D variant allele frequency (VAF) in circulating tumor DNA (ctDNA) was conducted in nine evaluable NSCLC patients. As shown in FIG. 16, 89% of patients exhibited >50% reduction in VAF, and 56% had complete (100%) clearance of KRAS G12D VAF at either Cycle 2 Day 1 or Cycle 3 Day 1.

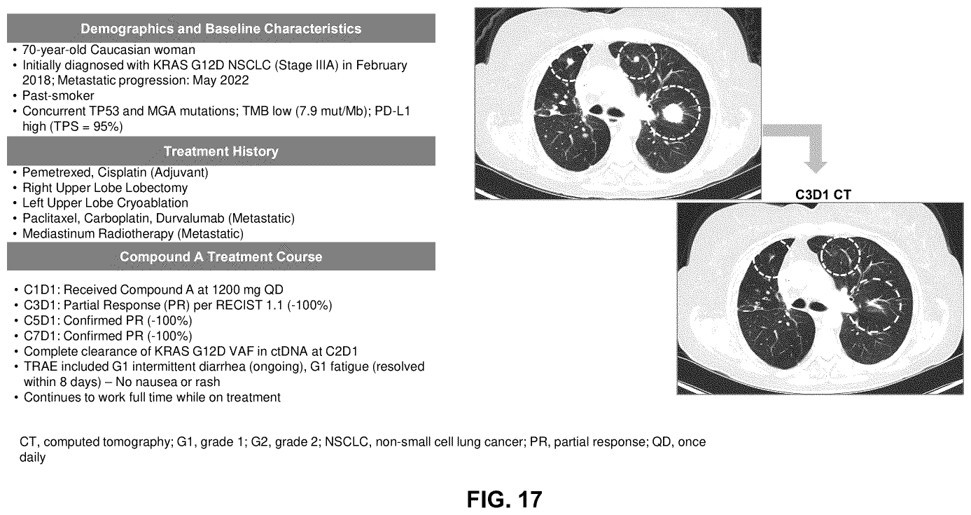

Clinical benefit was further illustrated by individual case reports. A case summary for a 70-year-old woman with metastatic KRAS G12D NSCLC is provided in FIG. 17. The patient is a past-smoker and has concurrent mutations in TP53 and MGA. The patient has low TMB and is PD-L1 high. The patient had previously received adjuvant chemotherapy and immune checkpoint therapy plus chemotherapy in the metastatic setting. Following treatment with Compound A 1200 mg QD, the patient experienced a 100% reduction in tumor size by Cycle 3 Day 1, which was confirmed at Cycle 5 Day 1. Complete clearance of KRAS G12D VAF was achieved by Cycle 2 Day 1. The patient's only TRAEs were Grade 1 intermittent diarrhea and fatigue.

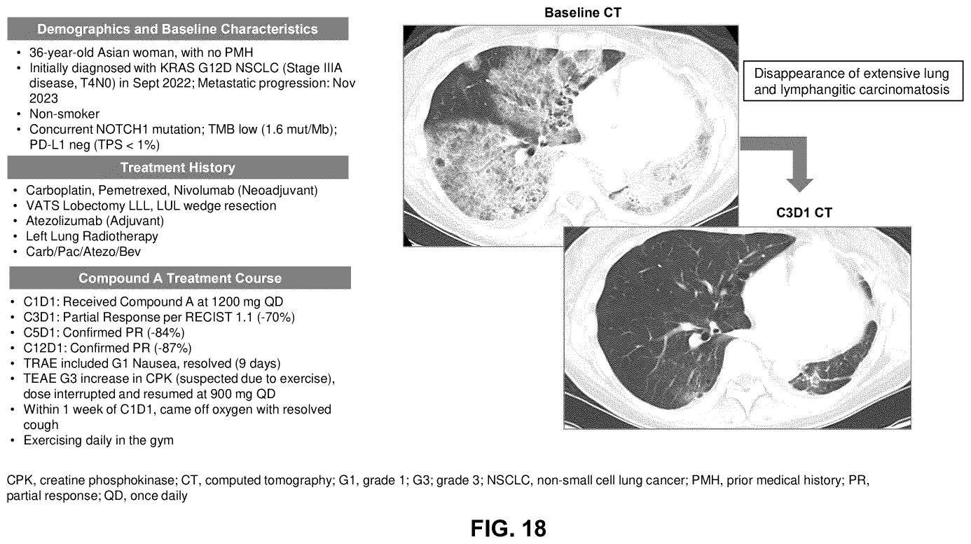

Another case report is shown in FIG. 18, describing a 36-year-old Asian female never-smoker with KRAS G12D NSCLC and a NOTCH1 co-mutation. The patient has low TMB and is PD-L1 negative. The patient prior to receiving Compound A presented with lymphangitic carcinomatosis and was oxygen-dependent at baseline. Within one week of initiating Compound A 1200 mg QD, the patient no longer required oxygen and reported resolution of cough. Imaging at Cycle 3 Day 1 demonstrated a 70% reduction in tumor size. At the most recent assessment, the patient had a confirmed 87% partial response. The patient's only TRAE was Grade 1 nausea which resolved after 9 days.

The present example supports Compound A as a potent, covalent, RAS(ON) G12D-selective inhibitor. The compound demonstrated dose-proportional pharmacokinetics, favorable tolerability, and clinically meaningful antitumor activity in patients with KRAS G12D-mutant NSCLC. Pharmacodynamic analyses supported molecular target engagement and tumor response. These findings support the continued development of Compound A as both monotherapy and in combination with agents such as daraxonrasib or standard-of-care regimens for RAS-driven malignancies.

1. A method of treating a cancer in a human subject in need thereof, the method comprising orally administering to the subject a total daily dose of 150 mg to 1200 mg of Compound A

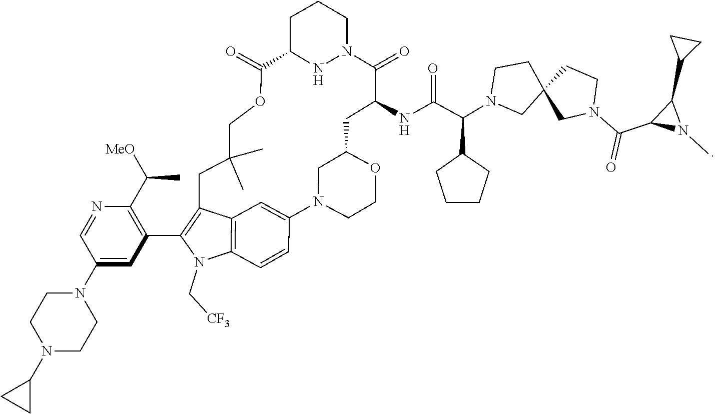

2. The method of claim 1, wherein the method comprises administering a total daily dose of 600 mg to 1200 mg of Compound A to the subject.

3. The method of claim 1, wherein the method comprises administering a total daily dose of 900 mg to 1200 mg of Compound A to the subject.

4. The method of claim 1, wherein the method comprises administering a total daily dose of 600 mg of Compound A to the subject.

5. The method of claim 1, wherein the method comprises administering a total daily dose of 900 mg of Compound A to the subject.

6. The method of claim 1, wherein the method comprises administering a total daily dose of 1200 mg of Compound A to the subject.

7. The method of claim 1, wherein 600 mg of Compound A is administered to the subject twice per day.

8. The method of any one of claims 1 to 7, wherein the cancer comprises a RAS mutation.

9. The method of claim 8, wherein the RAS mutation is G12D.

10. The method of any one of claims 1 to 9, wherein the cancer is pancreatic cancer.

11. The method of claim 10, wherein the pancreatic cancer is pancreatic adenocarcinoma.

12. The method of claim 10, wherein the pancreatic cancer is pancreatic ductal adenocarcinoma.

13. The method of any one of claims 1 to 9, wherein the cancer is lung cancer.

14. The method of claim 13, wherein the lung cancer is non-small cell lung cancer.

15. The method of any one of claims 1 to 9, wherein the cancer is colorectal cancer.

16. The method of any one of claims 9 to 15, wherein the RAS is KRAS.

17. The method of any one of claims 1 to 16, wherein the method further comprises administering an additional anticancer therapy.

18. The method of claim 17, wherein the additional anticancer therapy is an EGFR inhibitor, a second RAS inhibitor, a SHP2 inhibitor, a SOS1 inhibitor, a Raf inhibitor, a MEK inhibitor, an ERK inhibitor, a PI13K inhibitor, a PTEN inhibitor, an AKT inhibitor, an mTORC1 inhibitor, a BRAF inhibitor, an immunotherapy, an immune checkpoint inhibitor, a CDK4/6 inhibitor, a HER2 inhibitor, a PRMT5 inhibitor, or a combination thereof.

19. The method of claim 18, wherein the second RAS inhibitor is a RAS(ON) multi-selective inhibitor or a pan-KRAS inhibitor.

20. The method of claim 18, wherein the additional anticancer therapy is pembrolizumab or a biosimilar thereof.

21. The method of claim 18, wherein the additional anticancer therapy is ivonescimab or a biosimilar thereof.

22. The method of claim 20 or 21, further comprising administering to the subject one or more chemotherapeutic agents

23. The method of claim 18, wherein the additional anticancer therapy is cetuximab or a biosimilar thereof.

24. The method of claim 17, wherein the additional anticancer therapy is one or more chemotherapeutic agents.

25. The method of claim 18, wherein the additional anticancer therapy is a PRMT5 inhibitor.

26. A method of avoiding an adverse drug interaction with Compound A in a human subject in need of Compound A, the method comprising administering to the subject a therapeutically effective amount of Compound A, and avoiding co-administration of a cytochrome p450 (CYP) 3A4 inhibitor, wherein said subject is also in need of the CYP3A4 inhibitor.

27. A method of avoiding an adverse drug interaction with Compound A in a human subject in need of Compound A, comprising discontinuing administration of a CYP3A4 inhibitor to avoid an adverse drug interaction with Compound A, and administering to the subject a therapeutically effective amount of Compound A.

28. A method of administering Compound A to a human subject with cancer, wherein said subject is also in need of a CYP3A4 inhibitor, comprising administering to the subject a total daily dose between 100 mg to 1400 mg of Compound A while avoiding CYP3A4 inhibitor co-administration, and any one or more of the following:
(a) advising the subject that the CYP3A4 inhibitor should be avoided or discontinued,
(b) advising the subject that co-administration of Compound A with the CYP3A4 inhibitor can alter the therapeutic effect or adverse reaction profile of Compound A,
(c) advising the subject that use of Compound A in subjects being treated with the CYP3A4 inhibitor is contraindicated, or
(d) advising the subject that CYP3A4 inhibitors should be used with caution in subjects receiving Compound A due to the potential for an adverse reaction profile.

29. A method of avoiding an adverse drug interaction with Compound A in a human subject in need of Compound A, the method comprising administering to the subject a therapeutically effective amount of Compound A, and avoiding co-administration of a cytochrome p450 (CYP) 3A4 substrate, wherein said subject is also in need of the CYP3A4 substrate.

30. A method of avoiding an adverse drug interaction with Compound A in a human subject in need of Compound A, comprising discontinuing administration of a CYP3A4 substrate to avoid an adverse drug interaction with Compound A, and administering to the subject a therapeutically effective amount of Compound A.

31. A method of administering Compound A to a human subject with cancer, wherein said subject is also in need of a CYP3A4 substrate, comprising administering to the subject a total daily dose between 100 mg to 1400 mg of Compound A while avoiding CYP3A4 substrate co-administration, and any one or more of the following:
(a) advising the subject that the CYP3A4 substrate should be avoided or discontinued,
(b) advising the subject that co-administration of Compound A with the CYP3A4 substrate can alter the therapeutic effect or adverse reaction profile of Compound A,
(c) advising the subject that use of Compound A in subjects being treated with the CYP3A4 substrate is contraindicated, or
(d) advising the subject that CYP3A4 substrates should be used with caution in subjects receiving Compound A due to the potential for an adverse reaction profile.

32. A method of avoiding an adverse drug interaction with Compound A in a human subject in need of Compound A, the method comprising administering to the subject a therapeutically effective amount of Compound A, and avoiding co-administration of a cytochrome p450 (CYP) 3A4 inducer, wherein said subject is also in need of the CYP3A4 inducer.

33. A method of avoiding an adverse drug interaction with Compound A in a human subject in need of Compound A, comprising discontinuing administration of a CYP3A4 inducer to avoid an adverse drug interaction with Compound A, and administering to the subject a therapeutically effective amount of Compound A.

34. A method of administering Compound A to a human subject with cancer, wherein said subject is also in need of a CYP3A4 inducer, comprising administering to the subject a total daily dose between 100 mg to 1400 mg of Compound A while avoiding CYP3A4 inducer co-administration, and any one or more of the following:
(a) advising the subject that the CYP3A4 inducer should be avoided or discontinued,
(b) advising the subject that co-administration of Compound A with the CYP3A4 inducer can alter the therapeutic effect or adverse reaction profile of Compound A,
(c) advising the subject that use of Compound A in subjects being treated with the CYP3A4 inducer is contraindicated, or
(d) advising the subject that CYP3A4 inducers should be used with caution in subjects receiving Compound A due to the potential for an adverse reaction profile.

35. A method of avoiding an adverse drug interaction with Compound A in a human subject in need of Compound A, the method comprising administering to the subject a therapeutically effective amount of Compound A, and avoiding co-administration of a proton pump inhibitor (PPI), wherein said subject is also in need of the PPI.

36. A method of avoiding an adverse drug interaction with Compound A in a human subject in need of Compound A, comprising discontinuing administration of a PPI to avoid an adverse drug interaction with Compound A, and administering to the subject a therapeutically effective amount of Compound A.

37. A method of administering Compound A to a human subject with cancer, wherein said subject is also in need of a PPI, comprising administering to the subject a total daily dose between 100 mg to 1400 mg of Compound A while avoiding PPI co-administration, and any one or more of the following:
(a) advising the subject that the PPI should be avoided or discontinued,
(b) advising the subject that co-administration of Compound A with the PPI can alter the therapeutic effect or adverse reaction profile of Compound A,
(c) advising the subject that use of Compound A in subjects being treated with the PPI is contraindicated, or
(d) advising the subject that PPIs should be used with caution in subjects receiving Compound A due to the potential for an adverse reaction profile.

38. A method of avoiding an adverse drug interaction with Compound A in a human subject in need of Compound A, the method comprising administering to the subject a therapeutically effective amount of Compound A, and avoiding co-administration of a H-2 receptor antagonist (H2 blocker), wherein said subject is also in need of the H2 blocker.

39. A method of avoiding an adverse drug interaction with Compound A in a human subject in need of Compound A, comprising discontinuing administration of a H2 blocker to avoid an adverse drug interaction with Compound A, and administering to the subject a therapeutically effective amount of Compound A.

40. A method of administering Compound A to a human subject with cancer, wherein said subject is also in need of a H2 blocker, comprising administering to the subject a total daily dose between 100 mg to 1400 mg of Compound A while avoiding H2 blocker co-administration, and any one or more of the following:
(a) advising the subject that the H2 blocker should be avoided or discontinued,
(b) advising the subject that co-administration of Compound A with the H2 blocker can alter the therapeutic effect or adverse reaction profile of Compound A,
(c) advising the subject that use of Compound A in subjects being treated with the H2 blocker is contraindicated, or
(d) advising the subject that H2 blockers should be used with caution in subjects receiving Compound A due to the potential for an adverse reaction profile.

41. A method of avoiding an adverse drug interaction with Compound A in a human subject in need of Compound A, the method comprising administering to the subject a therapeutically effective amount of Compound A, and avoiding co-administration of a disease-modifying antirheumatic drug (DMARD), wherein said subject is also in need of the DMARD.

42. A method of avoiding an adverse drug interaction with Compound A in a human subject in need of Compound A, comprising discontinuing administration of a DMARD to avoid an adverse drug interaction with Compound A, and administering to the subject a therapeutically effective amount of Compound A.

43. A method of administering Compound A to a human subject with cancer, wherein said subject is also in need of a DMARD, comprising administering to the subject a total daily dose between 100 mg to 1400 mg of Compound A while avoiding DMARD co-administration, and any one or more of the following:
(a) advising the subject that the DMARD should be avoided or discontinued,
(b) advising the subject that co-administration of Compound A with the DMARD can alter the therapeutic effect or adverse reaction profile of Compound A,
(c) advising the subject that use of Compound A in subjects being treated with the DMARD is contraindicated, or
(d) advising the subject that DMARDs should be used with caution in subjects receiving Compound A due to the potential for an adverse reaction profile.

44. A method of avoiding an adverse drug interaction with Compound A in a human subject in need of Compound A, the method comprising administering to the subject a therapeutically effective amount of Compound A, and avoiding co-administration of an immune suppressive agent, wherein said subject is also in need of the immune suppressive agent.

45. A method of avoiding an adverse drug interaction with Compound A in a human subject in need of Compound A, comprising discontinuing administration of an immune suppressive agent to avoid an adverse drug interaction with Compound A, and administering to the subject a therapeutically effective amount of Compound A.

46. A method of administering Compound A to a human subject with cancer, wherein said subject is also in need of an immune suppressive agent, comprising administering to the subject a total daily dose between 100 mg to 1400 mg of Compound A while avoiding immune suppressive agent co-administration, and any one or more of the following:
(a) advising the subject that the immune suppressive agent should be avoided or discontinued,
(b) advising the subject that co-administration of Compound A with the immune suppressive agent can alter the therapeutic effect or adverse reaction profile of Compound A,
(c) advising the subject that use of Compound A in subjects being treated with the immune suppressive agent is contraindicated, or
(d) advising the subject that immune suppressive agents should be used with caution in subjects receiving Compound A due to the potential for an adverse reaction profile.

47. A method of avoiding an adverse drug interaction with Compound A in a human subject in need of Compound A, the method comprising administering to the subject a therapeutically effective amount of Compound A, and avoiding co-administration of a P-glycoprotein (P-gp) substrate, wherein said subject is also in need of the P-gp substrate.

48. A method of avoiding an adverse drug interaction with Compound A in a human subject in need of Compound A, comprising discontinuing administration of a P-gp substrate to avoid an adverse drug interaction with Compound A, and administering to the subject a therapeutically effective amount of Compound A.

49. A method of administering Compound A to a human subject with cancer, wherein said subject is also in need of a P-gp substrate, comprising administering to the subject a total daily dose between 100 mg to 1400 mg of Compound A while avoiding P-gp substrate co-administration, and any one or more of the following:
(a) advising the subject that the P-gp substrate should be avoided or discontinued,
(b) advising the subject that co-administration of Compound A with the P-gp substrate can alter the therapeutic effect or adverse reaction profile of Compound A,
(c) advising the subject that use of Compound A in subjects being treated with the P-gp substrate is contraindicated, or
(d) advising the subject that P-gp substrates should be used with caution in subjects receiving Compound A due to the potential for an adverse reaction profile.

50. A method of avoiding an adverse drug interaction with Compound A in a human subject in need of Compound A, the method comprising administering to the subject a therapeutically effective amount of Compound A, and avoiding co-administration of a P-glycoprotein (P-gp) inhibitor, wherein said subject is also in need of the P-gp inhibitor.

51. A method of avoiding an adverse drug interaction with Compound A in a human subject in need of Compound A, comprising discontinuing administration of a P-gp inhibitor to avoid an adverse drug interaction with Compound A, and administering to the subject a therapeutically effective amount of Compound A.

52. A method of administering Compound A to a human subject with cancer, wherein said subject is also in need of a P-gp inhibitor, comprising administering to the subject a total daily dose between 100 mg to 1400 mg of Compound A while avoiding P-gp inhibitor co-administration, and any one or more of the following:
(a) advising the subject that the P-gp inhibitor should be avoided or discontinued,
(b) advising the subject that co-administration of Compound A with the P-gp inhibitor can alter the therapeutic effect or adverse reaction profile of Compound A,
(c) advising the subject that use of Compound A in subjects being treated with the P-gp inhibitor is contraindicated, or
(d) advising the subject that P-gp inhibitors should be used with caution in subjects receiving Compound A due to the potential for an adverse reaction profile.

53. A method of avoiding an adverse drug interaction with Compound A in a human subject in need of Compound A, the method comprising administering to the subject a therapeutically effective amount of Compound A, and avoiding co-administration of an organic anion-transporting polypeptide 1B1 or 1B3 (OATP1B1 or OATP1B3) substrate, wherein said subject is also in need of the OATP1B1 or OATP1B3 substrate.

54. A method of avoiding an adverse drug interaction with Compound A in a human subject in need of Compound A, comprising discontinuing administration of an OATP1B1 or OATP1B3 substrate to avoid an adverse drug interaction with Compound A, and administering to the subject a therapeutically effective amount of Compound A.

55. A method of administering Compound A to a human subject with cancer, wherein said subject is also in need of a OATP1B1 or OATP1B3 substrate, comprising administering to the subject a total daily dose between 100 mg to 1400 mg of Compound A while avoiding OATP1B1 or OATP1B3 co-administration, and any one or more of the following:
(a) advising the subject that the OATP1B1 or OATP1B3 substrate should be avoided or discontinued,
(b) advising the subject that co-administration of Compound A with the OATP1B1 or OATP1B3 substrate can alter the therapeutic effect or adverse reaction profile of Compound A,
(c) advising the subject that use of Compound A in subjects being treated with the OATP1B1 or OATP1B3 substrate is contraindicated, or
(d) advising the subject that OATP1B1 or OATP1B3 substrates should be used with caution in subjects receiving Compound A due to the potential for an adverse reaction profile.

56. A method of avoiding an adverse drug interaction with Compound A in a human subject in need of Compound A, the method comprising administering to the subject a therapeutically effective amount of Compound A, and avoiding co-administration of a breast cancer resistance protein B3 (BCRP) substrate, wherein said subject is also in need of the BCRP substrate.

57. A method of avoiding an adverse drug interaction with Compound A in a human subject in need of Compound A, comprising discontinuing administration of a BCRP substrate to avoid an adverse drug interaction with Compound A, and administering to the subject a therapeutically effective amount of Compound A.

58. A method of administering Compound A to a human subject with cancer, wherein said subject is also in need of a BCRP substrate, comprising administering to the subject a total daily dose between 100 mg to 1400 mg of Compound A while avoiding BCRP substrate co-administration, and any one or more of the following:
(a) advising the subject that the BCRP substrate should be avoided or discontinued,
(b) advising the subject that co-administration of Compound A with the BCRP substrate can alter the therapeutic effect or adverse reaction profile of Compound A,
(c) advising the subject that use of Compound A in subjects being treated with the BCRP substrate is contraindicated, or
(d) advising the subject that BCRP substrates should be used with caution in subjects receiving Compound A due to the potential for an adverse reaction profile.

59. A method of avoiding an adverse drug interaction with Compound A in a human subject in need of Compound A, the method comprising administering to the subject a therapeutically effective amount of Compound A, and avoiding co-administration of a drug known to prolong QTc interval, wherein said subject is also in need of the drug known to prolong QTc interval.

60. A method of avoiding an adverse drug interaction with Compound A in a human subject in need of Compound A, comprising discontinuing administration of a drug known to prolong QTc interval to avoid an adverse drug interaction with Compound A, and administering to the subject a therapeutically effective amount of Compound A.

61. A method of administering Compound A to a human subject with cancer, wherein said subject is also in need of a drug known to prolong QTc interval, comprising administering to the subject a total daily dose between 100 mg to 1400 mg of Compound A while avoiding a drug known to prolong QTc interval co-administration, and any one or more of the following:
(a) advising the subject that the drug known to prolong QTc interval should be avoided or discontinued,
(b) advising the subject that co-administration of Compound A with the drug known to prolong QTc interval can alter the therapeutic effect or adverse reaction profile of Compound A,
(c) advising the subject that use of Compound A in subjects being treated with the drug known to prolong QTc interval is contraindicated, or
(d) advising the subject that drugs known to prolong QTc interval should be used with caution in subjects receiving Compound A due to the potential for an adverse reaction profile.

62. A method of treating a subject in need of a therapy comprising Compound A, the method comprising, administering Compound A at an initial dose and, upon the occurrence of an adverse event, administering a reduced dose of Compound A, wherein the dose-reduction sequence proceeds stepwise through one or more levels of about 1200 mg total daily dose, to about 900 mg total daily dose, to about 600 mg total daily dose, to about 300 mg total daily dose, as necessary to maintain tolerability while preserving clinical efficacy.
